(19) United States
(12) Patent Application Publication
(10) Pub. No.: US 2026/0132392 A1
(43) Pub. Date: May 14, 2026

(54) GENE EDITING SYSTEMS COMPRISING REVERSE TRANSCRIPTASES

(71) Applicant: Metagenomi, Inc., Emeryville, CA (US)

(72) Inventors: Brian C. Thomas, Berleley, CA (US); Lisa Alexander, Albany, CA (US); Ketaki Belsare, Emeryville, CA (US); Christopher Brown, Albany, CA (US); Cindy Castelle, San Francisco, CA (US); Daniela S.A. Goltsman, Oakland, CA (US); Sourab Kulkarni, Emeryville, CA (US); Sarah Laperriere, Berkeley, CA (US); Leanna Monteleone, Emeryville, CA (US); Maria Jose Soto Contreras, Berkeley, CA (US); Morayma Temoche-Diaz, Emeryville, CA (US); Anu Thomas, Albany, CA (US); Mary Kaitlyn Chui, San Francisco, CA (US); Rebecca Lamothe, Emeryville, CA (US)

(21) Appl. No.: 19/122,601

(22) PCT Filed: Oct. 18, 2023
(86) PCT No.: PCT/US2023/077228

§ 371 Date: Apr. 18, 2025

Related U.S. Application Data
(60) Provisional application No. 63/380,194, filed on Oct. 19, 2022.
(60) Provisional application No. 63/386,658, filed on Dec. 8, 2022.
(60) Provisional application No. 63/387,268, filed on Dec. 13, 2022.
(60) Provisional application No. 63/491,269, filed on Mar. 20, 2023.
(60) Provisional application No. 63/500,228, filed on May 4, 2023.
(60) Provisional application No. 63/500,509, filed on May 5, 2023.
(60) Provisional application No. 63/510,861, filed on Jun. 28, 2023.

(51) Int. Cl. C12N 9/22; C12N 1/16; C12N 5/04; C12N 5/07; C12N 9/12; C12N 15/10; C12N 15/113; C12N 15/62; C12N 15/85

(52) U.S. Cl. CPC C12N 9/226; C12N 1/16; C12N 5/04; C12N 5/0601; C12N 9/1276; C12N 15/10; C12N 15/113; C12N 15/62; C12N 15/85; C12Y 207/07049; C12N 2310/20; C12N 2510/04; C12N 2750/14143

(57) ABSTRACT

The disclosure relates generally to gene editing systems comprising reverse transcriptases and fusion proteins of reverse transcriptases with nickases or nucleases, methods of making such reverse transcriptases and fusion proteins, and methods of using such reverse transcriptases and fusion proteins for site directed genome editing in cells.

### CROSS-REFERENCE

This application claims the benefit of and priority to U.S. Provisional Patent Application No. 63/380,194 filed Oct. 19, 2022, U.S. Provisional Patent Application No. 63/386,658 filed Dec. 8, 2022, U.S. Provisional Patent Application No. 63/387,268 filed Dec. 13, 2022, U.S. Provisional Patent Application No. 63/491,269 filed Mar. 20, 2023, U.S. Provisional Patent Application No. 63/500,228 filed May 4, 2023, U.S. Provisional Patent Application No. 63/500,509 filed May 5, 2023, and U.S. Provisional Patent Application No. 63/510,861 filed Jun. 28, 2023, each of which is incorporated by reference in its entirety herein.

### BRIEF SUMMARY

The disclosure is based, in part, upon the development of a gene editing system comprising a reverse transcriptase, a nuclease or nickase, and a guide RNA or pegRNA.

Described herein are fusion proteins comprising a nickase linked to a reverse transcriptase using a linker, wherein the reverse transcriptase comprises at least about 80% sequence identity to any one of SEQ ID NOs: 161-629, 767-1220, 1959-2522, and 2582-2585.

Described herein are fusion proteins comprising a nuclease linked to a reverse transcriptase using a linker, wherein the reverse transcriptase comprises at least about 80% sequence identity to any one of SEQ ID NOs: 161-629, 767-1220, 1959-2522, and 2582-2585.

Described herein are fusion proteins comprising a catalytically dead nuclease linked to a reverse transcriptase using a linker, wherein the reverse transcriptase comprises at least about 80% sequence identity to any one of SEQ ID NOs: 161-629, 767-1220, 1959-2522, and 2582-2585.

Described herein are gene editing systems, comprising a) a nickase; b) a guide nucleic acid configured to form a complex with the nickase and to hybridize to a target nucleic acid sequence; and c) a reverse transcriptase having at least about 80% sequence identity to any one of SEQ ID NOs: 161-629, 767-1220, 1959-2522, and 2582-2585 and configured to form a complex with the nickase. In some embodiments, the gene editing system further comprises a nucleic acid template. In some embodiments, the nickase is a modified endonuclease. In some embodiments, the modified endonuclease is a Type II CRISPR endonuclease. In some embodiments, the modified endonuclease is a Type V CRISPR endonuclease. In some embodiments, the Type II CRISPR endonuclease or the Type V CRISPR endonuclease has nickase activity. In some embodiments, the modified endonuclease is selected from the group consisting of spCas9 (H840A), spCas9 (D10A), nMG3-6 (D13A), nMG3-6 (H586A), nMG3-6 (N609A), Cas12a, and MG29-1. In some embodiments, the modified endonuclease comprises at least about 80% sequence identity to any one of SEQ ID NOs: 152-154. In some embodiments, the nickase and the reverse transcriptase are linked. In some embodiments, the nickase and the reverse transcriptase are linked by a linker. In some embodiments, the linker comprises at least 10, 20, or 30 amino acids. In some embodiments, the linker comprises about 30-35 amino acids. In some embodiments, the linker comprises about 30 amino acids. In some embodiments, the linker comprises at least 80% sequence identity to SEQ ID NO: 103. In some embodiments, the linker comprises at least 80% sequence identity to any one of SEQ ID NOs: 155-160. In some embodiments, the nickase and the reverse transcriptase are not linked. In some embodiments, the guide nucleic acid comprises a spacer sequence and a crRNA. In some embodiments, the guide nucleic acid further comprises a reverse transcriptase template (RTT). In some embodiments, a base in the RTT comprises a bulky modification selected from the group of complex sugars, or complex amino groups, and/or other modifications compatible with RNA. In some embodiments, the guide nucleic acid further comprises a primer binding site. In some embodiments, the primer binding site is on a 3′ end of the guide nucleic acid. In some embodiments, the primer binding site comprises at least 2, 4, 6, 8, 10, 13, 16, 20, 24, 28, 32, 36, 40, 45, 50, 55, 60, or 65 nucleotides. In some embodiments, the gene editing system further comprises a transposase, integrase, or homing endonuclease. In some embodiments, the gene editing system further comprises a retrotransposon. In some embodiments, the reverse transcriptase comprises a processivity of at least about 2-fold more than Moloney Murine Leukemia Virus (MMLV) reverse transcriptase. In some embodiments, the reverse transcriptase comprises a processivity of at least about 2-fold less than Moloney Murine Leukemia Virus (MMLV) reverse transcriptase. In some embodiments, the reverse transcriptase comprises an error rate of less than about 2.5%, 2.0%, 1.5%, 1%, 0.5%, 0.25%, 0.10%, or 0.05%. In some embodiments, the reverse transcriptase comprises an error rate of less than about 2.5%, 2.0%, 1.5%, 1%, 0.5%, 0.25%, 0.10%, or 0.05% as compared to Moloney Murine Leukemia Virus (MMLV) reverse transcriptase.

Described herein are gene editing systems, comprising a) a nuclease; b) a guide nucleic acid configured to form a complex with the nuclease and to hybridize to a target nucleic acid sequence; and c) a reverse transcriptase having at least about 80% sequence identity to any one of SEQ ID NOs: 161-629, 767-1220, 1959-2522, and 2582-2585 and configured to form a complex with the nuclease. In some embodiments, the gene editing system further comprises a nucleic acid template. In some embodiments, the nuclease is a double strand nuclease. In some embodiments, the nuclease is a Type II CRISPR endonuclease. In some embodiments, the CRISPR endonuclease is Cas9. In some embodiments, the Cas9 is catalytically dead Cas9 (dCas9). In some embodiments, the nuclease and the reverse transcriptase are linked. In some embodiments, the nuclease and the reverse transcriptase are linked by a linker. In some embodiments, the linker comprises at least 10, 20, or 30 amino acids. In some embodiments, the linker comprises about 30-35 amino acids. In some embodiments, the linker comprises about 30 amino acids. In some embodiments, the linker comprises at least 80% sequence identity to SEQ ID NO: 103. In some embodiments, the linker comprises at least 80% sequence identity to any one of SEQ ID NOs: 155-160. In some embodiments, the nuclease and the reverse transcriptase are not linked. In some embodiments, the guide nucleic acid further comprises a primer binding site. In some embodiments, the primer binding site is on a 3′ end of the guide nucleic acid. In some embodiments, the primer binding site comprises at least 2, 4, 6, 8, 10, 13, 16, 20, 24, 28, 32, 36, 40, 45, 50, 55, 60, or 65 nucleotides. In some embodiments, the gene editing system further comprises a transposase, integrase, or homing endonuclease. In some embodiments, the gene editing system further comprises a retrotransposon. In some embodiments, the reverse transcriptase comprises a processivity of at least about 2-fold more than Moloney Murine Leukemia Virus (MMLV) reverse transcriptase. In some embodiments, the reverse transcriptase comprises a processivity of at least about 2-fold less than Moloney Murine Leukemia Virus (MMLV) reverse transcriptase. In some embodiments, the reverse transcriptase comprises an error rate of less than about 2.5%, 2.0%, 1.5%, 1%, 0.5%, 0.25%, 0.10%, or 0.05%. In some embodiments, the reverse transcriptase comprises an error rate of less than about 2.5%, 2.0%, 1.5%, 1%, 0.5%, 0.25%, 0.10%, or 0.05% as compared to Moloney Murine Leukemia Virus (MMLV) reverse transcriptase.

Described herein are gene editing systems, comprising a) a nickase, b) a guide nucleic acid configured to form a complex with the nickase and to hybridize to a target nucleic acid sequence; and c) a reverse transcriptase configured to form a complex with the nickase, the reverse transcriptase having a X₁X₂DD motif, wherein X₁ is F or Y, and wherein when X₁ is Y, X₂ is A, R, N, D, C, E, Q, G, H, I, L, K, M, F, P, S, T, V, W, or Y. In some embodiments, the X₂ is A or I. In some embodiments, the X₁X₂DD motif is YADD (SEQ ID NO: 2572) or YIDD (SEQ ID NO: 2573). In some embodiments, the X₁X₂DD motif is FADD (SEQ ID NO: 2574), FVDD (SEQ ID NO: 2575), FIDD (SEQ ID NO: 2576), or FLDD (SEQ ID NO: 2577). In some embodiments, the reverse transcriptase has at least about 80% sequence identity to any one of SEQ ID NOs: 161-629, 767-1220, 1959-2522 and 2582-2585.

Described herein are gene editing systems, comprising a) a nuclease; b) a guide nucleic acid configured to form a complex with the nuclease and to hybridize to a target nucleic acid sequence; and c) a reverse transcriptase configured to form a complex with the nuclease, the reverse transcriptase having a X₁X₂DD motif, wherein X₁ is F or Y, and wherein when X₁ is Y, X₂ is A, R, N, D, C, E, Q, G, H, I, L, K, M, F, P, S, T, V, W, or Y. In some embodiments, the X₂ is A or I. In some embodiments, the X₁X₂DD motif is YADD (SEQ ID NO: 2572) or YIDD (SEQ ID NO: 2573). In some embodiments, the X₁X₂DD motif is FADD (SEQ ID NO: 2574), FVDD (SEQ ID NO: 2575), FIDD (SEQ ID NO: 2576), or FLDD (SEQ ID NO: 2577). In some embodiments, the reverse transcriptase has at least about 80% sequence identity to any one of SEQ ID NOs: 161-629, 767-1220, 1959-2522 and 2582-2585.

Described herein are isolated reverse transcriptases having at least about 80% sequence identity to any one of SEQ ID NOs: 161-629, 767-1220, 1959-2522, and 2582-2585.

Described herein are nucleic acids encoding for a fusion protein or a gene editing system as described above. In some embodiments, the nucleic acid is a DNA or an RNA. In some embodiments, the RNA is an mRNA. In some embodiments, nucleic acid is comprised in a vector. In some embodiments, the nucleic acid or the vector comprising the nucleic acid is comprised in an adeno-associated virus or a lipid nanoparticle. In some embodiments, the nucleic acid or the vector comprising the nucleic acid is comprised in a cell. In some embodiments, the cell is a human cell.

Described herein are methods for modifying a double- and/or single-stranded nucleic acid, comprising contacting a cell using a fusion protein or a gene editing system as described above.

Described herein are methods for modifying a double- and/or single-stranded nucleic acid in a cell comprising a) providing a cell with a guide nucleic acid to bind to a target strand of the nucleic acid; b) providing the cell with a nuclease or nickase to cleave the nucleic acid at a location of binding of the guide nucleic acid; c) providing the cell with a reverse transcriptase to synthesize a modification in the target strand of the nucleic acid at a location of cleavage by the nickase and/or nuclease. In some embodiments, the reverse transcriptase has at least about 80% sequence identity to any one of SEQ ID NOs: 161-629, 767-1220, 1959-2522, and 2582-2585. In some embodiments, the modification is an insertion, deletion, or mutation. In some embodiments, the method further comprises providing an RNA or DNA template to the cell. In some embodiments, the nucleic acid is a genome or a vector. In some embodiments, the method further comprises providing the cell with a transposase, integrase, or homing endonuclease. In some embodiments, the method further comprises providing the cell with a retrotransposon.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1JJ are bar graphs showing the G-to-T conversion editing percentage of untethered reverse transcriptase (RT) candidates from the MG151 family with eight different primer binding site (PBS) nucleotides of varying length (PBS lengths of 2, 4, 6, 8, 10, 13, 16, and 20 nucleotides) in HEK293T cells. MG151 candidates 80-85 (FIGS. 1A-IF), 87-100 (FIGS. 1G-1T), and 102-117 (FIGS. 1U-1JJ) are shown with untreated samples, no RT, wild-type MMLV1, and wild-type MMLV2 as a control.

FIG. 2 is a bar graph showing the relative fold change of editing by untethered RT candidates from the MG151 family compared to wild-type MMLV editing normalized to 1. Seven untethered MG151 candidates (candidates 98, 100, 99, 102, 103, 104, and 105) with eight different PBS nucleotides of varying length (PBS lengths of 2, 4, 6, 8, 10, 13, 16, and 20 nucleotides) are shown. The bars represent a specific PBS length tested for each candidate.

FIGS. 3A-3W are bar graphs showing the G-to-T conversion editing percentage of untethered reverse transcriptase (RT) candidates from the MG153 family with eight different PBS nucleotides of varying length (PBS lengths of 2, 4, 6, 8, 10, 13, 16, and 20 nucleotides) in HEK293T cells. MG153 candidates 1-5, 7-13, 15, 16, and 21 (FIGS. 3A-30) and 14, 17-20, and 25-27 (FIGS. 3P-3W) are shown with untreated samples and wild-type MMLV1 as a control.

FIGS. 4A-4G are bar graphs showing the G-to-T conversion editing percentage of untethered reverse transcriptase (RT) candidates from the MG160 family with eight different PBS nucleotides of varying length (PBS lengths of 2, 4, 6, 8, 10, 13, 16, and 20 nucleotides) in HEK293T cells. MG160 candidates 1-6 and 8 (FIGS. 4A-4G) are shown with untreated samples and wild-type MMLV1 as a control.

FIGS. 5A-5G are bar graphs showing the G-to-T conversion editing percentage of RT candidates from the MG160 family tethered to spCas9(H840A). MG160 candidates 1-5 (FIGS. 5A-5G) tethered to spCas9(H840A) were tested in HEK293T cells for G-to-T conversion. The candidates are shown with untreated samples, wild-type MMLV1, wild-type MMLV2, spCas9(H840A)-MMLV1, and spCas9(H840A)-MMLV2 as controls.

FIGS. 6A-6D are bar graphs showing a blot of percent InDels after targeting the endogenous targets AAVS1 (FIG. 6A), B2M (FIG. 6B), CD5 (FIG. 6C), and CD38 (FIG. 6D) with the nuclease MG3-6 bound to pegRNA comprising PBS of different lengths (PBS lengths of 2, 4, 6, 8, 10, 13, 16, and 20 nucleotides) in HEK293T cells.

FIG. 7 depicts a schematic of an exemplary DNA construct for a GFP-based retrotransposition assay. The construct carries a cytomegalovirus promoter (CMVp) followed by the reverse transcriptase (RT-NLS) with an N-terminal tag (Flag-HA-NLS-MCP-linker). An EF1 alpha promoter (EF1α) in the reverse orientation drives the expression of GFP (GFPexon2 and GFPexon1) only following the successful retrotransposition of the construct into the target site specified by a nuclease (inverted intron). Target-primed reverse transcription is initiated following the binding of the primer binding site (PBS) with a 3′ overhang generated by the nuclease. NLS=Nuclear Localization Signal; MCP=MS2 coat protein; GFP=green fluorescent protein; pA=polyA sequence, MS2 loops=site for MS2 coat protein binding.

FIG. 8 depicts a diagram of a mechanism for targeted integration of retron-derived ssDNA by TnpA. The retron ncRNA (msr in grey and msd in black) contains the desired cargo flanked by structural motifs recognized by TnpA (top left, dashed box). The excised cargo (top right) is circularized by TnpA and finds the targeting motif on a ssDNA target, which is made available by binding of an RNA-guided effector (bottom right, grey). TnpA mediates integration of the ssDNA donor by cleavage of the target and the host repair machinery repairs the integrated edit (bottom left, dashed box).

FIGS. 9A-9R depict editing with untethered MG151 candidates MG151-118 through MG151-135 for G-to-T conversion across 8 different PBS lengths.

FIGS. 10A-10D depict editing with untethered MG151 candidates MG151-123 through MG151-126 for G-to-T conversion at PBS lengths 6, 8, 10, 13 nucleotides. Two biological replicates were performed for each candidate.

FIGS. 11A-11D depict editing with untethered MG151 family mutants for G-to-T conversion. FIG. 11A: MG151-98 wild type is shown in green bar alongside point mutations of MG151-98, combined mutations of MG151-98, and trimmed mutants of MG151-98. Single replicate is shown in FIG. 11A and additional replicate with various MG151-98 mutations are found in FIG. 11B. Mutations K297P and H171N significantly improve wild type MG151-98 activity. FIG. 11C: MG151-99 mutants and wild type MG151-99 have G-to-T conversion with some mutations increasing wild type activity. FIG. 11D: MG151-99 wild type is compared to trimmed versions of MG151-99. MG151-99 trimmed 152 AA significantly improves activity of G-to-T conversion, whereas trimming 136 AA inhibited editing activity. MMLV1 wildtype is shown in gold bars and MMLV2 (pentamutant) acts as controls for each experiment.

FIGS. 12A-12B depict untethered MG151 candidates (MG151-80 through MG151-135) tested for G-to-T conversion. Percent editing of G-to-T conversion (FIG. 12A) and fold change relative to MMLV wild type at PBS 13 (FIG. 12B). Each dot represents a different PBS length ranging from 2, 4, 6, 8, 10, 13, 16, and 20 nucleotides.

FIGS. 13A-13H depict editing with untethered MG153 candidates tested for G-to-T conversion across 8 different PBS lengths for different MG153 candidates. FIG. 13H shows MG153-53 editing when fused to Cas9.

FIGS. 14A-14B depict untethered MG153 candidates tested for G-to-T conversion. Percent editing of G-to-T conversion (FIG. 14A) and fold change to MMLV wild type at PBS 13 (FIG. 14A). Each dot represents a different PBS length ranging from 2, 4, 6, 8, 10, 13, 16, and 20 nucleotides.

FIGS. 15A-15U depict editing with spCas9(H840A) tethered to MG160 candidates for G-to-T conversion across 8 different PBS lengths.

FIGS. 16A-16B depict MG160 candidates tethered to spCas9(H840A) tested for G-to-T conversion. Percent editing of G-to-T conversion (FIG. 16A) and fold change to MMLV wild type at PBS 13 (FIG. 16B). Each dot represents a different PBS length ranging from 2, 4, 6, 8, 10, 13, 16, and 20 nucleotides.

FIGS. 17A-17D depict untethered candidates MG151-98 and MG151-99. RT candidates MG151-98 (FIG. 17A) and MG151-99 (FIG. 17B) were tested for performing 24 nt insertion. RT candidates MG151-98 (FIG. 17C) and MG151-99 (FIG. 17D) were tested for performing 15 nt deletion.

FIGS. 18A-18H depict MG151 candidates including MG151-123 (FIGS. 18A and 18E), MG151-124 (FIGS. 18B and 18F), MG151-125 (FIGS. 18C and 18G), and MG151-126 (FIGS. 18D and 18H) completing 24 nt insertion (FIGS. 18A-18D) and 15 nt deletion (FIGS. 18E-18H) over four PBS lengths.

FIGS. 19A-19D depict rational engineering of MG151-98. MG151-98 wild type is shown in the green bar alongside point mutations, combined mutations, and trimming of MG151-98. Performance of these mutations for 24 nt insertion (FIGS. 19A-19B), and 15 nt deletion (FIGS. 19C-19D) are represented above alongside controls MMLV1 and MMLV2.

FIGS. 20A-20D depict rational engineering of MG151-99. MG151-99 wild type shown in the green bar alongside point mutations, combined mutations, and trimming of MG151-99. Performance of these mutations for 24 nt insertion (FIGS. 20A-20B), and 15 nt deletion (FIGS. 20C-20D) are represented above alongside controls MMLLV1 and MMLV2.

FIGS. 21A-21H depict MG153 candidates tested for 24 nt flag insertion across 4 to 8 different PBS lengths.

FIGS. 22A-22H depict MG153 candidates tested for 15 nt deletion across 4 to 8 different PBS lengths.

FIGS. 23A-23H depict the editing efficiency of spCas9(H840A) tethered to MG160 candidates for 24 nt insertion at 4 to 8 different PBS lengths.

FIGS. 24A-24H depict the editing efficiency of spCas9(H840A) tethered to MG160 candidates for 15 nt insertion at 4 to 8 different PBS lengths.

FIGS. 25A-25D depict G-T transversion using RTs in combination with MG nickase MG3-6. Untethered (FIGS. 25A-25B) and tethered (FIGS. 25C-25D) systems were tested. RTs tested include MG151-98, MG151-24, MG153-53, MG160-4 and MG151-99.

FIGS. 26A-26C depict a screen of the ability of indicated control RTs and RT candidates to retrotranspose an RNA cargo containing GFP in mammalian cells at a target specified by Cas9. Successful retrotransposition was detected by measuring the percentage of GFP positive cells by flow cytometry in the indicated samples at 3, 6, and 8 days post-transfection of cells with RT-containing plasmid, Cas9-containing plasmid, and chemically synthesized guide RNAs. LINE-WT (WT LINE-1 RT), LINE-dead (D702Y LINE-1 RT, RT dead), NT (non-targeting guide), VEGFA (VEGFA targeting guide).

FIGS. 27A-27C depict the prime editing ability of the engineered RT's. FIG. 27A depicts prime editing percentage (y-axis) of MG160-4 RT across different PBS lengths (x-axis).

FIG. 27B depicts prime editing percentage (y-axis) of MG151-98 across different PBS lengths (x-axis). FIG. 27C depicts prime editing percentage (y-axis) of MG153-3RT across different PBS lengths (x-axis).

FIG. 28 depicts RT candidates' ability to efficiently generate full-length cDNA from large RNA templates in mammalian cells.

FIGS. 29A-29DD depict editing percentage of MG160 family candidates tethered to spCas9(H840A). Candidates from the MG160 family were tethered to spCas9(H840A) and were transfected in HEK293T cells to determine G-to-T editing on the VEGFA target. Chemically synthesized guides ranged from having primer binding site lengths from 2-20 nucleotides. Candidates MG160-473 (FIG. 29A), MG160-283(FIG. 29G), MG160-379 (FIG. 29L), MG160-395 (FIG. 29O), MG160-9 (FIG. 29P), and MG160-107 (FIG. 29CC) had comparable or better G-to-T editing levels (across multiple PBS lengths) to spCas9(H840A) tethered to MMLV WT. spCas9-PE1 and spCas9-PE2 were transfected alongside chemically synthesized pegRNA with PBS length of 13 nucleotides.

FIGS. 30A-30C depict editing percentage of G-to-T transversion, insertion, and deletion of selected MG160 candidates. With MG160 candidates tethered to spCas9(H840A), RTs were challenged to incorporate G-to-T transversion (FIG. 30A), 24 nucleotide insertion (FIG. 30B), and 15 nucleotide deletion (FIG. 30C) into the VEGFA target. MG160-107, MG160-473, MG160-283, MG160-379, and MG160-395 showed comparable or improved editing levels to spCas9(H840A) tethered to MMLV WT for all types of edits at various PBS lengths. MG160-473 showed comparable editing levels to spCas9(H840A) tethered to MMLV2 (hyperactive mutant). spCas9-PE1 and spCas9-PE2 were transfected alongside chemically synthesized pegRNA with PBS length of 13 nucleotides.

FIGS. 31A-31K depict editing percentage of unique reverse transcriptase candidates from MG retron families untethered with spCas9(H840A). Candidates from various MG retron families were transfected in an untethered format alongside nickase spCas9(H840A) into HEK293T cells to determine G-to-T editing on the VEGFA target. Chemically synthesized guides ranged from having primer binding site lengths from 2-20 nucleotides. Candidates MG173-1 (FIG. 31J) and MG173-2 (FIG. 31K) were active and showed above background levels of G-to-T editing across multiple PBS lengths. Controls MMLV1 and MMLV2 were untethered and transfected alongside spCas9(H840A) and chemically synthesized pegRNA with PBS length of 13 nucleotides.

FIGS. 32A-32D depict editing percentage of reverse transcriptase candidates from MG Group II intron families untethered with spCas9(H840A). Candidates from various MG group II intron families were transfected in an untethered format alongside nickase spCas9(H840A) into HEK293T cells to determine G-to-T editing on the VEGFA target. Chemically synthesized guides ranged from having primer binding site lengths from 2-20 nucleotides. Candidate MG169-1 (FIG. 32D) was slightly above background editing levels of G-to-T editing across multiple PBS lengths. Other MG candidates, MG164-5 (FIG. 32A), MG166-2 (FIG. 32B), and MG167-4 (FIG. 32C) did not show editing levels above background. Controls MMLV1 and MMLV2 were untethered and transfected alongside spCas9(H840A) and chemically synthesized pegRNA with PBS length of 13 nucleotides.

FIG. 33A-33D depict editing percentage of WT MG160-4 and engineered mutants tethered to spCas9(H840A). FIG. 33A shows editing percentage for seventeen engineered MG160-4 constructs tethered to spCas9(H840A) that were tested in HEK293T cells for G-to-T transversion on the VEGFA target. Chemically synthesized guides ranging from PBS lengths of 6 to 13 nucleotides were used to test conversion. Point mutations H230K and H230R showed a neutral change in G-to-T editing activity, whereas combining multiple mutations drastically reduced editing efficiency. FIG. 33B shows G-to-T conversion with selected point mutations, which show similar editing levels to WT MG160-4. MG160-4 (H230K) and MG160-4(H230R) were then tested for 24 nucleotide insertion (FIG. 33C) and 15 nucleotide deletion (FIG. 33D). MG160-4 (H230R) showed editing levels slightly better than MG160-4 WT and MG160-4 (H230K) at various desired edits. spCas9-PE1 and spCas9-PE2 were transfected alongside chemically synthesized pegRNA with PBS length of 13 nucleotides.

FIG. 34 depicts editing percentage of WT MG153-53 and engineered mutants. Six engineered MG153-53 constructs untethered and transfected alongside spCas9(H840A) were tested in HEK293T cells for G-to-T transversion on the VEGFA target. Chemically synthesized guides ranging from PBS lengths of 6 to 13 nucleotides were used to test conversion. Point mutations V200R showed an increase in G-to-T editing activity comparable to WT MG153-53, whereas combining multiple mutations drastically reduced editing efficiency. MG153-53 WT and engineered constructs had comparable or higher level editing than untethered controls TGIRT, marathon, and marathon mutant, but where drastically lower than untethered MMLV WT (MMLV1) and MMLV hyperactive mutant (MMLV2).

FIG. 35 depicts editing percentage of MG3-6(H586A) with selected RT candidates. MG3-6(H586A) nickase was combined with selected reverse transcriptases to make a desired correction in the AAVS1 target. Reverse transcriptases were either untethered (UT) and transfected alongside MG3-6(H586A) or tethered to MG3-6(H586A) either on the C terminus of the nickase I or the N terminus of the nickase (N). The pegRNA varied in PBS lengths from 8, 10, 13, and 20 nucleotides. Background editing was shown at less than 0.1% editing. All selected RTs showed above background editing with MG3-6(H586A) except for MG153-53. MG160-4 had improved activity when tethered to MG3-6(H586A) compared to an untethered condition. MG151-98 engineered candidates had slight preference for either being untethered or tethered to the N-terminus of MG3-6(H586A). WT MMLV (MMLV1) and hyperactive mutant MMLV (MMLV2) had highest editing levels when tethered to the C-terminus of MG3-6(H586A). Each data point represents a single biological replicate at different PBS lengths for each selected RT.

FIGS. 36A-36J depict editing percentage of untethered MG71-2(H883A) with selected RT candidates on AAVS1 target. FIG. 36A shows biological triplicate data for selected RT candidates performing a five nucleotide change on the AAVS1 target with untethered MG71-2n and chemically synthesized pegRNAs with PBS lengths 4, 6, 8, 10, 13, and 16 nucleotides. Select RT candidates were then tested for five nucleotide change (FIG. 36B), five nucleotide change with a modified scaffold in pegRNA (FIG. 36C), G-to-T transversion (FIG. 36D), 24 nucleotide insertion (FIG. 36E), and 15 nucleotide deletion (FIG. 36F). Additional engineered MG151-98 candidates, MG151-98 (166AA), MG151-98 (166AA, H171N), and MG151-98 (166AA, K297P), were tested for G-to-T change (FIG. 36G), 15 nucleotide deletion (FIG. 36H), 24 nucleotide insertion (FIG. 36I), and 5 nucleotide change (FIG. 36J) across PBS lengths 4, 6, 8, 10, 13, and 16 nucleotides. Above background levels of editing were defined as greater than 0.1%. Each graph represents a single biological replicate except for FIG. 36A which represents biological triplicate data.

FIGS. 37A-37C depict editing percentage of G-to-T transversion, insertion, and deletion of engineered MG151-98 mutants. With engineered MG151-98 candidates untethered to spCas9(H840A), RTs were challenged to incorporate G-to-T transversion (FIG. 37A), 24 nucleotide insertion (FIG. 37B), and 15 nucleotide deletion (FIG. 37C) into the VEGFA target. MG151-98 (Δ166AA) enhanced editing levels for most PBS lengths at all conditions. Specifically, combining trimmed MG151-98 constructs with point mutations H171N or K297P, editing levels further increased to achieve levels comparable or better that MMLV WT. spCas9(H840A) untethered with MMLV WT and MMLV2 were transfected alongside chemically synthesized pegRNA with PBS length of 13 nucleotides.

FIG. 38 depicts an overview of the mechanism to achieve programmable genome editing with Cas9, retron reverse transcriptase, and ssDNA transposase TnpA.

FIGS. 39A, 39B, and 39C depict an overview of the design principles used to generate engineered ncRNAs of Ec96. FIG. 39A depicts an overview of the 3 insertion sequences of 3 different lengths flanked by the LE/RE recognition motifs of Hp TnpA. FIG. 39B depicts a figure from the designated paper (Wang et al., Nature Microbiology (2022)) indicating the region of the msdDNA unresolved in the cryo-EM structure of Ec86 in complex with its product. FIG. 39C depicts the three different replaceable regions of the msd stem loop identified for Ec86 ncRNA.

FIG. 40 depicts the predicted secondary structures of engineered Ec86 ncRNAs which contain insertion of a 200nt or 500nt partial kanamycin gene flanked by the reverse complement (rc) LE/RE motifs of Hp TnpA. Motifs required for priming of reverse transcription, the msr and inverted repeats (IRs), are highlighted.

FIG. 41 depicts quantification of msdDNA production by qPCR in reactions that do or do not contain the Ec86 reverse transcriptase. WT is the wild-type ncRNA. LE40RE_v1 through v3, LE200RE_v1 and v3, and LE500RE v1 through v3 are engineered ncRNA designs.

FIG. 42 depicts confirmation of insertion by PCR of chimeric product generated by TnpA/retron system. PCR product indicated with arrow. Lane numbers correspond to the following: lane 1: LE200RE_v1 ncRNA, +RT, +TnpA; lane 2: LE200RE_v1 ncRNA, −RT, +TnpA; lane 3: LE200RE_v3 ncRNA, +RT, +TnpA; lane 4: LE200RE_v3 ncRNA, −RT, +TnpA; lane 5: LE500RE_v1 ncRNA, +RT, +TnpA; lane 6: LE500RE_v1 ncRNA, −RT, +TnpA; lane 7: LE500RE_v2 ncRNA, +RT, +TnpA; lane 8: LE500RE_v2 ncRNA, −RT, +TnpA; lane 9: LE500RE_v3 ncRNA, +RT, +TnpA; lane 10: LE500RE_v3 ncRNA, −RT, +TnpA; lane 11: LE200RE_v1 ncRNA, +RT, −TnpA; lane 12: LE200RE_v1 ncRNA, −RT, −TnpA; lane 13: LE200RE_v3 ncRNA, +RT, −TnpA; lane 14: LE200RE_v3 ncRNA, −RT, −TnpA; lane 15: LE500RE_v1 ncRNA, +RT, −TnpA; lane 16: LE500RE_v1 ncRNA, −RT, −TnpA; lane 17: LE500RE_v2 ncRNA, +RT, −TnpA; lane 18: LE500RE_v2 ncRNA, −RT, −TnpA; lane 19: LE500RE_v3 ncRNA, +RT, −TnpA; lane 20: LE500RE_v3 ncRNA, −RT, −TnpA.

FIG. 43 depicts Sanger sequencing results of the inserted ssDNA product made by TnpA, where the substrate for TnpA is generated by Ec86 retron. The highlighted region of the Sanger sequencing chromatogram shows the junction of the chimeric product where the 5′ sequence corresponds to the right end (RE) motif of Hp TnpA which is integrated along with the cargo and the 3′ sequence corresponds to the ssDNA target provided in the reaction mixture. FIG. 43 discloses SEQ ID NOs: 2578 and 2578, respectively, in order of appearance.

FIG. 44 depicts a method to confirm ncRNA prediction and msd insertion tolerance of retrons.

FIG. 45 depicts secondary structure predictions of retron ncRNAs from the MG154 family, highlighting the 5′ and 3′ inverted repeat elements (IRs) and msr required for priming of reverse transcription, along with the msd stem loop. Region of the msd stem loop that was replaced with an engineered sequence is indicated.

FIG. 46 depicts secondary structure predictions of retron ncRNAs from the MG155 family, highlighting the 5′ and 3′ inverted repeat elements (IRs) and msr required for priming of reverse transcription, along with the msd stem loop. Region of the msd stem loop that was replaced with an engineered sequence is indicated.

FIG. 47 depicts secondary structure predictions of retron ncRNAs from the MG156 family, highlighting the 5′ and 3′ inverted repeat elements (IRs) and msr required for priming of reverse transcription, along with the msd stem loop. Region of the msd stem loop that was replaced with an engineered sequence is indicated.

FIG. 48 depicts secondary structure predictions of retron ncRNAs from the MG157 family, highlighting the 5′ and 3′ inverted repeat elements (IRs) and msr required for priming of reverse transcription, along with the msd stem loop. Region of the msd stem loop that was replaced with an engineered sequence is indicated.

FIG. 49 depicts secondary structure predictions of retron ncRNAs from the MG158 family, highlighting the 5′ and 3′ inverted repeat elements (IRs) and msr required for priming of reverse transcription, along with the msd stem loop. Region of the msd stem loop that was replaced with an engineered sequence is indicated.

FIG. 50 depicts secondary structure predictions of retron ncRNAs from the MG159 family, highlighting the 5′ and 3′ inverted repeat elements (IRs) and msr required for priming of reverse transcription, along with the msd stem loop. Region of the msd stem loop that was replaced with an engineered sequence is indicated.

FIG. 51 depicts secondary structure predictions of retron ncRNAs from the MG173 family, highlighting the 5′ and 3′ inverted repeat elements (IRs) and msr required for priming of reverse transcription, along with the msd stem loop. Region of the msd stem loop that was replaced with an engineered sequence is indicated.

FIG. 52 depicts the detection of msdDNA production by qPCR. Ec86 is a positive control retron RT, and the corresponding ncRNA tested contained the ˜200nt insertion sequence at the replaceable position version 1 described previously. The ncRNAs for which activity was identified using the corresponding retron RT are colored in black (msdDNA production>10× above the no RT control). The ncRNAs for which activity was not identified using the corresponding retron RT are colored in light grey.

FIGS. 53A-53D depict editing percentage of a 5nt change on AAVS1 target using MG RTs and MG71-2(H883A). RTs were tested either in an untethered or tethered format (RT on C-term of MG71-2(H883A) indicated by nickase-RT and RT on N-term of MG71-2(H883A) indicated by RT-nickase). FIG. 53A: MMLV2-RT was tested untethered and tethered to MG71-2(H883A) with the highest levels of editing for untethered at PBS 13, nickase-RT PBS 16, and RT-nickase PBS 13. FIG. 53B: Engineered MG151-98 (K297P, Δ166AA) was tested untethered and tethered to MG71-2(H883A) with the highest levels of editing for untethered, nickase-RT, and RT-nickase at PBS 13 with the highest level of editing seen in the RT-nickase configuration. FIG. 53C: MG160-4(H1230R) was only tested in a tethered format with the highest levels of editing for nickase-RT at PBS 10 and RT-nickase at PBS 13. The highest level of editing was seen for the RT-nickase configuration. FIG. 53D: MG160-473 was tested in a tethered format with the highest level of editing for the RT-nickase configuration at PBS 13. The nickase-RT configuration for MG160-473 had low read count through NGS processing and percent editing was not determined. Correct edit indicates the intended correction with no errors found in the NGS amplicon. Incorrect edit refers to the intended edit being incorporated but also includes errors within the NGS amplicon and scaffold incorporation of the pegRNA.

FIGS. 54A-54S depict the editing percentage for G-to-T transversion of MG retron family candidates untethered to spCas9(H840A). FIG. 54A depicts a summary of untethered MG retron candidates from MG173 family and MG192 family percent editing for G-to-T transversion across eight different PBS lengths of 2, 4, 6, 8, 10, 13, 16, and 20 nucleotides. The MG173-8 candidate showed the highest levels of editing compared to the nine other retron candidates. Editing levels represented in FIGS. 54B-54J labeled “correct edit” represent the intended edit with no mistakes in the NGS amplicon, and bars labeled “incorrect edit” refer to the intended edit being incorporated and includes errors within the NGS amplicon, either originating in misincorporation by the RT and/or scaffold incorporation of the pegRNA. Editing levels represented in FIGS. 54K-54S show editing levels across eight different PBS lengths wherein bars labeled “editing” represent intended edit with errors in the NGS amplicon (excluding pegRNA scaffold incorporation) and bars labeled “scaffold incorporation” represent the intended edit and scaffold incorporation of the pegRNA.

FIGS. 55A-55AS depict the editing percentage for G-to-T transversion of MG160 family candidates tethered to spCas9(H840A). FIG. 55A depicts a summary of tethered MG160 candidates percent editing for G-to-T transversion across eight different PBS lengths of 2, 4, 6, 8, 10, 13, 16, and 20 nucleotides. MG160-45, MG160-121, MG160-136, MG160-193, MG160-232, and MG160-358 showed editing levels reaching 5% or higher at varying PBS lengths. Editing levels represented in FIGS. 55B-55W show editing levels across eight different PBS lengths. Bars labeled “correct edit” represent the intended edit with no mistakes in the NGS amplicon, and bars labeled “incorrect edit” refer to the intended edit being incorporated and includes errors within the NGS amplicon, either originating in misincorporation by the RT and/or scaffold incorporation of the pegRNA. Editing levels represented in FIGS. 55X-55AS show editing levels across eight different PBS wherein bars labeled “editing” represent the intended edit with errors in the NGS amplicon (excluding pegRNA scaffold incorporation) and bars labeled “scaffold incorporation” represent the intended edit and scaffold incorporation of the pegRNA.

FIGS. 56A-56F depict the percent editing for diverse edits with MG151-98 mutants untethered to spCas9(H840A) for a VEGFA target. MG151-98 wild-type and mutants MG151-98 (D166AA,H171N) and MG151-98(D166AA,K297P) were evaluated for correction of G-to-T transversion (FIGS. 56A and 56D), 24 nucleotide insertion (FIGS. 56B and 56E), and 15 nucleotide deletion (FIGS. 56C and 56F) on the VEGFA target with pegRNAs having varying PBS lengths of 6, 8, 10, and 13 nucleotides. FIGS. 56A-56C: Bars labeled “correct edit” represent the intended edit with no mistakes in the NGS amplicon, and bars labeled “incorrect edit” refer to the intended edit being incorporated and includes errors within the NGS amplicon, either originating in misincorporation by the RT and/or scaffold incorporation of the pegRNA. Controls MMLV1 and MMLV2 represent untethered spCas9(H840A), pegRNA at PBS 13, and RT plasmid encoding MMLV1 or MMLV2, respectively. Editing levels represented in FIGS. 56D-56F show editing levels across four different PBS lengths wherein bars labeled “editing” represent the intended edit with errors in the NGS amplicon (excluding pegRNA scaffold incorporation) and bars labeled “scaffold incorporation” represent the intended edit and scaffold incorporation of the pegRNA. Controls MMLV1 and MMLV2 represent untethered spCas9(H840A), pegRNA at PBS 13, and RT plasmid encoding MMLV1 or MMLV2, respectively.

FIGS. 57A-57H depict the percent editing for G-to-T transversion of MG151 family mutants and MG153 family mutants untethered to spCas9(H840A). Percent editing for G-to-T transversion on the VEGFA target with pegRNAs having varying PBS lengths of 6, 8, 10, and 13 nucleotides was evaluated for MG151-123 wild type and mutants (M304R, H287F, H178R, H178N, G279R, or G279N) (FIGS. 57A and 57E), MG151-126 wild type and mutants (H287F, G179R, G179N, A280R, A280K, or A276R) (FIGS. 57B and 57F), MG153-18 wild type and mutants (G119R, P242R, or double mutant G119R and P242R) (FIGS. 57C and 57G), and MG153-20 wild type and mutants (N55R, P226R, or double mutant N55R and P226R) (FIGS. 57D and 57H). FIGS. 57A-57D: Bars labeled “correct edit” represent the intended edit with no mistakes in the NGS amplicon, and bars labeled “incorrect edit” refer to the intended edit being incorporated and includes errors within the NGS amplicon, either originating in misincorporation by the RT and/or scaffold incorporation of the pegRNA. Editing levels represented in FIGS. 57E-57H show percent editing levels across four different PBS lengths wherein bars labeled “editing” represent the intended edit with errors in the NGS amplicon (excluding pegRNA scaffold incorporation) and bars labeled “scaffold incorporation” represent the intended edit and scaffold incorporation of the pegRNA. Controls including “no RT” represents untethered spCas9(H840A) with pegRNA at PBS 13 and MMLV1 and MMLV2 represent untethered spCas9(H840A), pegRNA at PBS 13, and RT plasmid encoding MMLV1 or MMLV2, respectively.

FIGS. 58A-58L depict the percent editing for diverse edits with MG160-473 mutants tethered to spCas9(H840A) for VEGFA target. MG160-473 wild type and mutants MG160-473(F231K) and MG160-473(F231R) were evaluated for correction of G-to-T transversion (FIGS. 58A, 58D, 58G, and 58J), 24 nucleotide insertion (FIGS. 58B, 58E, 58H, and 58K), and 15 nucleotide deletion (FIGS. 58C, 58F, 58I, and 58L) on the VEGFA target with pegRNAs having varying PBS lengths of 6, 8, 10, 13, and 16 nucleotides. FIGS. 58A-C and 58G-I: Bars labeled “correct edit” represent the intended edit with no mistakes in the NGS amplicon, and bars labeled “incorrect edit” refer to the intended edit being incorporated and includes errors within the NGS amplicon, either originating in misincorporation by the RT and/or scaffold incorporation of the pegRNA. Editing levels represented in FIGS. 58D-58F and 58J-L show percent editing levels across different PBS lengths wherein bars labeled “editing” representing the intended edit with errors in the NGS amplicon (excluding pegRNA scaffold incorporation) and bars labeled “scaffold incorporation” represent the intended edit and scaffold incorporation of the pegRNA. Controls including “untreated” represents cells with no treatment during transfection and cas-PE1 and cas-PE2 represent tethered spCas9(H840A) to MMLV1 or MMLV2 using pegRNA at PBS 13, respectively. Asterisks indicates NGS sample had less than 1000 reads.

FIGS. 59A-59P depict the percent editing of five nucleotide change on AAVS1 target with tethered MG reverse transcriptase and MG71-2n. The reverse transcriptase was tested either untethered to MG71-2n, tethered to the C-terminus of MG71-2n (nickase-RT), or tethered to the N-terminus of MG71-2n (RT-nickase) across six different PBS lengths (6, 8, 10, 13, 16, or 20 nucleotides) targeting a five nucleotide change on AAVS1 target. Reverse transcriptases tested for this correction include: MMLV1 (FIGS. 59A and 59D), MMLV2 (FIGS. 59B and 59E), MG160-4 (FIGS. 59C and 59F), MG151-98 (D166AA) (FIGS. 59G and 59J), MG151-98 (D166AA, H171N) (FIGS. 59H and 59K), MG151-98(D166AA, K297P) (FIGS. 59I and 59L), MG160-4(H230R) (FIGS. 59M and 59O), and MG160-473 (FIGS. 59N and 59P). Bars labeled “correct edit” represent the intended edit with no mistakes in the NGS amplicon and bars labeled “incorrect edit” refer to the intended edit being incorporated and includes errors within the NGS amplicon, either originating in misincorporation by the RT and/or scaffold incorporation of the pegRNA. Bars labeled “editing” represent the intended edit with errors in the NGS amplicon (excluding pegRNA scaffold incorporation) and bars labeled “scaffold incorporation” represent the intended edit and scaffold incorporation of the pegRNA. Low read count indicates NGS sample had less than 1000 reads.

FIGS. 60A-60H depict the percent editing of diverse edits on AAVS1 target with MG reverse transcriptases tethered to the N-terminus of MG71-2n. Reverse transcriptase MMLV1, MMLV2, MG160-4 wild type, or MG160-4 (H230R) was tethered by a 32 amino acid linker to the N-terminus of MG71-2n and challenged to either a G-to-T transversion (FIGS. 60A and 60E), a 24 nucleotide insertion (FIGS. 60B and 60F), a 15 nucleotide deletion (FIGS. 60C and 60G), or a five nucleotide change (FIGS. 60D and 60H) on the AAVS1 target using pegRNAs with PBS lengths of 8, 10, 13, and 16 nucleotides. MG160 candidates were also tested untethered (UT) to MG71-2n with a pegRNA at a PBS length of 13 nucleotides. FIGS. 60A-60D: Bars labeled “correct edit” represent the intended edit with no mistakes in the NGS amplicon and bars labeled “incorrect edit” refer to the intended edit being incorporated and includes errors within the NGS amplicon, either originating in misincorporation by the RT and/or scaffold incorporation of the pegRNA. Editing levels represented in FIGS. 60E-60H show percent editing levels across four different PBS lengths wherein bars labeled “editing” represent the intended edit with errors in the NGS amplicon (excluding pegRNA scaffold incorporation) and bars labeled “scaffold incorporation” represent the intended edit and scaffold incorporation of the pegRNA. Asterisks indicates NGS sample had less than 1000 reads.

FIGS. 61A-61L depict the percent editing of diverse edits on AAVS1 target with MG151-98 mutants untethered to MG71-2n. Reverse transcriptases MMLV1, MMLV2, MG151-98 (D166AA, H171N), MG151-98 (D166AA, K297P), MG151-98 (D166AA, H171N, K297P), and untethered MG71-2n were challenged to either a G-to-T transversion (FIGS. 61A and 61E), a 24 nucleotide insertion (FIGS. 61B and 61F), a 15 nucleotide deletion (FIGS. 61C and 61G), or a five nucleotide change (FIGS. 61D and 61H) on the AAVS1 target using pegRNAs with PBS lengths of 8, 10, 13, and 16 nucleotides. FIGS. 61A-61D: Bars labeled “correct edit” represent the intended edit with no mistakes in the NGS amplicon, and bars labeled “incorrect edit” refer to the intended edit being incorporated and includes errors within the NGS amplicon, either originating in misincorporation by the RT and/or scaffold incorporation of the pegRNA. Editing levels represented in FIGS. 61E-61H show percent editing levels across four different PBS lengths wherein bars labeled “editing” represent the intended edit with errors in the NGS amplicon (excluding pegRNA scaffold incorporation) and bars labeled “scaffold incorporation” represent the intended edit and scaffold incorporation of the pegRNA. FIGS. 61I-61K depict editing levels for each specific correction across four different PBS lengths (8, 10, 13, and 16 nucleotides) for each reverse transcriptase with line representing average median percent editing.

FIGS. 62A-62B depict modifications to the MG71-2 scaffold resulting in improved five nucleotide change editing percentage on AAVS1 target. The scaffold for MG71-2 contains 107 nucleotides, and two modified versions of the scaffold, D2 or D2C2, resulted in a shortened scaffold length of 85 nucleotides and 79 nucleotides, respectively. The D2 scaffold removed the last hairpin of the MG71-2 scaffold, and the D2C2 scaffold removed the last hairpin in combination with a small bulge of the MG71-2 scaffold. Editing levels for a five nucleotide change on the AAVS1 target were tested on the wild type and modified scaffold across PBS lengths of 8, 10, 13, and 16 nucleotides with reverse transcriptase MMLV2 or MG160-4 (H230R) tethered to the N-terminus of MG71-2n. FIG. 62A: Bars labeled “correct edit” represent the intended edit with no mistakes in the NGS amplicon and bars labeled “incorrect edit” refer to the intended edit being incorporated and includes errors within the NGS amplicon, either originating in misincorporation by the RT and/or scaffold incorporation of the pegRNA. Editing levels represented in FIG. 62B show percent editing levels across eight different PBS lengths wherein bars labeled “editing” represent intended edit with errors in the NGS amplicon (excluding pegRNA scaffold incorporation) and bars labeled “scaffold incorporation” represent intended edit and scaffold incorporation of the pegRNA.

FIGS. 63A-63H depict guide RNA optimization to improve editing levels for MG71-2n. FIGS. 63A-63D show reverse transcriptases MMLV1, MMLV2, MG151-98 (D166AA, H171N), MG151-98 (D166AA, K297P), MG151-98 (D166AA, H171N, K297P) and untethered MG71-2n challenged to a five nucleotide change on the AAVS1 target. FIGS. 63E-63H show reverse transcriptases MMLV1, MMLV2, MG160-4 and MG160-4 (H230R) tethered to the N-terminus of MG71-2n and MG160-4 and MG160-4 (H230R) untethered (UT) to MG71-2n challenged to a five nucleotide change on the AAVS1 target. Varying mismatches in the pegRNA across the PBS region were tested to determine if improvements on editing could be achieved. PBS lengths of 8, 10, 13, and 16 nucleotides in FIGS. 63A, 63C, 63E, and 63G had perfect complementarity to the target region. In FIGS. 63B, 63D, 63F, and 63H, PBS lengths of 10, 13, 16, and 20 nucleotides had perfect complementarity of 8 nucleotides in the region neighboring the reverse transcription template (RTT) and then had varying mismatches (mm) to achieve PBS lengths of 10 (2 mismatches), 13 (5 mismatches), 16 (8 mismatches), and 20 (12 mismatches) nucleotides. Bars labeled “correct edit” represent the intended edit with no mistakes in the NGS amplicon and bars labeled “incorrect edit” refer to the intended edit being incorporated and includes errors within the NGS amplicon, either originating in misincorporation by the RT and/or scaffold incorporation of the pegRNA. Bars labeled “editing” represent the intended edit with errors in the NGS amplicon (excluding pegRNA scaffold incorporation) and bars labeled “scaffold incorporation” represent the intended edit and scaffold incorporation of the pegRNA.

FIGS. 64A-64E depict guide RNA modifications of MG3-6 to improve editing levels in mammalian cells. FIG. 64A: MG3-6 wild type mRNA was used to determine percent modified (including SNPs and InDels) levels of target amplicon AAVS1 in NGS samples. Guide RNA is composed of the scaffold and spacer for the target and pegRNA includes the guide RNA with PBS and RTT sequence. Modifications modL1-modL4 have increased regions of GC content in hairpins 1 through 3 (modL1-modL3) of the scaffold, with modL4 combining modifications of all hairpins in the scaffold. FIGS. 64B-64C depict percent editing for a two nucleotide change in AAVS1 target measured across PBS lengths of 10 and 13 nucleotides with wild type scaffold and modified scaffolds modL1-modL4 using tethered MMLV2 to C-terminus of MG3-6(H586A). As controls, “untreated” represents cells with no treatment during transfection and MG3-6(H586A) represents nickase and pegRNA with no reverse transcriptase include in transfection of cells. FIGS. 64D-64E depict percent editing for a two nucleotide change in AAVS1 target measured across PBS lengths of 8, 10, 13 and 16 nucleotides with perfect complementarity to target or PBS lengths 10 (2 mismatches), 13 (5 mismatches), 16 (8 mismatches), and 20 (12 mismatches) using untethered MMLV1, MMLV2, MG151-98 (D166AA, H171N), and MG151-98 (D166AA, K297P) with nickase MG3-6(H586A). Bars labeled “correct edit” represent the intended edit with no mistakes in the NGS amplicon and bars labeled “incorrect edit” refer to the intended edit being incorporated and includes errors within the NGS amplicon, either originating in misincorporation by the RT and/or scaffold incorporation of the pegRNA. Bars labeled “editing” represent the intended edit with errors in the NGS amplicon (excluding pegRNA scaffold incorporation) and bars labeled “scaffold incorporation” represent the intended edit and scaffold incorporation of the pegRNA.

FIGS. 65A-65B depict comparison of MG3-6 and MG3-6/3-8 recognition of target with guide RNAs having varying PBS lengths. MG3-6 wild type and MG3-6/3-8 mRNA was used to determine percent modified (including SNPs and InDels) levels of target amplicon AAVS1 (FIG. 65A) and B2M (FIG. 65B) for guide RNA or pegRNA with PBS lengths of 2, 4, 6, 8, 10, 13, 16, and 20 nucleotides. The guide RNA is composed of the scaffold and spacer for the target and the pegRNA includes the guide RNA with PBS and RTT sequence. MG3-6/3-8 showed higher levels of modifications (including InDels) on target compared to MG3-6. Control “untreated” represents cells with no treatment during transfection.

FIGS. 66A-66D depict identification of MG14-241 targets for compatibility with prime editing system. FIG. 66A: Wild type MG14-241 mRNA or plasmid was used to determine percent modified (including SNPs and InDels) levels of various targets. Guide RNA for varying targets (G1, H1, B2, E2, F2, and G2) resulted in varying levels of percent modified with target E2 (region of AAVS1) resulted in the highest levels of InDels (reaching about 60%). FIG. 66B: mRNA of MG14-241 was used to determine percent modified (including SNPs and InDels) levels of target amplicon AAVS1 in NGS samples. The guide RNA is composed of the scaffold and spacer for the target and pegRNA includes the guide RNA with PBS and RTT sequence. As PBS length increased, percent modified decreased. Control “untreated” represents cells with no treatment during transfection. FIGS. 66C-66D: Percent editing of five nucleotide change on AAVS1 target across eight different PBS lengths (2, 4, 6, 8, 10, 13, 16, and 20 nucleotides) with untethered reverse transcriptases MMLV1, MMLV2, MG151-98 (D166AA, H171N), and MG151-98 (D166AA, K297P) with nickase MG14-241n. MG14-241n (no RT) represents nickase and pegRNA with no reverse transcriptase included in transfection of cells. Bars labeled “correct edit” represent the intended edit with no mistakes in the NGS amplicon and bars labeled “incorrect edit” refer to the intended edit being incorporated and includes errors within the NGS amplicon, either originating in misincorporation by the RT and/or scaffold incorporation of the pegRNA. Bars labeled “editing” represent the intended edit with errors in the NGS amplicon (excluding pegRNA scaffold incorporation) and bars labeled “scaffold incorporation” represent the intended edit and scaffold incorporation of the pegRNA.

FIGS. 67A-67D depict the design of an engineered cell line, RT-Cas chimeric proteins, and RNA cargo templates to assess integration by TPRT. FIG. 67A depicts a schematic showing the artificial sequence integrated into HEK293 cells via lentivirus to generate the engineered cell line with target sites for integration. FIG. 67B depicts the percentage of indels generated by five different sgRNAs targeting the engineered landing pad. FIG. 67C depicts a schematic showing four different conformations of each RT-Cas9WT/Nickase fusion generated for testing. FIG. 67D depicts six cargo designs generated for testing integration via TPRT.

FIG. 68 depicts a schematic representation of primers used for left end and right end PCRs to detect integrations.

FIGS. 69A-69C depict detection of cargo integration using Cas9 WT-MG140-3 and sg4 using Tapestation at LE (box shows band of interest; FIG. 69A), Sanger sequencing at LE PCR (Sequences matching landing pad and cargo are shown; FIG. 69B). and Sanger sequencing at RE PCR (Sequences showing matches to cargo, but also an insertion of another product (Cas9) is shown; FIG. 69C).

FIGS. 70A-70B depict detection of cargo integration using MG140-3-Cas9 WT and sg4. Tapestation at LE (FIG. 70A) and Sanger sequencing at LE PCR (FIG. 70B) show matches to landing pad and mCherry cargo.

FIG. 71 depicts detection of cargo integration using Cas9 WT-MG140-8 and sg4 by Sanger sequencing at LE.

FIGS. 72A-72B depict detection of cargo integration using MG153-18-CAs9 WT and sg4 by Tapestation at LE (FIG. 72A) and Sanger sequencing at LE (FIG. 72B).

FIGS. 73A-73C depict Retron RT activity on cognate ncRNAs loaded with 2.2 kb cargo. FIG. 73A depicts a schematic of substrate designs for testing activity and processivity of retron RTs. The generic template was used to test retron non-specific activity and was primed by a ssDNA priming oligo annealed to the 3′ end of the RNA. The retron ncRNA was primed with the 5′ and 3′ inverted repeats (IRs) facilitated by the presence of terminal 5′ and 3′ retron ncRNA elements. For both substrates, the cargo sequence was flanked by the reverse complements (rc) of the LE and RE recognition motifs for MG92-4 TnpA. This sequence was then flanked by ˜100 nt RNA sequences that when converted to cDNA can be quantified by multiplexed TaqMan qPCR to evaluate how much of the 5′ (FAM) and 3′ (HEX) end of the cDNA molecule was synthesized by the RT. For the retron ncRNA substrate, the sequence was inserted within a previously identified replaceable region of the ncRNA msd. FIG. 73B depicts the quantity of ssDNA detected by FAM and HEX by multiplexed TaqMan qPCR. The no RT control was generated by not adding any RT expression template to the cell-free expression system. The dashed line is 10-fold above the highest background no RT signal. TGIRT is a GII intron control RT, MMLV is a retroviral control RT, and Ec86 is a retron contro RT. The label “gen” denotes that the RT was tested with the generic template, while “ncRNA” indicates that the RT was tested with its cognate ncRNA loaded with cargo. FIG. 73C depicts confirmation of 2.2 kb ssDNA generated by RTs by tapestation D5000. Lanes correspond to the following: Lane 1: Ladder; Lane 2: no RT gen; Lane 3: TGIRT gen; Lane 4: MG154-1 nRNA; Lane 5: MG157-1 ncRNA; Lane 6: MG157-3 ncRNA; Lane 7: MG157-4 ncRNA; Lane 8: MG158-1 ncRNA; Lane 9: MG159-3 ncRNA; Lane 10: MG173-1 ncRNA.

FIGS. 74A-74B depict a screen for the ability of retron RT MG173-1 to synthesize cDNA in mammalian cells. FIG. 74A depicts a cartoon depicting the methodology used to detect cDNA synthesis in mammalian cells. The first (FAM) and last (HEX) 100 bps of a 4.1 kb RNA template are detected using Taqman based qPCR. FIG. 74B depicts Taqman qPCR detection of first (FAM probe) and last (HEX probe) 100 bp per products amplified from cDNA synthesized from a generic 4 kb template, a generic 2 kb template, and an MG173-1 specific template flanked by 5′ and 3′ terminal MG173-1 ncRNA elements.

FIGS. 75A-75B depict the insertion reaction and Sanger sequencing for PCR of TnpA 92-4 with 2.2 kb retron-produced cDNA cargo. FIG. 75A: Lane 1: PCR of no template control (NTC) insertion reaction with a ssDNA ultramer target and MG173-1 produced cDNA cargo. Lane 2: PCR of TnpA 92-4 insertion reaction with a ssDNA ultramer target and MG173-1 produced cDNA cargo. FIG. 75B: Sanger sequencing of chimeric insertion product generated by TnpA 92-4 mediated insertion of MG173-1 produced cargo into a ssDNA ultramer target. FIG. 75B discloses SEQ ID NO: 2579.

FIGS. 76A-76H depict the targeting of therapeutic sites with MG71-2. FIG. 76A: WT mRNA of MG71-2 having InDels on therapeutically relevant sites (hPDK1, G6PC1 Q347*, and PAH R408W) with various guide RNAs. Highest InDels seen are at guide 1 for hPDK1 gene and guide 2 for PAH gene targeting an R408W mutation. Other guides tested for G6PC1 had no InDel detection with these guides. The positive control contained a guide RNA targeting AAVS1. FIG. 76B: Targeting HBB gene mutation E7V with guide RNA and pegRNAs with varying PBS lengths of 8, 10, and 13 nucleotides. InDels slightly decrease with pegRNA compared to guide RNA. Editing levels across eight different PBS lengths are shown. FIGS. 76C-76H: Prime editing experiments were then performed with pegRNAs using the spacers from FIGS. 76A-76B. Prime editing systems were MG160-4(H230R) tethered to the N-term of MG71-2n (MG160-4(H230R)-MG71-2n) and MMLV2 tethered to the N-term of MG71-2n (MMLV2-MG71-2n). FIGS. 76C-76D: MG160-4(H230R)-MG71-2n and MMLV2-MG71-2n targeted disruption of a microRNA recognition site by using pegRNAs that contained 3 or 5 nucleotide (nt) mismatches incorporated into the RT template (RTT) of the pegRNA. Highest levels of editing were seen at PBS 10 for a 3nt mismatch incorporation into the hPDK1 microRNA recognition site. FIGS. 76E-76F: Prime editing systems targeting PAH R408W across PBS lengths 8, 10, and 13 nt with RTT varying in length of 29nt and 32nt showed no detectable levels of editing. FIGS. 76G-76H: MG160-4(H230R)-MG71-2n and MMLV2-MG71-2n targeted HBB E7V mutation across multiple PBS lengths and achieved above background level of editing. Bars labeled “correct edit” represent the intended edit with no mistakes in the NGS amplicon and bars labeled “incorrect edit” refer to intended edit being incorporated and includes errors within the NGS amplicon, either originating in misincorporation by the RT and/or scaffold incorporation of the pegRNA. Bars labeled “editing” represent the intended edit with errors in the NGS amplicon (excluding pegRNA scaffold incorporation) and bars labeled “scaffold incorporation” represent the intended edit and scaffold incorporation of the pegRNA. * indicates less than 1000 reads were obtained for that NGS sample, and error bars represent the standard deviation of two biological replicates.

FIGS. 77A-77D depict data demonstrating that MG71-2 recognizes multiple guide RNAs across various targets allowing for the incorporation of larger genomic changes. FIG. 77A: WT mRNA of MG71-2 having InDels on two targets (TRAC and AAVS1) with various guide RNAs. Target sites, D3 and D4, on AAVS1 showed some of the highest levels of editing and had a distance of 69nt apart on the AAVS1 target. Spacers for D3 and D4 were oriented in the correct orientation to be compatible for TWIN, PASTE, and template jumping (Tj) prime editing methods. FIG. 77B: Tape station gel image for confirming replacement of a 69nt sequence in the AAVS1 target with a 38nt Bxb1 sequence using a Bxb1 specific primer. Lanes G3 and H3 are two replicates for MMLV2-MG71-2n using pegRNA containing the Bxb1 sequence and a nicking guide (PASTE method), while lanes A4 and B4 represent two replicates for MMLV2-MG71-2n using pegRNA containing the Bxb1 sequence and no nicking guide. Lanes C4 and D4 are samples from MG151-98(H171N, K297P, 166AA)-MG71-2n using pegRNA containing the Bxb1 sequence and no nicking guide, while lanes E4 and D4 used pegRNA containing the Bxb1 sequence and a nicking guide (PASTE method). FIGS. 77C-77D: Tape station fragment analysis for lanes G3, H3, E4, and F4 confirming amplicon containing Bxb1 sequence.

FIGS. 78A-78L depict optimization of MG71-2n system with selected reverse transcriptases. FIGS. 78A-78D: MG160-4(H230R) was either cloned on the N- or C-terminus of MG71-2n with a 33 amino acid linker. In addition, MG160-4(H230R) and MG71-2n was inlaid at five different insertion sites (S311, S355, T396, I822, and V1176). Inlaid constructs had a 33 amino acid linker on the 5′ and 3′ end of MG160-4(H230R) at the insertion site. Inlaid constructs were tested for a 5nt change and a 24nt insertion on AAVS1 target across four different PBS lengths. MG160-4(H230R) on N-terminus of MG71-2n showed highest levels of editing. FIGS. 78E-78H: Various linker lengths (14AA, 15AA, 26AA, and 32AA) fusing MG160-4 to the N-terminus of MG71-2 were tested alongside the original 33AA linker. The 32AA and 33AA linker had similar levels of editing for both a 5nt change and a 24nt insertion on AAVS1 target. FIGS. 78I-78L: Various linker lengths (7AA, 14AA, 15AA, 16AA, 26AA, 32AA 44AA, and 58AA) fusing RT MG160-473 or MG151-98 (H171N, Δ166AA) to the N-terminus of MG71-2 were tested alongside the original 33AA linker and screened for incorporating a 5nt change and a 24nt insertion on AAVS1 target. Bars labeled “correct edit” represent the intended edit with no mistakes in the NGS amplicon and bars labeled “incorrect edit” refer to the intended edit being incorporated and includes errors within the NGS amplicon, either originating in misincorporation by the RT and/or scaffold incorporation of the pegRNA. Bars labeled “editing” represent the intended edit with errors in the NGS amplicon (excluding pegRNA scaffold incorporation) and bars labeled “scaffold incorporation” represent the intended edit and scaffold incorporation of the pegRNA.

FIGS. 79A-79O depict the targeting of therapeutic sites with MG3-6-3-8 and MG3-6. FIG. 79A: WT mRNA of MG3-6/3-8 having InDels on therapeutically relevant sites (A1AT, PAH R408W, G6PC1 Q347*, G6PC1 R83C, and hPDK1) with various guide RNAs. Guide RNAs represented with the dark grey bar indicates the chosen spacer sequence for designing pegRNAs. FIGS. 79B-79K: Prime editing systems MG160-4(H230R) tethered to the C-term of MG3-6-3-8n (MG3-6-3-8n-MG160-4(H230R) and MMLV2 tethered to the C-term of MG3-6-3-8n (MG3-6-3-8n-MMLV2-) were tested for prime editing at therapeutically relevant sites. No editing was detected at sites PAH R408W, G6PCI: R83C, and hPDK1. Some detectable levels of editing were seen for A1AT and G6PC1 Q347*. FIGS. 79L-79O: MG160-4(H230R) tethered to the N-terminus of MG3-6n or MG3-6-3-8n was compared to editing with MMLV2 tethered to the C-terminus of MG3-6n or MG3-6-3-8n. These constructs targeted four therapeutic sites A1A and hPDK1. Bars labeled “correct edit” represent the intended edit with no mistakes in the NGS amplicon and bars labeled “incorrect edit” refer to the intended edit being incorporated and includes errors within the NGS amplicon, either originating in misincorporation by the RT and/or scaffold incorporation of the pegRNA. Bars labeled “editing” represent the intended edit with errors in the NGS amplicon (excluding pegRNA scaffold incorporation) and bars labeled “scaffold incorporation” represent the intended edit and scaffold incorporation of the pegRNA. * indicates less than 1000 reads were obtained for that NGS sample.

FIGS. 80A-80D depict optimization of MG3-6n system with MG160-4 and MG160-4(H230R). FIGS. 80A-80B: MG160-4 was cloned to the N terminus of MG3-6n with various linker lengths of 33AA (the original linker length) as well as 32AA, 44AA, and 58AA. These prime editing systems were then tested to correct two STOP codons in a linker between hygromycin and BFP engineered cell line. pegRNAs with PBS lengths of 8, 10, and 13 nucleotides were tested. Using pegRNA with a PBS length of 8nt showed highest levels of editing using a fusion construct having a 58AA. As PBS length got longer, the difference between the linker systems showed less variability in editing levels when using prime editing systems with different linker lengths. FIGS. 80C-80D: In addition, MG160-4(H230R) and MG3-6n was inlaid at five different insertion sites (K115, V208, K368, D550, and L881). Inlaid constructs had a 33 amino acid linker on the 5′ and 3′ end of MG160-4(H230R) at the insertion site. Inlaid constructs were tested for correction of two STOP codons in a linker between hygromycin and BFP engineered cell line across three different PBS lengths. Highest levels of editing were seen when MG160-4(H230R) was tethered to the N-terminus of MG3-6n. Bars labeled “correct edit” represent the intended edit with no mistakes in the NGS amplicon and bars labeled “incorrect edit” refer to the intended edit being incorporated and includes errors within the NGS amplicon, either originating in misincorporation by the RT and/or scaffold incorporation of the pegRNA. Bars labeled “editing” represent the intended edit with errors in the NGS amplicon (excluding pegRNA scaffold incorporation) and bars labeled “scaffold incorporation” represent the intended edit and scaffold incorporation of the pegRNA.

FIGS. 81A-81C depict a screen of natural reverse transcriptases tethered to N-terminus of MG71-2n targeting AAVS1. FIG. 81A: Summary of MG198 candidates tethered to the N-terminus of MG71-2n targeting a 5nt change in AAVS1 using pegRNAs at varying PBS lengths (8, 10, 13, and 16nt). Editing levels above background were seen for candidates MG198-6 and MG198-7. FIGS. 81B-81C: MG160 candidates MG160-45, MG160-121, MG160-136, and MG160-232 were tethered to the N-terminus of MG71-2n targeting a 5nt change in AAVS1 using pegRNAs at varying PBS lengths (8, 10, 13nt). All MG160 candidates were slightly above background levels but showed poor activity compared to MG160-4(H230R) and MMLV2 tethered to the N-terminus of MG71-2n. Bars labeled “correct edit” represent the intended edit with no mistakes in the NGS amplicon and bars labeled “incorrect edit” refer to the intended edit being incorporated and includes errors within the NGS amplicon, either originating in misincorporation by the RT and/or scaffold incorporation of the pegRNA. Bars labeled “editing” represent the intended edit with errors in the NGS amplicon (excluding pegRNA scaffold incorporation) and bars labeled “scaffold incorporation” represent the intended edit and scaffold incorporation of the pegRNA. * indicates less than 1000 reads were obtained for that NGS sample

FIGS. 82A-82I depict a screen of MG160 ASR candidates tethered to N-terminus of MG71-2n for versatile edits on AAVS1 target. FIG. 82A: Summary of MG160 ASR candidates tethered to the N-terminus of MG71-2n targeting a 5nt change in AAVS1 using pegRNAs at varying PBS lengths (8, 10, 13, and 16nt). Editing levels above background were seen for candidates MG160-491, MG160-492, and MG160-493. FIGS. 82B-82C: MG160-491, MG160-492, and MG160-493 were then compared to wild type MG160-4, MG160-4(H230R), MMLV2, and EC86 for a 5nt change on AAVS1. All candidates were comparable to MG160-4(H230R). MG160-491, MG160-492, and MG160-493 were then tested for a G-to-T transvehrsion (FIGS. 82D and 82G), a 24nt insertion (FIGS. 82E and 82H), and a 15nt deletion (FIGS. 82F and 82I). Bars labeled “correct edit” represent the intended edit with no mistakes in the NGS amplicon and bars labeled “incorrect edit” refer to the intended edit being incorporated and includes errors within the NGS amplicon, either originating in misincorporation by the RT and/or scaffold incorporation of the pegRNA. Bars labeled “editing” represent the intended edit with errors in the NGS amplicon (excluding pegRNA scaffold incorporation) and bars labeled “scaffold incorporation” represent the intended edit and scaffold incorporation of the pegRNA. * indicates less than 1000 reads were obtained for that NGS sample.

FIGS. 83A-83D depict the impact of nicking guides on prime editing efficiency. FIGS. 83A-83B: Summary of prime editing efficiency with a panel of nicking guides in K562 cells with MG160-4 H230R-MG71-2n. Bars labeled “correct edit” represent the intended edit with no mistakes in the NGS amplicon and bars labeled “incorrect edit” refer to the intended edit being incorporated and includes errors within the NGS amplicon, either originating in misincorporation by the RT and/or scaffold incorporation of the pegRNA. Bars labeled “editing” represent the intended edit with errors in the NGS amplicon (excluding pegRNA scaffold incorporation) and bars labeled “scaffold incorporation” represent the intended edit and scaffold incorporation of the pegRNA. FIGS. 83C-83D: Summary of prime editing efficiency with a panel of nicking guides in K562 cells with MMLV2-MG71-2n. No nick bar indicates baseline editing with 5nt change guide, no guide indicated background editing in mRNA-only samples.

FIGS. 84A-84D depict the impact of nicking guides on prime editing efficiency in K562 and HEK293T cells. FIGS. 84A-84B: Summary of prime editing efficiency with nicking guides A2-H2 and A6-H6 from FIG. 78 in K562 cells with MG160-4 H230R-MG71-2n, MMLV2-MG71-2n and MG151-98-DM-SL1-MG71-2n. No nick bar indicates baseline editing with 5nt change guide, no guide indicates background editing in mRNA-only samples. Bars labeled “correct edit” represent the intended edit with no mistakes in the NGS amplicon and bars labeled “incorrect edit” refer to the intended edit being incorporated and includes errors within the NGS amplicon, either originating in misincorporation by the RT and/or scaffold incorporation of the pegRNA. Bars labeled “editing” represent the intended edit with errors in the NGS amplicon (excluding pegRNA scaffold incorporation) and bars labeled “scaffold incorporation” represent the intended edit and scaffold incorporation of the pegRNA. FIGS. 84C-84D: Summary of prime editing efficiency with nicking guides A2-H2 and A6-H6 from FIG. 78 in HEK293T cells with MG160-4 H230R-MG71-2n, MMLV2-MG71-2n, and MG151-98-DM-SL1-MG71-2n. No nick bar indicates baseline editing with 5nt change guide, no guide indicates background editing in mRNA-only samples.

FIGS. 85A-85B depict the impact of nicking guides on prime editing efficiency in K562 cells. FIGS. 85A-85B: Summary of prime editing efficiency with nicking guides A2-H2, A5-H5 and A6-H6 from FIG. 78 in K562 cells with MG160-4 H230R-MG71-2n, MMLV2-MG71-2n, and MG151-98-DM-SL1-MG71-2n. pegRNAs with PBS lengths 8, 10, 13, and 16 encoding for a single nucleotide G to T change at AAVS1 were used in these experiments. No nick bars indicate baseline editing with pegRNAs with the indicated PBS length, no guide indicates background editing in mRNA-only samples. Bars labeled “correct edit” represent the intended edit with no mistakes in the NGS amplicon and bars labeled “incorrect edit” refer to the intended edit being incorporated and includes errors within the NGS amplicon, either originating in misincorporation by the RT and/or scaffold incorporation of the pegRNA. Bars labeled “editing” represent the intended edit with errors in the NGS amplicon (excluding pegRNA scaffold incorporation) and bars labeled “scaffold incorporation” represent the intended edit and scaffold incorporation of the pegRNA.

FIGS. 86A-86B depict the optimization of prime editing efficiency with nicking guides. FIGS. 86A-86B: Summary of prime editing efficiency with nicking guide E6 from FIG. 78 in K562 cells with MG160-4 H230R-MG71-2n. No nick bar indicates baseline editing with 5nt change guide, no guide indicates background editing in mRNA-only samples. Different rations of pegRNA to nicking guides were tested and editing efficiency assessed. Bars labeled “correct edit” represent the intended edit with no mistakes in the NGS amplicon and bars labeled “incorrect edit” refer to the intended edit being incorporated and includes errors within the NGS amplicon, either originating in misincorporation by the RT and/or scaffold incorporation of the pegRNA. Bars labeled “editing” represent the intended edit with errors in the NGS amplicon (excluding pegRNA scaffold incorporation) and bars labeled “scaffold incorporation” represent the intended edit and scaffold incorporation of the pegRNA.

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

SEQ ID NOs: 1-37 show the full-length nucleic acid sequences of untethered MG151 family reverse transcriptases suitable for the gene editing systems described herein.

SEQ ID NOs: 38-61 show the full-length nucleic acid sequences of untethered MG153 family reverse transcriptases suitable for the gene editing systems described herein.

SEQ ID NOs: 62-68 show the full-length nucleic acid sequences of untethered MG160 family reverse transcriptases suitable for the gene editing systems described herein.

SEQ ID NOs: 69-75 show the full-length nucleic acid sequences of tethered MG160 family reverse transcriptases suitable for the gene editing systems described herein.

SEQ ID NOs: 76-83 show the RNA sequences of chemically modified guide RNAs with a single point mutation (VEGFA spacer G to T) with PBS of different lengths suitable for the gene editing systems described herein.

SEQ ID NOs: 84-91 show the RNA sequences of chemically modified guide RNAs with a single deletion (VEGFA spacer deletion change) with PBS of different lengths suitable for the gene editing systems described herein.

SEQ ID NOs: 92-99 show the RNA sequences of chemically modified guide RNAs with a single insertion (VEGFA spacer single insertion) with PBS of different lengths suitable for the gene editing systems described herein.

SEQ ID NOs: 100-101 show the sequences of primers suitable for conducting site-directed editing in the VEGFA site.

SEQ ID NO: 102 shows the nucleic acid sequence of the VEGFA target site.

SEQ ID NO: 103 shows the nucleic acid sequence of an exemplary RT-nickase linker.

SEQ ID NO: 104 shows the nucleic acid sequence of an MG3 effector nuclease suitable for the gene editing systems described herein.

SEQ ID NOs: 105-108 show the nucleic acid sequences of the endogenous targets AAVS1, B2M, CD5, and CD38.

SEQ ID NOs: 109-140 show the RNA sequences of chemically modified guide RNAs with spacers targeting AAVS1, B2M, CD5, and CD38 with PBS of different lengths suitable for the gene editing systems described herein.

SEQ ID NOs: 141-148 show the sequences of primers suitable for conducting site-directed editing in the AAVS1, B2M, CD5, and CD38 sites.

SEQ ID NO: 149 shows the RNA sequence of a chemically modified guide RNA with a spacer targeting VEGFA.

SEQ ID NOs: 150-151 and 2580-2581 show the sequences of two retrotransposition assay reporters.

SEQ ID NOs: 152-154 show the amino acid sequences of MG3-6 nucleases (nMG3-6 D13A, nMG3-6 H586A, and nMG3-6 N609A).

SEQ ID NOs: 155-160 show the amino acid sequences of exemplary RT-nickase linkers.

SEQ ID NOs: 161-291 show the amino acid sequences of MG140 family retrotransposition proteins suitable for the gene editing systems described herein.

SEQ ID NOs: 292-293 show the amino acid sequences of MG146 family retrotransposition proteins suitable for the gene editing systems described herein.

SEQ ID NOs: 294-317 show the amino acid sequences of MG148 family reverse transcriptase proteins suitable for the gene editing systems described herein.

SEQ ID NOs: 318-330 show the amino acid sequences of MG149 family reverse transcriptase proteins suitable for the gene editing systems described herein.

SEQ ID NOs: 331-445 show the amino acid sequences of MG151 family reverse transcriptase proteins suitable for the gene editing systems described herein.

SEQ ID NOs: 446-499 show the amino acid sequences of MG153 family reverse transcriptases proteins suitable for the gene editing systems described herein.

SEQ ID NOs: 500-501 show the amino acid sequences of MG154 family reverse transcriptase proteins suitable for the gene editing systems described herein.

SEQ ID NOs: 502-506 show the amino acid sequences of MG155 family reverse transcriptase proteins suitable for the gene editing systems described herein.

SEQ ID NOs: 507-508 show the amino acid sequences of MG156 family reverse transcriptase proteins suitable for the gene editing systems described herein.

SEQ ID NOs: 509-513 show the amino acid sequences of MG157 family reverse transcriptase proteins suitable for the gene editing systems described herein.

SEQ ID NO: 514 shows the amino acid sequences of MG158 family reverse transcriptase proteins suitable for the gene editing systems described herein.

SEQ ID NOs: 515-517 show the amino acid sequences of MG159 family reverse transcriptase proteins suitable for the gene editing systems described herein.

SEQ ID NOs: 518-566 show the amino acid sequences of MG160 family reverse transcriptase proteins suitable for the gene editing systems described herein.

SEQ ID NOs: 567-571 show the amino acid sequences of MG163 family reverse transcriptase proteins suitable for the gene editing systems described herein.

SEQ ID NOs: 572-576 show the amino acid sequences of MG164 family reverse transcriptase proteins suitable for the gene editing systems described herein.

SEQ ID NOs: 577-585 show the amino acid sequences of MG165 family reverse transcriptase proteins suitable for the gene editing systems described herein.

SEQ ID NOs: 586-590 show the amino acid sequences of MG166 family reverse transcriptase proteins suitable for the gene editing systems described herein.

SEQ ID NOs: 591-595 show the amino acid sequences of MG167 family reverse transcriptase proteins suitable for the gene editing systems described herein.

SEQ ID NOs: 596-600 show the amino acid sequences of MG168 family reverse transcriptase proteins suitable for the gene editing systems described herein.

SEQ ID NOs: 601-611 show the amino acid sequences of MG169 family reverse transcriptase proteins suitable for the gene editing systems described herein.

SEQ ID NOs: 612-621 show the amino acid sequences of MG170 family reverse transcriptase proteins suitable for the gene editing systems described herein.

SEQ ID NOs: 622-626 show the amino acid sequences of MG172 family reverse transcriptase proteins suitable for the gene editing systems described herein.

SEQ ID NOs: 627-628 show the amino acid sequences of MG173 family reverse transcriptase proteins suitable for the gene editing systems described herein.

SEQ ID NO: 629 shows the amino acid sequence of an MG176 family retrotransposition protein suitable for the gene editing systems described herein.

SEQ ID NOs: 630-645 show nuclear localization signals (NLS) suitable for the gene editing systems described herein.

SEQ ID NO: 646 shows the amino acid sequence of an MG3-6 nuclease suitable for the gene editing systems described herein.

SEQ ID NO: 647 shows the amino acid sequence of an MG29-1 nuclease suitable for the gene editing systems described herein.

SEQ ID NO: 648 shows the nucleotide sequence of an RNA template for cDNA synthesis.

SEQ ID NO: 653 shows the nucleotide sequence of MG3-6 (H586A).

SEQ ID NOs: 654-655 shows the nucleotide sequences of cDNAs encoding gene targets.

SEQ ID NOs: 656-697 show the full-length peptide sequences of chemically modified guide RNAs.

SEQ ID NOs: 698-701 show the nucleotide sequences of primers.

SEQ ID NOs: 702-709 show the nucleotide sequences of reverse transcriptases cloned into a tethered MG3-6(H586A) plasmid.

SEQ ID NOs: 710-727 show the nucleotide sequences of genes encoding MG151 reverse transcriptase proteins optimized for expression in mammalian cells and cloned into an untethered plasmid.

SEQ ID NOs: 728-749 show the nucleotide sequences of genes encoding MG160 reverse transcriptase proteins optimized for expression in mammalian cells and cloned into a tethered spCas9(H840A) plasmid.

SEQ ID NOs: 750-766 show the nucleotide sequences of genes encoding MG151 reverse transcriptase proteins optimized for expression in mammalian cells and cloned into an untethered plasmid.

SEQ ID NOs: 767-784 show the full-length peptide sequences of MG151 reverse transcriptase proteins.

SEQ ID NOs: 786-1220 show the full-length peptide sequences of MG160 reverse transcriptase proteins.

SEQ ID NOs: 1221-1226, and 1299 show the nucleotide sequences of genes encoding MG153 reverse transcriptase proteins optimized for expression in mammalian cells and cloned into an untethered plasmid.

SEQ ID NOs: 1227-1243, 1250-1256, and 1265-1271 show the nucleotide sequences of genes encoding MG160 reverse transcriptase proteins optimized for expression in mammalian cells and cloned into a tethered spCas9 (H840A) plasmid.

SEQ ID NOs: 1245-1246 show the nucleotide sequences of RT linkers.

SEQ ID NOs: 1257-1264 and 1272-1279 show the nucleotide sequences of genes encoding MG160 reverse transcriptase proteins optimized for expression in mammalian cells and cloned into an untethered plasmid.

SEQ ID NOs: 1280-1292, and 1299 show the nucleotide sequences of genes encoding reverse transcriptase proteins optimized for expression in mammalian cells and cloned into an untethered plasmid.

SEQ ID NOs: 1293-1295, and 1300 show the nucleotide sequences of genes encoding reverse transcriptase proteins optimized for expression in mammalian cells and cloned into a tethered or untethered plasmid.

SEQ ID NOs: 1301-1304, and 1309 show the nucleotide sequences of genes encoding mutant reverse transcriptase proteins optimized for expression in mammalian cells and cloned into a tethered or untethered plasmid.

SEQ ID NOs: 1336-1341 show the nucleotide sequences of chemically modified guide RNAs with a single point mutation (AAVS1 spacer G to T) with PBS of different lengths suitable for the gene editing systems described herein.

SEQ ID NOs: 1330-1335 show the nucleotide sequences of chemically modified guide RNAs with a single deletion (AAVS1 spacer deletion change) with PBS of different lengths suitable for the gene editing systems described herein.

SEQ ID NOs: 1324-1329 show the nucleotide sequences of chemically modified guide RNAs with a single insertion (AAVS1 spacer single insertion) with PBS of different lengths suitable for the gene editing systems described herein.

SEQ ID NOs: 1310-1315 show the nucleotide sequences of chemically modified guide RNAs (for targeting AAVS1) with a 5 nucleotide change with PBS of different lengths suitable for the gene editing systems described herein.

SEQ ID NOs: 1317-1323 show the nucleotide sequences of chemically modified guide RNAs (for targeting AAVS1) with a modified backbone with PBS of different lengths suitable for the gene editing systems described herein.

SEQ ID NOs: 1342-1343 show the nucleotide sequence of MG71-2 AAVS1 primers.

SEQ ID NO: 1344 shows the nucleotide sequence of a cDNA encoding a gene target.

SEQ ID NO: 1247 shows the nucleotide sequence of a spCas9(H840A) untethered or tethered plasmid.

SEQ ID NO: 1248 shows the nucleotide sequence of MMLV1 codon optimized for expression in mammalian cells and cloned into a tethered or untethered plasmid.

SEQ ID NO: 1249 shows the nucleotide sequence of MMLV2 codon optimized for expression in mammalian cells and cloned into a tethered or untethered plasmid.

SEQ ID NOs: 1345-1353 show the nucleotide sequences of ncRNAs.

SEQ ID NOs: 1354-1361 show the nucleotide sequences of primers.

SEQ ID NOs: 1362-1393 show the nucleotide sequences of ncRNAs.

SEQ ID NOs: 1394-1401 show the nucleotide sequences of MG173 family reverse transcriptases codon optimized for expression in mammalian cells and cloned into an untethered plasmid.

SEQ ID NO: 1402 shows the nucleotide sequence of an MG192 family reverse transcriptase codon optimized for expression in mammalian cells and cloned into an untethered plasmid.

SEQ ID NOs: 1403-1424 show the nucleotide sequences of MG160 family reverse transcriptases codon optimized for expression in mammalian cells and cloned into a tethered plasmid.

SEQ ID NOs: 1426-1438 show the nucleotide sequences of MG151 family reverse transcriptases codon optimized for expression in mammalian cells and cloned into a tethered or untethered plasmid.

SEQ ID NOs: 1439-1444 show the nucleotide sequences of MG153 family reverse transcriptases codon optimized for expression in mammalian cells and cloned into a tethered or untethered plasmid.

SEQ ID NOs: 1445-1446 show the nucleotide sequences of MG160 family reverse transcriptases codon optimized for expression in mammalian cells and cloned into a tethered plasmid.

SEQ ID NOs: 1447 show the nucleotide sequence of an MG151 family reverse transcriptase codon optimized for expression in mammalian cells and cloned into a tethered or untethered plasmid.

SEQ ID NOs: 1448-1450 show the nucleotide sequences of MG71-2 scaffolds.

SEQ ID NOs: 1451-1462 show the nucleotide sequences of chemically modified guide RNAs (for targeting AAVS1) with a 5 nucleotide change with PBS of different lengths suitable for the gene editing systems described herein.

SEQ ID NOs: 1463-1470 show the nucleotide sequences of chemically modified guide RNAs (for targeting AAVS1) with a modified scaffold with PBS of different lengths suitable for the gene editing systems described herein.

SEQ ID NOs: 1471-1474 show the nucleotide sequences of chemically modified guide RNAs (for targeting AAVS1) with a 2 nucleotide change with PBS of different lengths suitable for the gene editing systems described herein.

SEQ ID NO: 1475 shows the nucleotide sequence of an mRNA encoding MG3-6 codon optimized for expression in mammalian cells.

SEQ ID NO: 1476 shows the nucleotide sequence of an mRNA encoding MG3-6/3-8 codon optimized for expression in mammalian cells.

SEQ ID NO: 1477 shows the nucleotide sequence of an mRNA encoding MG14-241 codon optimized for expression in mammalian cells.

SEQ ID NO: 1478 shows the nucleotide sequence of an mRNA encoding MG14-241 (H596A) codon optimized for expression in mammalian cells.

SEQ ID NOs: 1479-1492 show the nucleotide sequences of chemically modified guide RNAs (for targeting AAVS1) with a 5 nucleotide change with PBS of different lengths suitable for the gene editing systems described herein.

SEQ ID NOs: 1493-1504 show the nucleotide sequences of NGS primers.

SEQ ID NOs: 1505-1510 show the nucleotide sequences of cDNAs for endogenous targets.

SEQ ID NO: 1511 shows the nucleotide sequence of an engineered landing pad.

SEQ ID NOs: 1512-1516 show the nucleotide sequences of Cas9 guides targeting the engineered site.

SEQ ID NOs: 1518-1519 show the nucleotide sequences of primers.

SEQ ID NOs: 1520-1531 show nucleotide sequences encoding MG RT/Cas9 fusion proteins codon optimized for expression in mammalian systems.

SEQ ID NOs: 1532-1540 show the nucleotide sequences of RNA cargoes for integration.

SEQ ID NOs: 1541-1547 show the nucleotide sequences of primers.

SEQ ID NOs: 1548-1555 show the nucleotide sequences of RNA templates.

SEQ ID NOs: 1557-1560 show the nucleotide sequences of primers.

SEQ ID NOs: 1561-1562 show the nucleotide sequences of Taqman probes.

SEQ ID NO: 1563 shows the nucleotide sequence of an nMRA encoding MG71-2 codon optimized for expression in mammalian systems.

SEQ ID NO: 1564 shows the nucleotide sequence of an MG71-2 guide.

SEQ ID NOs: 1566-1567 show the nucleotide sequences of NGS primers.

SEQ ID NOs: 1568-1573 show the nucleotide sequences of MG71-2 guides.

SEQ ID NOs: 1574-1576 show the nucleotide sequences of MG71-2 pegRNAs.

SEQ ID NOs: 1577-1578 show the nucleotide sequences of NGS primers.

SEQ ID NO: 1579 shows the nucleotide sequence of a cDNA encoding an endogenous target.

SEQ ID NOs: 1580-1581 show the nucleotide sequences of NGS primers.

SEQ ID NO: 1582 shows the nucleotide sequence of a cDNA encoding an endogenous target.

SEQ ID NOs: 1583-1584 show the nucleotide sequences of NGS primers.

SEQ ID NO: 1585 shows the nucleotide sequence of a cDNA encoding an endogenous target.

SEQ ID NOs: 1586-1587 show the nucleotide sequences of NGS primers.

SEQ ID NO: 1588 shows the nucleotide sequence of a cDNA encoding an endogenous target.

SEQ ID NOs: 1589-1590 show the nucleotide sequences of NGS primers.

SEQ ID NO: 1591 shows the nucleotide sequence of a cDNA encoding an endogenous target.

SEQ ID NOs: 1592-1593 show the nucleotide sequences of reverse transcriptases codon optimized for expression in mammalian cells and cloned into a tethered plasmid.

SEQ ID NOs: 1596-1597 show the nucleotide sequence of reverse transcriptases codon optimized for expression in mammalian cells and cloned into a tethered or untethered plasmid.

SEQ ID NOs: 1598-1609 show the nucleotide sequences of MG71-2 pegRNAs.

SEQ ID NOs: 1610-1620 show the nucleotide sequences of MG71-2 guides.

SEQ ID NOs: 1621-1622 show the nucleotide sequences of NGS primers.

SEQ ID NO: 1623 shows the nucleotide sequence of a cDNA encoding an endogenous target.

SEQ ID NOs: 1624-1625 show the nucleotide sequences of NGS primers.

SEQ ID NO: 1626 shows the nucleotide sequence of a cDNA encoding an endogenous target.

SEQ ID NOs: 1627-1628 show the nucleotide sequences of NGS primers.

SEQ ID NO: 1629 shows the nucleotide sequence of a cDNA encoding an endogenous target.

SEQ ID NOs: 1630-1631 show the nucleotide sequences of NGS primers.

SEQ ID NO: 1632 shows the nucleotide sequence of a cDNA encoding an endogenous target.

SEQ ID NOs: 1633-1634 show the nucleotide sequences of NGS primers.

SEQ ID NO: 1635 shows the nucleotide sequence of a cDNA encoding an endogenous target.

SEQ ID NOs: 1636-1637 show the nucleotide sequences of NGS primers.

SEQ ID NO: 1638 shows the nucleotide sequence of a cDNA encoding an endogenous target.

SEQ ID NOs: 1639-1640 show the nucleotide sequences of NGS primers.

SEQ ID NO: 1641 shows the nucleotide sequence of a cDNA encoding an endogenous target.

SEQ ID NOs: 1642-1643 show the nucleotide sequences of NGS primers.

SEQ ID NO: 1644 shows the nucleotide sequence of a cDNA encoding an endogenous target.

SEQ ID NOs: 1645-1646 show the nucleotide sequences of NGS primers.

SEQ ID NO: 1647 shows the nucleotide sequence of a cDNA encoding an endogenous target.

SEQ ID NOs: 1648-1649 show the nucleotide sequences of NGS primers.

SEQ ID NO: 1650 shows the nucleotide sequence of a cDNA encoding an endogenous target.

SEQ ID NOs: 1651-1652 show the nucleotide sequences of NGS primers.

SEQ ID NO: 1653 shows the nucleotide sequence of a cDNA encoding an endogenous target.

SEQ ID NO: 1654 shows the nucleotide sequence of reverse transcriptases codon optimized for expression in mammalian cells and cloned into plasmids.

SEQ ID NOs: 1656-1681 show the nucleotide sequences of MG71-2 pegRNAs.

SEQ ID NO: 1682 shows the nucleotide sequence of a primer.

SEQ ID NOs: 1683-1690 show the nucleotide sequences of MG71-2 pegRNAs.

SEQ ID NOs: 1691-1720 show nucleotide sequences of reverse transcriptases codon optimized for expression in mammalian cells and cloned into plasmids.

SEQ ID NOs: 1722-1749 show the nucleotide sequences of MG3-6/3-8 guides.

SEQ ID NOs: 1750-1751 show the nucleotide sequences of NGS primers.

SEQ ID NO: 1752 shows the nucleotide sequence of a cDNA encoding an endogenous target.

SEQ ID NOs: 1753-1754 show the nucleotide sequences of reverse transcriptases codon optimized for expression in mammalian cells and cloned into plasmids.

SEQ ID NOs: 1755-1774 show the nucleotide sequences of MG3-6/3-8 pegRNAs.

SEQ ID NOs: 1776-1778 show the nucleotide sequences of reverse transcriptases codon optimized for expression in mammalian cells and cloned into plasmids.

SEQ ID NO: 1779 shows the nucleotide sequence of a target codon optimized for expression in mammalian cells.

SEQ ID NOs: 1780-1783 show the nucleotide sequences of reverse transcriptases codon optimized for expression in mammalian cells and cloned into plasmids.

SEQ ID NOs: 1784-1786 show the nucleotide sequences of MG3-6 pegRNAs.

SEQ ID NOs: 1787-1788 show the nucleotide sequences of NGS primers.

SEQ ID NO: 1789 shows the nucleotide sequence of a cDNA encoding an endogenous target.

SEQ ID NOs: 1790-1847 show the nucleotide sequences of reverse transcriptases codon optimized for expression in mammalian cells and cloned into plasmids.

SEQ ID NOs: 1848-1855 show the nucleotide sequences of MG71-2 pegRNAs.

SEQ ID NOs: 1856-1858 show the nucleotide sequences of reverse transcriptases codon optimized for expression in mammalian cells and cloned into plasmids.

SEQ ID NOs: 1859-1862 show the nucleotide sequences of plasmids encoding MG nickases codon optimized for expression in mammalian cells.

SEQ ID NOs: 1863-1910 show the nucleotide sequences of MG71-2 guide RNAs targeting AAVS1.

SEQ ID NOs: 1911-1958 show the DNA sequences of AAVS1 target sites.

SEQ ID NOs: 1959-2002 show the full-length peptide sequences of MG140 reverse transcriptase proteins.

SEQ ID NOs: 2003-2084 show the full-length peptide sequences of MG153 reverse transcriptase proteins.

SEQ ID NOs: 2085-2092 show the full-length peptide sequences of MG157 reverse transcriptase proteins.

SEQ ID NOs: 2093-2112 show the full-length peptide sequences of MG165 reverse transcriptase proteins.

SEQ ID NOs: 2113-2156 show the full-length peptide sequences of MG166 reverse transcriptase proteins.

SEQ ID NOs: 2157-2186 show the full-length peptide sequences of MG167 reverse transcriptase proteins.

SEQ ID NOs: 2187-2223 show the full-length peptide sequences of MG169 reverse transcriptase proteins.

SEQ ID NO: 2224 shows the full-length peptide sequence of an MG176 reverse transcriptase protein.

SEQ ID NOs: 2225-2252 show the full-length peptide sequences of MG198 reverse transcriptase proteins.

SEQ ID NOs: 2253-2256 show the full-length peptide sequences of MG173 reverse transcriptase proteins.

SEQ ID NOs: 2257-2289 show the full-length peptide sequences of MG140 reverse transcriptase proteins.

SEQ ID NOs: 2290-2471 and 2582-2585 show the full-length peptide sequences of MG160 reverse transcriptase proteins.

SEQ ID NOs: 2472-2517 show the full-length peptide sequences of MG140 retrotransposition proteins.

SEQ ID NOs: 2518-2520 show the full-length peptide sequences of MG160 retrotransposition proteins.

SEQ ID NO: 2522 shows the full-length peptide sequence of an MG153 reverse transcriptase protein.

SEQ ID NOs: 2523-2530 show the nucleotide sequences of MG140 UTRs.

SEQ ID NOs: 2531-2540 show the nucleotide sequences of MG153 RNAs.

SEQ ID NOs: 2541-2571 show the nucleotide sequences of MG140 UTRs.

### DETAILED DESCRIPTION

Site-directed gene editing systems are powerful tools for site-directed genome engineering in cells. Programmable nucleases such as Clustered Regularly Interspaced Short Palindromic Repeats (CRISPR) nucleases have been used recently for diverse DNA manipulation and gene editing applications. CRISPR nucleases can be used with or without a repair template to introduce site-directed insertions and deletions (indels) or varying length as well as point mutations. Single nucleotide point (SNP) mutations, deletions, and insertions represent over 80% of disease-causing mutations. However, not all of these mutations can be accurately repaired with the available gene editing systems. Clinical genome editing applications with a higher efficiency and fidelity of the system are needed.

Additionally, the repair or insertion of longer pieces of DNA has remained challenging, and a safe and efficient way of targeted integration of large templates into a genome, for example for gene therapies or engineered cell therapies, is lacking. To date, lentiviruses or adeno-associated viruses (AAV) in combination with a CRISPR nuclease are used to insert large pieces of DNA, for example whole genes. However, lentiviral-mediated integration lacks the targetability feature, as integration occurs mostly randomly in open chromatin. AAV-mediated delivery has a limited cargo capacity and is not available for all cell types. A safe and efficient targeted genome editing system that allows for large template integration is needed.

The present disclosure is based, in part, upon the development of a gene editing system comprising a reverse transcriptase, a nuclease or nickase, and a guide RNA or pegRNA. The gene editing system can be used to introduce site-directed insertions, deletions, and mutations in the genome of cells. Furthermore, it is contemplated that the gene editing system can be used in combination with a nucleic acid template to facilitate site-directed insertions into the genome of a cell, as well as for large template integration.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. The section headings used herein are for organizational purposes only and are not to be construed as limiting the subject matter described.

The practice of some methods disclosed herein employ, unless otherwise indicated, techniques of immunology, biochemistry, chemistry, molecular biology, microbiology, cell biology, genomics, and recombinant DNA. See for example Sambrook and Green, Molecular Cloning: A Laboratory Manual 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

The term “nucleotide,” as used herein, refers to a base-sugar-phosphate combination. Contemplated nucleotides include naturally occurring nucleotides and synthetic nucleotides. Nucleotides are monomeric units of a nucleic acid sequence (e.g., deoxyribonucleic acid (DNA) and ribonucleic acid (RNA)). The term nucleotide includes ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP, and nucleotide derivatives that confer nuclease resistance on the nucleic acid molecule containing them. The term nucleotide as used herein encompasses dideoxyribonucleoside triphosphates (ddNTPs) and their derivatives. Illustrative examples of ddNTPs include, but are not limited to, ddATP, ddCTP, ddGTP, ddITP, and ddTTP. A nucleotide may be unlabeled or detectably labeled, such as using moieties comprising optically detectable moieties (e.g., fluorophores) or quantum dots. Detectable labels include, for example, radioactive isotopes, fluorescent labels, chemiluminescent labels, bioluminescent labels, and enzyme labels. Fluorescent labels of nucleotides include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif, FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, IL; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. The term nucleotide encompasses chemically modified nucleotides. An exemplary chemically-modified nucleotide is biotin-dNTP. Non-limiting examples of biotinylated dNTPs include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. Contemplated polynucleotides include a gene or fragment thereof. Exemplary polynucleotides include, but are not limited to, DNA, RNA, coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, cell-free polynucleotides including cell-free DNA (cfDNA) and cell-free RNA (cfRNA), nucleic acid probes, and primers. In a polynucleotide when referring to a T, a T means U (Uracil) in RNA and T (Thymine) in DNA. A polynucleotide can be exogenous or endogenous to a cell and/or exist in a cell-free environment. The term polynucleotide encompasses modified polynucleotides (e.g., altered backbone, sugar, or nucleobase). If present, modifications to the nucleotide structure are imparted before or after assembly of the polymer. Non-limiting examples of modifications include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol-containing nucleotides, biotin-linked nucleotides, fluorescent base analogs, CpG islands, methyl-7-guanosine, methylated nucleotides, inosine, thiouridine, pseudouridine, dihydrouridine, queuosine, and wyosine. The sequence of nucleotides may be interrupted by non-nucleotide components.

The terms “transfection” or “transfected” refer to introduction of a polynucleotide into a cell by non-viral or viral-based methods. The polynucleotides may be gene sequences encoding complete proteins or functional portions thereof. See, e.g., Sambrook et al., 1989, Molecular Cloning: A Laboratory Manual, 18.1-18.88.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer is interrupted by non-amino acids. The terms include amino acid chains of any length, including full length proteins, and proteins with or without secondary or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, refer to natural and non-natural amino acids, including, but not limited to, modified amino acids. Modified amino acids include amino acids that have been chemically modified to include a group or a chemical moiety not naturally present on the amino acid. The term “amino acid” includes both D-amino acids and L-amino acids.

As used herein, the “non-native” refers to a nucleic acid or polypeptide sequence that is non-naturally occurring. Non-native refers to a non-naturally occurring nucleic acid or polypeptide sequence that comprises modifications such as mutations, insertions, or deletions. The term non-native encompasses fusion nucleic acids or polypeptides that encodes or exhibits an activity (e.g., enzymatic activity, methyltransferase activity, acetyltransferase activity, kinase activity, ubiquitinating activity, etc.) of the nucleic acid or polypeptide sequence to which the non-native sequence is fused. A non-native nucleic acid or polypeptide sequence includes those linked to a naturally-occurring nucleic acid or polypeptide sequence (or a variant thereof) by genetic engineering to generate a chimeric nucleic acid or polypeptide sequence encoding a chimeric nucleic acid or polypeptide.

The term “promoter”, as used herein, refers to the regulatory DNA region which controls transcription or expression of a polynucleotide (e.g., a gene) and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. Eukaryotic basal promoters typically, though not necessarily, contain a TATA-box and/or a CAAT box.

The term “expression”, as used herein, refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, the term expression includes splicing of the mRNA in a eukaryotic cell.

As used herein, “operably linked”, “operable linkage”, “operatively linked”, or grammatical equivalents thereof refer to an arrangement of genetic elements, e.g., a promoter, an enhancer, a polyadenylation sequence, etc., wherein an operation (e.g., movement or activation) of a first genetic element has some effect on the second genetic element. The effect on the second genetic element can be, but need not be, of the same type as operation of the first genetic element. For example, two genetic elements are operably linked if movement of the first element causes an activation of the second element. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, refers to a macromolecule or association of macromolecules that comprises or associates with a polynucleotide and which mediates delivery of the polynucleotide to a cell. Examples of vectors include nucleic-based vectors (e.g., plasmids and viral vectors) and liposomes. An exemplary nucleic-acid based vector comprises genetic elements, e.g., regulatory elements, operatively linked to a gene to facilitate expression of the gene in a target.

As used herein, “expression cassette” and “nucleic acid cassette” are used interchangeably to refer to a component of a vector comprising a combination of nucleic acid sequences or elements (e.g., therapeutic gene, promoter, and a terminator) that are expressed together or are operably linked for expression. The terms encompass an expression cassette including a combination of regulatory elements and a gene or genes to which they are operably linked for expression.

A “functional fragment” of a DNA or protein sequence refers to a fragment that retains a biological activity (either functional or structural) that is substantially similar to a biological activity of the full-length DNA or protein sequence. A biological activity of a DNA sequence includes its ability to influence expression in a manner attributed to the full-length sequence.

The terms “engineered,” “synthetic,” and “artificial” are used interchangeably herein to refer to an object that has been modified by human intervention. For example, the terms refer to a polynucleotide or polypeptide that is non-naturally occurring. An engineered peptide has, but does not require, low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains. Non-limiting examples include the following: a nucleic acid modified by changing its sequence to a sequence that does not occur in nature; a nucleic acid modified by ligating it to a nucleic acid that it does not associate with in nature such that the ligated product possesses a function not present in the original nucleic acid; an engineered nucleic acid synthesized in vitro with a sequence that does not exist in nature; a protein modified by changing its amino acid sequence to a sequence that does not exist in nature; an engineered protein acquiring a new function or property. An “engineered” system comprises at least one engineered component.

As used herein, a “guide nucleic acid” or “guide polynucleotide” refers to a nucleic acid that may hybridize to a target nucleic acid and thereby directs an associated nuclease to the target nucleic acid. A guide nucleic acid is, but is not limited to, RNA (guide RNA or gRNA), DNA, or a mixture of RNA and DNA. A guide nucleic acid can include a crRNA or a tracrRNA or a combination of both. The term guide nucleic acid encompasses an engineered guide nucleic acid and a programmable guide nucleic acid to specifically bind to the target nucleic acid. A portion of the target nucleic acid may be complementary to a portion of the guide nucleic acid. The strand of a double-stranded target polynucleotide that is complementary to and hybridizes with the guide nucleic acid is the complementary strand. The strand of the double-stranded target polynucleotide that is complementary to the complementary strand, and therefore is not complementary to the guide nucleic acid is called noncomplementary strand. A guide nucleic acid having a polynucleotide chain is a “single guide nucleic acid.” A guide nucleic acid having two polynucleotide chains is a “double guide nucleic acid.” If not otherwise specified, the term “guide nucleic acid” is inclusive, referring to both single guide nucleic acids and double guide nucleic acids. A guide nucleic acid may comprise a segment referred to as a “nucleic acid-targeting segment” or a “nucleic acid-targeting sequence,” or a “spacer.” A nucleic acid-targeting segment can include a sub-segment referred to as a “protein binding segment” or “protein binding sequence” or “Cas protein binding segment.”

The term “tracrRNA” or “tracr sequence” means trans-activating CRISPR RNA. tracrRNA interacts with the CRISPR (cr) RNA to form a guide nucleic acid (e.g., guide RNA or gRNA) that may hybridize to a target nucleic acid and thereby directs an associated nuclease to the target nucleic acid.

As used herein, the term “RuvC_III domain” refers to a third discontinuous segment of a RuvC endonuclease domain (the RuvC nuclease domain being comprised of three discontiguous segments, RuvC_I, RuvC_II, and RuvC_III). A RuvC domain or segments thereof can generally be identified by alignment to documented domain sequences, structural alignment to proteins with annotated domains, or by comparison to Hidden Markov Models (HMMs) built based on documented domain sequences (e.g., Pfam HMM PF18541 for RuvC_III).

As used herein, the term “HNH domain” refers to an endonuclease domain having characteristic histidine and asparagine residues. An HNH domain can generally be identified by alignment to documented domain sequences, structural alignment to proteins with annotated domains, or by comparison to Hidden Markov Models (HMMs) built based on documented domain sequences (e.g., Pfam HMM PF01844 for domain HNH).

As used herein, the term “transposon” refers to mobile elements that move in and out of genomes carrying “cargo DNA” with them. These transposons can differ on the type of nucleic acid to transpose, the type of repeat at the ends of the transposon, the type of cargo to be carried, or by the mode of transposition (i.e., self-repair or host-repair).

As used herein, the term “transposase” or “transposases” refers to an enzyme that binds to the end of a transposon and catalyzes its movement to another part of the genome. Types of movement include a cut and paste mechanism and a replicative transposition mechanism.

As used herein, the term “Tn7” or “Tn7-like transposase” refers to a family of transposases comprising three main components: a heteromeric transposase (TnsA and/or TnsB) alongside a regulator protein (TnsC). In addition to the TnsABC transposition proteins, Tn7 elements can encode dedicated target site-selection proteins, TnsD and TnsE. In conjunction with TnsABC, the sequence-specific DNA-binding protein TnsD directs transposition into a conserved site referred to as the “Tn7 attachment site,” attTn7. TnsD is a member of a large family of proteins that also includes TniQ. TniQ has been shown to target transposition into resolution sites of plasmids.

As used herein, the terms “gene editing” and “genome editing” can be used interchangeably. Gene editing or genome editing means to change the nucleic acid sequence of a gene or a genome. Genome editing can include, for example, insertions, deletions, and mutations. Genome editing can be performed by a gene editing system, for example a nuclease, a reverse transcriptase, a recombinase, or a base editor.

As used herein, the term “recombinase” refers to an enzyme that mediates the recombination of DNA fragments located between recombinase recognition sequences, which results in the excision, insertion, inversion, exchange or translocation) of the DNA fragments located between the recombinase recognition sequences.

As used herein, the term “recombine,” or “recombination,” in the context of a nucleic acid modification (e.g., a genomic modification), refers to the process by which two or more nucleic acid molecules, or two or more regions of a single nucleic acid molecule, are modified by the action of a recombinase protein. Recombination can result in, inter alia, the insertion, inversion, excision, or translocation of a nucleic acid sequence, e.g., in or between one or more nucleic acid molecules.

As used herein, the term “complex” refers to a joining of at least two components. The two components may each retain the properties/activities they had prior to forming the complex or gain properties as a result of forming the complex. The joining includes, but is not limited to, covalent bonding, non-covalent bonding (i.e., hydrogen bonding, ionic interactions, Van der Waals interactions, and hydrophobic bond), use of a linker, fusion, or any other suitable method. Contemplated components of the complex include polynucleotides, polypeptides, or combinations thereof. For example, a complex comprises an endonuclease and a guide polynucleotide.

The term “sequence identity” or “percent identity” in the context of two or more nucleic acids or polypeptide sequences, refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with the Smith-Waterman homology search algorithm parameters with a match of 2, a mismatch of −1, and a gap of −1; MUSCLE with default parameters; MAFFT with parameters of a retree of 2 and max iterations of 1000; Novafold with default parameters; HNMER hmmalign with default parameters.

The term “optimally aligned” in the context of two or more nucleic acids or polypeptide sequences, refers to two (e.g., in a pairwise alignment) or more (e.g., in a multiple sequence alignment) sequences that have been aligned to maximal correspondence of amino acids residues or nucleotides, for example, as determined by the alignment producing a highest or “optimized” percent identity score.

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of the reverse transcriptases protein sequences described herein (e.g., MG140, MG146, MG148, MG149, MG151, MG153, MG154, MG155, MG156, MG157, MG158, MG159, MG160, MG163, MG164, MG165, MG166, MG167, MG168, MG169, MG170, MG172, MG173, and MG176 family reverse transcriptases or retrotransposases described herein, or any other family reverse transcriptases or retrotransposases described herein). In some embodiments, such conservatively substituted variants are functional variants. Such functional variants can encompass sequences with substitutions such that the activity of one or more critical active site residues are not disrupted.

Also included in the current disclosure are variants of any of the enzymes described herein with substitution of one or more catalytic residues to decrease or eliminate activity of the enzyme (e.g., decreased-activity variants). In some embodiments, a decreased activity variant as a protein described herein comprises a disrupting substitution of at least one, at least two, or all three catalytic residues (for example a programmable nuclease MG3 family nickase with a D13A mutation, a H586A mutation, or a N609A mutation).

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T), and
8) Cysteine (C), Methionine (M)

### Gene Editing Systems

Described herein are gene editing systems, comprising: a) a nickase; b) a guide nucleic acid (e.g., pegRNA or other guide RNA) configured to form a complex with the nickase and to hybridize to a target nucleic acid sequence; and c) a reverse transcriptase having at least about 80% sequence identity to any one of SEQ ID NOs: 161-629, 767-1220, 1959-2522, and 2582-2585 and configured to form a complex with the nickase. Further described herein are gene editing systems, comprising: a) a nuclease; b) a guide nucleic acid (e.g., pegRNA or other guide RNA) configured to form a complex with the nuclease and to hybridize to a target nucleic acid sequence; and c) a reverse transcriptase having at least about 80% sequence identity to any one of SEQ ID NOs: 161-629, 767-1220, 1959-2522, and 2582-2585 and configured to form a complex with the nuclease. Further described herein are gene editing systems, comprising: a) a nickase; b) a guide nucleic acid (e.g., pegRNA) configured to form a complex with the nickase and to hybridize to a target nucleic acid sequence; and c) a reverse transcriptase configured to form a complex with the nickase, the reverse transcriptase having a X₁X₂DD motif, wherein X₁ is F or Y, and wherein when X₁ is Y, X₂ is A, R, N, D, C, E, Q, G, H, I, L, K, M, F, P, S, T, V, W, or Y. Further described herein are gene editing systems, comprising: a) a nuclease; b) a guide nucleic acid (e.g., pegRNA) configured to form a complex with the nuclease and to hybridize to a target nucleic acid sequence; and c) a reverse transcriptase configured to form a complex with the nuclease, the reverse transcriptase having a X₁X₂DD motif, wherein X₁ is F or Y, and wherein when X₁ is Y, X₂ is A, R, N, D, C, E, Q, G, H, I, L, K, M, F, P, S, T, V, W, or Y.

Gene editing systems as described herein, in some embodiments, comprising a nickase, a nuclease, a reverse transcriptase, or combinations thereof are capable of introduction of site-directed insertions, deletions, and mutations. In some embodiments, the nickase, the nuclease, the reverse transcriptase, or combinations thereof are capable of integration of polynucleotides of large sizes. In some embodiments, the integrated polynucleotide comprises a size of at least about 1 kilobase (kb), 2 kb, 3 kb, 4 kb, 5 kb, 6 kb, 7 kb, 8 kb, 9 kb, 10 kb, or more than 10 kb.

### Reverse Transcriptases

Reverse transcription is the translation of an RNA template into a complementary DNA. Reverse transcription is performed by enzymes termed reverse transcriptases (RT) that are enzymes with RNA-dependent DNA polymerase activity that create the complementary DNA (cDNA) strand from a RNA template. Some of the RT enzymes also have DNA-dependent DNA polymerase activity to create a double-stranded dsDNA. Reverse transcriptases can be of viral origin (for example HIV, hepatitis B, Moloney murine leukemia virus (MMLV), or avian myeloblastosis virus (AMV)) or bacterial origin (for example group II introns, retrons/retron-like RTs, diversity-generating retroelements (DGRs), Abi-like RTs, CRISPR-associated RTs, and group II-like RTs (G2L)). Reverse transcriptases of eukaryotic origin comprise the telomerase reverse transcriptase that maintains the telomeres of eukaryotic chromosomes. Reverse transcription allows the introduction of site-directed insertions, deletions, and mutations into the cDNA by encoding them in the RNA template.

In some embodiments, the reverse transcriptase is a viral, prokaryotic, or eukaryotic reverse transcriptase. In some embodiments, the reverse transcriptase comprises a sequence of SEQ ID NOs: 161-629, 767-1220, 1959-2522, and 2582-2585, a variant thereof, or a functional fragment thereof. In some embodiments, the reverse transcriptase comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of SEQ ID NOs: 161-629, 767-1220, 1959-2522, and 2582-2585, a variant thereof, or a functional fragment thereof. In some embodiments, the reverse transcriptase comprises a sequence having at least about 70% identity to any one of SEQ ID NOs: 161-629, 767-1220, 1959-2522, and 2582-2585. In some embodiments, the reverse transcriptase comprises a sequence having at least about 75% identity to any one of SEQ ID NOs: 161-629, 767-1220, 1959-2522, and 2582-2585. In some embodiments, the reverse transcriptase comprises a sequence having at least about 80% identity to any one of SEQ ID NOs: 161-629, 767-1220, 1959-2522, and 2582-2585. In some embodiments, the reverse transcriptase comprises a sequence having at least about 85% identity to any one of SEQ ID NOs: 161-629, 767-1220, 1959-2522, and 2582-2585. In some embodiments, the reverse transcriptase comprises a sequence having at least about 90% identity to any one of SEQ ID NOs: 161-629, 767-1220, 1959-2522, and 2582-2585. In some embodiments, the reverse transcriptase comprises a sequence having at least about 95% identity to any one of SEQ ID NOs: 161-629, 767-1220, 1959-2522, and 2582-2585. In some embodiments, the reverse transcriptase comprises a sequence having at least about 96% identity to any one of SEQ ID NOs: 161-629, 767-1220, 1959-2522, and 2582-2585. In some embodiments, the reverse transcriptase comprises a sequence having at least about 97% identity to any one of SEQ ID NOs: 161-629, 767-1220, 1959-2522, and 2582-2585. In some embodiments, the reverse transcriptase comprises a sequence having at least about 98% identity to any one of SEQ ID NOs: 161-629, 767-1220, 1959-2522, and 2582-2585. In some embodiments, the reverse transcriptase comprises a sequence having at least about 99% identity to any one of SEQ ID NOs: 161-629, 767-1220, 1959-2522, and 2582-2585. In some embodiments, the reverse transcriptase comprises a sequence having 100% identity to any one of SEQ ID NOs: 161-629, 767-1220, 1959-2522, and 2582-2585.

In some embodiments, the reverse transcriptase is a MG151, MG153, or MG160 family reverse transcriptase. In some embodiments, the reverse transcriptase is a MG140, MG146, MG148, MG149, MG151, MG153, MG154, MG155, MG156, MG157, MG158, MG159, MG160, MG163, MG164, MG165, MG166, MG167, MG168, MG169, MG170, or MG176 family reverse transcriptase. In some embodiments, the reverse transcriptase comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of the MG140, MG146, MG148, MG149, MG151, MG153, MG154, MG155, MG156, MG157, MG158, MG159, MG160, MG163, MG164, MG165, MG166, MG167, MG168, MG169, MG170, MG172, MG173, or MG176 family reverse transcriptase or retrotransposase. In some embodiments, the reverse transcriptase comprises a sequence with at least 80% sequence identity to any one of MG140, MG146, MG148, MG149, MG151, MG153, MG154, MG155, MG156, MG157, MG158, MG159, MG160, MG163, MG164, MG165, MG166, MG167, MG168, MG169, MG170, MG172, MG173, or MG176 family reverse transcriptase or retrotransposase or a variant thereof.

In some embodiments, the reverse transcriptase is encoded by a nucleic acid sequence having at least 80% sequence identity with the nucleic acid sequence of any one of SEQ ID NOs: 1-75, 702-766, 1221-1243, 1299, 1249-1295, 1300-1304, 1309, 1394-1447, 1592-1593, 1596-1597, 1654, 1691-1720, 1753-1754, 1776-1778, 1780-1783, 1790-1847, and 1856-1858. In some embodiments, the reverse transcriptase is encoded by a nucleic acid sequence having at least 85% sequence identity with the nucleic acid sequence of any one of SEQ ID NOs: 1-75, 702-766, 1221-1243, 1299, 1249-1295, 1300-1304, 1309, 1394-1447, 1592-1593, 1596-1597, 1654, 1691-1720, 1753-1754, 1776-1778, 1780-1783, 1790-1847, and 1856-1858. In some embodiments, the reverse transcriptase is encoded by a nucleic acid sequence having at least 90% sequence identity with the nucleic acid sequence of any one of SEQ ID NOs: 1-75, 702-766, 1221-1243, 1299, 1249-1295, 1300-1304, 1309, 1394-1447, 1592-1593, 1596-1597, 1654, 1691-1720, 1753-1754, 1776-1778, 1780-1783, 1790-1847, and 1856-1858. In some embodiments, the reverse transcriptase is encoded by a nucleic acid sequence having at least 95% sequence identity with the nucleic acid sequence of any one of SEQ ID NOs: 1-75, 702-766, 1221-1243, 1299, 1249-1295, 1300-1304, 1309, 1394-1447, 1592-1593, 1596-1597, 1654, 1691-1720, 1753-1754, 1776-1778, 1780-1783, 1790-1847, and 1856-1858. In some embodiments, the reverse transcriptase is encoded by a nucleic acid sequence having at least 96% sequence identity with the nucleic acid sequence of any one of SEQ ID NOs: 1-75, 702-766, 1221-1243, 1299, 1249-1295, 1300-1304, 1309, 1394-1447, 1592-1593, 1596-1597, 1654, 1691-1720, 1753-1754, 1776-1778, 1780-1783, 1790-1847, and 1856-1858. In some embodiments, the reverse transcriptase is encoded by a nucleic acid sequence having at least 97% sequence identity with the nucleic acid sequence of any one of SEQ ID NOs: 1-75, 702-766, 1221-1243, 1299, 1249-1295, 1300-1304, 1309, 1394-1447, 1592-1593, 1596-1597, 1654, 1691-1720, 1753-1754, 1776-1778, 1780-1783, 1790-1847, and 1856-1858. In some embodiments, the reverse transcriptase is encoded by a nucleic acid sequence having at least 98% sequence identity with the nucleic acid sequence of any one of SEQ ID NOs: 1-75, 702-766, 1221-1243, 1299, 1249-1295, 1300-1304, 1309, 1394-1447, 1592-1593, 1596-1597, 1654, 1691-1720, 1753-1754, 1776-1778, 1780-1783, 1790-1847, and 1856-1858. In some embodiments, the reverse transcriptase is encoded by a nucleic acid sequence having at least 99% sequence identity with the nucleic acid sequence of any one of SEQ ID NOs: 1-75, 702-766, 1221-1243, 1299, 1249-1295, 1300-1304, 1309, 1394-1447, 1592-1593, 1596-1597, 1654, 1691-1720, 1753-1754, 1776-1778, 1780-1783, 1790-1847, and 1856-1858. In some embodiments, the reverse transcriptase is encoded by a nucleic acid sequence of any one of SEQ ID NOs: 1-75, 702-766, 1221-1243, 1299, 1249-1295, 1300-1304, 1309, 1394-1447, 1592-1593, 1596-1597, 1654, 1691-1720, 1753-1754, 1776-1778, 1780-1783, 1790-1847, and 1856-1858.

Reverse transcriptases typically have an active site core tetrad motif of the amino acid sequence XXDD. In some embodiments, the reverse transcriptase has an active site tetrad motif of X₁X₂DD wherein X₁ is F or Y, and wherein when X₁ is Y, X₂ is A, R, N, D, C, E, Q, G, H, I, L, K, M, F, P, S, T, V, W, or Y. In some embodiments, X₂ is A or I. In some embodiments, the X₁X₂DD motif is YADD (SEQ ID NO: 2572) or YIDD (SEQ ID NO: 2573). In some embodiments, the X₁X₂DD motif is FADD (SEQ ID NO: 2574), FVDD (SEQ ID NO: 2575), FIDD (SEQ ID NO: 2576), or FLDD (SEQ ID NO: 2577). In some embodiments, the reverse transcriptase is isolated. In some embodiments, the reverse transcriptase is a MG140, MG146, MG148, MG149, MG151, MG153, MG154, MG155, MG156, MG157, MG158, MG159, MG160, MG163, MG164, MG165, MG166, MG167, MG168, MG169, MG170, MG172, MG173, or MG176 family reverse transcriptase or retrotransposase and the X₁X₂DD motif is YADD (SEQ ID NO: 2572) or YIDD (SEQ ID NO: 2573). In some embodiments, the reverse transcriptase is isolated. In some embodiments, the reverse transcriptase is a MG140, MG146, MG148, MG149, MG151, MG153, MG154, MG155, MG156, MG157, MG158, MG159, MG160, MG163, MG164, MG165, MG166, MG167, MG168, MG169, MG170, MG172, MG173, or MG176 family reverse transcriptase or retrotransposase and the X₁X₂DD motif is FADD (SEQ ID NO: 2574), FVDD (SEQ ID NO: 2575), FIDD (SEQ ID NO: 2576), or FLDD (SEQ ID NO: 2577).

In some embodiments, the reverse transcriptase is smaller than 300 amino acids. In some embodiments, the reverse transcriptase is smaller than 250 amino acids. In some embodiments, the reverse transcriptase comprises at least about 50, 75, 100, 125, 150, 175, 200, 225, 250, 275, 300, or more than 300 amino acids. In some embodiments, the reverse transcriptase comprises a range of about 50 to about 300, about 75 to about 300, about 100 to about 300, about 125 to about 300, about 150 to about 300, about 175 to about 300, about 200 to about 300, about 225 to about 300, about 250 to about 300, about 275 to about 300, about 100 to about 300, about 125 to about 300, about 150 to about 300, about 175 to about 300, about 200 to about 300, about 225 to about 300, about 250 to about 300, or about 275 to about 300 amino acids.

In some embodiments, the reverse transcriptase comprises a processivity of at least about 2-fold more than Moloney Murine Leukemia Virus (MMLV) reverse transcriptase. In some embodiments, the reverse transcriptase comprises a processivity of at least about 2-fold less than Moloney Murine Leukemia Virus (MMLV) reverse transcriptase. In some embodiments, the reverse transcriptase comprises an error rate of less than about 2.5%, 2.0%, 1.5%, 1%, 0.5%, 0.25%, 0.10%, or 0.05%. In some embodiments, the reverse transcriptase comprises an error rate of less than about 2.5%, 2.0%, 1.5%, 1%, 0.5%, 0.25%, 0.10%, or 0.05% as compared to Moloney Murine Leukemia Virus (MMLV) reverse transcriptase. Methods to measure reverse transcriptase processivity are known in the art or are described herein, for example in Example 2.

In some embodiments, the reverse transcriptase is targetable. Targetable reverse transcriptases are engineered ribonucleoprotein complexes that act as tools for genome editing in cells and organisms. In some embodiments, targetable reverse transcriptases are created by fusing a reverse transcriptase and a site-directed CRISPR nuclease variant that nicks the non-targeting strand of dsDNA, such that a guide RNA or pegRNA comprising a primer binding site (PBS) sequence can find and hybridize with its complementary target sequence to prime the reverse transcriptase reaction using a reverse transcriptase template (RTT) as the template. Two DNA flaps are produced, one containing the desired change encoded in the RTT, and the other with the original sequence; post-equilibration, the change is incorporated into the genomic DNA when the DNA flap with the desired edit is repaired by the cellular host repair machinery.

In some embodiments, the gene editing system comprises a reverse transcriptase described herein and a nickase. In some embodiments, the gene editing system comprises a reverse transcriptase described herein and a nuclease. In some embodiments, the gene editing system comprises a reverse transcriptase described herein and a modified nuclease. In some embodiments, the gene editing system is programmable. In some embodiments, the modified nuclease is a site-directed nickase.

In some embodiments, the reverse transcriptase and the nuclease or nickase are linked or tethered. In some embodiments, the gene editing system comprises a fusion protein of a reverse transcriptase and a nuclease or nickase. In some embodiments, the gene editing system comprises a fusion protein comprising a nickase linked to a reverse transcriptase using a linker, wherein the reverse transcriptase comprises at least about 80% sequence identity to any one of SEQ ID NOs: 161-629, 767-1220, 1959-2522, and 2582-2585. In some embodiments, the gene editing system comprises a fusion protein comprising a nuclease linked to a reverse transcriptase using a linker, wherein the reverse transcriptase comprises at least about 80% sequence identity to any one of SEQ ID NOs: 161-629, 767-1220, 1959-2522, and 2582-2585. In some embodiments, the gene editing system comprises a fusion protein comprising a catalytically dead nuclease linked to a reverse transcriptase using a linker, wherein the reverse transcriptase comprises at least about 80% sequence identity to any one of SEQ ID NOs: 161-629, 767-1220, 1959-2522, and 2582-2585.

In some embodiments, the reverse transcriptase and the nuclease or nickase is linked or fused using a linker. In some embodiments, the linker comprises at least 10, 20, or 30 amino acids. In some embodiments, the linker comprises about 30-35 amino acids. In some embodiments, the linker comprises about 30 amino acids.

In some embodiments, the linker comprises at least 80% sequence identity to SEQ ID NO: 103. In some embodiments, the linker comprises at least 80% sequence identity to SEQ ID NO: 103. In some embodiments, the linker comprises a sequence having at least about 85% identity to SEQ ID NO: 103. In some embodiments, the linker comprises a sequence having at least about 90% identity to SEQ ID NO: 103. In some embodiments, the linker comprises a sequence having at least about 91% identity to SEQ ID NO: 103. In some embodiments, the linker comprises a sequence having at least about 92% identity to SEQ ID NO: 103. In some embodiments, the linker comprises a sequence having at least about 93% identity to SEQ ID NO: 103. In some embodiments, the linker comprises a sequence having at least about 94% identity to SEQ ID NO: 103. In some embodiments, the linker comprises a sequence having at least about 95% identity to SEQ ID NO: 103. In some embodiments, the linker comprises a sequence having at least about 96% identity to SEQ ID NO: 103. In some embodiments, the linker comprises a sequence having at least about 97% identity to SEQ ID NO: 103. In some embodiments, the linker comprises a sequence having at least about 98% identity to SEQ ID NO: 103. In some embodiments, the linker comprises a sequence having at least about 99% identity to SEQ ID NO: 103. In some embodiments, the linker comprises a sequence having 100% identity to SEQ ID NO: 103.

Suitable linkers are known in the art and comprise, for example, any one of SEQ ID NOs: 155-160. In some embodiments, the linker comprises at least 80% sequence identity to any one of SEQ ID NOs: 155-160. In some embodiments, linkers joining any of the enzymes or domains described herein comprise one or multiple copies of a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SGGSSGGSSGSETPGTSESATPESSGGSSGGSSAC (SEQ ID NO: 155), KLGGGAPAVGGGPK(SEQ ID NO: 156), (GGGGS)3(SEQ ID NO: 157), (GGGGS)2EAAAK(GGGGS)2 (SEQ ID NO: 158), (GGGGS)2(EAAAK)2(GGGGS)2 (SEQ ID NO: 159), or SGSETPGTSESATPES (SEQ ID NO: 160), or any other linker sequence described herein. In some embodiments, the linker comprises a sequence having at least about 85% identity to any one of SEQ ID NOs: 155-160. In some embodiments, the linker comprises a sequence having at least about 90% identity to any one of SEQ ID NOs: 155-160. In some embodiments, the linker comprises a sequence having at least about 91% identity to any one of SEQ ID NOs: 155-160. In some embodiments, the linker comprises a sequence having at least about 92% identity to any one of SEQ ID NOs: 155-160. In some embodiments, the linker comprises a sequence having at least about 93% identity to any one of SEQ ID NOs: 155-160. In some embodiments, the linker comprises a sequence having at least about 94% identity to any one of SEQ ID NOs: 155-160. In some embodiments, the linker comprises a sequence having at least about 95% identity to any one of SEQ ID NOs: 155-160. In some embodiments, the linker comprises a sequence having at least about 96% identity to any one of SEQ ID NOs: 155-160. In some embodiments, the linker comprises a sequence having at least about 97% identity to any one of SEQ ID NOs: 155-160. In some embodiments, the linker comprises a sequence having at least about 98% identity to any one of SEQ ID NOs: 155-160. In some embodiments, the linker comprises a sequence having at least about 99% identity to any one of SEQ ID NOs: 155-160. In some embodiments, the linker comprises a sequence having 100% identity to any one of SEQ ID NOs: 155-160.

In some embodiments, the nickase or nuclease and the reverse transcriptase are not linked.

In some embodiments, the reverse transcriptase, nuclease, nickase, or fusion protein described herein comprises one or more nuclear localization sequences (NLSs) proximal to an N- or C-terminus of the reverse transcriptase, nuclease, nickase, or fusion protein.

In some embodiments, the NLS comprises any of the sequences in Table 1 below, or a combination thereof:

TABLE 1
Example NLS Sequences

| | | SEQ |
|---|---|---|
| | NLS amino acid | ID |
| Source | sequence | NO: |
| SV40 | PKKKRKV | 630 |
| nucleoplasmin | KRPAATKKAGQAKKKK | 631 |
| bipartite NLS | | |
| c-myc NLS | PAAKRVKLD | 632 |
| c-myc NLS | RQRRNELKRSP | 633 |
| hRNPA1 M9 NLS | NQSSNFGPMKGGNFGGR | 634 |
| | SSGPYGGGGQYFAKPRN | |
| | QGGY | |
| Importin-alpha | RMRIZFKNKGKDTAELR | 635 |
| IBB domain | RRRVEVSVELRKAKKDE | |
| | QILKRRNV | |
| Myoma T protein | VSRKRPRP | 636 |
| Myoma T protein | PPKKARED | 637 |
| p53 | PQPKKKPL | 638 |
| mouse c-abl IV | SALIKKKKKMAP | 639 |
| influenza virus NS1 | DRLRR | 640 |
| influenza virus NS1 | PKQKKRK | 641 |
| Hepatitis virus | RKLKKKIKKL | 642 |
| delta antigen | | |
| mouse Mx1 protein | REKKKFLKRR | 643 |
| human poly(ADP-ribose) | KRKGDEVDGVDEVAKK | 644 |
| polymerase | KSKK | |
| steroid hormone receptor | RKCLQAGMNLEARKTKK | 645 |
| (human) glucocorticoid | | |

In some embodiments, the reverse transcriptase comprises a tag. In some embodiments, the nuclease comprises a tag. In some embodiments, the nickase comprises a tag. In some embodiments, the fusion protein comprises a tag. In some embodiments, the tag is an affinity tag. Exemplary affinity tags include, but are not limited to, His-tag, a Flag tag, a Myc-tag, an MBP-tag, and a GST-tag.

In some embodiments, the reverse transcriptase comprises a protease cleavage site. In some embodiments, the nuclease comprises a protease cleavage site. In some embodiments, the nickase comprises a protease cleavage site. In some embodiments, the fusion protein comprises a protease cleavage site. Exemplary protease cleavage sites include, but are not limited to, a TEV site, a C3 site, a Factor Xa site, and an Enterokinase site.

In some embodiments, the gene editing system comprises a) a nickase; b) a guide nucleic acid (e.g., pegRNA or other guide RNA); and c) a reverse transcriptase having at least about 80% sequence identity to any one of SEQ ID NOs: 161-629, 767-1220, 1959-2522, and 2582-2585.

In some embodiments, the gene editing system comprises a) a nuclease; b) a guide nucleic acid (e.g., pegRNA or other guide RNA); and c) a reverse transcriptase having at least about 80% sequence identity to any one of SEQ ID NOs: 161-629, 767-1220, 1959-2522, and 2582-2585.

In some embodiments, the gene editing system comprises a) a nickase b) a guide nucleic acid (e.g., pegRNA); and c) a reverse transcriptase having a X₁X₂DD motif, wherein X₁ is F or Y, and wherein when X₁ is Y, X₂ is A, R, N, D, C, E, Q, G, H, I, L, K, M, F, P, S, T, V, W, or Y.

In some embodiments, the gene editing system comprises a) a nuclease; b) a guide nucleic acid (e.g., pegRNA); and c) a reverse transcriptase having a X₁X₂DD motif, wherein X₁ is F or Y, and wherein when X₁ is Y, X₂ is A, R, N, D, C, E, Q, G, H, I, L, K, M, F, P, S, T, V, W, or Y. In some embodiments, the X₂ is A or I. In some embodiments, the X₁X₂DD motif is YADD (SEQ ID NO: 2572) or YIDD (SEQ ID NO: 2573). In some embodiments, the X₁X₂DD motif is FADD (SEQ ID NO: 2574), FVDD (SEQ ID NO: 2575), FIDD (SEQ ID NO: 2576), or FLDD (SEQ ID NO: 2577). In some embodiments, the reverse transcriptase has at least about 80% sequence identity to any one of SEQ ID NOs: 161-629, 767-1220, 1959-2522, and 2582-2585.

In some embodiments, the nuclease is configured to cleave one strand of a double-stranded target deoxyribonucleic acid (nickase). In some embodiment, the nickase or nuclease is a CRISPR nuclease described herein. In some embodiments, the nickase or nuclease is encoded by a nucleic acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of SEQ ID NOs: 104 and 1859-1862 or a variant thereof. In some embodiments, the nickase or nuclease is encoded by a nucleic acid sequence having at least about 70% identity to any one of SEQ ID NOs: 104 and 1859-1862. In some embodiments, the nickase or nuclease is encoded by a nucleic acid sequence having at least about 75% identity to any one of SEQ ID NOs: 104 and 1859-1862. In some embodiments, the nickase or nuclease is encoded by a nucleic acid sequence having at least about 80% identity to any one of SEQ ID NOs: 104 and 1859-1862. In some embodiments, the nickase or nuclease is encoded by a nucleic acid sequence having at least about 85% identity to any one of SEQ ID NOs: 104 and 1859-1862. In some embodiments, the nickase or nuclease is encoded by a nucleic acid sequence having at least about 90% identity to any one of SEQ ID NOs: 104 and 1859-1862. In some embodiments, the nickase or nuclease is encoded by a nucleic acid sequence having at least about 95% identity to any one of SEQ ID NOs: 104 and 1859-1862. In some embodiments, the nickase or nuclease is encoded by a nucleic acid sequence having at least about 96% identity to any one of SEQ ID NOs: 104 and 1859-1862. In some embodiments, the nickase or nuclease is encoded by a nucleic acid sequence having at least about 97% identity to any one of SEQ ID NOs: 104 and 1859-1862. In some embodiments, the nickase or nuclease is encoded by a nucleic acid sequence having at least about 98% identity to any one of SEQ ID NOs: 104 and 1859-1862. In some embodiments, the nickase or nuclease is encoded by a nucleic acid sequence having at least about 99% identity to any one of SEQ ID NOs: 104 and 1859-1862. In some embodiments, the nickase or nuclease is encoded by a nucleic acid sequence having 100% identity to any one of SEQ ID NOs: 104 and 1859-1862.

In some embodiments, the system further comprises a source of Mg²⁺.

In some embodiments, the nuclease is a modified endonuclease. In some embodiments, the modified endonuclease is a Type II CRISPR endonuclease or a Type V CRISPR endonuclease. In some embodiments, the Type II or Type V CRISPR endonuclease comprises double-stranded cutting activity, nickase activity, or can be catalytically dead. In some embodiments, the CRISPR nuclease has a modification in the HNH domain or in the RuvC domain.

In some embodiments, the modified endonuclease comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NOs: 152-154 or a variant thereof. In some embodiments, the modified endonuclease comprises at least about 80% sequence identity to any one of SEQ ID NOs: 152-154. In some embodiments, the modified endonuclease comprises a sequence having at least about 70% identity to any one of SEQ ID NOs: 152-154. In some embodiments, the modified endonuclease comprises a sequence having at least about 75% identity to any one of SEQ ID NOs: 152-154. In some embodiments, the modified endonuclease comprises a sequence having at least about 80% identity to any one of SEQ ID NOs: 152-154. In some embodiments, the modified endonuclease comprises a sequence having at least about 85% identity to any one of SEQ ID NOs: 152-154. In some embodiments, the modified endonuclease comprises a sequence having at least about 90% identity to any one of SEQ ID NOs: 152-154. In some embodiments, the modified endonuclease comprises a sequence having at least about 95% identity to any one of SEQ ID NOs: 152-154. In some embodiments, the modified endonuclease comprises a sequence having at least about 96% identity to any one of SEQ ID NOs: 152-154. In some embodiments, the modified endonuclease comprises a sequence having at least about 97% identity to any one of SEQ ID NOs: 152-154. In some embodiments, the modified endonuclease comprises a sequence having at least about 98% identity to any one of SEQ ID NOs: 152-154. In some embodiments, the modified endonuclease comprises a sequence having at least about 99% identity to any one of SEQ ID NOs: 152-154. In some embodiments, the modified endonuclease comprises a sequence having 100% identity to any one of SEQ ID NOs: 152-154.

In some embodiments, the modified endonuclease is selected from the group consisting of: spCas9 (H840A), spCas9 (D10A), nMG3-6 (D13A), nMG3-6 (H586A), nMG3-6 (N609A), Cas12a, and MG29-1.

In some embodiments, the gene editing system comprises a nucleic acid template. The nucleic acid template can be an RNA or a DNA. The nucleic acid template can be 10, 20, 30, 40, 50, 60, 70, 80, 90, 100, 110, 120, 130, 140, 150, 160, 170, 180, 190, or 200 bases long. The nucleic acid template can be 100, 200, 300, 400, 500, 600, 700, 800, 900, or 1000 bases long. In some embodiments, the nucleic acid template has a homology region that is homologous to a site in the genome. In some embodiments, the homology region is 10, 20, 30, 40, 50, 60, 70, 80, 90, 100, 110, 120, 130, 140, 150, 160, 170, 180, 190, or 200 bases long.

In some embodiments, the gene editing system further comprises a transposase, an integrase, or a homing endonuclease. In some embodiments, the transposase is transposase (Tnp) Tn5, Sleeping Beauty transposase, or a Tn7 transposon. In some embodiments, the gene editing system comprises an enzyme with transposase activity. Additional enzymes with transposase activity include, but are not limited to, retrons and IS200/IS605 transposons.

In some embodiments, the gene editing system further comprises a retrotransposon of the disclosure. In some embodiments, the retrotransposon is a MG140, MG146, or a MG176 family retrotransposon. In some embodiments, the retrotransposon comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NOs: 161-629, 767-1220, 1959-2522, and 2582-2585, or a variant thereof.

### CRISPR Nucleases

Described herein, in some embodiments, are nickases or endonucleases, wherein the nickase or endonuclease is a CRISPR nuclease. In some embodiments, the CRISPR nuclease is a modified nuclease.

CRISPR systems are RNA-directed nuclease complexes that have been described to function as an adaptive immune system in microbes. In their natural context, CRISPR systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the nuclease polypeptide directed by the RNA-based targeting element alongside accessory proteins/enzymes. Efficient nuclease targeting of a particular target nucleic acid sequence generally requires both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR systems are commonly organized into 2 classes, 5 types, and 16 subtypes based on shared functional characteristics and evolutionary similarity.

Class 1 CRISPR systems have large, multi-subunit effector complexes, and comprise Types I, III, and IV. Class 2 CRISPR systems generally have single-polypeptide multidomain nuclease effectors, and comprise Types II, V, and VI.

Type II CRISPR systems are considered the simplest in terms of components. In Type II CRISPR systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g., Cas9) and the repeat sequence to form a precursor dsRNA structure, which is cleaved by endogenous RNAse III to generate a mature effector enzyme loaded with both tracrRNA and crRNA. Type II nucleases are known as DNA nucleases. Type II nucleases generally exhibit a structure consisting of a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand. Exemplary CRISPR Cas9 proteins include, but are not limited to, Cas9 from Streptococcus pyogene—(UniProtKB—Q99ZW2 (CAS9 STRP1)), Streptococcus thermophilu—(UniProtKB—G3ECR1 (CAS9 STRTR)), Staphylococcus aureu—(UniProtKB—J7RUA5 (CAS9 STAAU), Campylobacter jejun—(UniProtKB—Q0P897 (CAS9 CAMJE)), Campylobacter lar—(UniProtKB—A0A0A8HTA3 (A0A0A8HTA3 CAMLA), and Helicobacter canadensi—(UniProtKB—C5ZYI3 (C5ZYI3 9HELI)), Francisella tularensis subsp. Novicid—(UniProtKB—A0Q5Y3 (CAS9_FRATN). Additional Type II nucleases are described in International Patent Application Publication WO 2021/226363, WO 2022/159758, and WO 2022/056324.

Type V CRISPR systems are characterized by a nuclease effector (e.g., Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, Type V systems are capable of using the effector nuclease itself to cleave pre-crRNAs. Like Type II CRISPR systems, Type V CRISPR systems are known as DNA nucleases. Unlike Type II CRISPR systems, some Type V enzymes (e.g., Cas12a) appear to have a robust single-stranded nonspecific deoxyribonuclease activity that is activated by the first crRNA-directed cleavage of a double-stranded target sequence.

In some embodiments, the nuclease or nickase is a CRISPR nuclease. In some embodiments, the CRISPR nuclease is a Class 2 Type II SpCas9 or a Class 2 Type V-A Cas12a (previously Cpf1). In some embodiments, the Type V-A nuclease has a guide RNA of 42-44 nucleotides compared with approximately 100 nt for SpCas9. In some embodiments, the Type V-A nuclease results in staggered cut sites. In some embodiments, the Type V-A nuclease results in staggered cut sites to facilitate directed repair pathways, such as microhomology-dependent targeted integration (MITI).

The most commonly used Type V-A enzymes require a 5′ protospacer adjacent motif (PAM) next to the chosen target site. 5′-TTTV-3′ for Lachnospiraceae bacterium ND2006 LbCas12a and Acidaminococcus sp. AsCas12a; and 5′-TTV-3′ for Francisella novicida FnCas12a. In some embodiments the PAM sequence is YTV, YYN, or TTN. Additional Type II nucleases are described in International Patent Application Publication WO 2021/226363.

In some embodiments, the nickase is a modified nuclease. In some embodiments, the modified endonuclease is a Type II CRISPR endonuclease. In some embodiments, the modified endonuclease is a Type II CRISPR endonuclease or a Type V endonuclease. In some embodiments, the Type II CRISPR endonuclease or the Type V endonuclease has nickase activity.

In some embodiments, the modified endonuclease is selected from the group consisting of: spCas9 (H840A), spCas9 (D10A), nMG3-6 (D13A), nMG3-6 (H586A), nMG3-6 (N609A), Cas12a, and MG29-1. In some embodiments, the modified endonuclease comprises at least about 80% sequence identity to any one of SEQ ID NOs: 152-154. In some embodiments, the nuclease comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of SEQ ID NOs: 152-154 or a variant thereof. In some embodiments, the modified endonuclease comprises a sequence having at least about 70% identity to any one of SEQ ID NOs: 152-154. In some embodiments, the modified endonuclease comprises a sequence having at least about 75% identity to any one of SEQ ID NOs: 152-154. In some embodiments, the modified endonuclease comprises a sequence having at least about 80% identity to any one of SEQ ID NOs: 152-154. In some embodiments, the modified endonuclease comprises a sequence having at least about 85% identity to any one of SEQ ID NOs: 152-154. In some embodiments, the modified endonuclease comprises a sequence having at least about 90% identity to any one of SEQ ID NOs: 152-154. In some embodiments, the modified endonuclease comprises a sequence having at least about 95% identity to any one of SEQ ID NOs: 152-154. In some embodiments, the modified endonuclease comprises a sequence having at least about 96% identity to any one of SEQ ID NOs: 152-154. In some embodiments, the modified endonuclease comprises a sequence having at least about 97% identity to any one of SEQ ID NOs: 152-154. In some embodiments, the modified endonuclease comprises a sequence having at least about 98% identity to any one of SEQ ID NOs: 152-154. In some embodiments, the modified endonuclease comprises a sequence having at least about 99% identity to any one of SEQ ID NOs: 152-154. In some embodiments, the modified endonuclease comprises a sequence having 100% identity to any one of SEQ ID NOs: 152-154.

In some embodiments, the nuclease comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NOs: 646 or SEQ ID NO: 647 or a variant thereof. In some embodiments, the nuclease comprises a sequence having at least about 70% identity to SEQ ID NO: 646 or SEQ ID NO: 647. In some embodiments, the nuclease comprises a sequence having at least about 75% identity to SEQ ID NO: 646 or SEQ ID NO: 647. In some embodiments, the nuclease comprises a sequence having at least about 80% identity to SEQ ID NO: 646 or SEQ ID NO: 647. In some embodiments, the nuclease comprises a sequence having at least about 85% identity to SEQ ID NO: 646 or SEQ ID NO: 647. In some embodiments, the nuclease comprises a sequence having at least about 90% identity to SEQ ID NO: 646 or SEQ ID NO: 647. In some embodiments, the nuclease comprises a sequence having at least about 95% identity to SEQ ID NO: 646 or SEQ ID NO: 647. In some embodiments, the nuclease comprises a sequence having at least about 96% identity to SEQ ID NO: 646 or SEQ ID NO: 647. In some embodiments, the nuclease comprises a sequence having at least about 97% identity to SEQ ID NO: 646 or SEQ ID NO: 647. In some embodiments, the nuclease comprises a sequence having at least about 98% identity to SEQ ID NO: 646 or SEQ ID NO: 647. In some embodiments, the nuclease comprises a sequence having at least about 99% identity to SEQ ID NO: 646 or SEQ ID NO: 647. In some embodiments, the nuclease comprises a sequence having 100% identity to SEQ ID NO: 646 or SEQ ID NO: 647.

In some embodiments, the nuclease is encoded by a nucleic acid sequence having at least 80% sequence identity with the nucleic acid sequence of SEQ ID NO: 653. In some embodiments, the nuclease is encoded by a nucleic acid sequence having at least 85% sequence identity with the nucleic acid sequence of SEQ ID NO: 653. In some embodiments, the nuclease is encoded by a nucleic acid sequence having at least 90% sequence identity with the nucleic acid sequence of SEQ ID NO: 653. In some embodiments, the nuclease is encoded by a nucleic acid sequence having at least 95% sequence identity with the nucleic acid sequence of SEQ ID NO: 653. In some embodiments, the nuclease is encoded by a nucleic acid sequence having at least 96% sequence identity with the nucleic acid sequence of SEQ ID NO: 653. In some embodiments, the nuclease is encoded by a nucleic acid sequence having at least 97% sequence identity with the nucleic acid sequence of SEQ ID NO: 653. In some embodiments, the nuclease is encoded by a nucleic acid sequence having at least 98% sequence identity with the nucleic acid sequence of SEQ ID NO: 653. In some embodiments, the nuclease is encoded by a nucleic acid sequence having at least 99% sequence identity with the nucleic acid sequence of SEQ ID NO: 653. In some embodiments, the nuclease is encoded by a nucleic acid sequence of SEQ ID NO: 653.

In some embodiments, the RuvC domain lacks nuclease activity. In some embodiments, the HNH domain lack nuclease activity. In some embodiments, the modified nuclease has a modification corresponding to position H840A in S. pyogenes Cas9. In some embodiments, the modified nuclease has a modification corresponding to position D10A in S. pyogenes Cas9. In some embodiments, the modified nuclease has a modification corresponding to position D13A in MG3-6 (SEQ ID NO: 646) termed nMG3-6 (D13A) (SEQ ID NO: 152). In some embodiments, the modified nuclease has a modification corresponding to position H586A in MG3-6 (SEQ ID NO: 646) termed nMG3-6 (H586A) (SEQ ID NO: 153). In some embodiments, the modified nuclease has a modification corresponding to position N609A in MG3-6 (SEQ ID NO: 646) termed nMG3-6 (N609A) (SEQ ID NO: 154). In some embodiments, the modified nuclease is configured to cleave one strand of a double-stranded target deoxyribonucleic acid. In some embodiments, the ribonucleic acid sequence configured to bind to the endonuclease comprises a tracr sequence.

In some embodiments, the nickase or nuclease comprises one or more nuclear localization sequences (NLSs) proximal to an N- or C-terminus of the nickase or nuclease.

In some embodiments, the NLS comprises any of the sequences in Table 1 above, or a combination thereof

### Guide Nucleic Acids

In some embodiments, provided herein are guide nucleic acids such as guide RNAs (gRNAs) or prime editing guide RNAs (pegRNAs). In a polynucleotide when referring to a T, a T means U (Uracil) in RNA and T (Thymine) in DNA.

Prime editing enables the installation of virtually any combination of point mutations, small insertions, or small deletions in the genome of living cells. A prime editing guide RNA (pegRNA) directs the prime editor protein to the targeted locus and also encodes the desired edit.

In some embodiments, the guide RNA targets a gene in a cell. In some embodiments, the guide RNA targets a gene in a mammalian cell. In some embodiments, the target gene is TRAC, VEGFA, AAVS1, B2M, CD5, or CD38. Exemplary guide RNAs are shown in SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910.

In some embodiments, the guide RNA is encoded by any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910, a sequence having at least about 80%, 85%, 90%, 95%, 97%, 98%, or 99% sequence identity to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910, or a reverse complement thereof. In some embodiments, the guide RNA is encoded by a sequence having at least about 80% sequence identity to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910 or a reverse complement thereof. In some embodiments, the guide RNA is encoded by a sequence having at least about 85% sequence identity to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910 or a reverse complement thereof. In some embodiments, the guide RNA is encoded by a sequence having at least about 90% sequence identity to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910 or a reverse complement thereof. In some embodiments, the guide RNA is encoded by a sequence having at least about 95% sequence identity to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910 or a reverse complement thereof. In some embodiments, the guide RNA is encoded by a sequence having at least about 97% sequence identity to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910 or a reverse complement thereof. In some embodiments, the guide RNA is encoded by a sequence having at least about 98% sequence identity to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910 or a reverse complement thereof. In some embodiments, the guide RNA is encoded by a sequence having at least about 99% sequence identity to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910 or a reverse complement thereof. In some embodiments, the guide RNA is encoded by a sequence according to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910 or a reverse complement thereof.

In some embodiments, the one or more guide RNAs are encoded by a sequence comprising at least about 80%, 85%, 90%, 95%, 97%, 98%, or 99% sequence identity to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910 or a reverse complement thereof. In some embodiments, the one or more guide RNAs are encoded by a sequence comprising at least about 80% sequence identity to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910 or a reverse complement thereof. In some embodiments, the one or more guide RNAs are encoded by a sequence comprising at least about 85% sequence identity to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910 or a reverse complement thereof. In some embodiments, the one or more guide RNAs are encoded by a sequence comprising at least about 90% sequence identity to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910 or a reverse complement thereof. In some embodiments, the one or more guide RNAs are encoded by a sequence comprising at least about 95% sequence identity to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910 or a reverse complement thereof. In some embodiments, the one or more guide RNAs are encoded by a sequence comprising at least about 97% sequence identity to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910 or a reverse complement thereof. In some embodiments, the one or more guide RNAs are encoded by a sequence comprising at least about 98% sequence identity to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910 or a reverse complement thereof. In some embodiments, the one or more guide RNAs are encoded by a sequence comprising at least about 99% sequence identity to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910 or a reverse complement thereof. In some embodiments, the guide RNA is encoded by a sequence according to any one of the nucleic acid sequences of SEQ ID NOs: 76-99, 109-140, 149, 656-697, 1310-1315, 1317-1341, 1451-1474, 1479-1492, 1564, 1568-1576, 1598-1620, 1656-1681, 1683-1690, 1722-1749, 1755-1774, 1784-1786, 1848-1855, and 1863-1910 or a reverse complement thereof, or a reverse complement thereof.

In some embodiments, guide RNAs or pegRNAs comprise various structural elements including but not limited to: a spacer sequence which binds to the protospacer sequence (target sequence), a crRNA, and an optional tracrRNA. In some embodiments, the genome editing system comprises a CRISPR guide RNA. In some embodiments, the guide RNA comprises a crRNA comprising a spacer sequence. In some embodiments, the guide RNA additionally comprises a tracrRNA or a modified tracrRNA.

In some embodiments, the compositions and methods provided herein comprise one or more guide RNAs. In some embodiments, the guide RNA comprises a sense sequence. In some embodiments, the guide RNA comprises an anti-sense sequence. In some embodiments, the guide RNA comprises nucleotide sequences other than the region complementary to or substantially complementary to a region of a target sequence. For example, a guide RNA is part or considered part of a crRNA, or is comprised in a crRNA, e.g., a crRNA:tracrRNA chimera.

In some embodiments, the guide RNA (e.g., gRNA) comprises synthetic nucleotides or modified nucleotides. In some embodiments, the guide RNA comprises one or more inter-nucleoside linkers modified from the natural phosphodiester. In some embodiments, all of the inter-nucleoside linkers of the guide RNA, or contiguous nucleotide sequence thereof, are modified. For example, in some embodiments, the inter nucleoside linkage comprises Sulphur (S), such as a phosphorothioate inter-nucleoside linkage.

In some embodiments, the guide RNA (e.g., gRNA) comprises modifications to a ribose sugar or nucleobase. In some embodiments, the guide RNA comprises one or more nucleosides comprising a modified sugar moiety, wherein the modified sugar moiety is a modification of the sugar moiety when compared to the ribose sugar moiety found in deoxyribose nucleic acid (DNA) and RNA. In some embodiments, the modification is within the ribose ring structure. Exemplary modifications include, but are not limited to, replacement with a hexose ring (HNA), a bicyclic ring having a biradical bridge between the C2 and C4 carbons on the ribose ring (e.g., locked nucleic acids (LNA)), or an unlinked ribose ring which typically lacks a bond between the C2 and C3 carbons (e.g., UNA). In some embodiments, the sugar-modified nucleosides comprise bicyclohexose nucleic acids or tricyclic nucleic acids. In some embodiments, the modified nucleosides comprise nucleosides where the sugar moiety is replaced with a non-sugar moiety, for example peptide nucleic acids (PNA) or morpholino nucleic acids.

In some embodiments, the guide RNA comprises one or more modified sugars. In some embodiments, the sugar modifications comprise modifications made by altering the substituent groups on the ribose ring to groups other than hydrogen, or the 2′-OH group naturally found in DNA and RNA nucleosides. In some embodiments, substituents are introduced at the 2′, 3′, 4′, 5′ positions, or combinations thereof. In some embodiments, nucleosides with modified sugar moieties comprise 2′ modified nucleosides, e.g., 2′ substituted nucleosides. A 2′ sugar modified nucleoside, in some embodiments, is a nucleoside that has a substituent other than H or —OH at the substitute (2′ substituted nucleoside) or comprises a 2′ linked biradical, and comprises 2′ substituted nucleosides and LNA (2′-4′ biradical bridged) nucleosides. Examples of 2′-substituted modified nucleosides comprise, but are not limited to, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. In some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the guide RNA comprises one or more modified sugars. In some embodiments, the guide RNA comprises only modified sugars. In certain embodiments, the guide RNA comprises greater than about 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the guide RNA comprises both inter-nucleoside linker modifications and nucleoside modifications.

In some embodiments, the guide RNA comprises about 15 nucleotides to about 28 nucleotides. In some embodiments, the guide RNA comprises at least about 15 nucleotides. In some embodiments, the guide RNA comprises at most about 28 nucleotides. In some embodiments, the guide RNA comprises about 15 nucleotides to about 16 nucleotides, about 15 nucleotides to about 17 nucleotides, about 15 nucleotides to about 18 nucleotides, about 15 nucleotides to about 19 nucleotides, about 15 nucleotides to about 20 nucleotides, about 15 nucleotides to about 21 nucleotides, about 15 nucleotides to about 22 nucleotides, about 15 nucleotides to about 23 nucleotides, about 15 nucleotides to about 24 nucleotides, about 15 nucleotides to about 25 nucleotides, about 15 nucleotides to about 28 nucleotides, about 16 nucleotides to about 17 nucleotides, about 16 nucleotides to about 18 nucleotides, about 16 nucleotides to about 19 nucleotides, about 16 nucleotides to about 20 nucleotides, about 16 nucleotides to about 21 nucleotides, about 16 nucleotides to about 22 nucleotides, about 16 nucleotides to about 23 nucleotides, about 16 nucleotides to about 24 nucleotides, about 16 nucleotides to about 25 nucleotides, about 16 nucleotides to about 28 nucleotides, about 17 nucleotides to about 18 nucleotides, about 17 nucleotides to about 19 nucleotides, about 17 nucleotides to about 20 nucleotides, about 17 nucleotides to about 21 nucleotides, about 17 nucleotides to about 22 nucleotides, about 17 nucleotides to about 23 nucleotides, about 17 nucleotides to about 24 nucleotides, about 17 nucleotides to about 25 nucleotides, about 17 nucleotides to about 28 nucleotides, about 18 nucleotides to about 19 nucleotides, about 18 nucleotides to about 20 nucleotides, about 18 nucleotides to about 21 nucleotides, about 18 nucleotides to about 22 nucleotides, about 18 nucleotides to about 23 nucleotides, about 18 nucleotides to about 24 nucleotides, about 18 nucleotides to about 25 nucleotides, about 18 nucleotides to about 28 nucleotides, about 19 nucleotides to about 20 nucleotides, about 19 nucleotides to about 21 nucleotides, about 19 nucleotides to about 22 nucleotides, about 19 nucleotides to about 23 nucleotides, about 19 nucleotides to about 24 nucleotides, about 19 nucleotides to about 25 nucleotides, about 19 nucleotides to about 28 nucleotides, about 20 nucleotides to about 21 nucleotides, about 20 nucleotides to about 22 nucleotides, about 20 nucleotides to about 23 nucleotides, about 20 nucleotides to about 24 nucleotides, about 20 nucleotides to about 25 nucleotides, about 20 nucleotides to about 28 nucleotides, about 21 nucleotides to about 22 nucleotides, about 21 nucleotides to about 23 nucleotides, about 21 nucleotides to about 24 nucleotides, about 21 nucleotides to about 25 nucleotides, about 21 nucleotides to about 28 nucleotides, about 22 nucleotides to about 23 nucleotides, about 22 nucleotides to about 24 nucleotides, about 22 nucleotides to about 25 nucleotides, about 22 nucleotides to about 28 nucleotides, about 23 nucleotides to about 24 nucleotides, about 23 nucleotides to about 25 nucleotides, about 23 nucleotides to about 28 nucleotides, about 24 nucleotides to about 25 nucleotides, about 24 nucleotides to about 28 nucleotides, or about 25 nucleotides to about 28 nucleotides. In some embodiments, the guide RNA comprises about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, or about 28 nucleotides.

In some embodiments, the guide nucleic acid further comprises a primer binding site (PBS). In some embodiments, the primer binding site is on a 3′ of the guide nucleic acid. In some embodiments, the primer binding site comprises at least 2, 4, 6, 8, 10, 13, 16, 20, 24, 28, 32, 36, 40, 45, 50, 55, 60, or 65 nucleotides. In some embodiments, the primer binding site comprises less than 2, 4, 6, or 8, nucleotides.

In some embodiments, the guide nucleic acid further comprises a reverse transcriptase template (RTT). In some embodiments, a base in the RTT comprises a bulky modification selected from the group of complex sugars, complex amino groups, and/or other modifications compatible with RNA. In some embodiments, the RTT is fused to the guide RNA. In some embodiments, the guide nucleic acid further comprises a homology sequence that is complementary to a region in the non-edited DNA strand. In some embodiments, the guide nucleic acid comprises a nucleic acid template. In some embodiments, the RTT has a length of at least about 10, 20, 30, 40, 50, 60, 70, 80, 90, or 100 nucleotides. In some the RTT has a length of at least about 100, 200, 300, 400, 500, 600, 700, 800, 900, or 1000 nucleotides. In some embodiments, the RTT has a length of at least about 1000, 2000, 3000, 4000, or 5000 nucleotides. In some embodiments, the RTT has a length between about 10 and about 20, 30, 40, 50, 60, 70, 80, 90, 100, 120, 140, 160, 180, 200, or more than 200 nucleotides. In some embodiments, the RTT has a length between about 20 and about 30, 40, 50, 60, 70, 80, 90, 100, 120, 140, 160, 180, 200, or more than 200 nucleotides. In some embodiments, the RTT has a length between about 30 and about 40, 50, 60, 70, 80, 90, 100, 120, 140, 160, 180, 200, or more than 200 nucleotides. In some embodiments, the RTT has a length between about 40 and about 50, 60, 70, 80, 90, 100, 120, 140, 160, 180, 200, or more than 200 nucleotides. In some embodiments, the RTT has a length between about 50 and about 60, 70, 80, 90, 100, 120, 140, 160, 180, 200, or more than 200 nucleotides. In some embodiments, the RTT has a length between about 60 and about 70, 80, 90, 100, 120, 140, 160, 180, 200, or more than 200 nucleotides. In some embodiments, the RTT has a length between about 70 and about 80, 90, 100, 120, 140, 160, 180, 200, or more than 200 nucleotides. In some embodiments, the RTT has a length between about 80 and about 100, 120, 140, 160, 180, 200, or more than 200 nucleotides. In some embodiments, the RTT has a length between about 100 and about 120, 140, 160, 180, 200, or more than 200 nucleotides. In some embodiments, the RTT has a length between about 100 and about 4000 nucleotides. In some embodiments, the RTT has a length between about 100 and about 200, 300, 400, 500, 600, 700, 800, 900, 1000, 1500, 2000, 2500, 3000, 3500, or 4000 nucleotides. In some embodiments, the RTT has a length between about 500 and about 600, 700, 800, 900, 1000, 1500, 2000, 2500, 3000, 3500, or 4000 nucleotides. In some embodiments, the RTT has a length between about 1000 and about 1500, 2000, 2500, 3000, 3500, or 4000 nucleotides. In some embodiments, the RTT has a length between about 2000 and about 2500, 3000, 3500, or 4000 nucleotides. In some embodiments, the RTT has a length between about 3000 and about 3500, or 4000 nucleotides.

Methods of making guide nucleic acids are known in the art. For example, guide RNAs and pegRNAs, as well as and modified guide RNAs and pegRNAs, can be chemically synthesized. Additionally, nucleic sequences encoding guide nucleic acids can be cloned into a vector and transcribed from the vector in vitro or in vivo using RNA polymerases.

### Cells

Described herein, in certain embodiments, is a cell comprising gene editing systems described herein.

In some embodiments, the cell is a eukaryotic cell (e.g., a plant cell, an animal cell, a protist cell, or a fungi cell), a mammalian cell (a Chinese hamster ovary (CHO) cell, baby hamster kidney (BHK), human embryo kidney (HEK), mouse myeloma (NS0), or human retinal cells), an immortalized cell (e.g., a HeLa cell, a COS cell, a HEK-293T cell, a MDCK cell, a 3T3 cell, a PC12 cell, a Huh7 cell, a HepG2 cell, a K562 cell, a N2a cell, or a SY5Y cell), an insect cell (e.g., a Spodoptera frugiperda cell, a Trichoplusia ni cell, a Drosophila melanogaster cell, a S2 cell, or a Heliothis virescens cell), a yeast cell (e.g., a Saccharomyces cerevisiae cell, a Cryptococcus cell, or a Candida cell), a plant cell (e.g., a parenchyma cell, a collenchyma cell, or a sclerenchyma cell), a fungal cell (e.g., a Saccharomyces cerevisiae cell, a Cryptococcus cell, or a Candida cell), or a prokaryotic cell (e.g., a E. coli cell, a streptococcus bacterium cell, a streptomyces soil bacteria cell, or an archaea cell). In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell.

In some embodiments, the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, a primary cell, or derivative thereof.

In some embodiments, the present disclosure provides a cell comprising a vector or a nucleic acid described herein. In some embodiments, the cell expresses a gene editing system or parts thereof. In some embodiments, the cell is a human cell. In some embodiments, the genome is edited ex vivo. In some embodiments, the genome is edited in vivo.

### Delivery and Vectors

Disclosed herein, in some embodiments, are nucleic acid sequences encoding a gene editing system comprising a nickase, a reverse transcriptase, and a guide polynucleotide, a fusion protein comprising a nickase and a reverse transcriptase, or a guide polynucleotide.

In some embodiments, the nucleic acid encoding the gene editing system, fusion protein, or guide polynucleotide is a DNA, for example a linear DNA, a plasmid DNA, or a minicircle DNA. In some embodiments, the nucleic acid encoding the gene editing system, fusion protein, or guide polynucleotide is an RNA, for example a mRNA.

In some embodiments, the nucleic acid encoding the gene editing system, fusion protein, or guide polynucleotide is delivered by a nucleic acid-based vector. In some embodiments, the nucleic acid encoding the gene editing system, fusion protein, or guide polynucleotide is delivered by a plasmid (e.g., circular DNA molecules that can autonomously replicate inside a cell), cosmid (e.g., pWE or sCos vectors), artificial chromosome, human artificial chromosome (HAC), yeast artificial chromosomes (YAC), bacterial artificial chromosome (BAC), P1-derived artificial chromosomes (PAC), phagemid, phage derivative, bacmid, or virus. In some embodiments, the nucleic acid is comprised in a vector selected from the list consisting of: pSF-CMV-NEO-NH2-PPT-3XFLAG, pSF-CMV-NEO-COOH-3XFLAG, pSF-CMV-PURO-NH2-GST-TEV, pSF-OXB20-IH-TEV-FLAG(R)-6His, pCEP4 pDEST27, pSF-CMV-Ub-KrYFP, pSF-CMV-FMDV-daGFP, pEF1a-mCherry-N1 vector, pEFla-tdTomato vector, pSF-CMV-FMDV-Hygro, pSF-CMV-PGK-Puro, pMCP-tag(m), pSF-CMV-PURO-NH2-CMYC, pSF-OXB20-BetaGal, pSF-OXB20-Fluc, pSF-OXB20, pSF-Tac, pRI 101-AN DNA, pCambia2301, pTYB21, pKLAC2, pAc5.1/V5-His A, and pDEST8.

In some embodiments, the nucleic acid-based vector comprises a promoter. In some embodiments, the promoter is selected from the group consisting of a mini promoter, an inducible promoter, a constitutive promoter, and derivatives thereof. In some embodiments, the promoter is selected from the group consisting of CMV, CBA, EF1a, CAG, PGK, TRE, U6, UAS, T7, Sp6, lac, araBad, trp, Ptac, p5, p19, p40, Synapsin, CaMKII, GRK1, and derivatives thereof. In some embodiments the promoter is a U6 promoter. In some embodiments, the promoter is a CAG promoter.

In some embodiments, the nucleic acid-based vector is a virus. In some embodiments, the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus. In some embodiments, the virus is an alphavirus. In some embodiments, the virus is a parvovirus. In some embodiments, the virus is an adenovirus. In some embodiments, the virus is an AAV. In some embodiments, the virus is a baculovirus. In some embodiments, the virus is a Dengue virus. In some embodiments, the virus is a lentivirus. In some embodiments, the virus is a herpesvirus. In some embodiments, the virus is a poxvirus. In some embodiments, the virus is an anellovirus. In some embodiments, the virus is a bocavirus. In some embodiments, the virus is a vaccinia virus. In some embodiments, the virus is or a retrovirus.

In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LKO3, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, AAV-HSC16, or a derivative thereof. In some embodiments, the herpesvirus is HSV type 1, HSV-2, VZV, EBV, CMV, HHV-6, HHV-7, or HHV-8.

In some embodiments, the virus is AAV1 or a derivative thereof. In some embodiments, the virus is AAV2 or a derivative thereof. In some embodiments, the virus is AAV3 or a derivative thereof. In some embodiments, the virus is AAV4 or a derivative thereof. In some embodiments, the virus is AAV5 or a derivative thereof. In some embodiments, the virus is AAV6 or a derivative thereof. In some embodiments, the virus is AAV7 or a derivative thereof. In some embodiments, the virus is AAV8 or a derivative thereof. In some embodiments, the virus is AAV9 or a derivative thereof. In some embodiments, the virus is AAV10 or a derivative thereof. In some embodiments, the virus is AAV11 or a derivative thereof. In some embodiments, the virus is AAV12 or a derivative thereof. In some embodiments, the virus is AAV13 or a derivative thereof. In some embodiments, the virus is AAV14 or a derivative thereof. In some embodiments, the virus is AAV15 or a derivative thereof. In some embodiments, the virus is AAV16 or a derivative thereof. In some embodiments, the virus is AAV-rh8 or a derivative thereof. In some embodiments, the virus is AAV-rh10 or a derivative thereof. In some embodiments, the virus is AAV-rh20 or a derivative thereof. In some embodiments, the virus is AAV-rh39 or a derivative thereof. In some embodiments, the virus is AAV-rh74 or a derivative thereof. In some embodiments, the virus is AAV-rhM4-1 or a derivative thereof. In some embodiments, the virus is AAV-hu37 or a derivative thereof. In some embodiments, the virus is AAV-Anc80 or a derivative thereof. In some embodiments, the virus is AAV-Anc80L65 or a derivative thereof. In some embodiments, the virus is AAV-7m8 or a derivative thereof. In some embodiments, the virus is AAV-PHP-B or a derivative thereof. In some embodiments, the virus is AAV-PHP-EB or a derivative thereof. In some embodiments, the virus is AAV-2.5 or a derivative thereof. In some embodiments, the virus is AAV-2tYF or a derivative thereof. In some embodiments, the virus is AAV-3B or a derivative thereof. In some embodiments, the virus is AAV-LK03 or a derivative thereof. In some embodiments, the virus is AAV-HSC1 or a derivative thereof. In some embodiments, the virus is AAV-HSC2 or a derivative thereof. In some embodiments, the virus is AAV-HSC3 or a derivative thereof. In some embodiments, the virus is AAV-HSC4 or a derivative thereof. In some embodiments, the virus is AAV-HSC5 or a derivative thereof. In some embodiments, the virus is AAV-HSC6 or a derivative thereof. In some embodiments, the virus is AAV-HSC7 or a derivative thereof. In some embodiments, the virus is AAV-HSC8 or a derivative thereof. In some embodiments, the virus is AAV-HSC9 or a derivative thereof. In some embodiments, the virus is AAV-HSC10 or a derivative thereof. In some embodiments, the virus is AAV-HSC11 or a derivative thereof. In some embodiments, the virus is AAV-HSC12 or a derivative thereof. In some embodiments, the virus is AAV-HSC13 or a derivative thereof. In some embodiments, the virus is AAV-HSC14 or a derivative thereof. In some embodiments, the virus is AAV-HSC15 or a derivative thereof. In some embodiments, the virus is AAV-TT or a derivative thereof. In some embodiments, the virus is AAV-DJ/8 or a derivative thereof. In some embodiments, the virus is AAV-Myo or a derivative thereof. In some embodiments, the virus is AAV-NP40 or a derivative thereof. In some embodiments, the virus is AAV-NP59 or a derivative thereof. In some embodiments, the virus is AAV-NP22 or a derivative thereof. In some embodiments, the virus is AAV-NP66 or a derivative thereof. In some embodiments, the virus is AAV-HSC16 or a derivative thereof.

In some embodiments, the virus is HSV-1 or a derivative thereof. In some embodiments, the virus is HSV-2 or a derivative thereof. In some embodiments, the virus is VZV or a derivative thereof. In some embodiments, the virus is EBV or a derivative thereof. In some embodiments, the virus is CMV or a derivative thereof. In some embodiments, the virus is HHV-6 or a derivative thereof. In some embodiments, the virus is HHV-7 or a derivative thereof. In some embodiments, the virus is HHV-8 or a derivative thereof.

In some embodiments, the nucleic acid encoding the gene editing system, fusion protein, or guide polynucleotide is delivered by a non-nucleic acid-based delivery system (e.g., a non-viral delivery system). In some embodiments, the nucleic acid is comprised in a liposome. In some embodiments, the nucleic acid is associated with a lipid. The nucleic acid associated with a lipid, in some embodiments, is encapsulated in the aqueous interior of a liposome, interspersed within the lipid bilayer of a liposome, attached to a liposome via a linking molecule that is associated with both the liposome and the nucleic acid, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, the nucleic acid is comprised in a lipid nanoparticle (LNP).

In some embodiments, the nucleic acid encoding the gene editing system, fusion protein, or guide polynucleotide is introduced into the cell in any suitable way, either stably or transiently. In some embodiments, a fusion protein or genome editing system is transfected into the cell. In some embodiments, the cell is transduced or transfected with a nucleic acid construct that encodes a fusion protein or genome editing system. For example, a cell is transduced (e.g., with a virus encoding a fusion protein or genome editing system), or transfected (e.g., with a plasmid encoding a fusion protein or genome editing system) with a nucleic acid that encodes a fusion protein or genome editing system, or the translated fusion protein or genome editing system. In some embodiments, the transduction is a stable or transient transduction. In some embodiments, cells expressing a fusion protein or genome editing system or containing a fusion protein or genome editing system are transduced or transfected with one or more gRNA or pegRNA molecules, for example when the fusion protein or genome editing system comprises a CRISPR nuclease. In some embodiments, a plasmid expressing a fusion protein or genome editing system is introduced into cells through electroporation, transient (e.g., lipofection) and stable genome integration (e.g., piggybac) and viral transduction (for example lentivirus or AAV) or other methods known to those of skill in the art. In some embodiments, the gene editing system is introduced into the cell as one or more polypeptides. In some embodiments, delivery is achieved through the use of RNP complexes. Delivery methods to cells for polypeptides and/or RNPs are known in the art, for example by electroporation or by cell squeezing.

Exemplary methods of delivery of nucleic acids include lipofection, nucleofection, electroporation, stable genome integration (e.g., piggybac), microinjection, biolistics, virosomes, liposomes, immunoliposomes, polycation or lipid nucleic acid conjugates, naked DNA, artificial virions, and agent-enhanced uptake of DNA. Lipofection is described in e.g., U.S. Pat. Nos. 5,049,386; 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of WO 91/17424 and WO 91/16024. In some embodiments, the delivery is to cells (e.g., in vitro or ex vivo administration) or target tissues (e.g., in vivo administration). In some embodiments, the nucleic acid is comprised in a liposome or a nanoparticle that specifically targets a host cell.

Additional methods for the delivery of nucleic acids to cells are known to those skilled in the art. See, for example, US 2003/0087817.

### Methods of Use

Described herein, in some embodiments, are methods for modifying a double- and/or single-stranded nucleic acid, comprising a) providing a cell with a guide nucleic acid to bind to a target strand of the double-stranded nucleic acid; b) providing a cell with a nuclease or nickase to cleave the double-stranded nucleic acid at a location of binding of the guide nucleic acid; c) providing a cell with a reverse transcriptase to synthesize a modification in the target strand of the double-stranded nucleic acid at a location of cleavage by the nickase and/or double strand nuclease.

In some embodiments, the methods are used to introduce a modification in the genome of a cell. In some embodiments, the modification is an insertion, deletion, or mutation. In some embodiments, the methods are used to introduce site-directed insertions, deletions, and/or mutations in the genome of a cell (for example an insertion and a mutation). In some embodiments, the methods are used in combination with a nucleic acid template to facilitate site-directed insertions into the genome of a cell. In some embodiments, the cell is a human cell. In some embodiments, the cell genome or a vector comprised in the cell is modified. In some embodiments, the cell genome is modified ex vivo. In some embodiments, the cell genome is modified in vivo.

In some embodiments, the methods further comprise providing the cell a transposase, integrase, or homing endonuclease. In some embodiments, the methods further comprise providing the cell a retrotransposon. In some embodiments, the method further comprises providing an RNA or DNA insertion template.

In some embodiments, the methods described herein further comprise detecting the genome modifications. In some embodiments, after the cell genome is modified, the cell is cultured for a certain amount of time. In some embodiments, the DNA or RNA is extracted and sequenced, and modified sequence areas are mapped and compared with an unmodified sequence. In some embodiments, cells are stained with antibodies for protein products that are translated from the modified nucleic acid, and the resulting stained proteins or polypeptides in the cell are analyzed, for example by flow cytometry.

The methods described herein can be used, for example, for targeted SNP corrections, small insertions, or small deletions. Additionally, the methods described herein can be used for targeted insertion of large templates into the genome of a cell by using a suitable RTT.

### Kits

In some embodiments, this disclosure provides kits comprising one or more nucleic acid constructs encoding the various components of the fusion protein or genome editing system described herein, e.g., comprising a nucleotide sequence encoding the components of the fusion protein or genome editing system capable of modifying a target DNA sequence. In some embodiments, the nucleotide sequence comprises a heterologous promoter that drives expression of the RNA genome editing system components.

In some embodiments, any of the targetable reverse transcriptases or genome editing systems disclosed herein is assembled into a pharmaceutical, diagnostic, or research kit to facilitate its use in therapeutic, diagnostic, or research applications. A kit may include one or more containers housing any of the vectors disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions, in some embodiments, are in a form prescribed by a governmental agency regulating the manufacture, use, or sale of pharmaceuticals or biological products, which instructions can also reflect approval by the agency of manufacture, use, or sale for animal administration.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1. Bioinformatic Identification of Reverse Transcriptases from Metagenomic Databases

This example describes the identification of proteins with reverse transcriptase function by a bioinformatic approach.

An extensive assembly-driven metagenomic database of microbial, viral, and eukaryotic genomes was bioinformatically analyzed for proteins with putative reverse transcriptase function. The analysis uncovered millions of proteins with predicted reverse transcriptase function. The predicted RT hits were then bioinformatically filtered for complete open reading frames (ORFs) with a high quality RT domain hit covering over 70% of the reference RT domains, and containing expected catalytic residues. After filtering, 468 RTs were selected for their potential to develop gene editing tools (SEQ ID NOs: 161-629). For all of these identified putative RTs, the predicted active site tetrad motif is [Y/F]XDD, where the most frequent amino acid at position one of the tetrad is tyrosine (Y, 85.2%) or phenylalanine (F, 14.5%). The second position of the tetrad is much more diverse, with the most frequent residues being alanine (A, 55.5%), isoleucine (I, 9.3%), and valine (V, 19.3%). The aspartate dyad (DD) is the most conserved feature for RT activity.

### Example 2. Reverse Transcriptases (RTs) for Short Corrections, Small Insertions, and Deletions

This example describes the use of untethered reverse transcriptases in combination with pegRNAs for targeted genome editing in HEK293T cells.

Testing Reverse Transcriptase Candidates with Untethered Nickase

Reverse transcriptase (RT) candidates from the MG151 (SEQ ID NOs: 1-37), MG153 (SEQ ID NOs: 38-61), and MG160 families (SEQ ID NOs: 62-75) were cloned into a plasmid where expression of the RT candidate is driven by the CMV promoter. The plasmid was isolated for transfection into HEK293T cells. A second plasmid containing a nickase spCas9 (H840A) where the expression was driven by a CMV promoter, and the RT-containing plasmid were co-transfected. Chemically synthesized pegRNAs (SEQ ID NOs: 76-99) containing the desired edit in the RT template were transfected. All components (plasmids and pegRNAs) were reverse transfected into 150,000 HEK293T cells in a 24 well plate. 72 hours post-transfection, cells were lysed in 100 L of solution. Primers containing barcodes for next generation sequencing (NGS) (SEQ ID NOs: 100-101) were used to amplify a ˜250 bp target (SEQ ID NO: 102) with mastermix. PCR clean-up was then performed, and samples were NGS sequenced. FASTQ files were then processed using prime editing to determine the percentage of reads with desired change.

### MG151Family

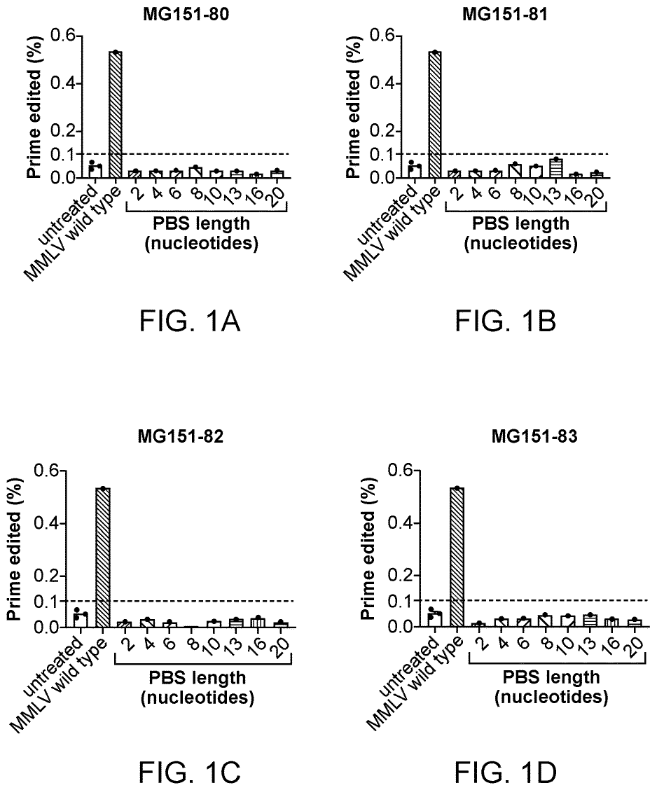

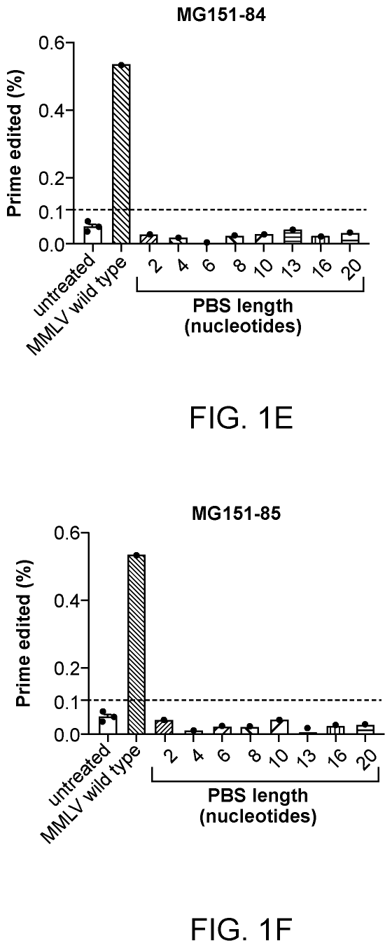

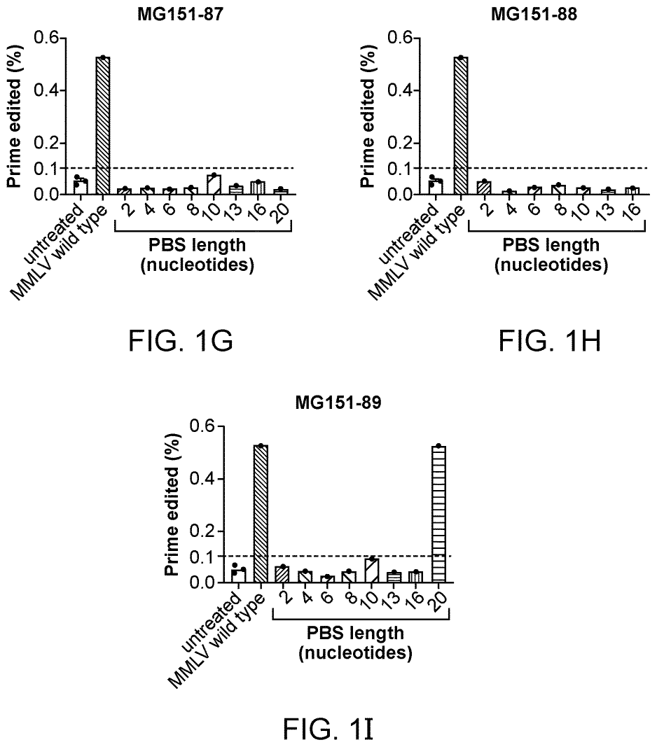

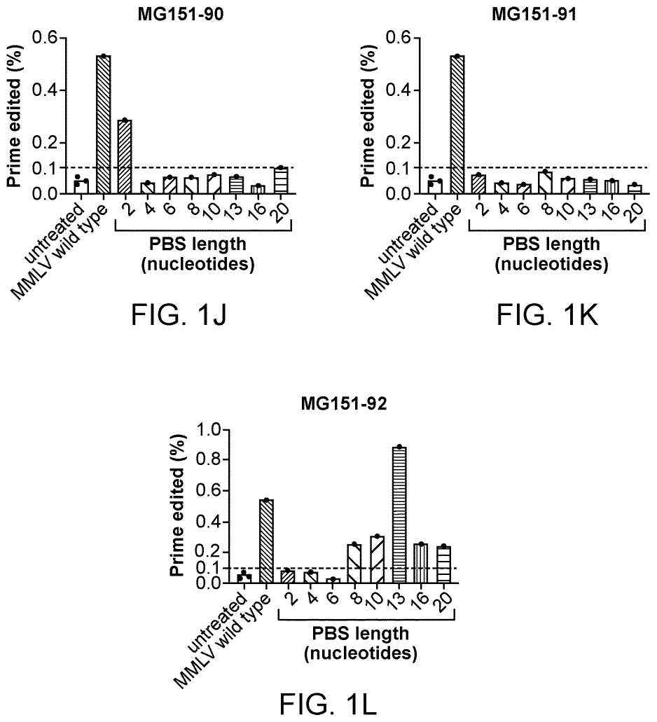

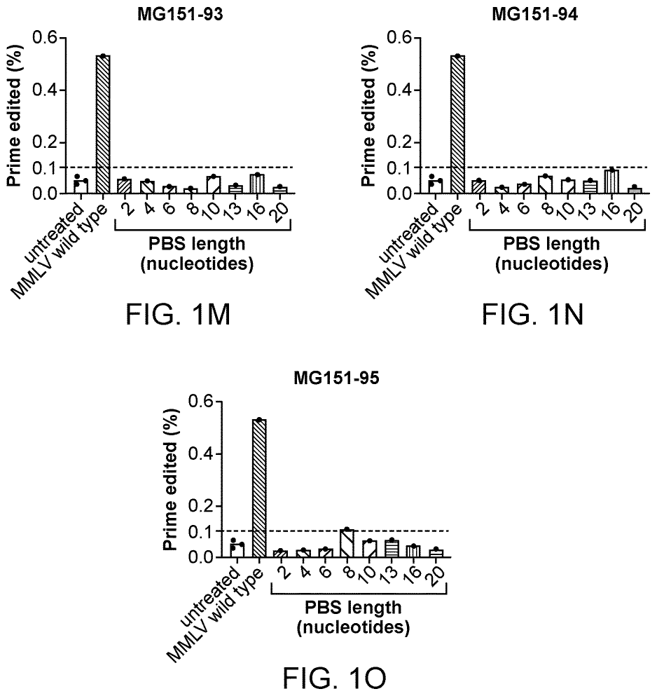

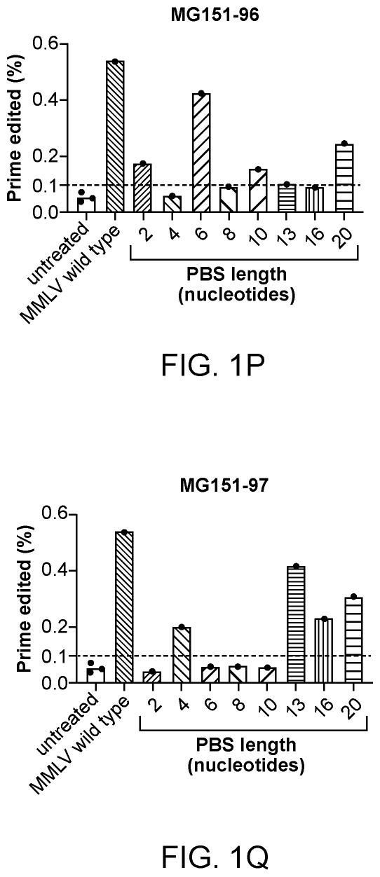

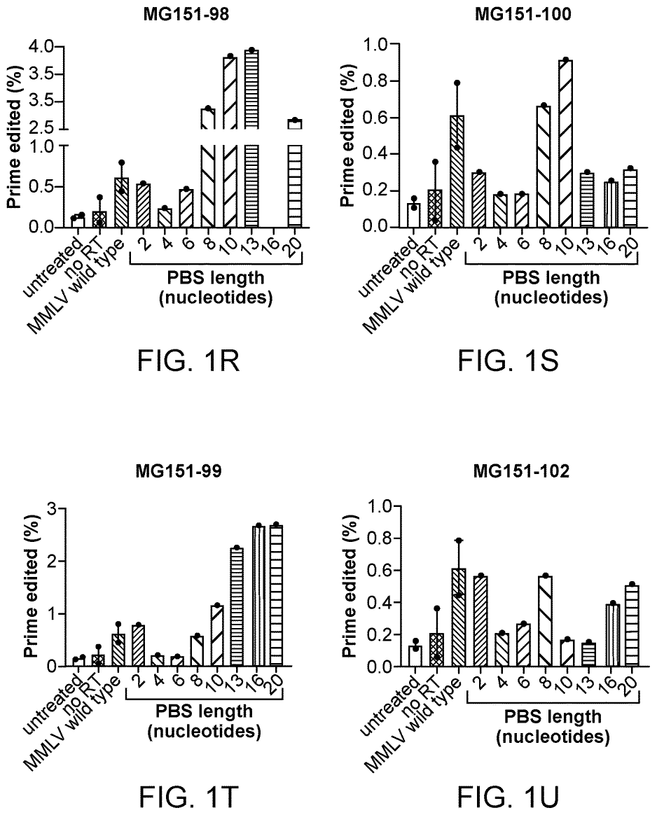

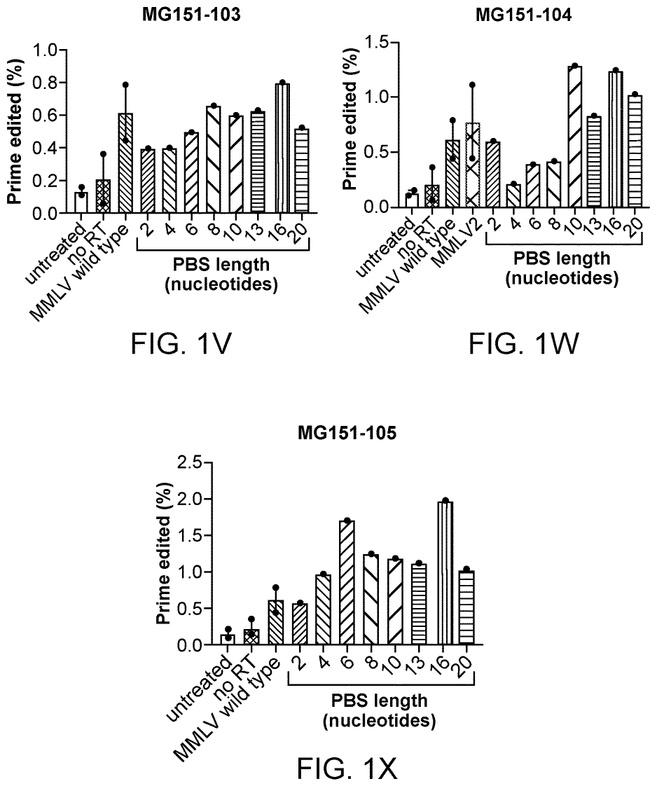

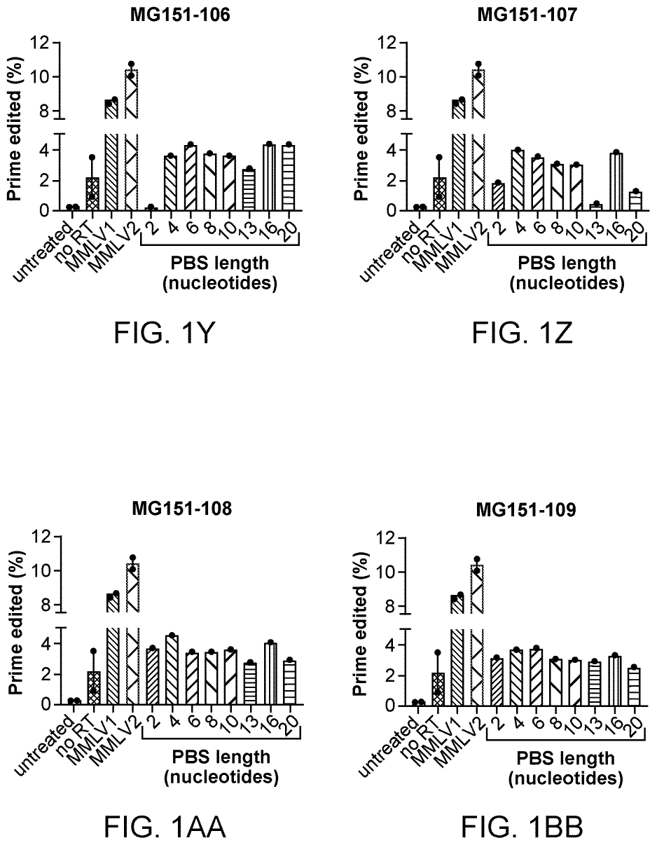

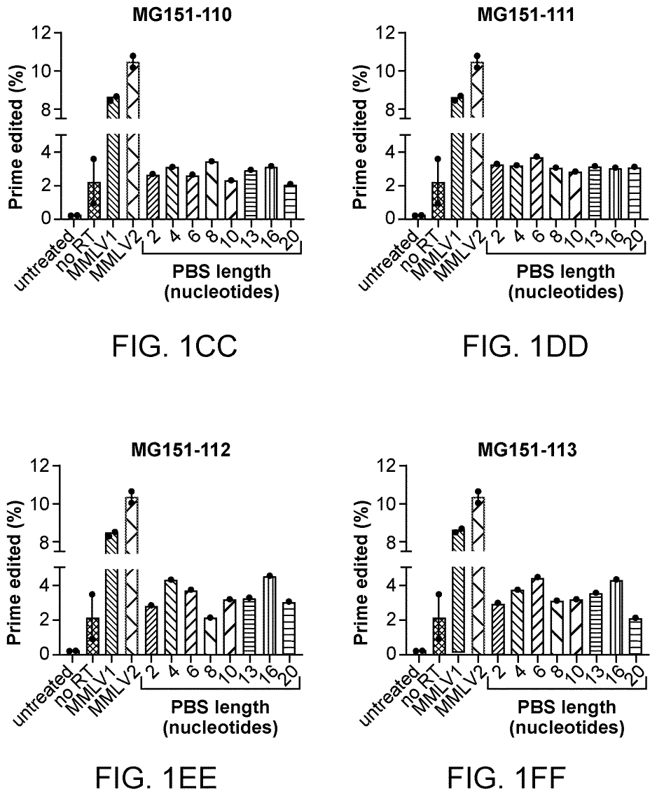

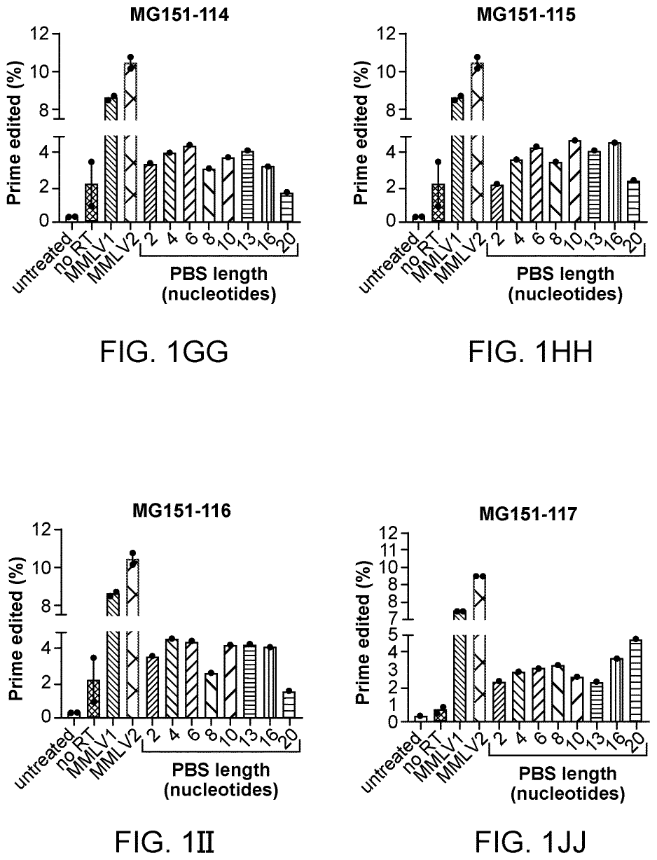

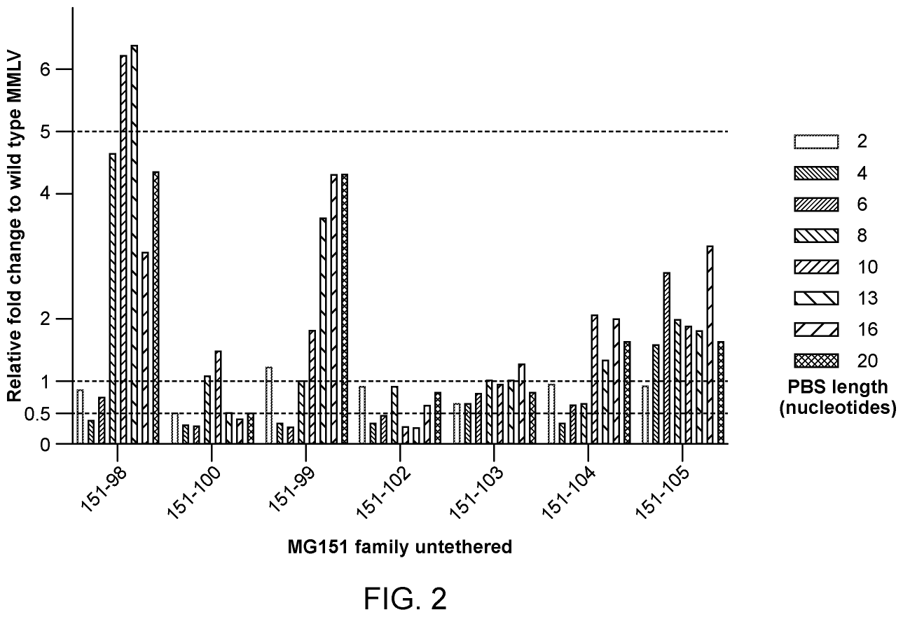

Untethered MG151 candidates 80-85 (SEQ ID NOs: 1-6), 87-100 (SEQ ID NOs: 7-20), and 102-117 (SEQ ID NOs: 22-37) were tested for prime editing in HEK293T cells to determine percent change of desired correction. Percent editing for each RT is shown in FIGS. 1A-1JJ for each pegRNA with varying PBS lengths (2, 4, 6, 8, 10, 13, 16, 20 nucleotides) (SEQ ID NOs: 76-83). In a single replicate, MG151-98 (SEQ ID NO: 18) and MG151-99 (SEQ ID NO: 19) had six-fold and four-fold higher editing than the wild-type MMLV, respectively (FIG. 2). MG151 candidates MG151-100 (SEQ ID NO: 19), MG151-103 (SEQ ID NO: 23), MG151-104 (SEQ ID NO: 24), and MG151-105 (SEQ ID NO: 25) had half as much or equivalent editing levels to wild-type MMLV (FIG. 2).

### MG153Family

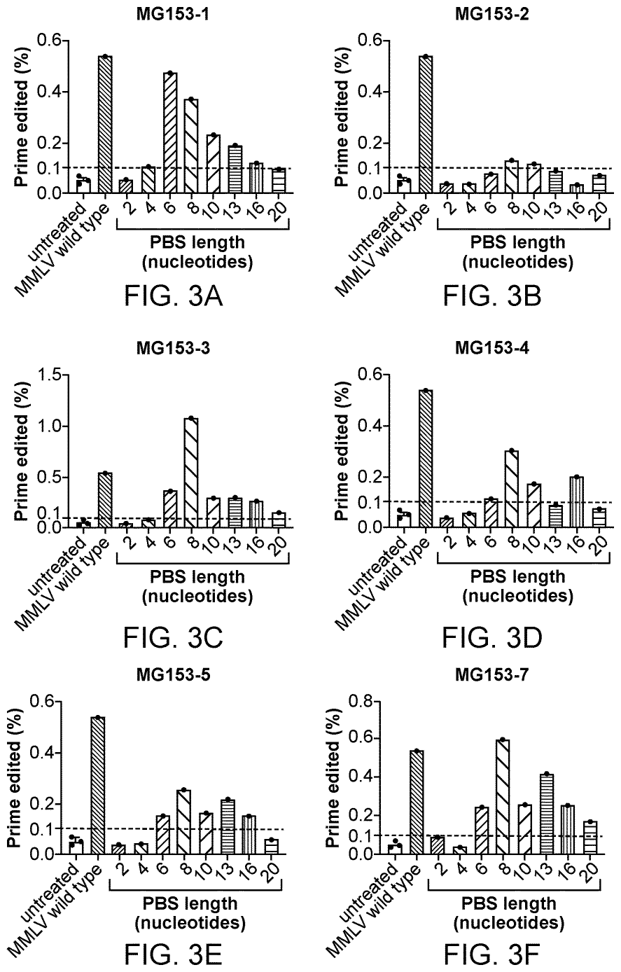

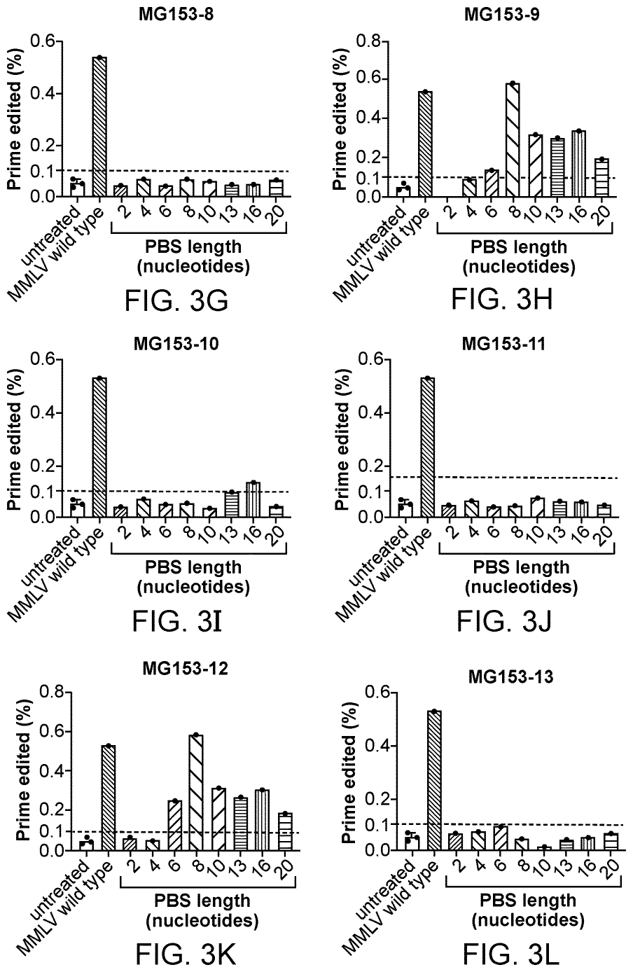

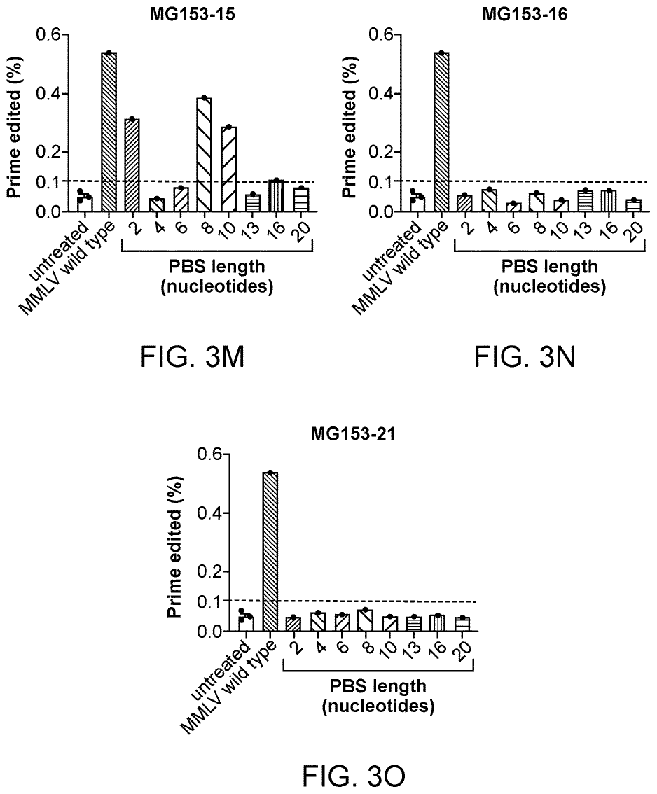

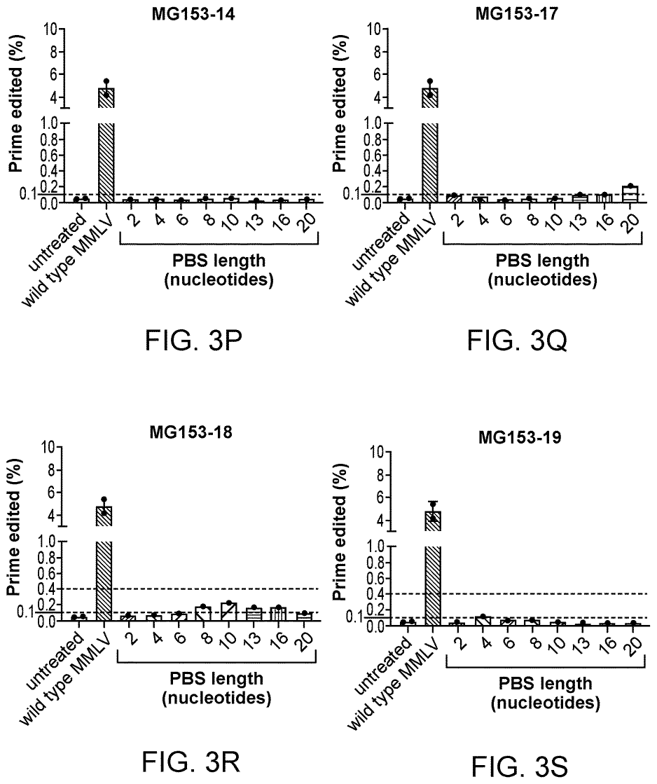

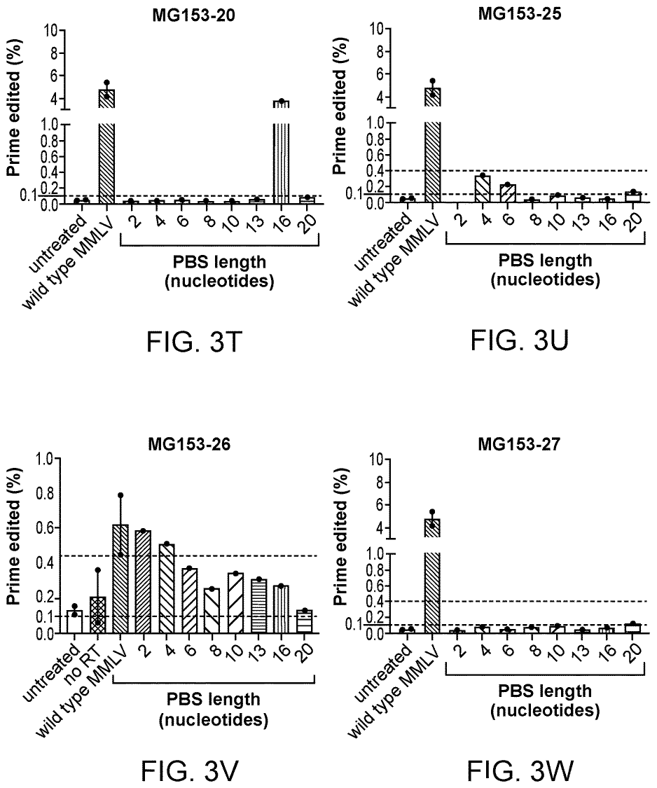

Untethered MG153 candidates 1-5 (SEQ ID NOs: 38-42), 7-21 (SEQ ID NOs: 44-58), and 25-27 (SEQ ID NOs: 59-61) were tested for prime editing in HEK293T cells to determine percent change of desired correction. Percent editing for each RT is shown in FIGS. 3A-30 and 3P-3W for each pegRNA with varying PBS lengths (2, 4, 6, 8, 10, 13, 16, 20 nucleotides) (SEQ ID NOs: 76-83). MG153-1 (SEQ ID NO: 38), MG153-3 (SEQ ID NO: 40), MG153-7 (SEQ ID NO: 44), MG153-9 (SEQ ID NO: 46), MG153-12 (SEQ ID NO: 49), and MG153-15 (SEQ ID NO: 52) have shown editing levels above background or comparable to MMLLV wild-type.

### MG160Family

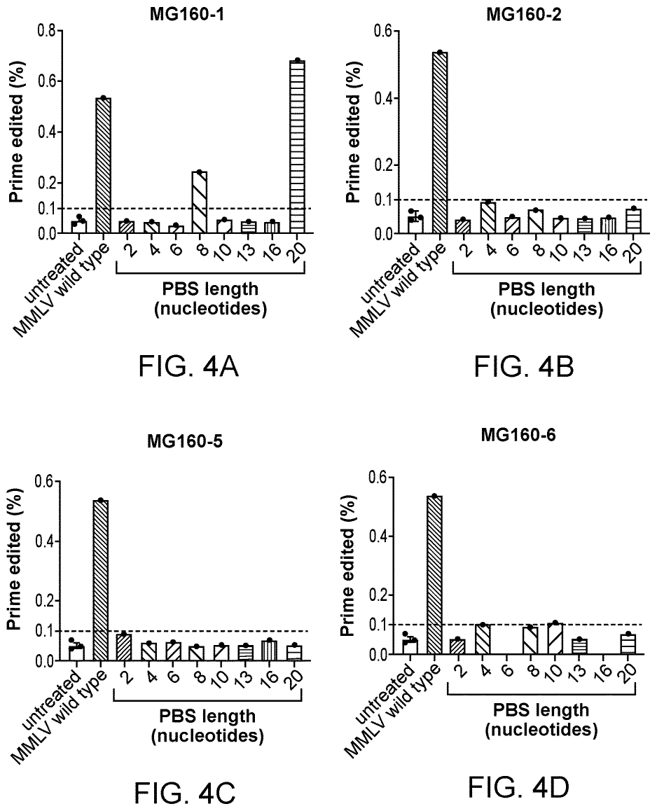

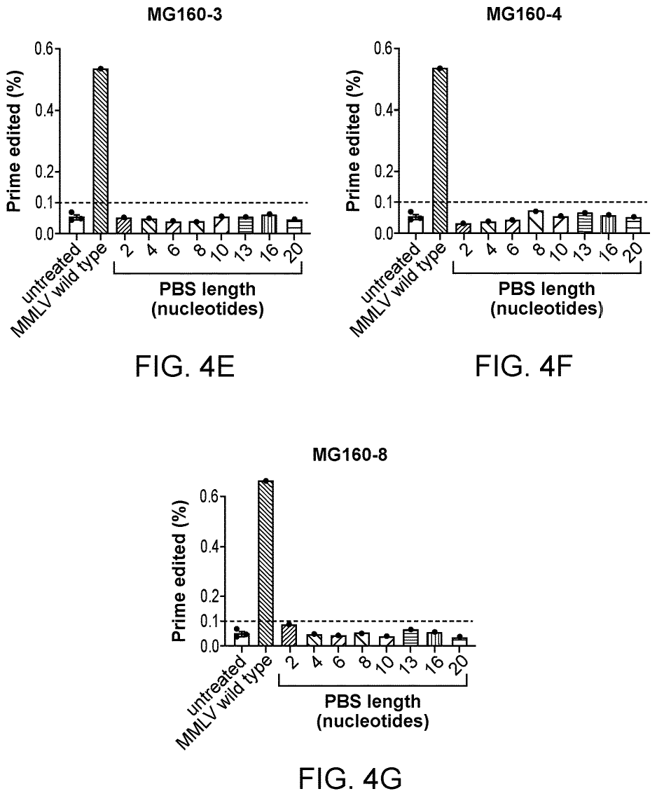

Untethered MG160 family candidates MG160-1 through MG160-8 (SEQ ID NOs: 62-68) were tested in mammalian cells for activity as described above. Activity above background was seen for untethered candidates MG160-1 (SEQ ID NO: 62) and MG160-4 (SEQ ID NO: 65). (FIGS. 4A-4G).

### Testing Reverse Transcriptase Candidates Tethered to a Nickase

The activity of diverse RT classes with CRISPR Type II nucleases was evaluated. RT candidates were cloned into a plasmid containing the nickase spCas9(H840A) to generate an RT-nickase fusion. The CMV promoter drove the expression of the RT-Nickase fusion protein, which contained a thirty three amino acid linker (SEQ ID NO: 103) between the nickase and the RT candidate. The fusion protein was then transfected into HEK293T cells and processed for NGS as described above.

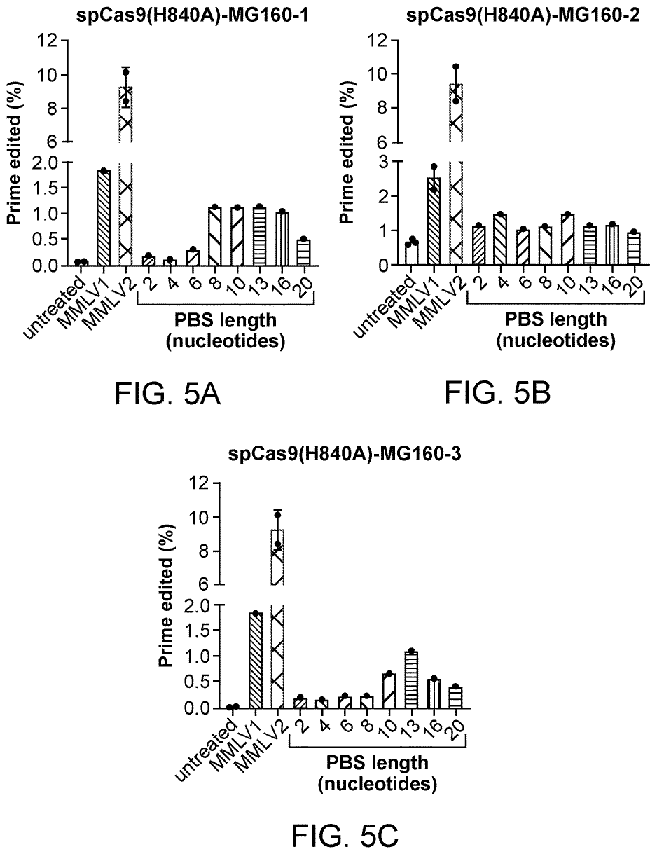

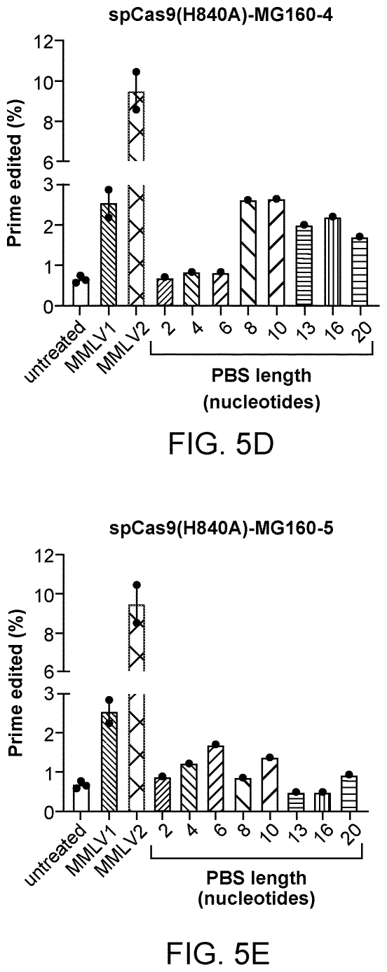

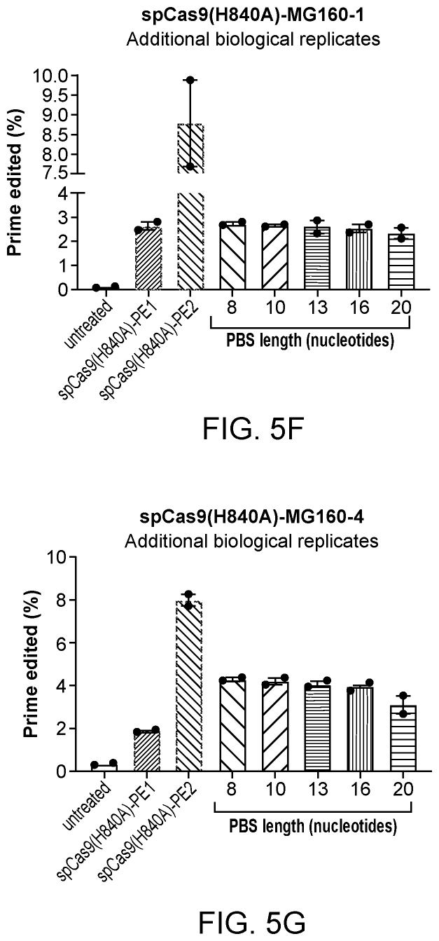

The activity of tethered MG160 candidates 1-5 (SEQ ID NOs: 69-73) is shown in FIGS. 5A-5E. Specifically, candidate MG160-4 (SEQ ID NO: 72) had comparable levels to wild-type MMLV (FIG. 5D). All other MG160 candidates (SEQ ID NOs: 69-72) had at least half the activity of wild-type MMLV at a specific PBS length. Moreover, when MG160-1 (SEQ ID NO: 69) and MG160-4 (SEQ ID NO: 72) were repeated with two additional biological replicates, the editing was comparable to MMLV WT for MG160-1 (SEQ ID NO: 69), and the editing was 2-fold higher for MG160-4 (SEQ ID NO: 72) (FIGS. 5F-5G).

The data above demonstrates that several RTs from different phylogenetic families were identified that showed comparable or higher activity than MMLV WT in a prime editing context. Having activity across a broad range of families allows the identification of RT candidates for different kinds of genomic modifications (i.e., SNP corrections, insertions, or deletions). At least 2 RTs with sizes ˜250 aa that perform similarly or outperform MMLV WT (MG160-1 (SEQ ID NO: 69) and MG160-4 (SEQ ID NO: 72)) were identified. The small size of the RT (⅓ of MMLV WT) allows an efficient delivery using adeno-associated viruses (AAVs) and lipid nanoparticles (LNPs).

### Example 3. RTs for Short Corrections, Small Insertions and Deletions (Prophetic)

This example describes the use of additional reverse transcriptases in combination with pegRNAs for targeted genome editing in HEK293T cells.

Additional RTs from the MG151 and MG153 families, including MG151-101 (SEQ ID NO: 21), MG153-6 (SEQ ID NO: 43), or additional candidates are tested as described in Example 2 in the untethered format. This allows for the identification of additional RT candidates for small corrections, insertions, and deletions.

RTs from the MG160 family which include MG160-6 (SEQ ID NO: 74), MG160-8 (SEQ ID NO: 75), and other candidates are tested for editing as described above in the tethered system. This allows to for the identification of additional miniature (˜250aa) RT systems that may mediate small corrections, insertions, and deletions.

### Example 4. Nucleases for Mediating Short Corrections, Small Insertions, and Deletions in Conjunction with Reverse Transcriptases

This example describes the use of an RNA-guided nuclease in combination with pegRNAs for targeted genome editing in HEK293T cells.

To evaluate the requirements for designing pegRNAs (gRNA with 3′ extension) for the nucleases of the disclosure, a number of PBS with varying lengths were assessed for maintaining a proper nuclease-gRNA interaction. To test for nuclease activity in combination with the pegRNA designs, InDel formation in HEK293T cells was tested. MG3-6 (SEQ ID NO: 104) was used as a nuclease with a combination of pegRNAs with various PBS lengths. Four endogenous genomic target sites (AAVS1 (SEQ ID NO: 105), B2M (SEQ ID NO: 106), CD5 (SEQ ID NO: 107), and CD38 (SEQ ID NO: 108) that were known to be recognized by wild-type MG3-6 (SEQ ID NO: 104) were targeted with chemically synthesized pegRNAs with varying PBS lengths: 2, 4, 8, 10, 13, 16, and 20 nucleotides (SEQ ID NOs: 109-140). MG3-6 mRNA (SEQ ID NO: 104) was co-transfected with guide RNA (control) or pegRNA (of various PBS lengths). The RNA was reverse transfected with 50,000 HEK293T cells into a 24-well plate. 48 hours post-transfection, cells were lysed in 100 μL of solution. Primers (SEQ ID NOs: 141-148) were used to amplify ˜700 bp of target product (SEQ ID NOs: 105-108) with mastermix. Samples were then cleaned up and Sanger sequenced. Sanger sequences were then processed for ICE analysis to calculate InDel percentage at each target site.

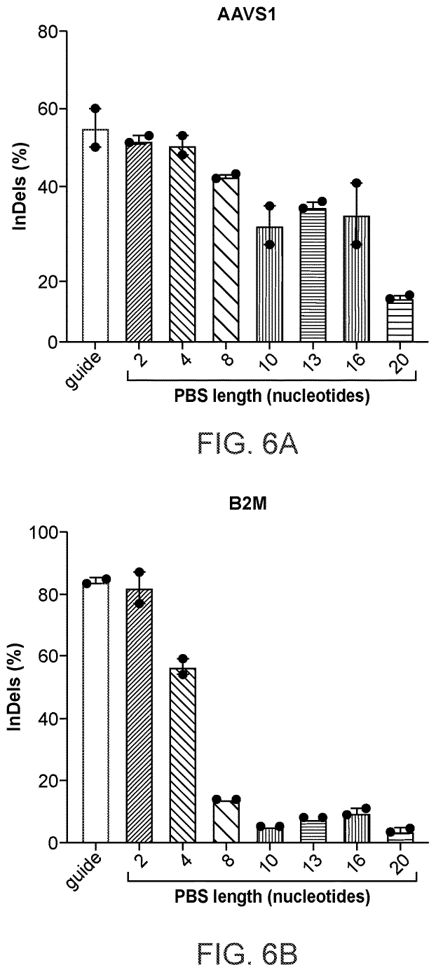

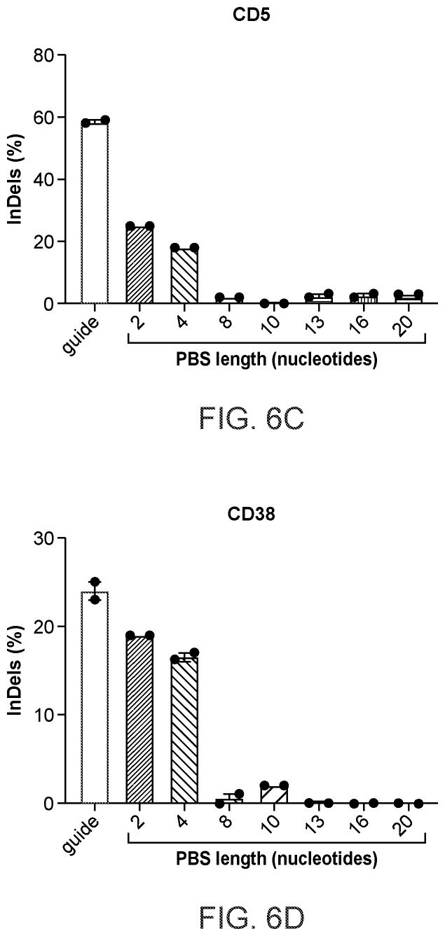

Sanger sequencing traces using ICE analysis showed that wild-type MG3-6 prefers pegRNAs with PBS lengths equal to or less than eight nucleotides (FIGS. 6A-6D). WT guide RNA (without a PBS region) in conjunction with MG3-6 mRNA (SEQ ID NO: 104) gave the highest InDel percentage for all endogenous targets (55% AAVS1, 84%1B2M, 58.5% CD5, and 24% CD38) in comparison to pegRNAs for the corresponding target genes. As the PBS length increased from two to twenty nucleotides, InDel percentage decreased at all endogenous targets (SEQ ID NOs: 105-108). For example, InDel percentage at target site AAVS1 (SEQ ID NO: 105) (FIG. 6A) with a PBS length of 2 nucleotides (SEQ ID NO: 109) (53%) was similar to what was seen with the WT guide RNA (SEQ ID NO: 116) (55%), but with a PBS length of 20 nucleotides (SEQ ID NO: 115), the InDel percentage dropped to ˜11%. Thus, the results show the general rules for pegRNA design for the MG3-6 gene editing system and highlight the importance of identifying RTs with shorter PBS lengths requirements.

### Example 5. Use of Processive RTs in Combination with a Modified pegRNA for Short Corrections, Small Insertions and Deletions (Prophetic)

This example describes the use of reverse transcriptases in combination with a CRISPR nickase and a pegRNA for targeted genome editing in HEK293T cells.

The current setting for prime editing requires a pegRNA that consists of a spacer followed by crRNA, tracr, RTT, and PBS (from 5′-3′). It has been demonstrated that MMLV WT (MMLV1) and MMLV pentamutant (MMLV2) have some level of pegRNA readthrough, thus incorporating parts of the tracr sequence into the genomic DNA (gDNA), a non-desired characteristic as this design creates unwanted mutations in the genomic DNA. RTs from the GII intron family that are expressed well and show high activity for cDNA synthesis in mammalian cells were identified. The RTs from the GII intron family generally show higher processivity than retroviral RTs. Higher processivity translates to RTs being able to read through structured RNA (for example: the crRNA-tracr portion of the pegRNA) and being able to read through small/mid-size chemical modifications in the RNA. Since RTs from the GII intron show good cDNA synthesis activity and good expression in mammalian cells, they are used in a prime editing context to generate small genomic corrections, small insertions, and/or deletions. In order to use processive RTs in the prime editing context, pegRNA readthrough as described above needs to be avoided. To achieve pegRNA readthrough by the RT, bulky modifications are incorporated in the pegRNA, for example into the last base of the RTT if read from 3′ to 5′ (or first base of RTT if read from 5′ to 3′). Bulky modifications include, for example, complex sugars, or complex amino groups, and/or other modifications compatible with RNAs.

Plasmids containing the nickase and any processive RTs to be tested for activity are transfected into cells, for example HEK293T cells, using lipofectamine 2000. Chemically synthesized RNAs (with or without the bulky modifications included) are transfected into the cells using lipofectamine messenger max. 72 hours post-transfection, cells are lysed in 100 L of solution. Primers containing barcodes for next generation sequencing (NGS) (SEQ ID NOs: 100-101) are used to amplify a ˜250 bp target (SEQ ID NO: 102). PCR cleanup is then performed, and samples are NGS sequenced. The resulting FASTQ files are processed using prime editing to determine the percentage of reads with desired change.

The experiments described above allow the use of high-performing RTs in a mammalian cell context for prime editing with little or no pegRNA readthrough.

### Example 6. RTs for Programmable, Large Cargo Integrations Via Target-Primed Reverse Transcription (Prophetic)

This example describes the use of reverse transcriptases with retrotransposase activity in combination with a CRISPR nickase and a pegRNA for targeted genome editing.

Targetable integration of large cargo into human genomic DNA in living cells has been a long sought goal for gene editing. To date, the most efficient way to achieve large cargo integration into the genome of a cell is by using lentiviruses. However, lentiviral-mediated integration lacks the targetability feature, as integration occurs mostly randomly in the open chromatin of a cell. For large cargo integration, RTs with high processivity and high fidelity in conjunction with nucleases are advantageous. The nuclease provides targetability in the gDNA, whereas the RT utilizing a target-primed reverse transcription mechanism can integrate the large RNA cargo into the mammalian gDNA.

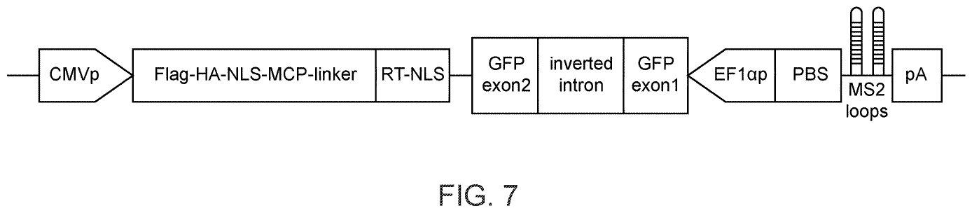

The potential of RT candidates to generate large integrations is tested by their ability to retrotranspose an RNA template containing a GFP cassette that can only produce GFP (and therefore fluorescence) upon successful retrotransposition. The target for retrotransposition is determined by a nuclease. This nuclease creates the primer site through a double-strand break event. Type II nucleases (alternatively Type V nucleases) are tested to identify the best nuclease for gDNA primer generation. The VEGFA gene is chosen for target integration and is targeted by the nuclease together with a chemically synthesized VEGFA guide (SEQ ID NO: 149). The candidate reverse transcriptases are cloned into a plasmid for mammalian expression under the CMV promoter. To localize the RT to the nucleus upon expression, one or more nuclear localization signal (NLS) sequences are added on both N- and C-termini of the RT. Additionally, an MS2 coat protein (MCP) sequence and a Flag-HA (FH) tag are fused to the N-terminus of the RT. MCP is a protein derived from the MS2 bacteriophage that recognizes a 20 nucleotide RNA stem loop (MS2 loop) with high affinity (in the subnanomolar Kd range). Adding MS2 loops to the RT template encoded within the same plasmid ensures that the expressed MCP-RT fusion protein finds the RNA template for reverse transcription. Additionally, a 20 nucleotide sequence complementary to the 3′ overhang generated by the nuclease serves as the primer binding site (PBS) for initiating reverse transcription. To quantify the efficiency of retrotransposition, an inverted GFP cassette driven by an EF1 alpha promoter is cloned downstream of the RT fusion. The GFP is interrupted by an intron (two different intron sequences, named normal intron and chimeric intron, are tested) oriented such that it can only be spliced out from the transcript driven by the CMV promoter and not the EF1 alpha promoter (FIG. 7). Therefore, the cells can express GFP fluorescence only upon the successful retrotransposition of this spliced RNA. The PBS and MS2 loops are cloned downstream of the EF1 alpha promoter, followed by a polyA sequence to stabilize the RNA template. This design ensures that the GFP fluorescence exhibited by cells expressing this plasmid correlates with the efficiency of retrotransposition, and thereby gives a measure of the ability of the RT candidates to reverse transcribe and integrate large stretches of DNA.

RT candidates are cloned into the GFP-based retrotransposition plasmid (SEQ ID NOs: 150-151 and 2580-2581) and isolated for transfection into HEK293T cells. Transfection is performed using Lipofectamine 2000. 24 hours later, cells are split into a medium containing Puromycin to select for transfected cells expressing the plasmid. Five days later, cells are flowed on a cell sorter, and the percentage of GFP positive cells in the population is quantified.

To test hundreds or thousands of RTs and/or conditions (engineered systems) the method above also allows for high-throughput testing. Hundreds or thousands of conditions are pooled together and a single pooled plasmid transfection is performed. Cells expressing GFP are sorted five days post transfection. Identification of best performing RTs is made by sequencing GFP-positive cells and mapping the RTs by using a combination of random primers and primers matching the second exon of GFP. Enriched RTs by this pooled method are then validated individually.

This methodology allows for the identification of RTs capable of large cargo integration mediated by a target-primed reverse transcription mechanism. The engineered nuclease/RT constructs thus allow the development of an RNA-mediated large cargo integration into genomic DNA of mammalian cells.

### Example 7. RTs for Programmable, Large Cargo Integrations Mediated by a Single-Stranded DNA Transposase (Prophetic)

This example describes the use of reverse transcriptases with retrotransposase activity in combination with TnpA for targeted genome editing.

Retrons are DNA elements that contain an RT enzyme encoded downstream of a conserved non-coding structural RNA. The non-coding RNA consists of two inverted regions, referred to as msr and msd. When the retron RT recognizes the folded ncRNA, it reverse transcribes the msd portion (template) producing ssDNA.

IS200/IS605 transposons are a type of mobile genetic element that integrate ssDNA at specific target sites by a TnpA transposase. TnpA excises a donor by recognizing structural motifs at each donor end, integrating it at a recognized target site accessible as ssDNA.

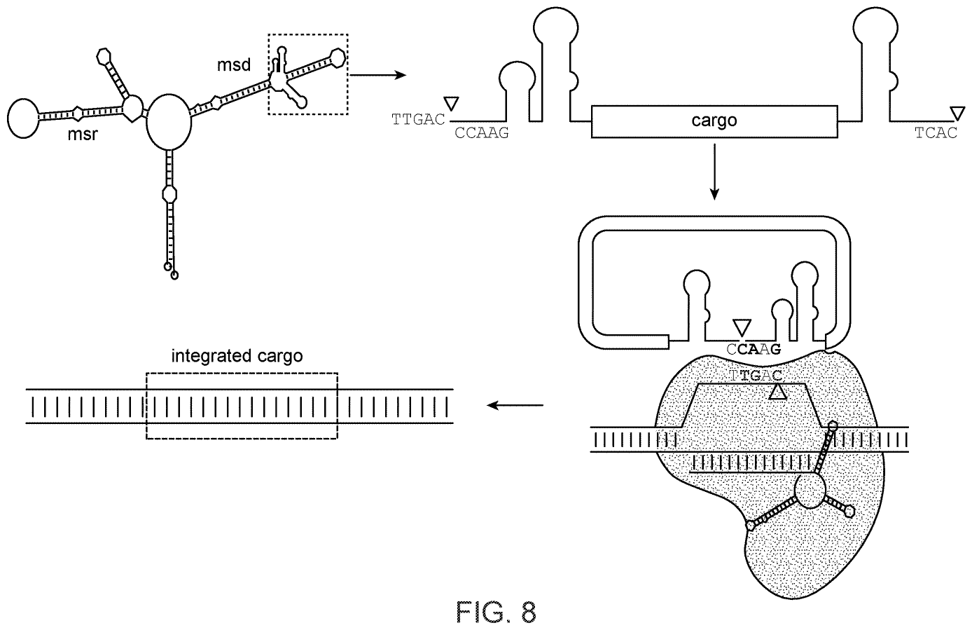

An ssDNA produced by a retron RT can be used as a template by TnpA for programmable integration of desired cargo into a specific target site. Specifically, the retron msd can contain the desired cargo (for example, an antibiotic resistance cassette or fluorescent marker) flanked by LE and RE structural motifs recognizable by TnpA. The TnpA transposase excises and circularizes the ssDNA donor, and integration into a target occurs via recognition of a specific motif available through an R-loop formed by the RNA-guided recognition and binding of an engineered (nickase or dead) effector (for example, MG3-6) (FIG. 8).

### Example 8. RTs for Short Corrections, Small Insertions, and Deletions

Testing Reverse Transcriptase Candidates Untethered from the Cas Nickase

Reverse transcriptase (RT) candidates from the MG151 and MG153 families were cloned into a plasmid where expression of the RT candidate is driven by the CMV promoter. The plasmid was then isolated for transfection in HEK293T cells. Another plasmid containing a nickase spCas9 (H840A) driven via CMV promoter and the RT-containing plasmid were co-transfected. A chemically synthesized pegRNA (SEQ ID NOs: 656-697) containing the desired edit in the RT template was transfected. All components (plasmids and pegRNAs) were reverse transfected into 150,000 HEK293T cells in a 24 well plate. 72 hours post-transfection, cells were lysed in 100 μL of solution. Primers containing barcodes for next generation sequencing (NGS) (SEQ ID NOs: 649-650) were used to amplify a ˜250 bp target (SEQ ID NOs: 654-655) with mastermix. PCR cleanup was then performed, and samples were sent for NGS sequencing. FASTQ files were then processed using prime editing to determine the percentage of reads with the desired change.

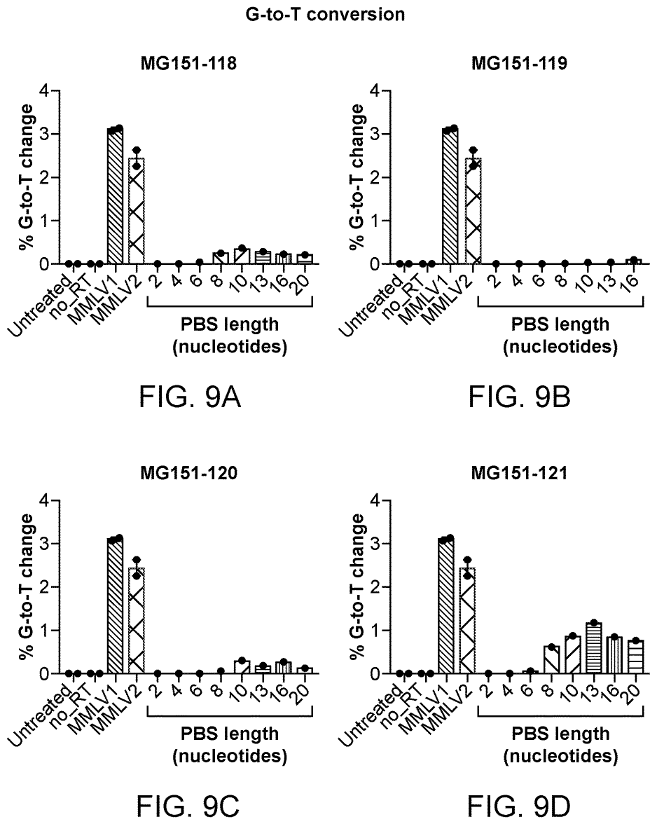

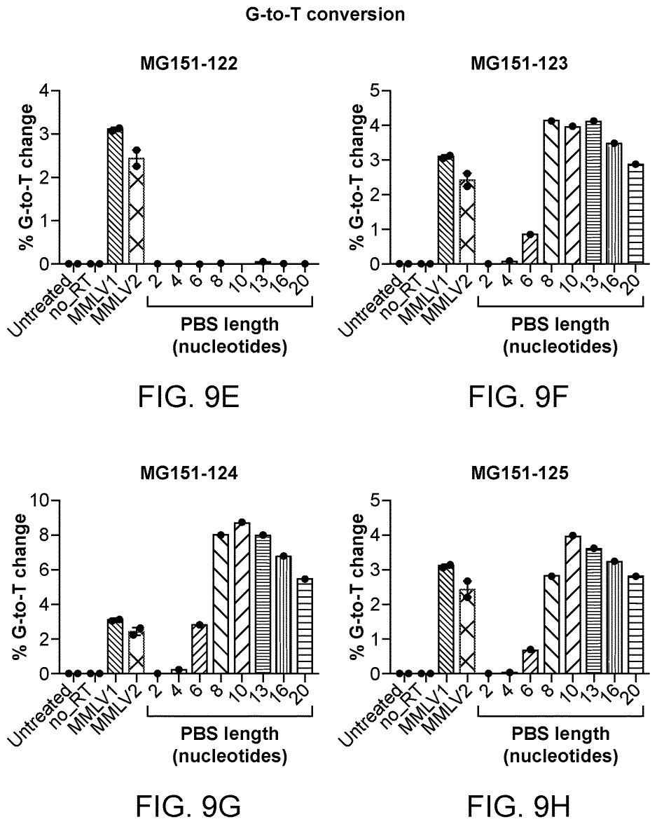

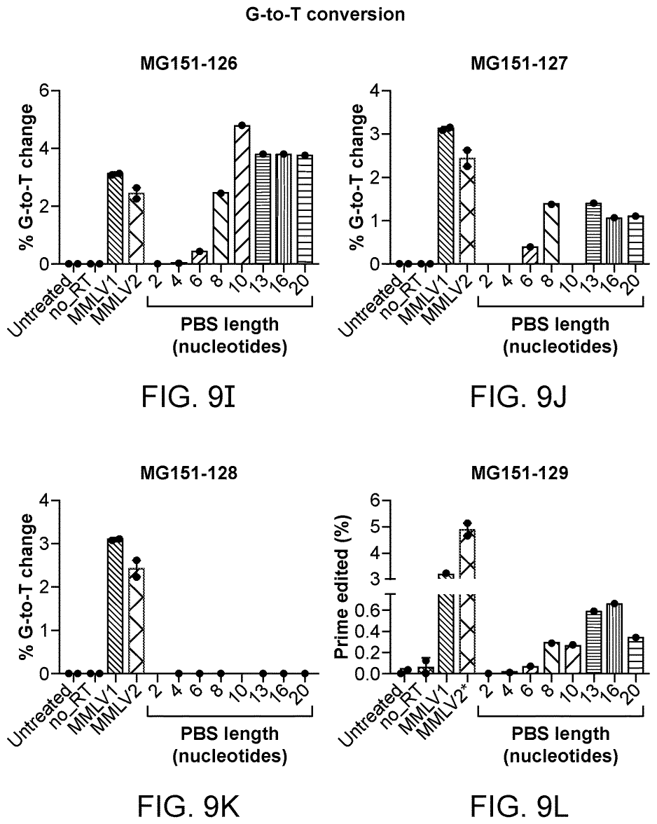

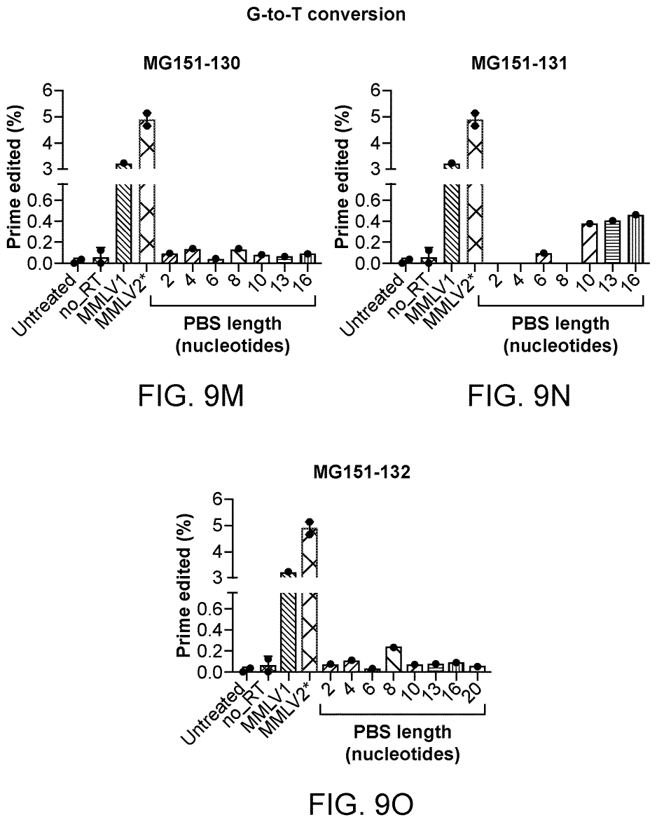

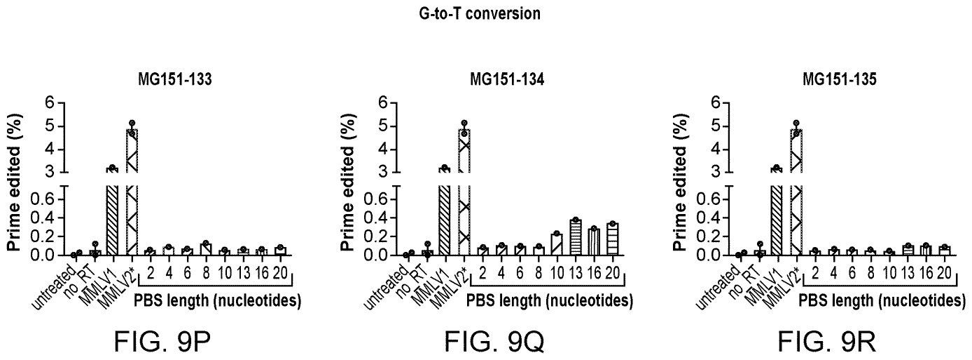

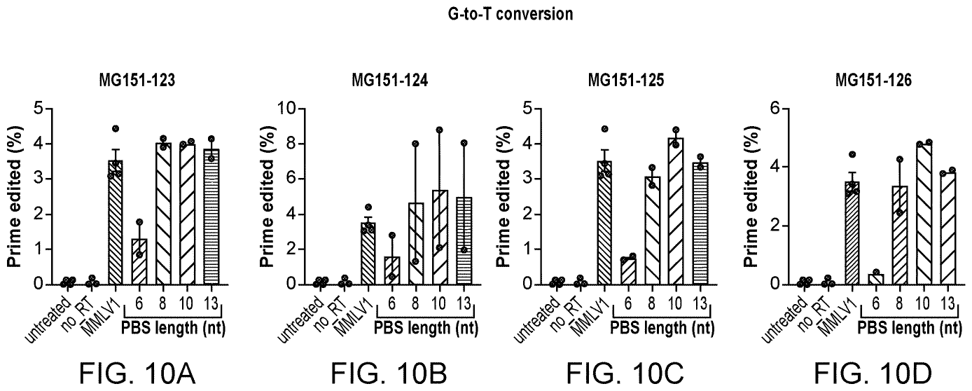

Untethered MG151 candidates MG118-MG135 (SEQ ID NOs: 710-727) were tested for prime editing in HEK293T cells to determine percent change of a desired correction. Percent editing for each RT is shown in FIGS. 9A-9R for each pegRNA with varying PBS lengths (2, 4, 6, 8, 10, 13, 16, and 20 nucleotides). In a single replicate, MG151-123 through MG151-126 had equivalent or superior editing efficiencies as compared to MMLV WT RT (FIGS. 9F-9I). These results were reproduced, and the biological replicates are shown in FIGS. 10A-1OD, with the four candidates editing at comparable levels to MMLV WT when challenged to perform a G-T transversion. Importantly, all of these candidates performed well with PBS lengths of 8nt, enabling the shortening of the pegRNA and the PBS-spacer hybridization window.

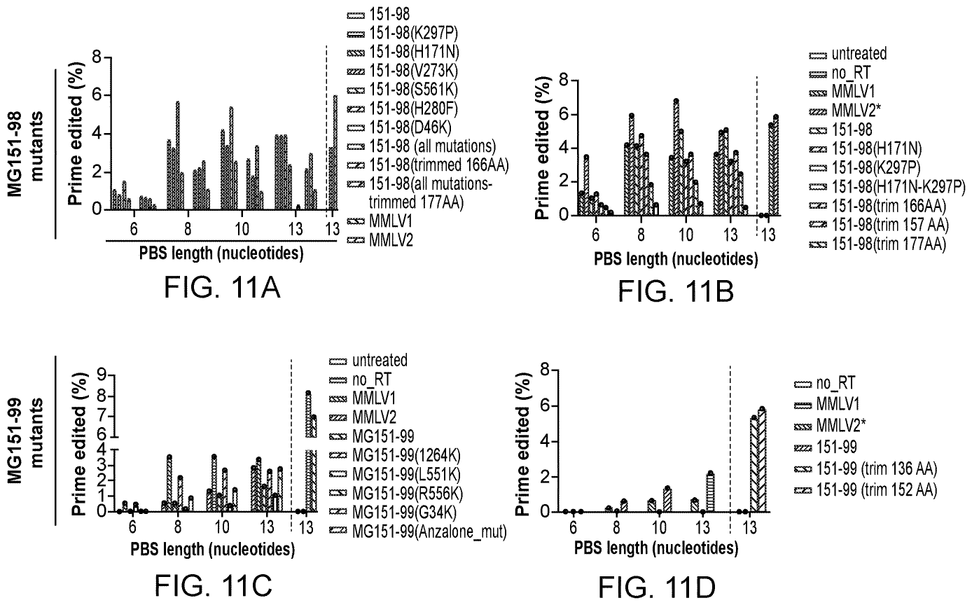

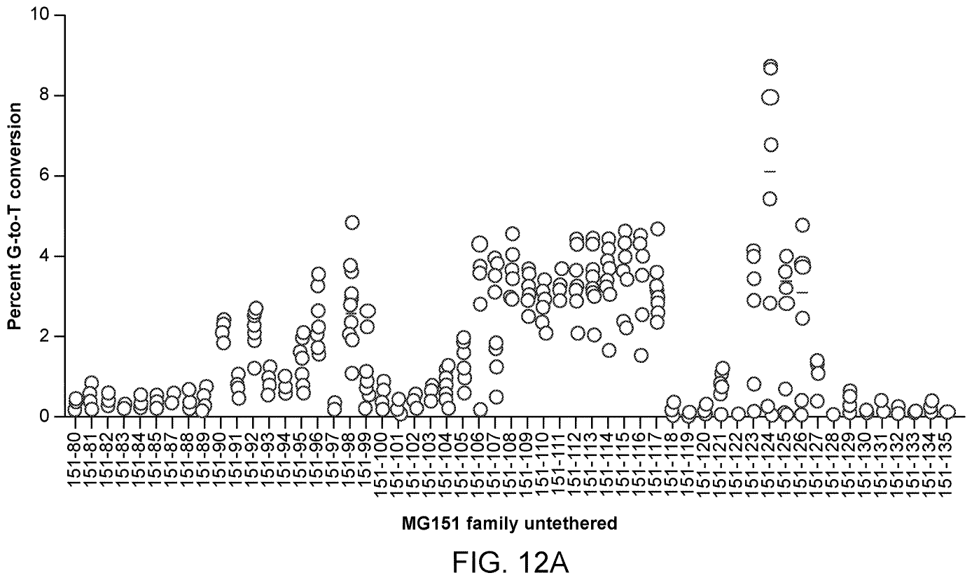

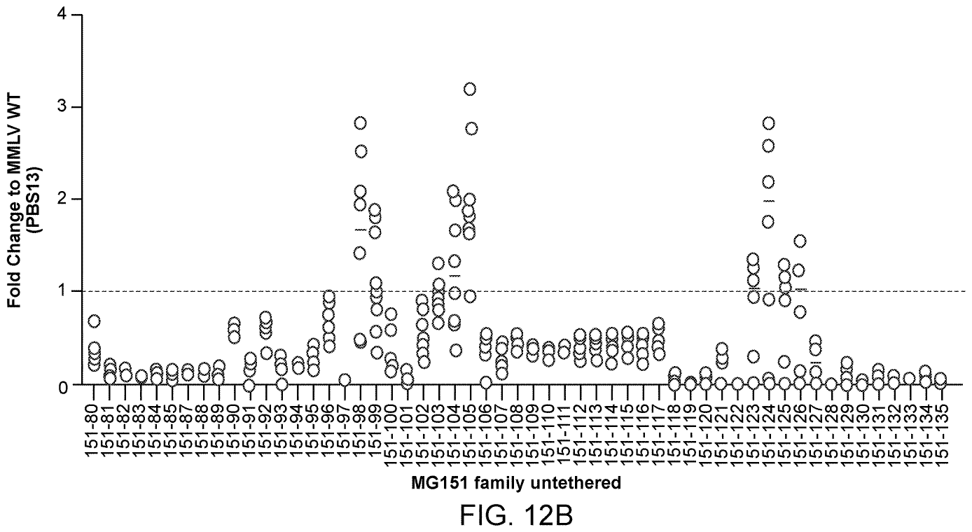

Moreover, two candidates from the 151 family (MG151-98 and MG151-99) were subjected to rational engineering to install beneficial mutations observed in other RTs (Anzalone et al, 2022). Various point mutations by themselves or combined, as well as truncations of the RNaseH domain, were evaluated (SEQ ID NOs: 750-766). Mutations H171N, K297P, and trimming the last 166aa of MG151-98 improved prime editing efficiency, with some of those mutations outperforming MMLV pentamutant (FIGS. 11A-11B). For MG151-99, I264K, R556K, and the trimming the last 152 aa proved to be beneficial (FIGS. 11C-11D). An overview of MG151 candidates evaluated for G-T transversion in HEK293T cells targeting the VEGFA gene is shown in FIGS. 12A-12B.

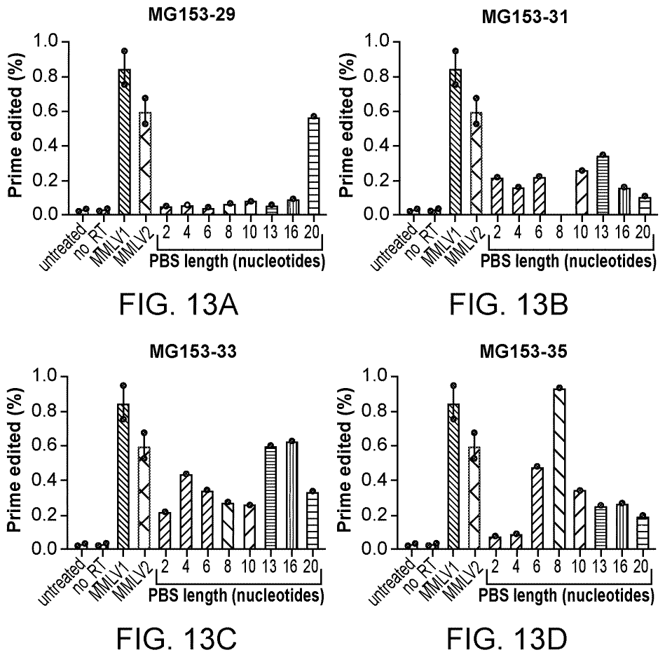

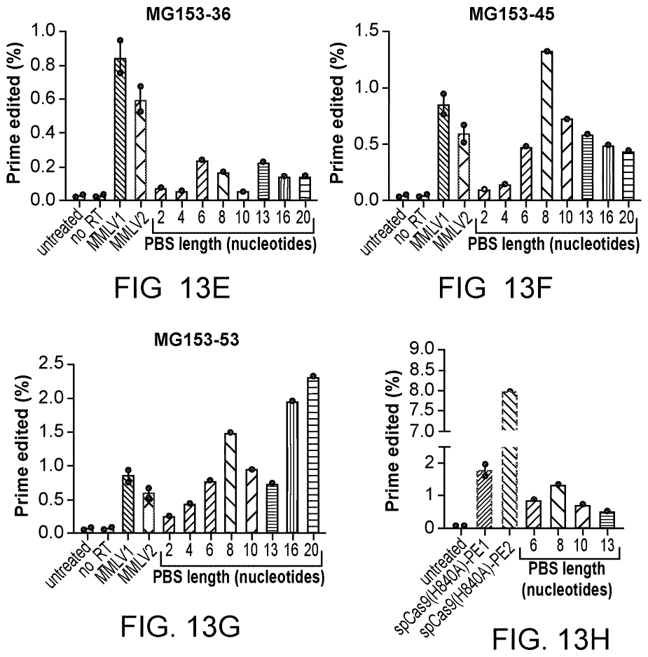

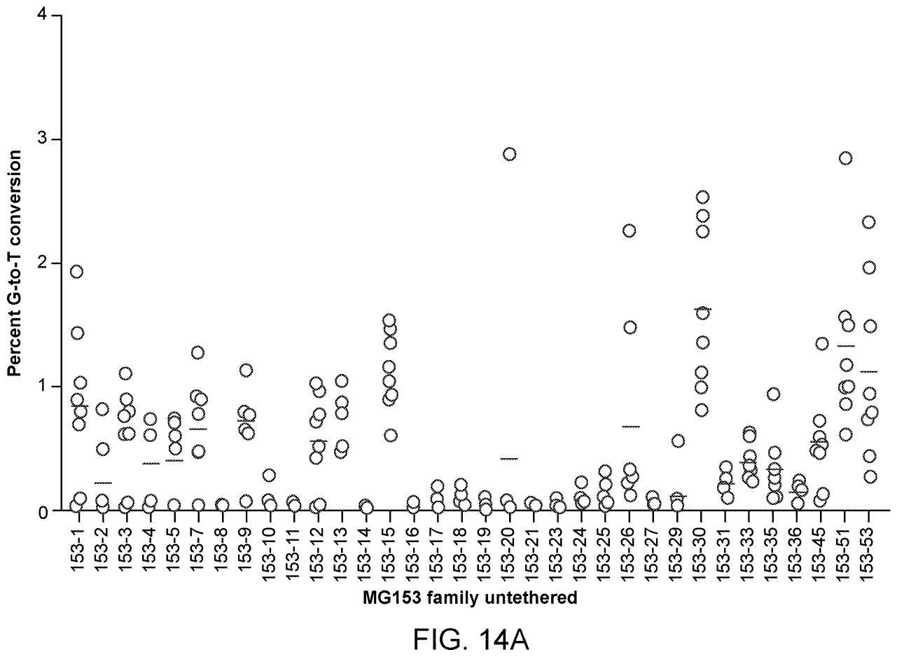

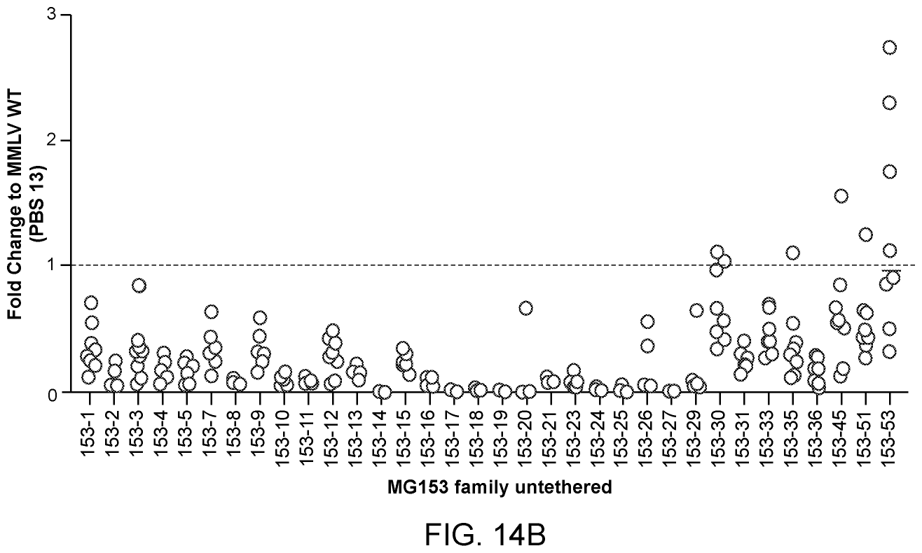

Untethered MG153 candidates MG153-29, MG153-31, MG153-33, MG153-35, MG153-36, MG153-45, and MG153-53 were tested for prime editing in HEK293T cells to determine the percent change of a desired correction. Percent editing for each RT is shown in FIGS. 13A-13H for each pegRNA with varying PBS lengths (2, 4, 6, 8, 10, 13, 16, and 20 nucleotides). Several RTs, including MG153-33, MG153-35, MG153-45, and MG153-53, are active at comparable or superior levels as compared to MMLV WT RT (FIGS. 13C-13D and FIGS. 13F-13G). Importantly, MG153-53 outperformed MMLV WT by over 2-fold (FIG. 13G). This candidate was also active when tested as a fusion protein with Cas9 (FIG. 13H), demonstrating its versatility. An overview of MG153 candidates evaluated for G-T transversion in HEK293T cells targeting the VEGFA gene is shown in FIGS. 14A-14B.

### Testing Reverse Transcriptase Candidates Tethered to a Cas Nickase

RT candidates were cloned into a plasmid containing the nickase spCas9(H840A) to generate an RT-nickase fusion. The CMV promoter drove the expression of the fusion protein, which contained a thirty three amino acid linker (SEQ ID NO: 103) between the nickase and RT candidate. The fusion protein was then transfected into HEK293T cells and processed for NGS as described above.

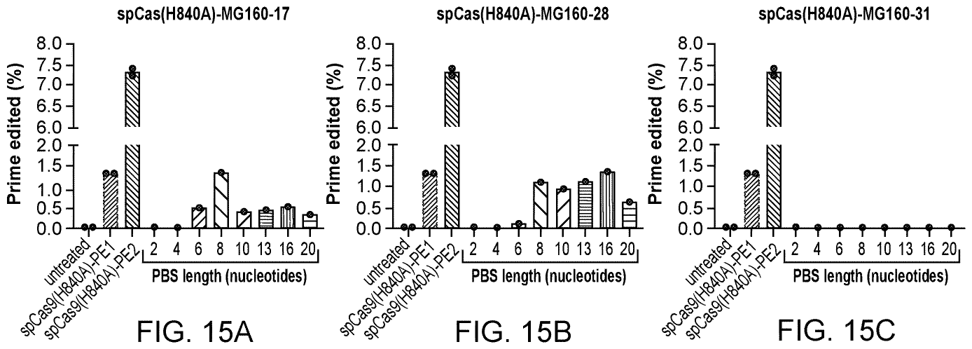

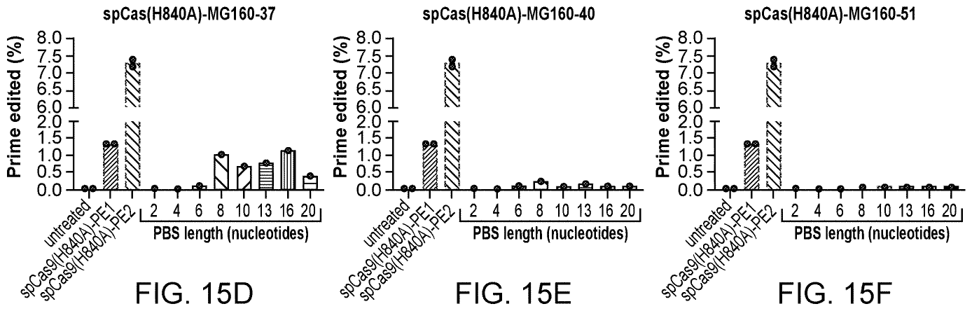

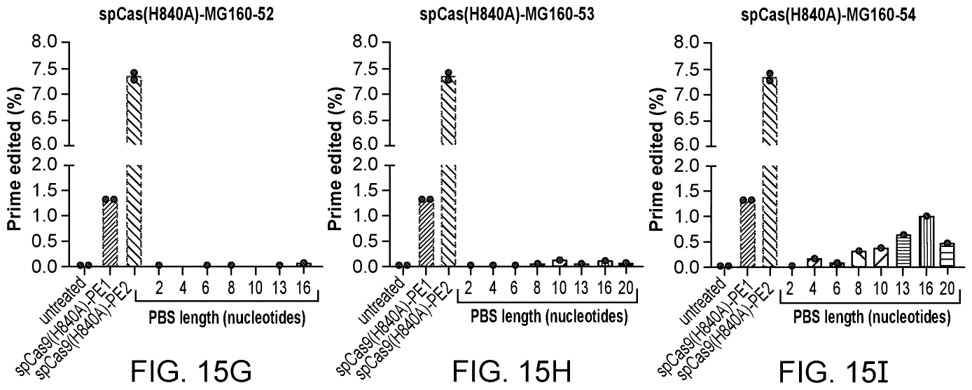

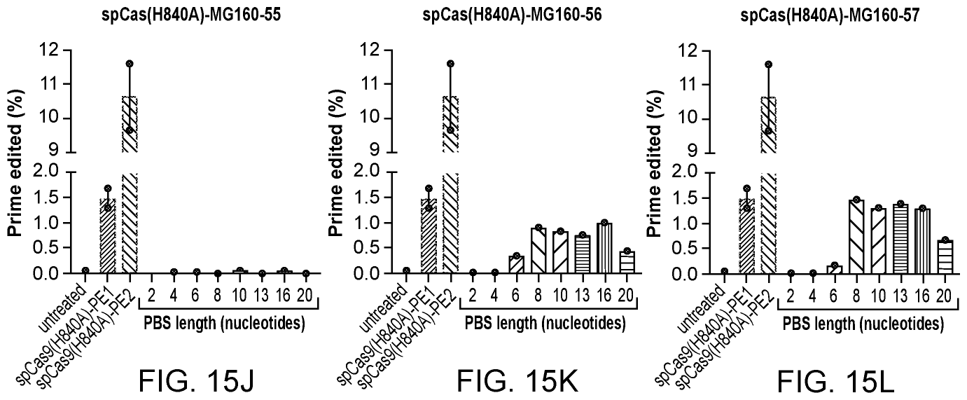

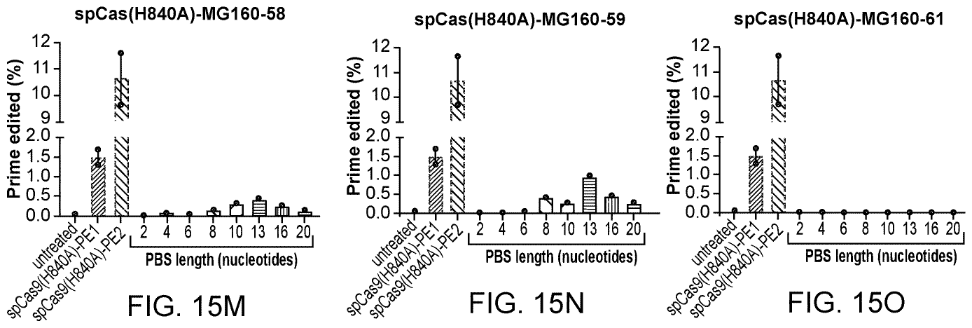

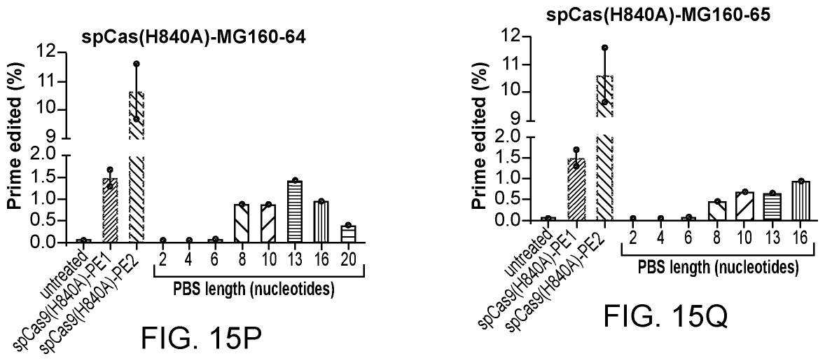

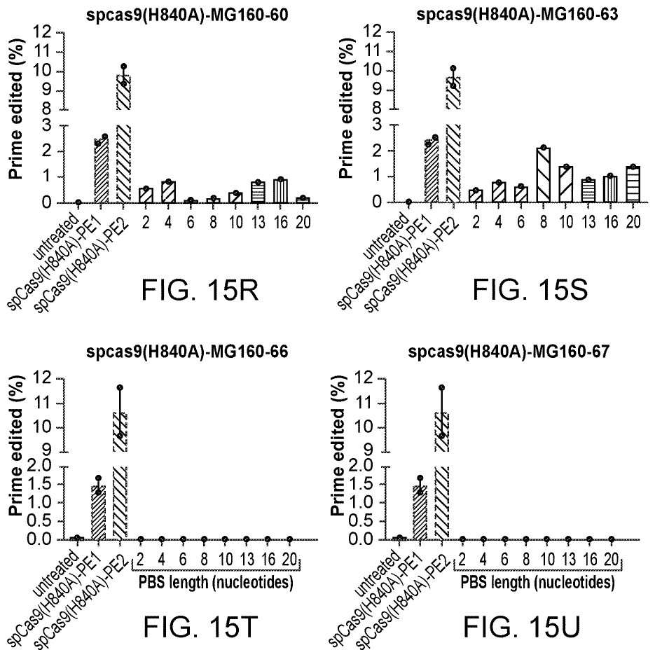

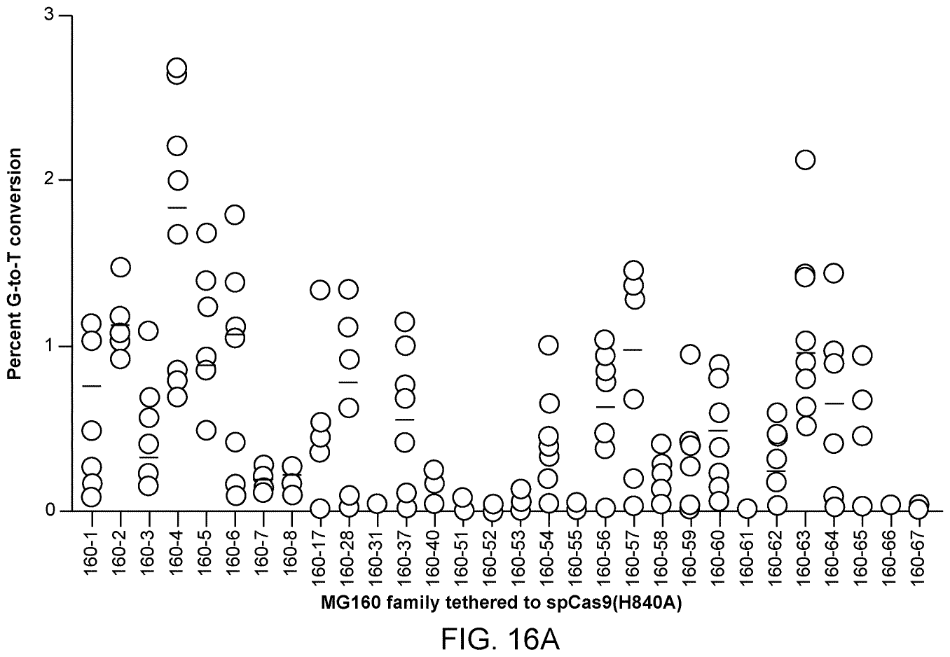

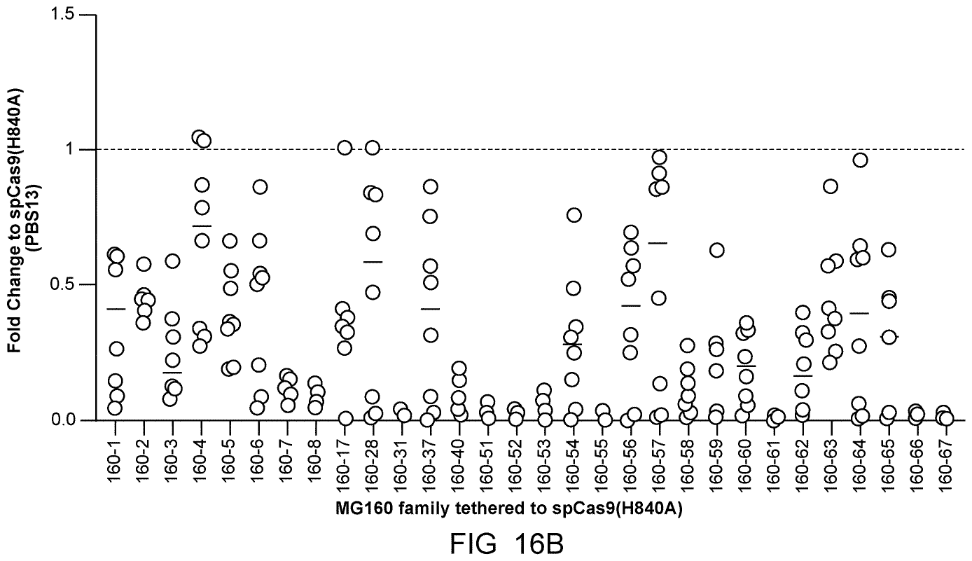

Editing activity of RT candidates MG160-17, MG160-28, MG160-31, MG160-37, MG160-40, and MG160-51 through MG160-67 is shown in FIGS. 15A-15U. Several candidates showed comparable editing levels to MMLV WT, including MG160-17, MG160-28, MG160-37, MG160-54, MG160-56, MG160-57, MG160-59, MG160-64, MG160-65, and MG160-63. An overview of MG153 candidates evaluated for G-T transversion in HEK293T cells targeting the VEGFA gene is shown in FIGS. 16A-16B.

The results demonstrate that several RTs from different phylogenetic families exhibited similar or higher activity than MMLV WT RT in a prime editing context. Having activity across a broad range of families allows for the nomination of RT candidates which may be best suited for different kinds of modifications (i.e., SNP corrections, insertions, or deletions). Moreover, several RTs with sizes ˜250 aa were identified that perform similarly to or outperform MMLV WT. Their small size (about one third of the size of the MMLV WT RT) makes them promising candidates for development of compact systems that can enable efficient delivery using adenoviruses (AAVs) and lipid nanoparticles (LNPs).

### RTs for Small Insertions and Deletions

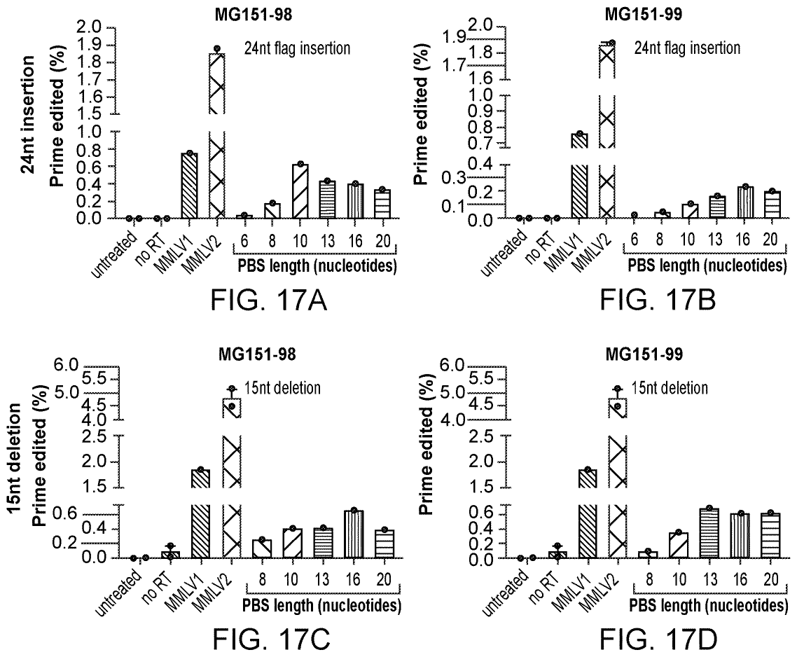

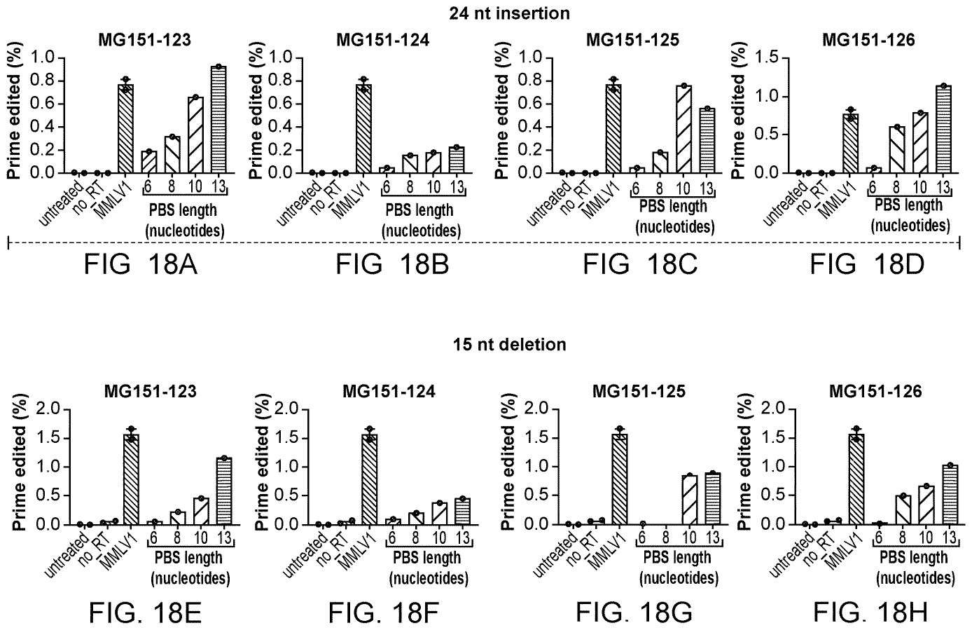

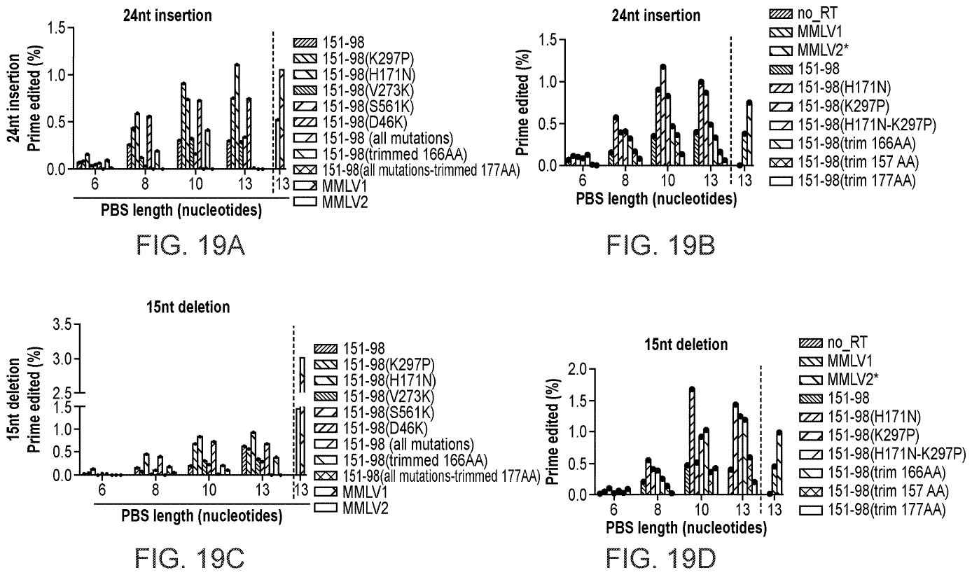

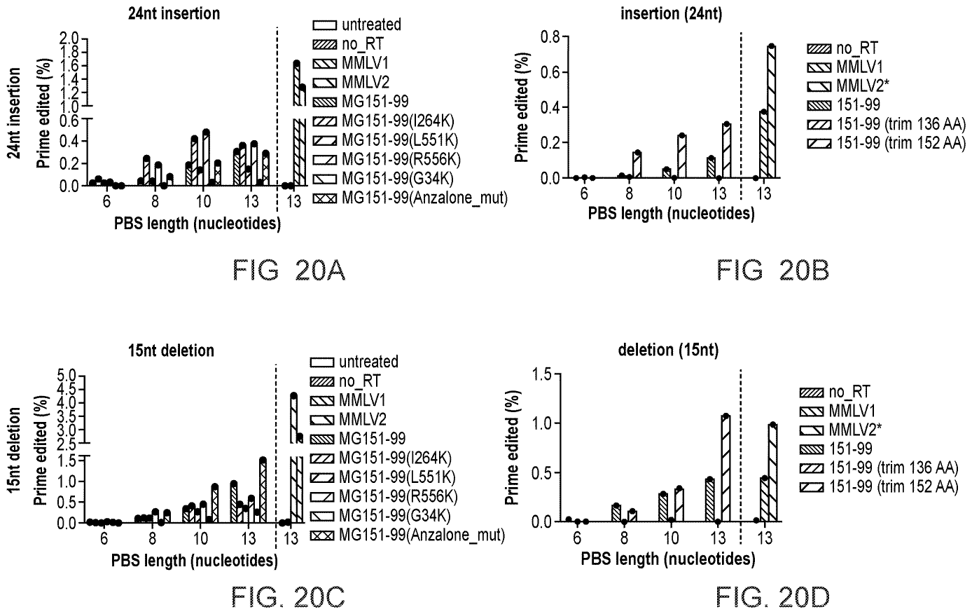

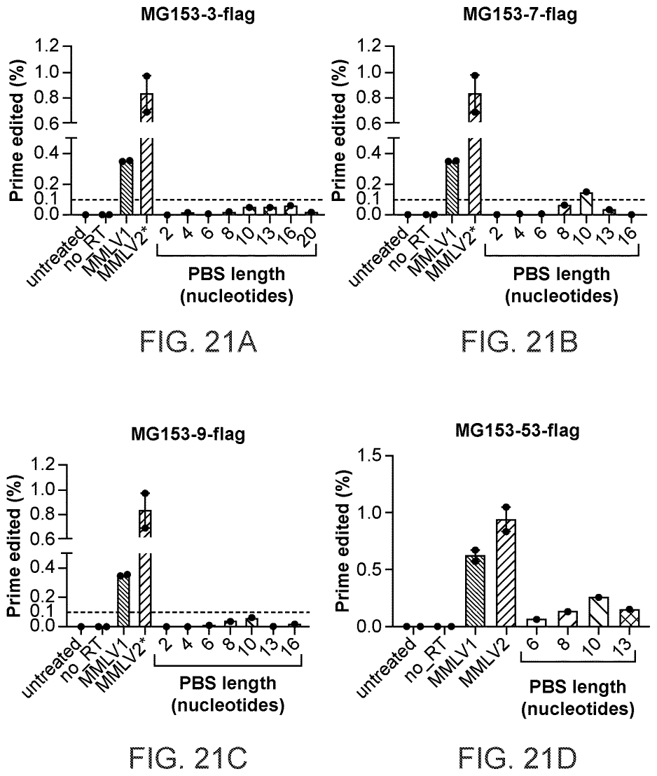

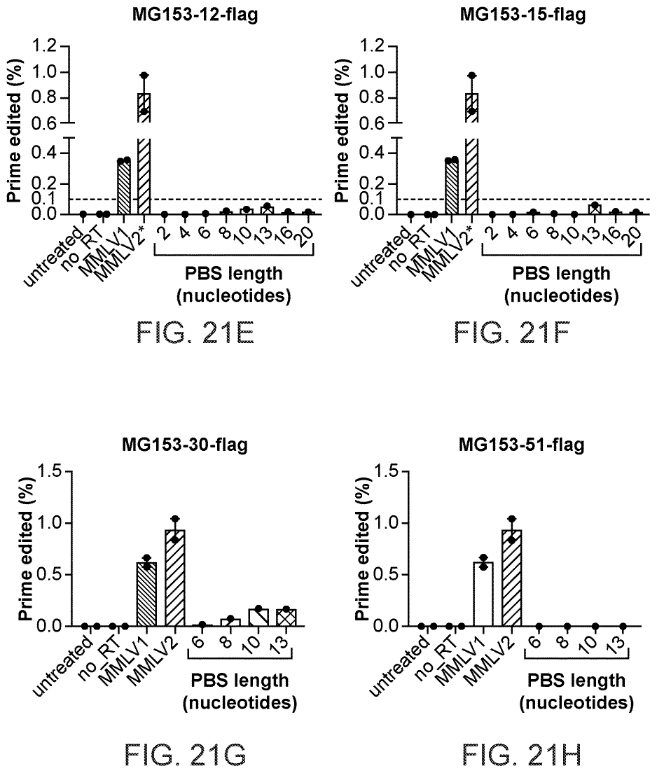

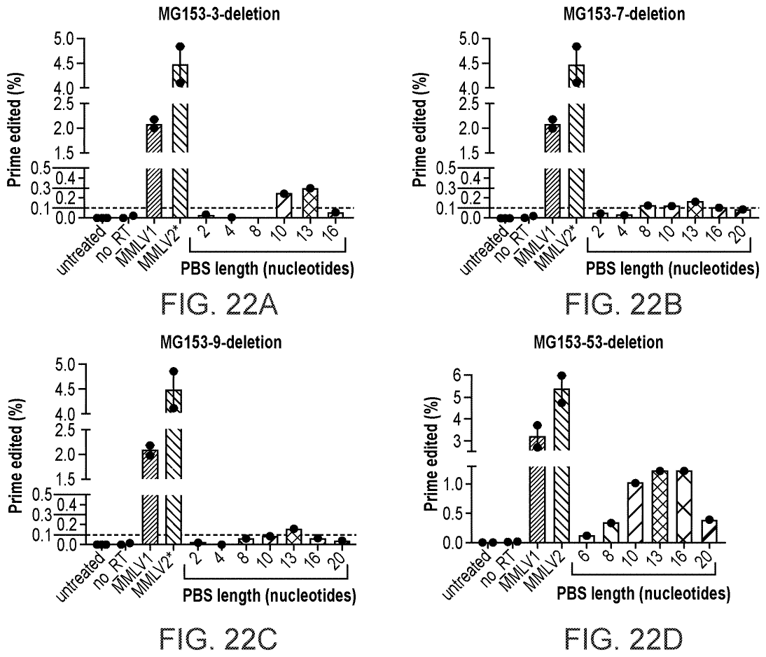

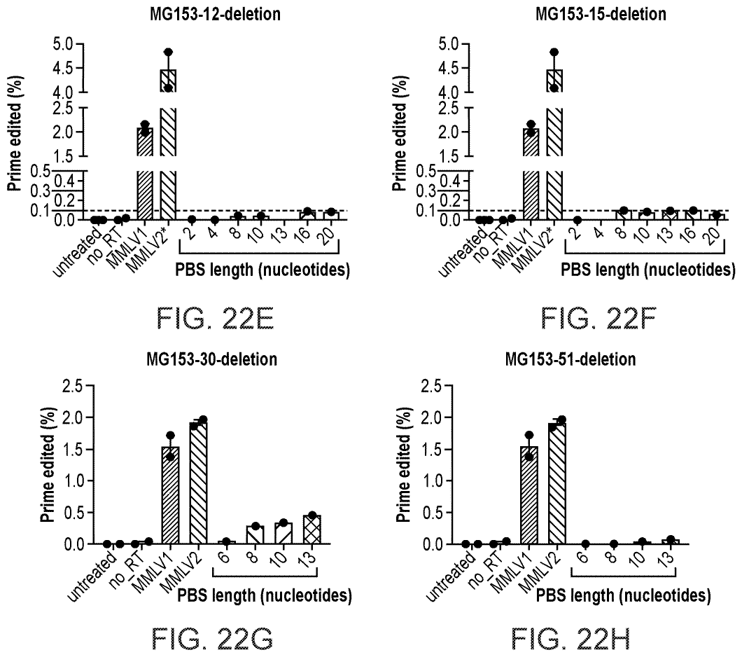

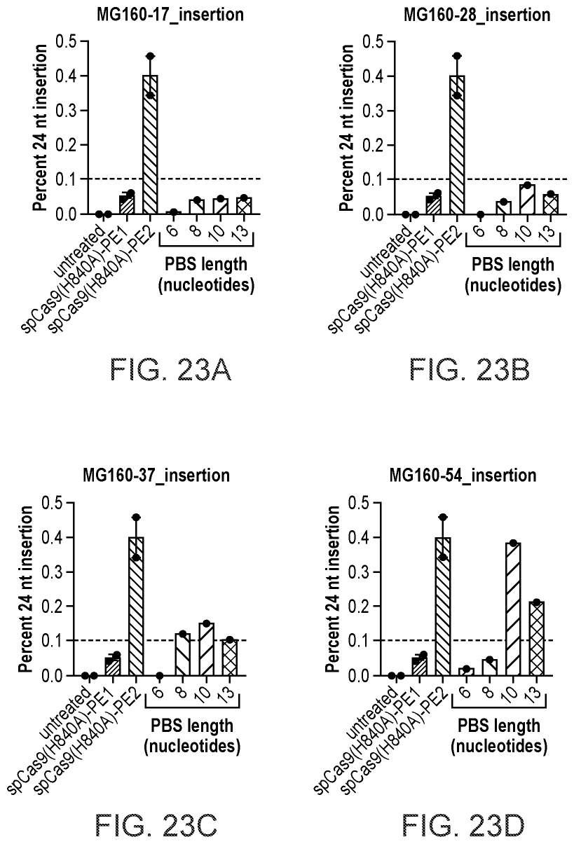

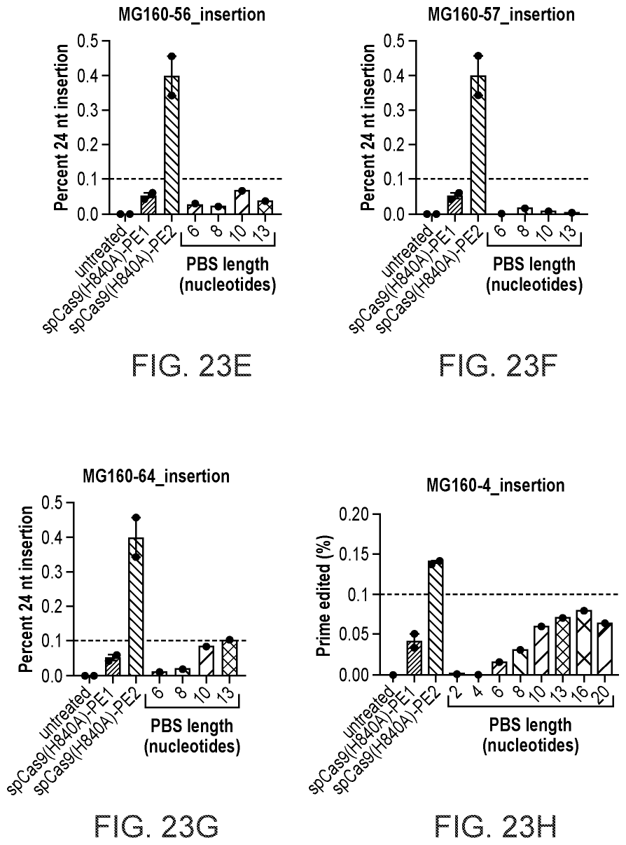

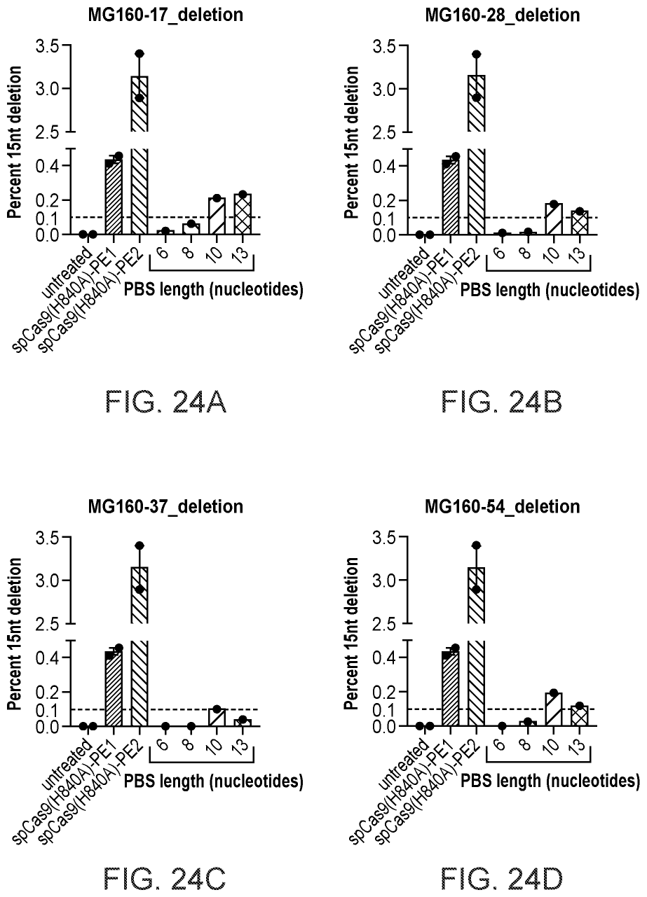

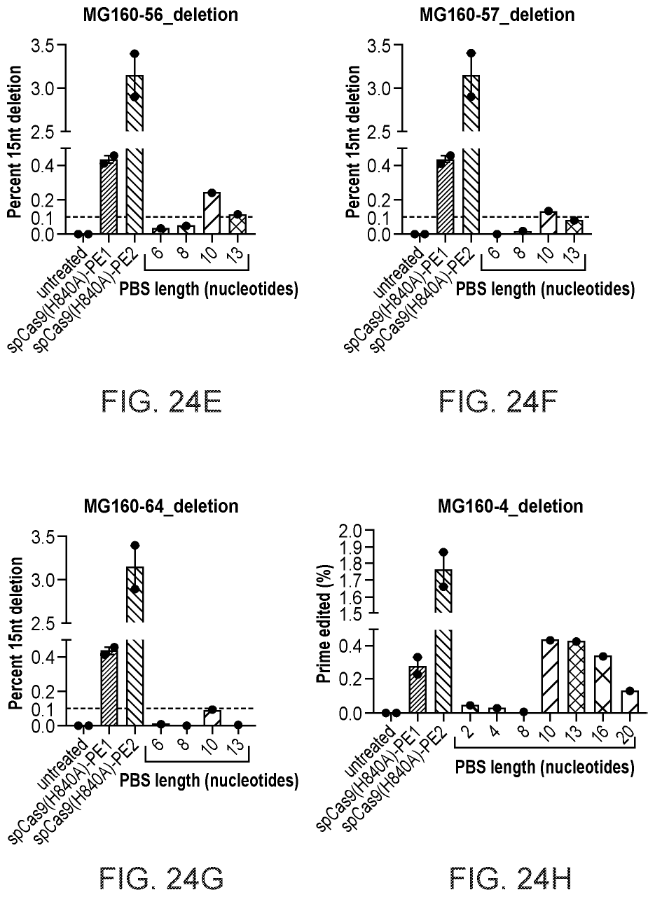

RT candidates from the MG151, MG153, and MG160 families were challenged to perform 24nt insertions, as well as 15nt deletions, in the VEGFA gene to test their ability to perform small and mid-size corrections (FIGS. 17A-24H). Most candidates that performed well in the G-T transversion experiments were able to also perform insertions and deletions efficiently. For example, well performing candidates from the MG151 family included MG151-98, MG151-99 (FIGS. 17A-17D), MG151-23 (FIGS. 18A and 18E), and MG151-26 (FIGS. 18D and 18H), as well as the engineered variants K297P, H171N, and 166 aa trimmed for MG151-98 (FIGS. 19A-19D) and I264K, R556K, and 152 aa trimmed for MG151-99 (FIGS. 20A-20D). MG153-53 was a well performing candidate from the MG153 family (FIGS. 21D and 22D). Well performing candidates from the MG160 family included MG160-4 (FIGS. 23H and 24H), MG160-37 (FIGS. 23C and 24C), MG160-54 (FIGS. 23D and 24D), and MG160-64 (FIGS. 23G and 24G).

### Example 9. Nucleases for Mediating Short Genomic Corrections in Conjunction with Reverse Transcriptases

The targetability required for the installation of genomic corrections, insertions, or deletions using RTs can be provided by a nickase. The nickase nicks the non-targeting strand, creating a primer for reverse transcription. The gRNA that accompanies the nickase is a modified version (pegRNA) that consists of a 3′ extension containing the RNA template (RTT) and the PBS. The PBS and the spacer may be complementary to each other, and this complementarity can cause gRNA structural disruption, leading to disruption of pegRNA interaction with its nickase and, ultimately, failure to target the gene of interest. Because each nuclease interacts with its own gRNA, the pegRNA design and requirements will vary from system to system.

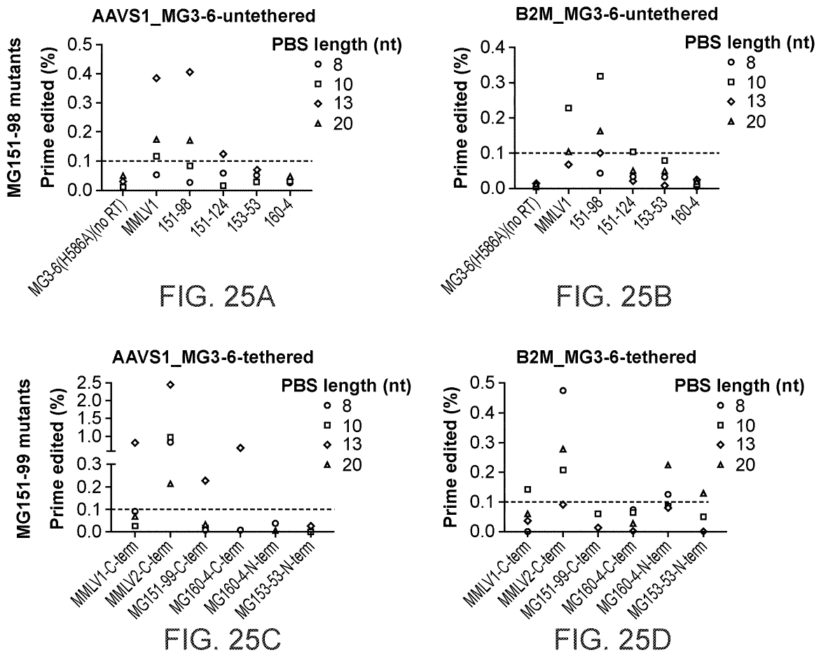

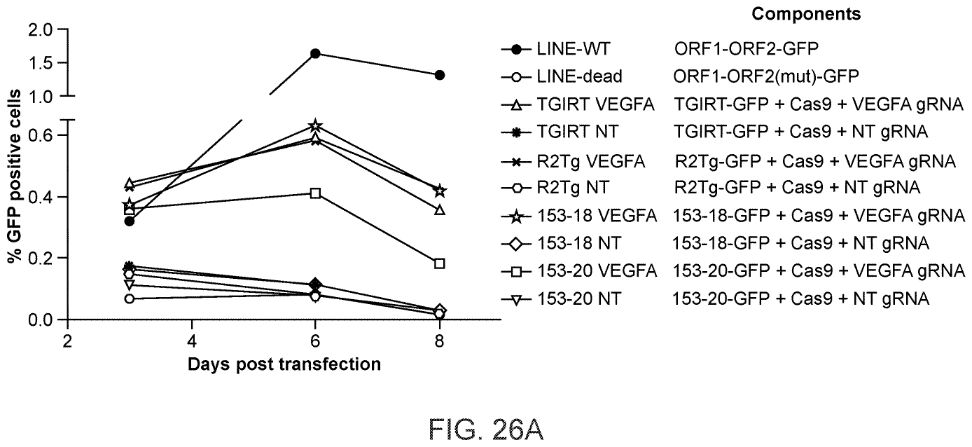

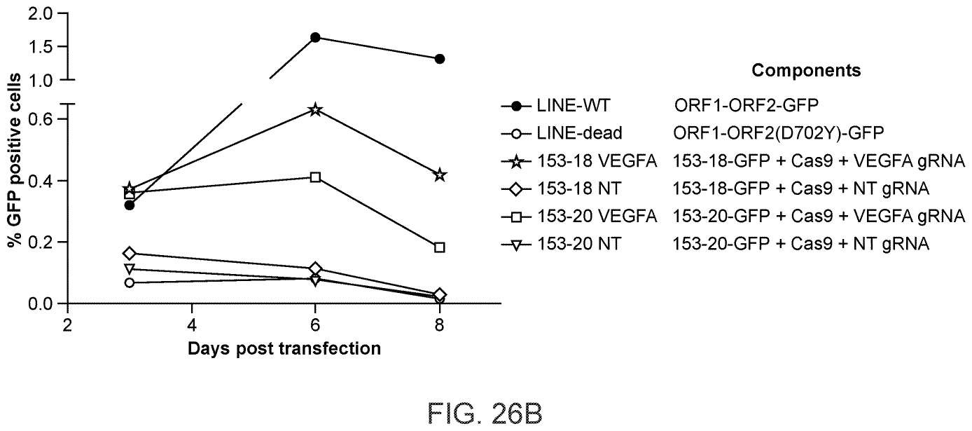

In order to test the versatility of RT candidates in conjunction with nucleases, we tested several RTs with MG3-6 H586A nickase, either untethered or tethered (fused) (FIGS. 25A-25D). A modest level of editing was detected in both tethered and untethered systems. The levels of editing can be improved by optimizing the constructs as well as the delivery methods.

### Example 10. RTs for Programmable Large Cargo Integrations Via Target-Primed Reverse Transcription

The ability of RT candidates to generate large integrations was tested by their ability to retrotranspose an RNA template containing a GFP cassette that can only produce GFP (and therefore fluorescence) upon successful retrotransposition. The target for retrotransposition is determined by a Cas nuclease.

RT candidates were cloned into a GFP-based retrotransposition plasmid and isolated for transfection into HEK293T cells. Plasmid transfection was performed using Lipofectamine 2000, while Cas9 mRNA and chemically synthesized guides were transfected using Lipofectamine messenger max. 24 hours later, cells were split into a medium containing Puromycin to select for transfected cells expressing the plasmid. Three, six, and eight days later, cells were flowed on a cell sorter, and the percentage of GFP positive cells in the population was quantified.

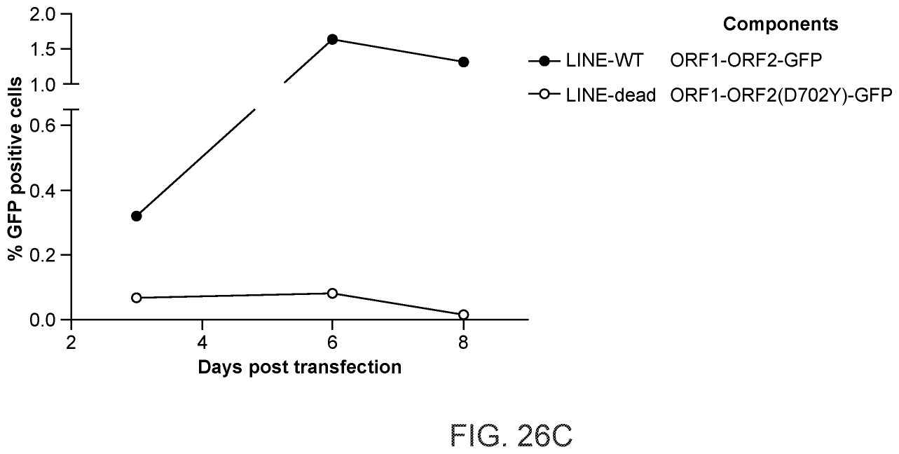

MG candidates MG153-18 and MG153-20 showed GFP fluorescence increasing from D3 to D6, above the non-targeting background, indicating successful retrotransposition in the VEGFA gene (FIGS. 26A-26C). These results show that the MG RTs are capable of long (>1 kb) targeted integrations in the human genome.

### Example 11. Prime Editing of Engineered RTs

Reverse transcriptase (RT) candidates from the MG151 family, MG160 and MG153 families, were cloned into a plasmid where expression of RT candidate is driven by the CMV promoter. The plasmid was then isolated for transfection in HEK293T cells. Another plasmid containing a nickase spCas9 (H840A) driven via CMV promoter, and the RT containing plasmid were cotransfected. Chemically synthesized pegRNA containing the desired edit in the RT template was transfected. All components (plasmids and pegRNAs) were reverse transfected into 150,000 HEK293T cells in a 24 well plate. 72 hours post transfection, cells were lysed in 100 μL of solution. Primers containing barcodes for next generation sequencing (NGS) were used to amplify a ˜250 bp target. PCR clean up was then performed and samples were sent for NGS sequencing. FASTQ files were then processed using prime editing to determine the percentage of reads with desired change.

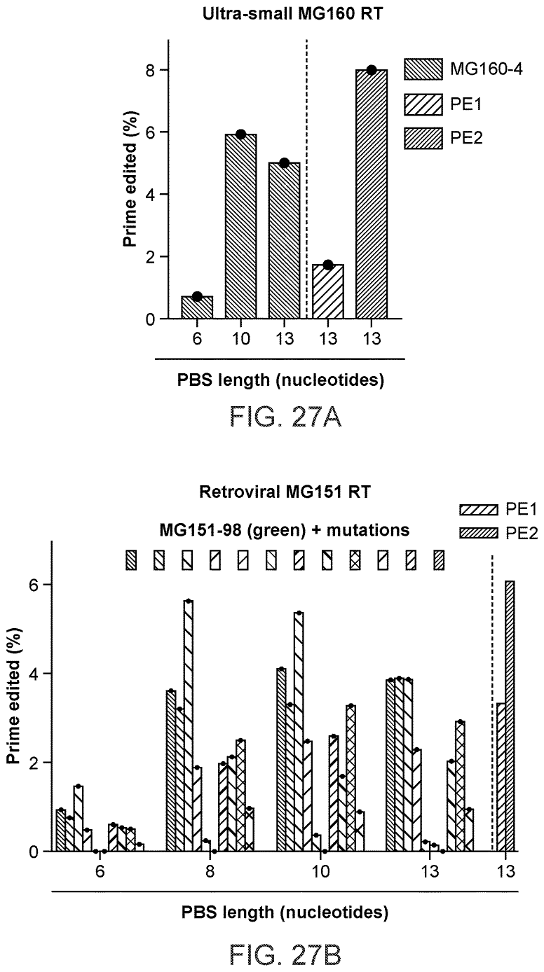

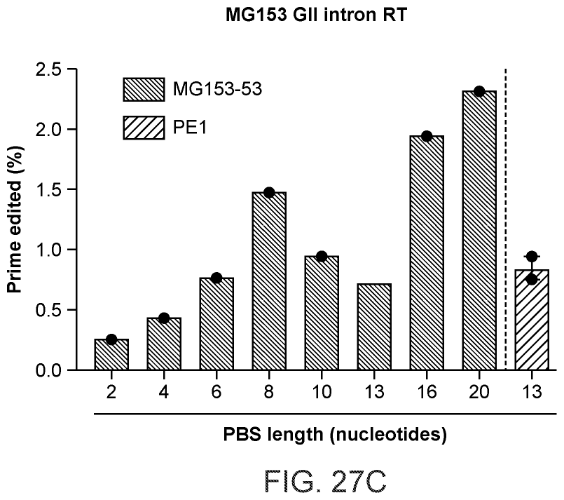

Data is seen in FIGS. 27A-27C. G-T transversion in the VEGFA gene is shown for 3 RTs from different families across multiple sizes of primer binding sites (PBS length). The ultra small MG160-4 candidate outperformed MMLV WT (PE1) and performed closely similar to the gold standard MMLV pentamutant (PE2). The MG151-98 candidate in its WT form performed closely to PE1. The mid size 153-53 candidate outperformed PE1 across a variety of PBS lengths. Moreover, MG151-98 was subjected to rational engineering to install beneficial mutations observed in other RTs. Various point mutations by themselves or combined, as well as truncations of the RNaseH domain were evaluated. Mutations H171N, K297P and trimming the last 166 aa of MG151-98 improved prime editing efficiency, with some of those mutations outperforming MMLV pentamutant.

### Example 12. Processive RT for Large RNA-Templated Integrations

The ability of candidate reverse transcriptase enzymes to produce cDNA in a mammalian environment was tested by expressing them in mammalian cells and detecting cDNA synthesis by qPCR. Reverse transcriptases were cloned in a plasmid for mammalian expression under the CMV promoter. A 4 kb RNA template was generated by in vitro transcription and hybridized to a DNA primer.

A plasmid containing MCP fused to the RT candidate under CMV promoter was cloned and isolated for transfection in HEK293T cells. Transfection was performed using lipofectamine 2000. mRNA codifying dCas9 fused to nanoluciferase was made. In order to degrade any DNA template left in the mRNA preparation the reaction was treated DNase for 1.5 hour and the mRNA was cleaned. The mRNA was hybridized to a complementary DNA primer in 10 mM Tris pH 7.5, 50 mM NaCl at 95 C for 2 min and cooled to 4 at the rate of 0.1 C/s. The mRNA/DNA hybrid was transfected into HEK293T cells 6 hours after the plasmid containing the MCP-RT fusion was transfected. 18 hours post mRNA/DNA transfection cells were lysed using solution, 100 ul of quick extract is added per 24 well in a 24 well plate. The RNA template was ˜4247 nt. Primers to amplify first and last 100 bps products from the newly synthesized cDNA (4100 bp) were designed, along with TaqMan probes to quantify their amplification.

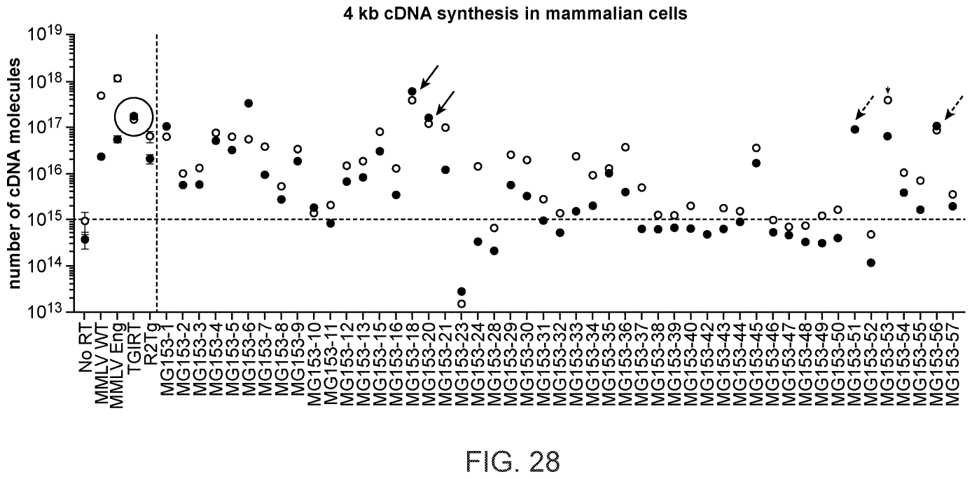

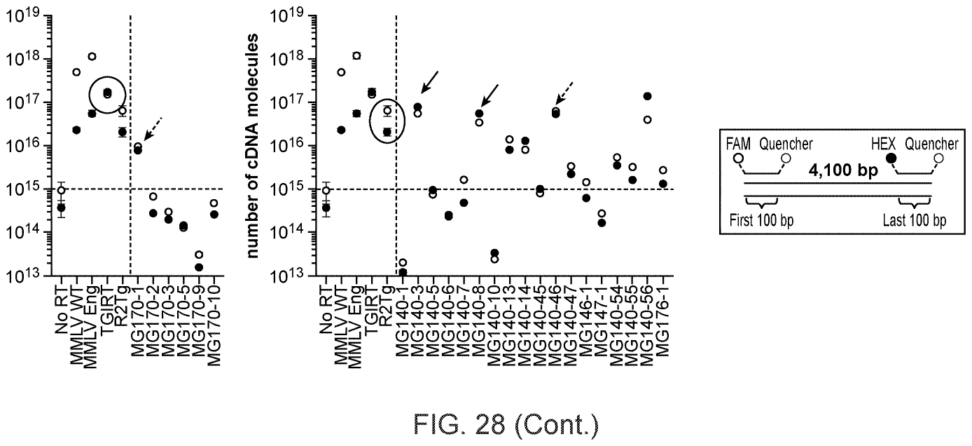

Data is seen in FIG. 28. Activity for the control GII intron RT TGIRT, the retroviral MMLV (WT and penta-mutant) as well as a positive control for R2, R2Tg, was detected, as shown by an early amplification of the first and last 100 bp products. As expected for a low processivity RT, the retroviral RTs (MMLVs) show high amplification levels of the first 100 bps (FAM signal) but the levels at which they complete cDNA synthesis (the last 100 bps) is lower (20 fold lower than first 100 bp, as observed by the FAM/HEX ratio signal). Group II intron-derived RTs such as MG153-18, MG153-20, MG153-51, MG153-56, MG170-1 and R2 non-LTR retrotransposon RTs such as MG140-3, MG140-8, and MG140-46 show a closer FAM/HEX ratio, demonstrating their high processivity.

### Example 13. RTs for Short Corrections, Small Insertions, and Deletions

Testing Reverse Transcriptases Tethered to spCas9(H840A) Nickase

RT candidates were cloned into a plasmid containing the nickase spCas9(H840A) (SEQ ID NO: 1247) to generate a RT-nickase fusion. The CMV promoter drove the expression of the fusion protein, which contained a 33 amino acid linker (SEQ ID NO: 103) between the nickase and the RT candidate (SEQ ID NOs: 1250-1279). The fusion protein was then transfected into HEK293T cells. Chemically synthesized pegRNA (SEQ ID NOs: 656-679) containing the desired edit in the RT template was transfected. All components (plasmid and pegRNAs) were reverse transfected into 50,000 HEK293T cells in a 24 well plate. 72 hour post transfection, cells were lysed in 100 μL of extraction solution. Primers containing barcodes for next generation sequencing (NGS; SEQ ID NOs: 100-101) were used to amplify a ˜250 bp target (SEQ ID NO: 102). PCR clean-up was then performed and samples were sent for NGS sequencing. FASTQ files were then processed using the prime editing setting to determine the percentage of reads with desired change.

### Results

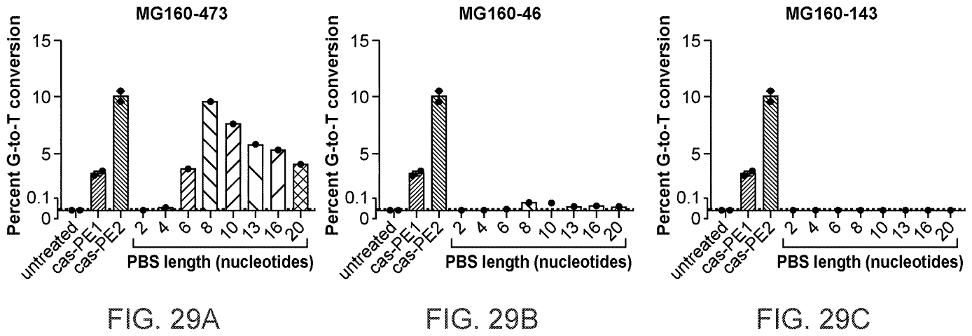

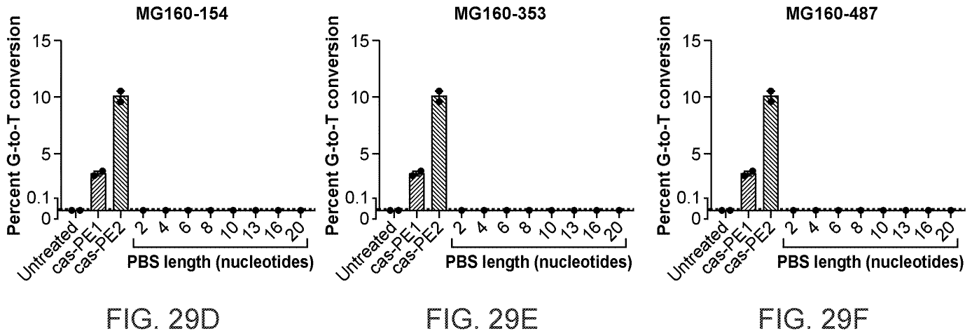

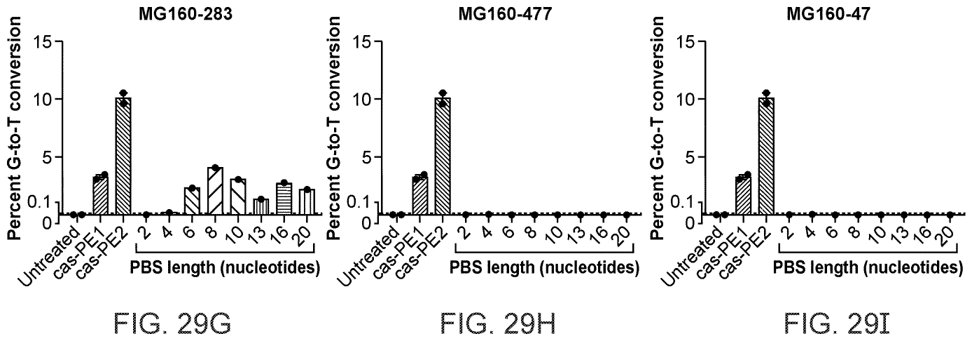

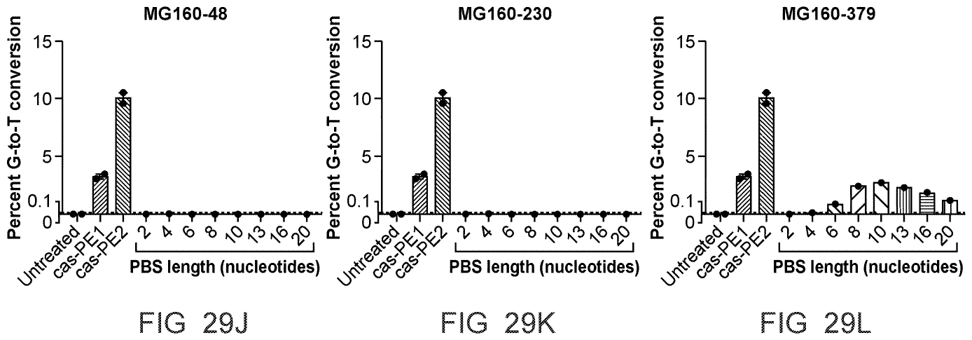

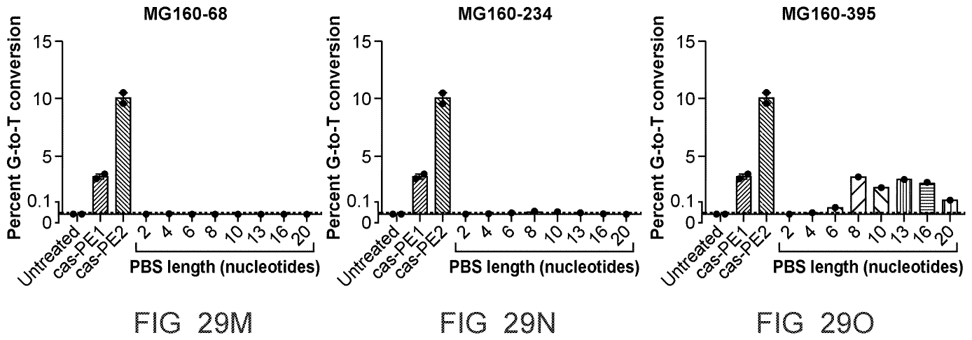

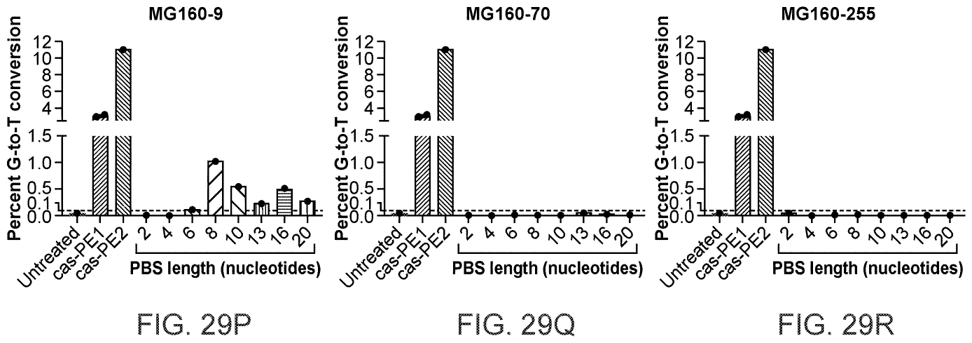

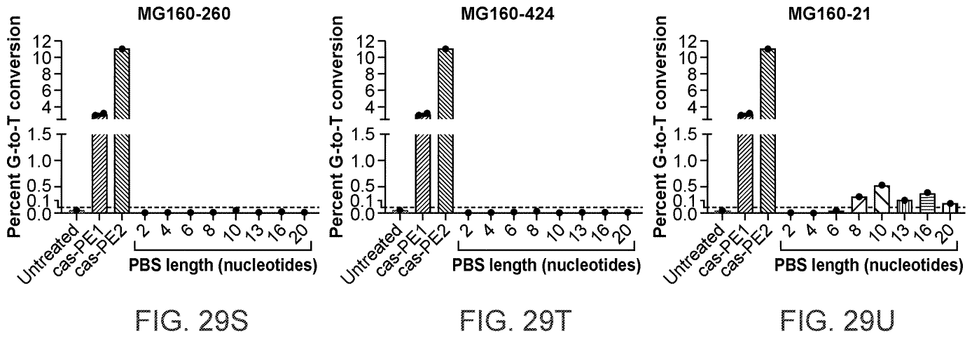

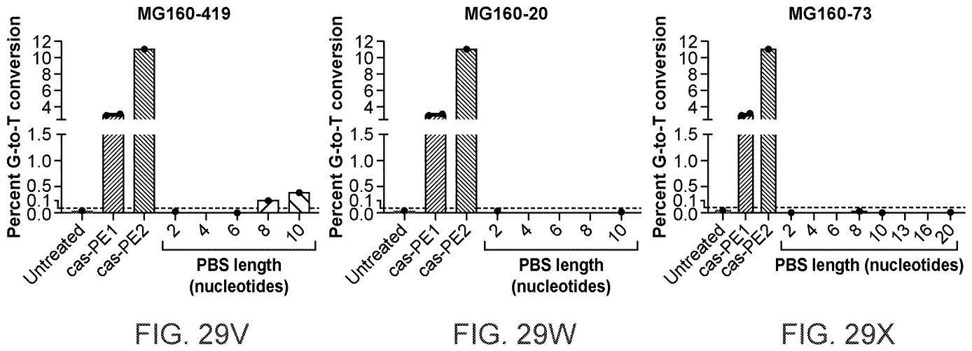

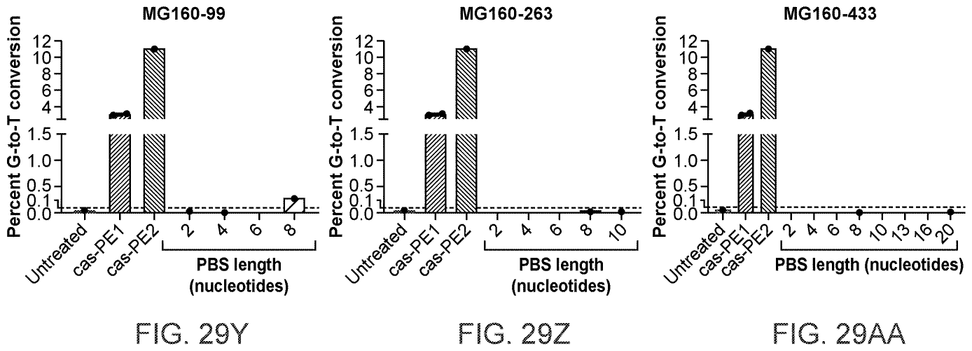

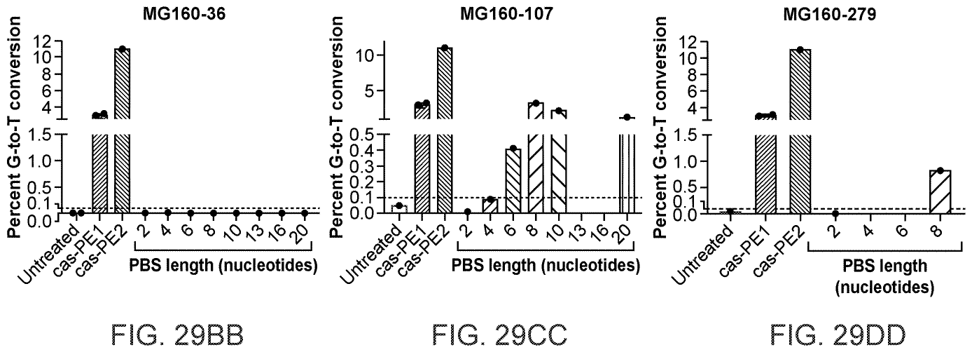

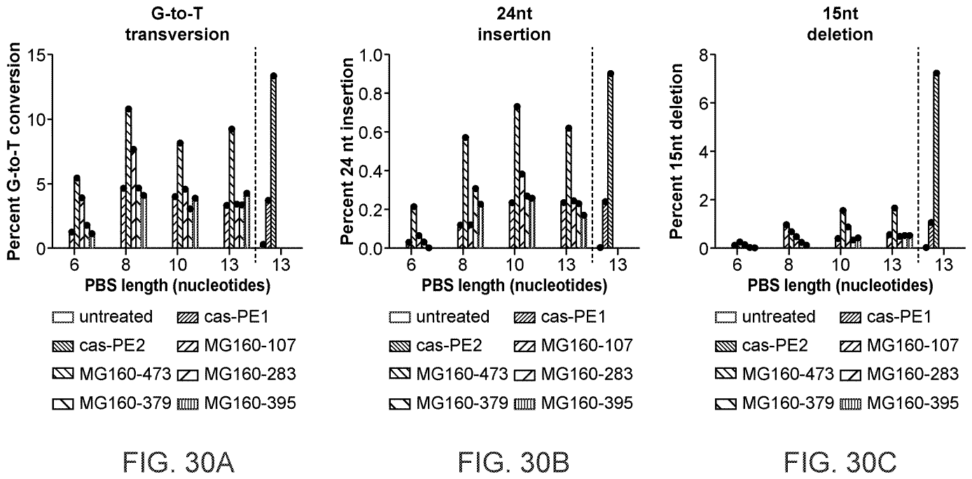

MG160 candidates tethered to spCas9(H840A) were tested for G-to-T conversion on the VEGFA target in HEK293T cells (FIGS. 29A-29DD). Percent editing for each RT with pegRNAs at varying PBS lengths (2, 4, 6, 8, 10, 13, 16, and 20 nucleotides, SEQ ID NOs: 656-679) is shown in FIGS. 29A-29DD. Editing levels for each RT candidate represent a single biological replicate. MG160-473, MG160-283, MG160-379, MG160-395 and MG160-107 (SEQ ID NOs: 1206, 1017, 1112, 1128 and 841) showed equivalent or improved editing efficiency relative to the control spCas9(H840A) tethered to MMLV WT (FIGS. 29A, 29G, 29L, 29O, and 29CC, respectively). Furthermore, candidate MG160-473 (SEQ ID NO: 1206) editing levels were comparable to the control spCas9(H840A) (SEQ ID NO: 1247) tethered to the hyperactive mutant MMLV (MMLV2, PE2) (SEQ ID NO: 1249; FIG. 29A). Additionally, candidates MG160-46, MG160-9, MG160-21, MG160-419, MG160-99 and MG160-279 (SEQ ID NOs: 1251, 1265, 1270, 1271, 1274 and 1279) showed activity above background (FIGS. 29B, 29P, 29U, 29V, 29Y and 29DD respectively). The five MG160 candidates with high G-to-T conversion were then repeated to confirm G-to-T conversion (FIG. 30A), as well as for their ability to perform a 24 nucleotide insertion (FIG. 30B) and 15 nucleotide deletion (FIG. 30C) with chemically synthesized pegRNAs ranging from PBS lengths of 6, 8, 10, and 13 nucleotides (SEQ ID NOs: 76-99). MG160-283, MG160-379, MG160-395 and MG160-107 (SEQ ID NOs: 1017, 1112, 1128 and 841) showed similar editing levels to control MMLV WT (SEQ ID NO: 1248) for all desired edits, while candidate MG160-473 (SEQ ID NO: 1206) exhibited high editing levels, comparable to the hyperactive mutant MMLV2 (SEQ ID NO: 1249) for G-to-T conversion and 24 nucleotide insertion.

Testing Reverse Transcriptase Candidates Untethered from the Cas Nickase

Reverse transcriptase (RT) candidates from diverse retron families MG155, MG156, MG157, MG159, and MG173, and from MG Group II intron families MG164, MG166, MG167 and MG169 (SEQ ID NOs: 1280-1294) were cloned into a plasmid with a CMV promoter driving expression of RT. The plasmid was then isolated for transfection in HEK293T cells. Another plasmid containing a nickase spCas9 (H840A) (SEQ ID NO: 1247) driven by a CMV promoter and the RT containing plasmid were cotransfected. Chemically synthesized pegRNA (SEQ ID NOs: 656-679) containing the desired edit in the RT template was transfected. All components (plasmids and pegRNAs) were reverse transfected into 50,000 HEK293T cells in a 24 well plate. 72 h post transfection, cells were lysed in 100 pL solution. Primers containing barcodes for NGS (SEQ ID NOs: 100-101) were used to amplify a ˜250 bp target (SEQ ID NO: 102). PCR clean-up was then performed, and the samples were sent for NGS sequencing. FASTQ files were then processed using the prime editing setting to determine the percentage of reads with the desired change.

### Results

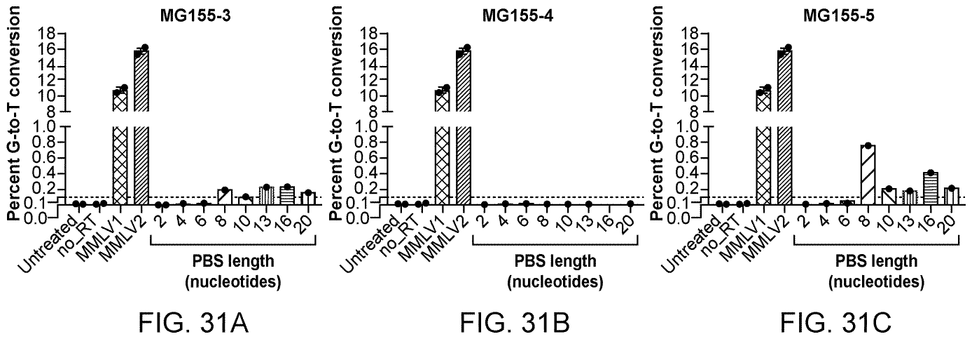

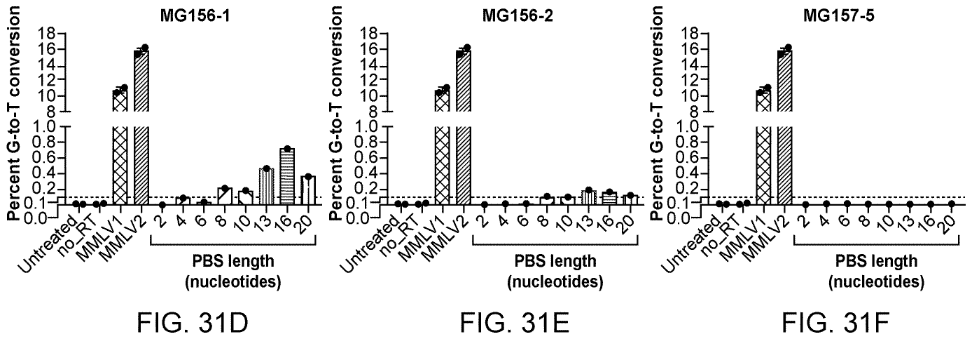

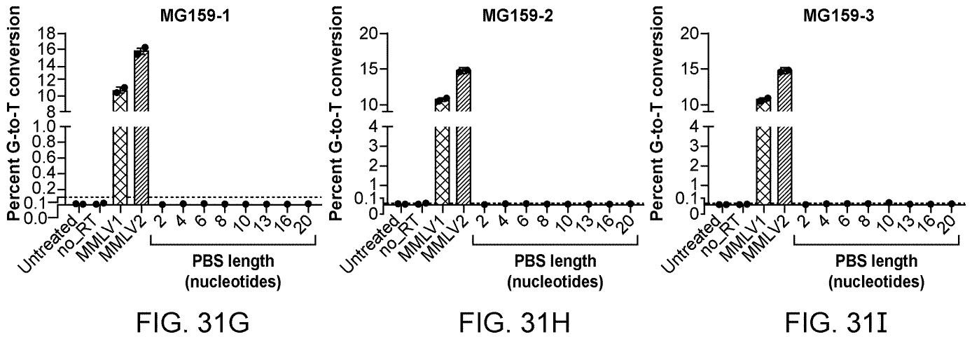

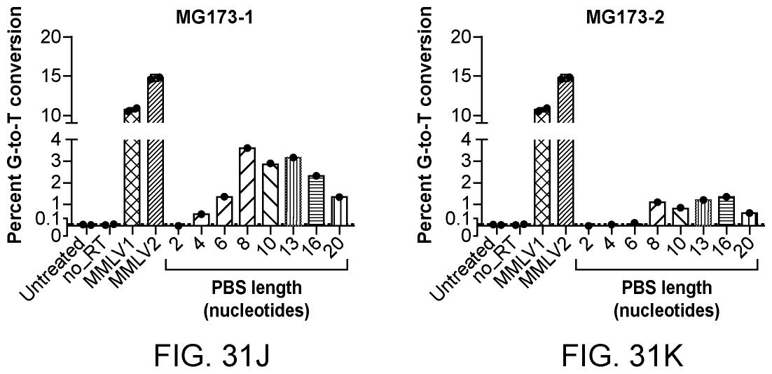

Untethered retron candidates from families MG155, MG156, MG157, MG159 and MG173 were tested for a G-to-T change using pegRNAs with varying PBS lengths (2, 4, 6, 8, 10, 13, 16, and 20 nucleotides; SEQ ID NOs: 76-83; FIGS. 31A-31K). In a single biological replicate experiment, retron RT candidates MG173-1 (SEQ ID NO: 627; FIG. 31J) and MG173-2 (SEQ ID NO: 628; FIG. 31K) showed editing levels reaching about 4.5% at PBS 8 and 1.5% at PBS 13, respectively, and had editing levels above background at various PBS lengths. Editing levels above the background were also seen for MG155-3 (SEQ ID NO: 504; FIG. 31A), MG155-5 (SEQ ID NO: 506; FIG. 31C), and MG156-1 (SEQ ID NO: 507; FIG. 31D).

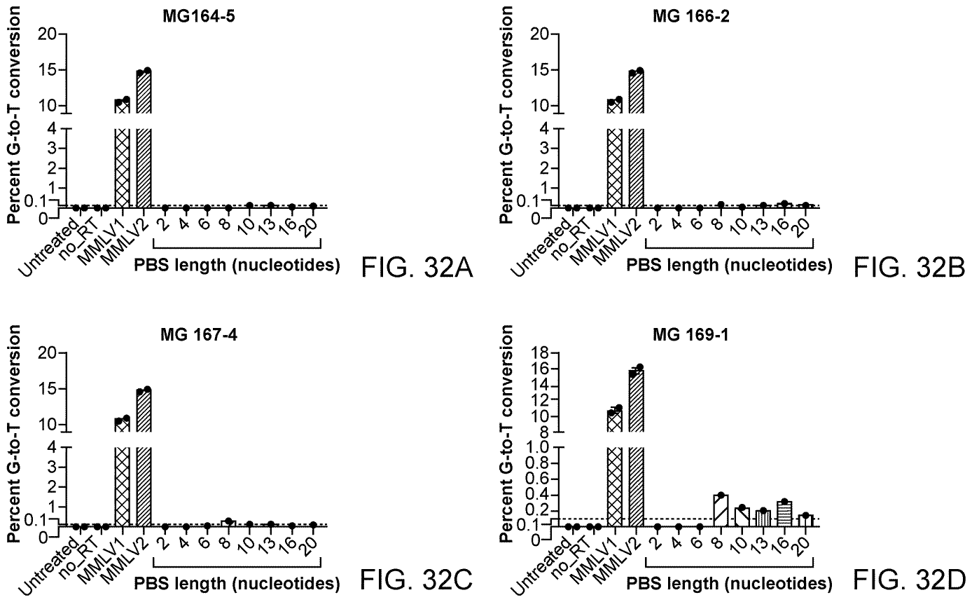

Untethered Group II intron families, MG164, MG166, MG167 and MG169 were tested with editing levels shown in FIGS. 32A-32D. Most of these candidates did not show detectable activity, but some editing above the background was seen for MG169-1 (SEQ ID NO: 601) at varying PBS lengths (FIG. 32D).

### Example 14. Short Corrections, Small Insertions, and Deletions with Engineered RTs

Editing with Engineered MG160-4 and MG153-53 RT Candidates

The selected RT candidates MG160-4 (SEQ ID NO: 521) and MG153-53 (SEQ ID NO: 496) were subjected to rational engineering to improve editing efficiencies. Various point mutations (SEQ ID NOs: 1221-1243) were tested individually, as well as combined to determine which engineered candidates could improve editing activity. Different combinations of MG160-4 and MG153-53 mutations tethered (MG160-4) or untethered (MG153-53) to spCas9(H840A) were tested for G-to-T conversion on the VEGFA target using chemically synthesized pegRNAs with PBS lengths of 6, 8, 10, and 13 nucleotides. Primers containing barcodes for NGS (SEQ ID NOs: 100-101) were used to amplify a ˜250 bp target (SEQ ID NO: 102). PCR clean-up was then performed and samples were sent for NGS sequencing. FASTQ files were then processed to determine the percentage of reads with desired change. Single biological replicates were tested alongside untethered controls MMLV1 and MMLV2 (SEQ ID NOs: 1248 and 1249), as well as control RTs TGIRT, Marathon, and Marathon mutant (SEQ ID NOs: 1296-1298).

### Results

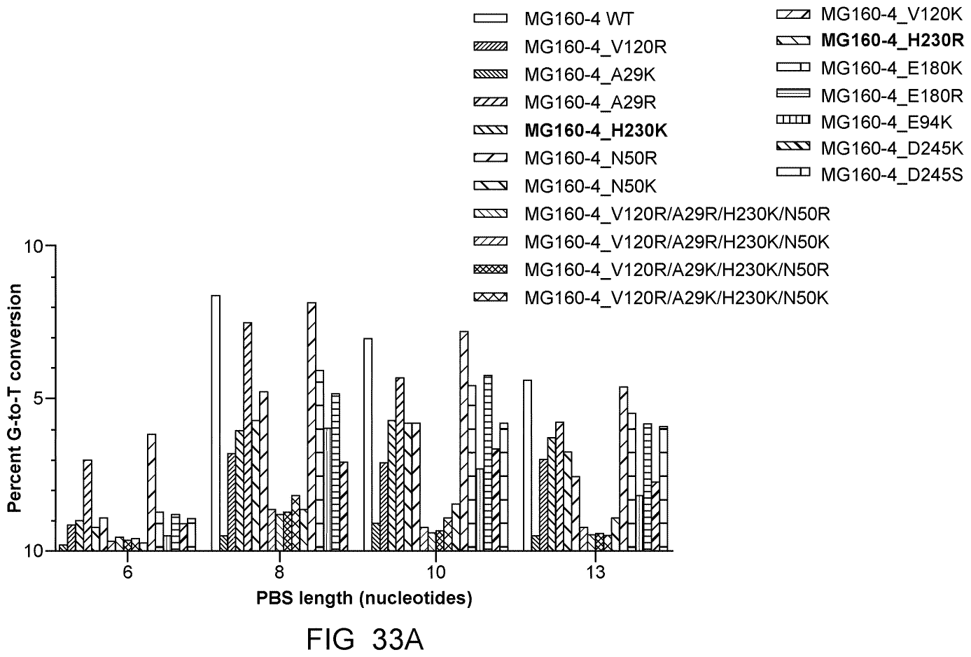

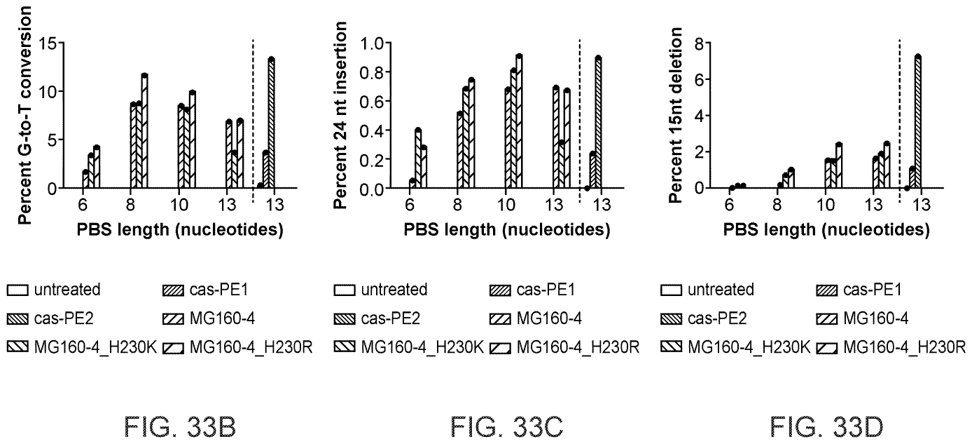

Combining four different point mutations in various combinations for MG160-4 led to a sharp decrease in editing efficiency (FIG. 33A). However, single point mutations H230K (MG160-4 H230K; SEQ ID NO: 1230) and H230R (MG160-4 H230R; SEQ ID NO: 1234) showed a neutral change in editing levels for G-to-T conversion (FIG. 33A). These engineered constructs were further tested for G-to-T transversion (FIG. 33B), as well as for 24 nucleotide insertion (FIG. 33C), and 15 nucleotide deletion (FIG. 33D). As observed in the first replicate, MG160-4-H230K and MG160-4 H230R showed a neutral change in editing levels for G-to-T transversion (FIG. 33B) but an increase in editing levels for MG160-4 H230R (SEQ ID NO: 1230) compared to the wild type MG160-4 for 24 nucleotide insertion (FIG. 33C) and deletion (FIG. 33D). MG160-4 H230R (SEQ ID NO: 1234) showed slightly improved editing compared to engineered MG160-4-H230K (SEQ ID NO: 1230) when editing involved incorporating 24 nucleotide insertions and 15 nucleotide deletions.

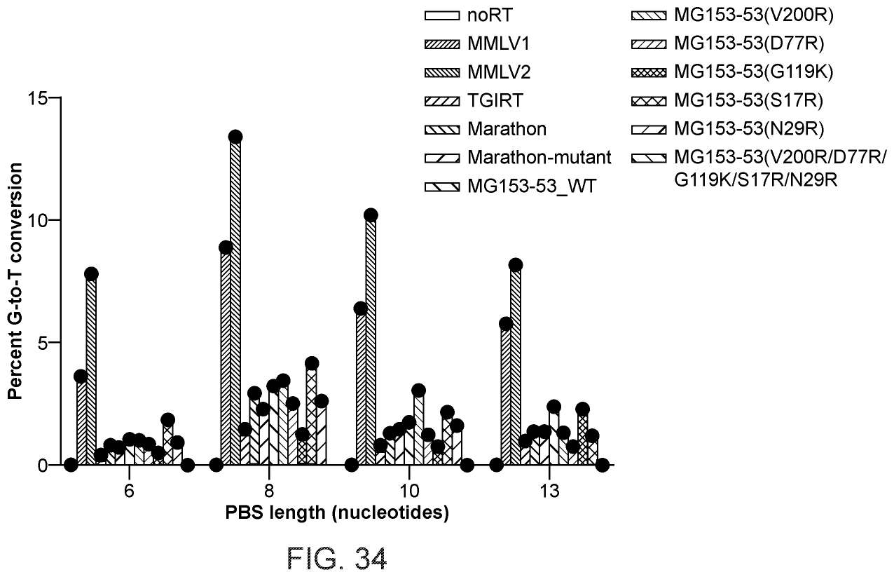

The construct combining all suggested mutations of MG153-53 (SEQ ID NO: 1226) abolished editing activity (FIG. 34). The single point mutation V200R (MG153-53 (V200R); SEQ ID NO: 1225), slightly enhanced G-to-T transversion compared to WT MG153-53 (SEQ ID NO: 496; FIG. 34). WT MG153-53 (SEQ ID NO: 496) did not perform better than controls MMLV1 and MMLV2 (SEQ ID NO: 1248 and 1249), but did outperform control RTs TGIRT, Marathon, and Marathon mutant (SEQ ID NOs: 1296-1298).

### Example 15. Nickases for Mediating Short Corrections, Small Insertions and Deletions in Conjunction with Reverse Transcriptases

Installing site-directed genomic corrections, insertions, or deletions using RTs requires the RT system to be targetable. This example describes the use of a targetable RT system comprising an RT and a Cas nickase. The Cas nickase guided by a gRNA site-specifically nicks the non-target strand, thus creating a primer for the reverse transcription reaction. The gRNA that accompanies the Cas nickase is a modified version (pegRNA) that comprises a 3′ extension containing the RTT and the PBS. The PBS and the spacer are complementary to each other. It is contemplated that this complementarity can cause gRNA structure disruption, causing the pegRNA to interact with the Cas inhibiting the Cas from finding the target genet. Each Cas nuclease interacts with its own gRNA, as such the pegRNA design and requirements vary from system to system.

### Testing Selected Reverse Transcriptases Untethered and Tethered to MG3-6(H586A) and MG71-2(H883A) Nickases

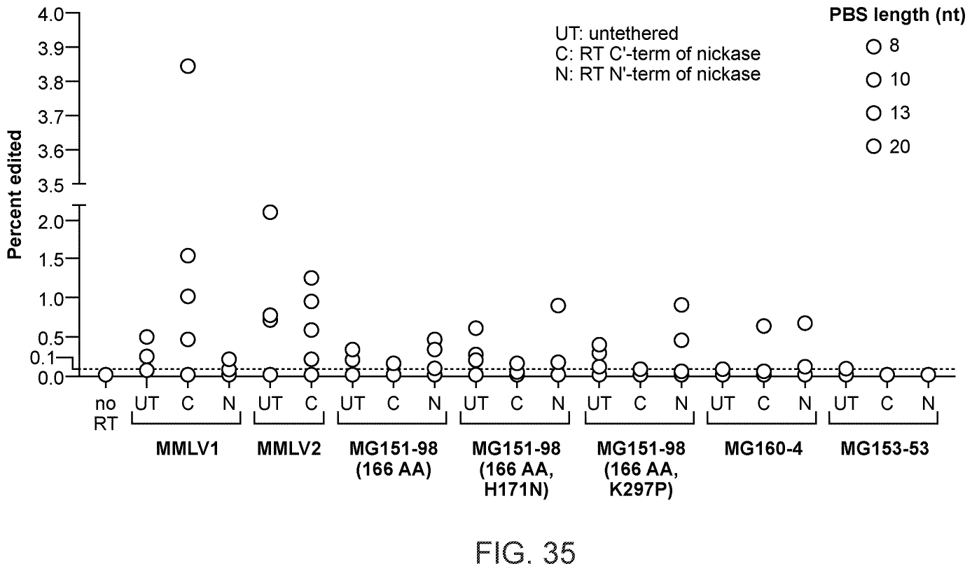

An MG3-6(H586A) (SEQ ID NO: 653) or MG71-2(H883A) (SEQ ID NO: 1309) nickase was challenged to introduce genomic corrections with reverse transcriptases on an AAVS1 target site (SEQ ID NO: 654 or 1344). Selected MG RT candidates (SEQ ID NOs: 1295, and 1299-1304) were transfected into HEK293T cells either untethered with the MG3-6(H586A) (SEQ ID NO: 653) plasmid (FIG. 35) or tethered to MG3-6(H586A) (SEQ ID NO: 653) with the selected RTs fused to the N-terminus or C-terminus (FIG. 35). For MG3-6(H586A) (SEQ ID NO: 653) genomic corrections were targeted with chemically synthesized pegRNAs with PBS lengths of 8, 10, 13, and 20 nucleotides (SEQ ID NOs: 682-684, and 686) and for MG71-2(H883A) (SEQ ID NO: 1309) genomic corrections were targeted with chemically synthesized pegRNAs with PBS lengths of 6, 8, 10, 13, 16, and 20 nucleotides (SEQ ID NOs: 1310-1341). Primers containing barcodes for NGS (SEQ ID NO: 698-699 for MG3-6(H586A (SEQ ID NO: 653) or SEQ ID NOs: 1342-1343 for MG71-2(H883A) (SEQ ID NO: 1309)) were used to amplify a ˜250 bp MG3-6(H586A) AAVS1 target (SEQ ID 654) or MG71-2(H883A) AAVS1 target (SEQ ID NO: 1344). PCR clean-up was then performed and samples were sent for NGS sequencing. FASTQ files were then processed using the prime editing setting to determine the percentage of reads with desired change.

### Results

Above background editing (>0.1%) was seen at PBS lengths 8, 10, 13, and 20 nucleotides for selected RT candidates for G-to-T transversion (FIG. 35). Interestingly, when MG3-6(H586A) (SEQ ID NO: 653) and MMLV WT (MMLV1) (SEQ ID NO: 1248) was tethered to the C-terminus of MG3-6(H586A), editing levels were in general higher than in the untethered approach or when the RT was tethered to the N-terminus of the nickase. When MG3-6(H586A) (SEQ ID NO: 653) and hyperactive mutant MMLV (MMLV2) (SEQ ID NO: 1249) was tethered to the C-terminus of MG3-6(H586A), editing levels were similar in the untethered approach. Contrastingly, MG151-98 engineered mutants (SEQ ID NOs: 1302-1304) resulted in higher levels of editing when either tethered to the N-terminus of MG3-6(H586A) (SEQ ID NO: 653) or in the untethered approach (FIG. 35). MG160-4 (SEQ ID NO: 1295) achieved similar editing levels when either tethered to the N or C terminus of MG3-6(H586A) (SEQ ID NO: 653) but did not have above background editing in the untethered approach. MG153-53 (SEQ ID NO: 1299) with all three different approaches with MG3-6(H586A) (SEQ ID NO: 653) showed no editing activity above background levels (FIG. 35).

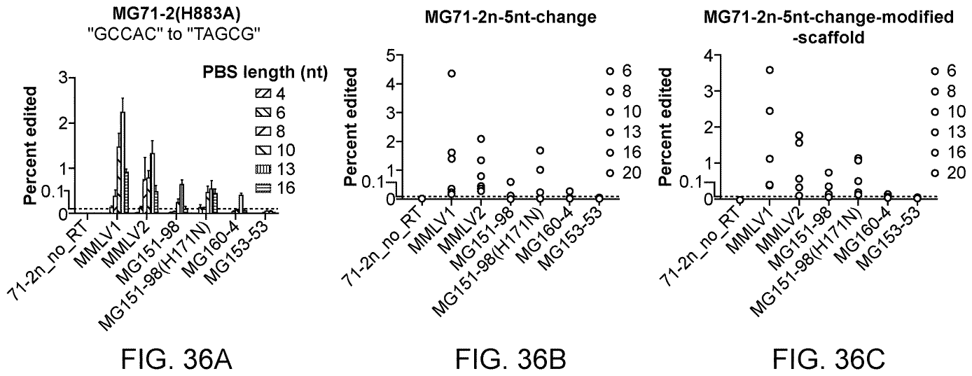

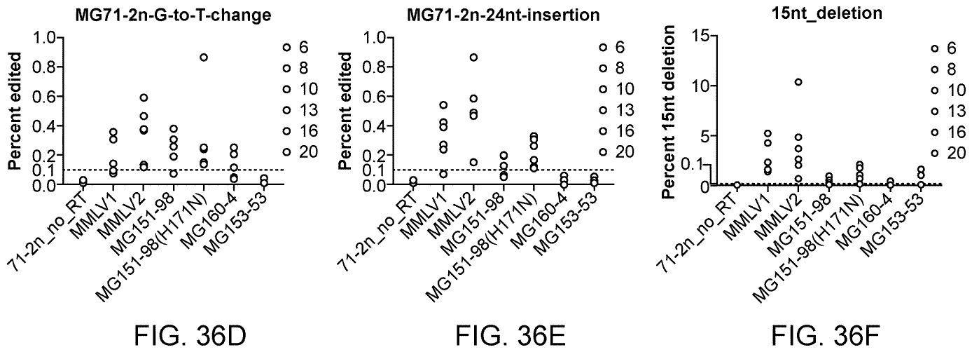

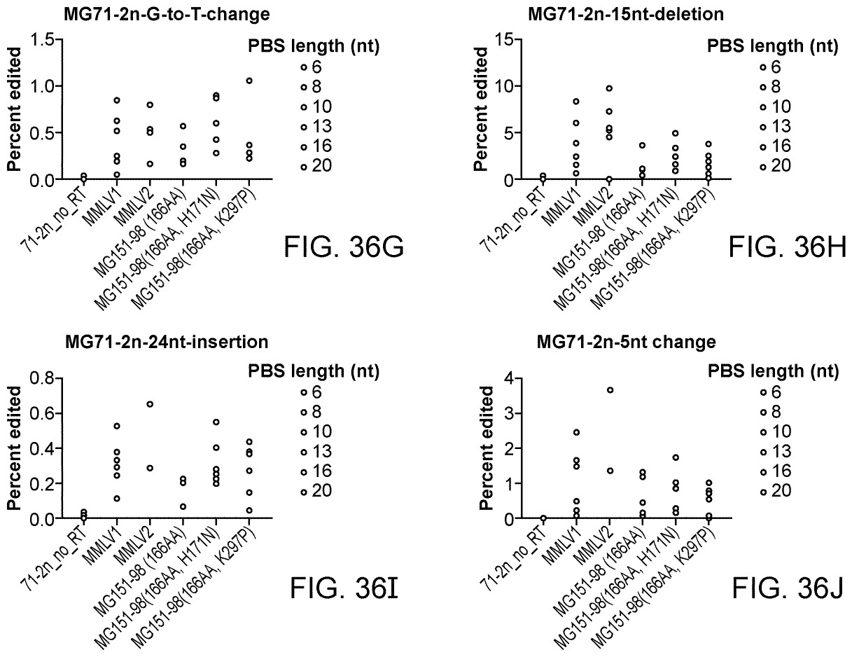

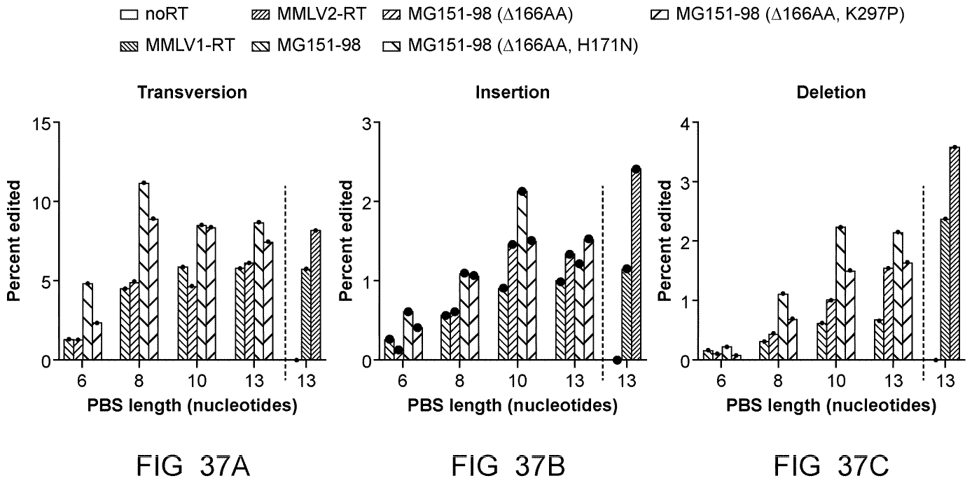

Untethered MG71-2(H883A) (SEQ ID NO: 1309) with selected RTs showed editing levels for various edits including five nucleotide changes (FIGS. 36A-36C, and 36J), single G-to-T nucleotide transversion (FIGS. 36D and 36G), 24 nucleotide insertion (FIGS. 36E and 36I), and 15 nucleotide deletion (FIGS. 36F and 36H). Biological triplicate data for correcting five nucleotide changes in AAVS1 target (SEQ ID NO: 1344) was shown with selected RTs. Untethered MMLV1 and MMLV2 (SEQ ID NOs: 1248 and 1249) with MG71-2(H883A) (SEQ ID NO: 1309) showed high levels of editing for all corrections (FIGS. 36A-36J). MG153-53(SEQ ID NO: 1299) showed above background editing only when trying to correct a 15 nucleotide deletion (FIG. 36F). When comparing FIG. 36B and FIG. 36C, pegRNA scaffold went from four consecutive Ts to a modified scaffold with four consecutive Gs. Editing levels between the original scaffold and modified scaffold did not have any significant changes in editing levels, so the original scaffold was kept when correcting for other changes (insertion, deletion, and SNP). Interestingly, editing levels were higher for correcting a five nucleotide change (FIG. 36B) than a single G-to-T transversion (FIG. 36D). In general, MG71-2(H883A) (SEQ ID NO: 1309) and select RTs (SEQ ID NOs: 1295, and 1299-1301) showed highest editing levels for all corrections when pegRNA PBS lengths were between 8 to 16 nucleotides. Engineered MG151-98 candidates (SEQ ID NO: 1302-1304) were then tested with untethered MG71-2(H883A) (SEQ ID NO: 1309) to correct various changes on the AAVS1 target (SEQ ID NO: 1344; FIGS. 36G-36J). All MG151-98 engineered candidates (SEQ ID NOs: 1302-1304) showed comparable editing levels to MMLV1 and MMLV2 (SEQ ID NOs: 1248 and 1249) for all corrections.

Both MG3-6(H586A) (SEQ ID NO: 653) and MG71-2(H883A) (SEQ ID NO: 1309) have shown to be effective nickases compatible with RTs reverse transcribing small corrections into genomic targets.

### Example 16. Retron RTs for Programmable, Large Cargo Integrations Mediated by a Single Stranded DNA Transposase (TnpA)

Retrons are DNA retro-elements that contain a reverse transcriptase (RT) gene located downstream of a conserved non-coding structural RNA. The non-coding RNA consists of two inverted regions, referred to as msr and msd. When the retron RT recognizes the msr folded into a specific secondary structure (specific recognition motifs), it initiates reverse transcription of the msd portion (template), thus producing multicopy of single stranded DNA (ssDNA). Overall, retrons have RT capabilities that are primed by a specific RNA recognition motif (msr), and produces a covalently bound complementary ssDNA molecule. Thus, dependence on recognition motifs in the mrs should reduce off target priming and provide a mechanism for localizing the template RNA/DNA to a specific genomic target.

Precise genome editing with scarless replacement of alleles or insertion of synthetic sequences requires in vitro delivery of donor DNA. However, many challenges to induce cells to utilize donor DNA to conduct Homology Directed Repair (HDR) exist. For this end, it may be possible that retrons could be harnessed to produce high copy number intracellular DNA molecules in human hosts. Early experiments showed that the msd could be variable and can encode an in-situ DNA with an artificial sequence of interest. Hence, retrons could be repurposed as a source of donor DNA for genome engineering. This biological solution could enable in-nucleo donor generation that would improve the scalability and multiplexing capabilities for genomic knock-ins. Recently, it has been shown that retrons coupled with Cas9 improved the efficiency of precise genome editing via HDR in HEK293T and K563 with HDR rates of up to ˜11%. While these findings represent first steps in retron-based gene editing in human cells, low editing efficiency due to the limitation of HDR in non-cycling cells remains a challenge. Coupling a Retron-Cas9-like fusion with a ssDNA integrase such ssDNA transposase TnpA may circumvent the reliance of the HDR pathway and improve DNA integration. For example, IS200/IS605 transposons are a type of mobile genetic element that integrate ssDNA at specific target sites by a TnpA transposase. TnpA excises a donor by recognizing structural motifs at each donor end, integrating it at a recognized target site accessible as ssDNA.

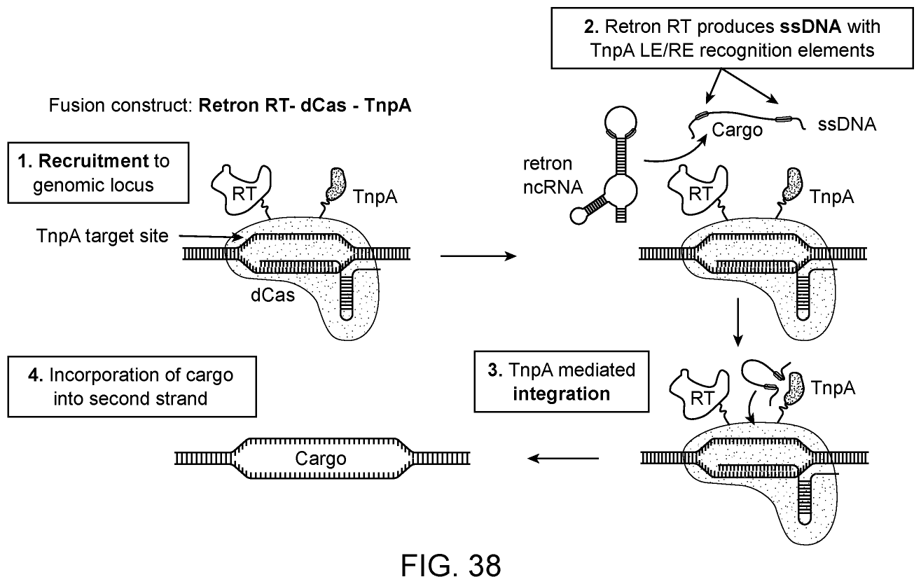

The ssDNA produced by a retron RT can be used as a template by TnpA for programmable integration of desired cargo into a specific target site (FIG. 38). Specifically, the retron msd contains the desired cargo (for example, an antibiotic resistance cassette or fluorescent marker) flanked by LE and RE structural motifs recognizable by TnpA. The TnpA transposase excises and circularizes the ssDNA donor, and integration into a target occurs via recognition of a specific motif available through an R-loop formed by the RNA-guided recognition and binding of an engineered (nickase or dead) effector, for example, MG3-6.

### Example 17. Engineering of ncRNAs-Associated Retron RTs to Include LE, RE, and Cleavage Motif of TnpA

Reverse Transcription of Engineered ncRNAs that Contain the LE/RE Motifs of Hp TnpA by Ec86

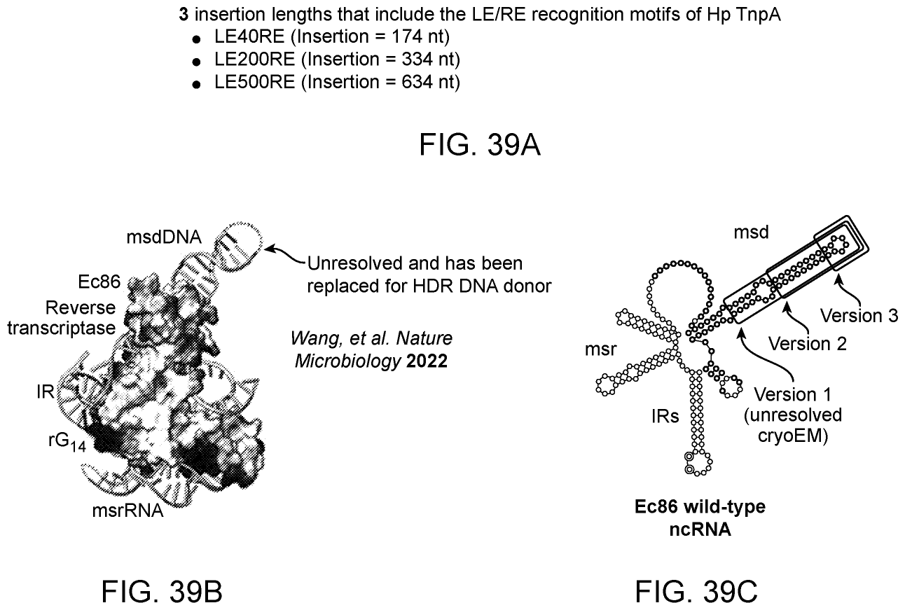

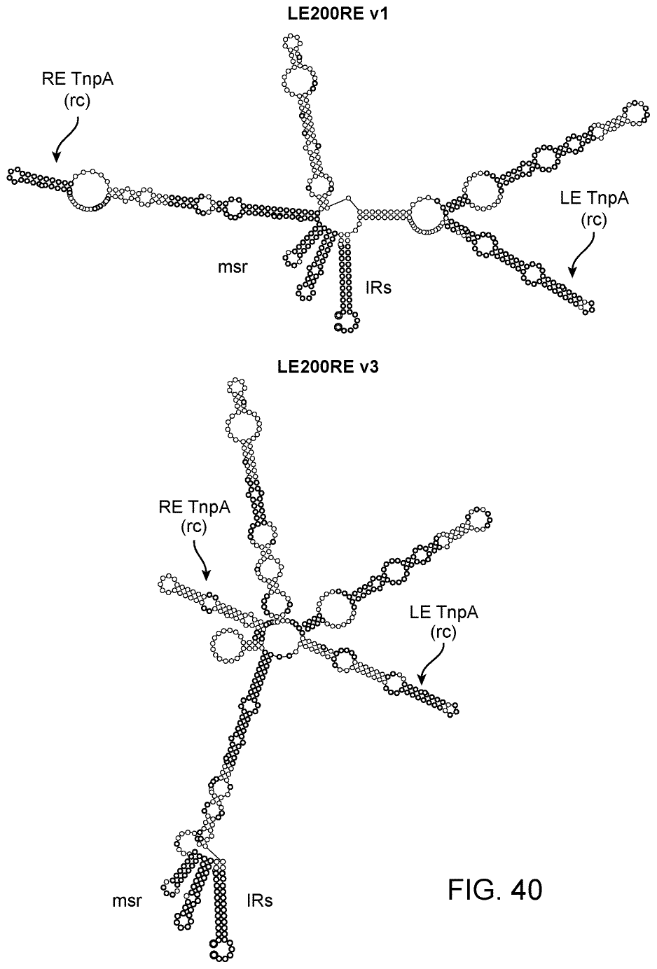

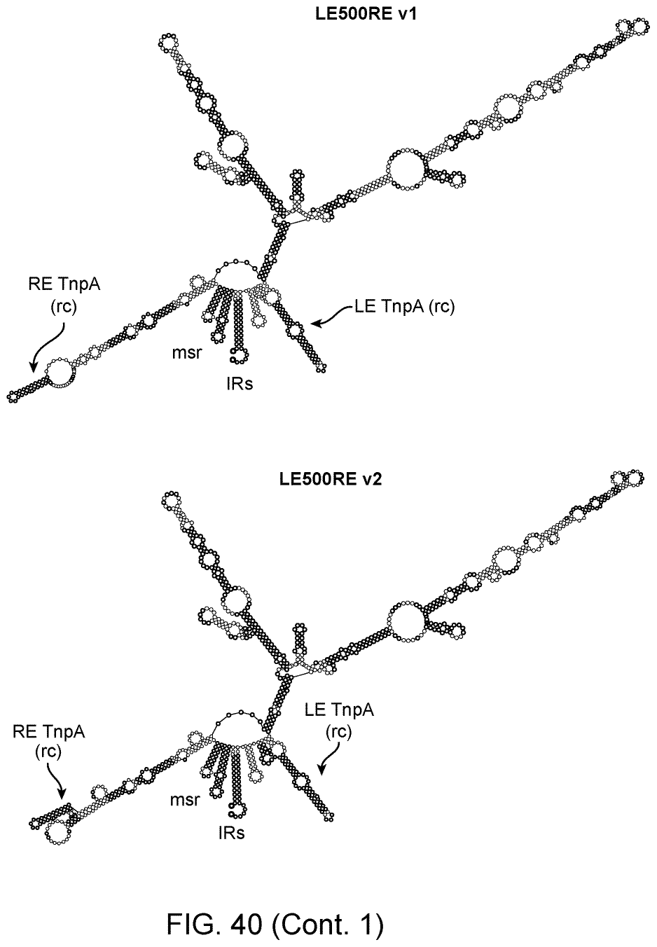

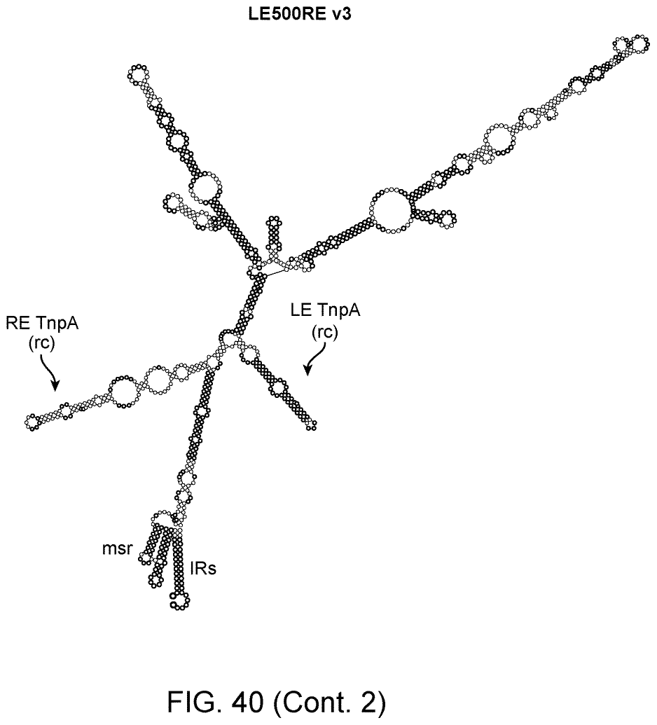

Eight engineered variants of the Ec86 ncRNA were designed (SEQ ID NOs: 1346-1353; FIGS. 39-40) using the cryo-EM structure of Ec86 in complex with its product and previous work which has identified a replaceable region of the Ec86 msd stem loop to facilitate homology-directed repair. Insertion sequences of three different lengths were designed, all of which contain the reverse-complement of the left end (LE) and right end (RE) recognition motifs of the H. pylori (Hp) TnpA ssDNA transposase at the 3′ and 5′ flanking regions. The insertion sequence designated at LE40RE contains a 40 nt sequence flanked by the LE and RE of Hp TnpA, giving a total insertion length of 174 nt. The insertion sequence designated at LE200RE and LE500RE contains a 200 nt or 500 nt partial kanamycin gene flanked by the LE/RE motifs, giving a total insertion length of 334 nt and 634 nt, respectively. These three different sequences were inserted at two or three different potential replaceable regions within the msd stem loop (FIG. 40). The version designated as version 1 replaces the entire msd region that was not resolved in the cryo-EM structure of Ec86 bound to its msdDNA with the engineered sequence. Versions 2 and 3 are more progressively more conservative replacement designs, with version 2 replacing the msd region after the a bubble in the msd stem loop, and version 3 retaining most of the msd stem loop for the terminal 8 nucleotides.

The reverse-complement of the LE and RE motifs of Hp TnpA are predicted to adopt distinct secondary structures within the engineered ncRNAs (FIG. 40). However, the predicted RNA folds do suggest that the key recognition motifs required for recognition by the Ec86 RT, including the terminal inverted repeats and msr hairpins, are maintained, suggesting that the LE/RE motifs of TnpA may not disrupt the fold of key ncRNA features required for priming.

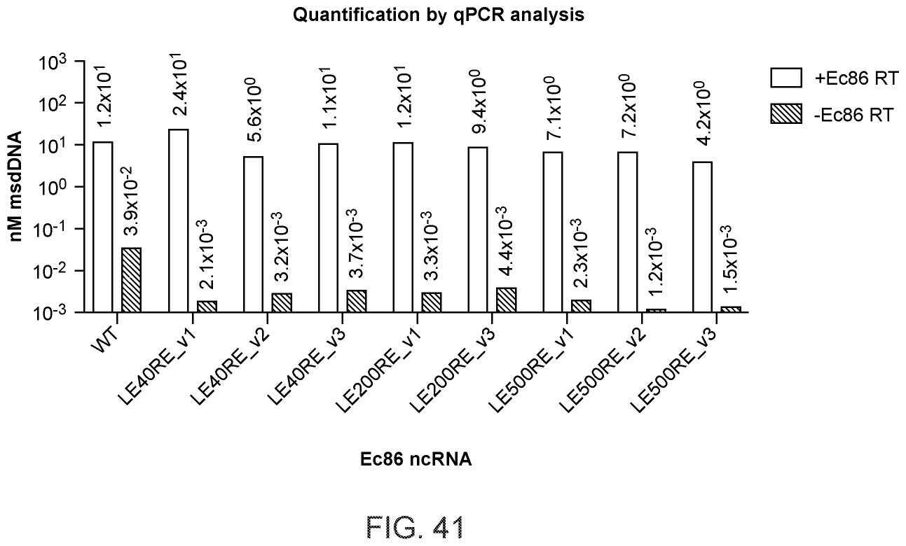

Reverse transcription of engineered ncRNAs by Ec86 was determined in vitro. The Ec86 RT was co-expressed with the ncRNA substrate (final 100 nM) in a cell-free expression system) supplemented with dNTPs (final 0.3 mM). Expression constructs were codon-optimized for E. coli and contained an N-terminal single Strep tag. After incubation for 2 h at 37° C., the reaction was quenched by heat denaturation at 95° C. for 2 min, followed by treatment by RNase A for 30 min at 37° C. Ec86 activity was assessed by qPCR using primers (SEQ ID NOs: 1354-1355) that amplify either the product generated from the wild-type ncRNA (SEQ ID NO: 1345), or from the engineered 40nt partial kanamycin gene (SEQ ID NOs: 1356-1357) or 200nt and 500nt partial kanamycin gene (SEQ ID NOs: 1358-1359). The resulting reverse transcription products, herein referred to as msdDNA, were diluted prior to qPCR to ensure msdDNA concentrations were within the linear range of detection. The amount of msdDNA was quantified by extrapolating values from a standard curve generated with a DNA template of known concentrations. Based on these results, Ec86 RT was capable of producing appreciable amounts of msdDNA from all eight engineered ncRNA designs and at levels comparable to that of the wild-type ncRNA (FIG. 41). This data indicates that Ec86 is tolerant to insertions as large as 634 nt at 3 different replaceable regions within the msd stem loop.

Insertion of ssDNA Produced by Retron RT Ec86 by Hp TnpA

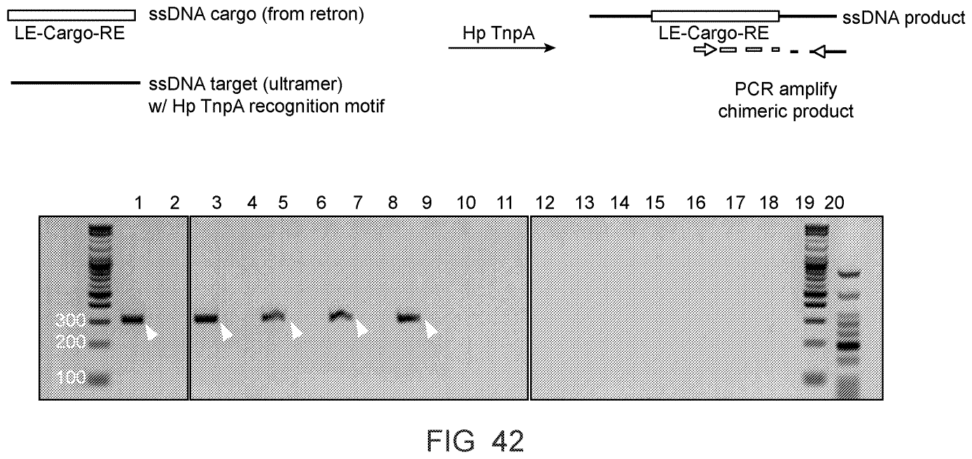

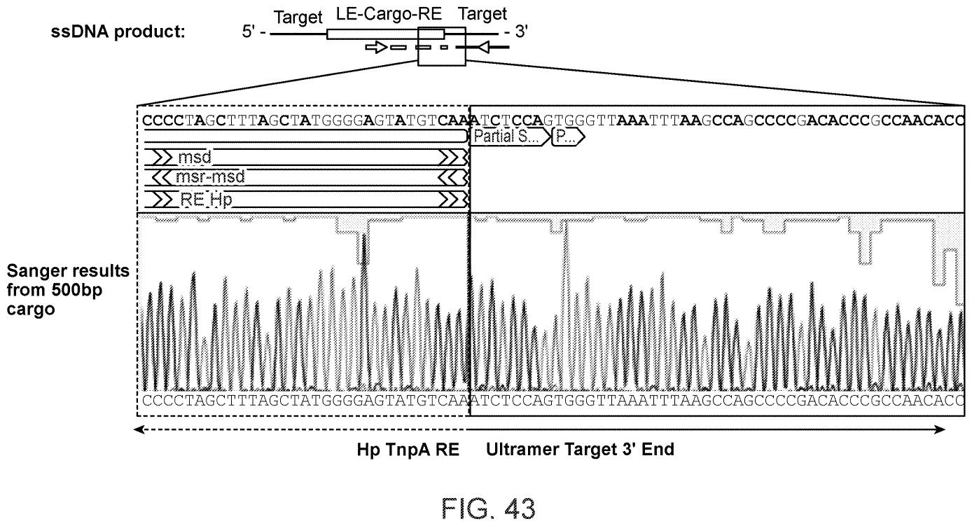

Insertion of ssDNA produced by a retron by Hp TnpA was determined. Briefly, Ec86 was co-expressed the engineered ncRNA substrate (LE200RE_v1/v3 or LE500RE_v1/v2/v3) in a cell-free expression system as described above, followed by quenching by heat denaturation and RNase A treatment, also as described above. RNase A treatment removes any RNA in heteroduplex with the generated msdDNA, thereby making the product available as ssDNA for TnpA. Subsequently, the generated ssDNA, which contained the LE/RE motifs of Hp TnpA, was mixed with Hp TnpA protein that was also generated in a cell-free expression system in reaction buffer containing 20 mM HEPES (pH 7.5), 160 mM NaCl, 5 mM MgCl₂, 5 mM TCEP, 20 g/mL BSA, 0.5 pg/mL of poly-dIdC, and 20% glycerol. The reaction also contained 50 nM of a ssDNA insertion target which included the Hp TnpA targeting motif (TTAC). The TnpA insertion reaction was allowed to proceed for 1 hour at 37° C., after which successful insertion by TnpA was confirmed by PCR of the chimeric product (expected amplicon size of ˜300 bp) using primers that anneal to the partial kanamycin gene cargo and the ssDNA target (SEQ ID NOs: 1360-1361). Insertion was further confirmed by Sanger sequencing. Based on these results, Hp TnpA can insert ssDNA produced by Ec86 from all of the 5 engineered ncRNAs tested (LE200RE_v1/v3 and LE500RE_v1/v2/v3) and in a manner that is both RT- and TnpA-dependent (FIGS. 42-43).

MG154-159 and MG173 Family Tolerance to Insertion within the Msd of the ncRNA

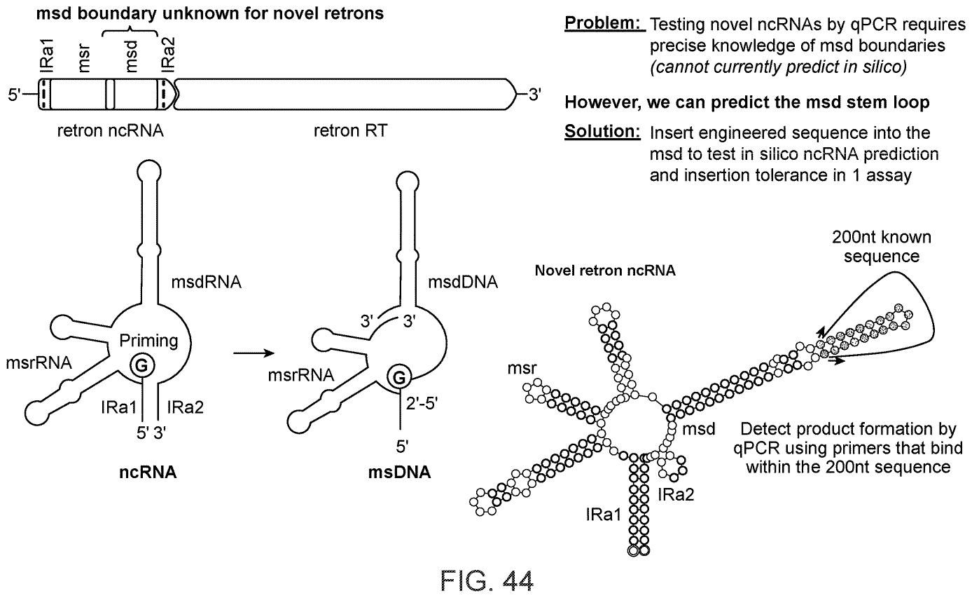

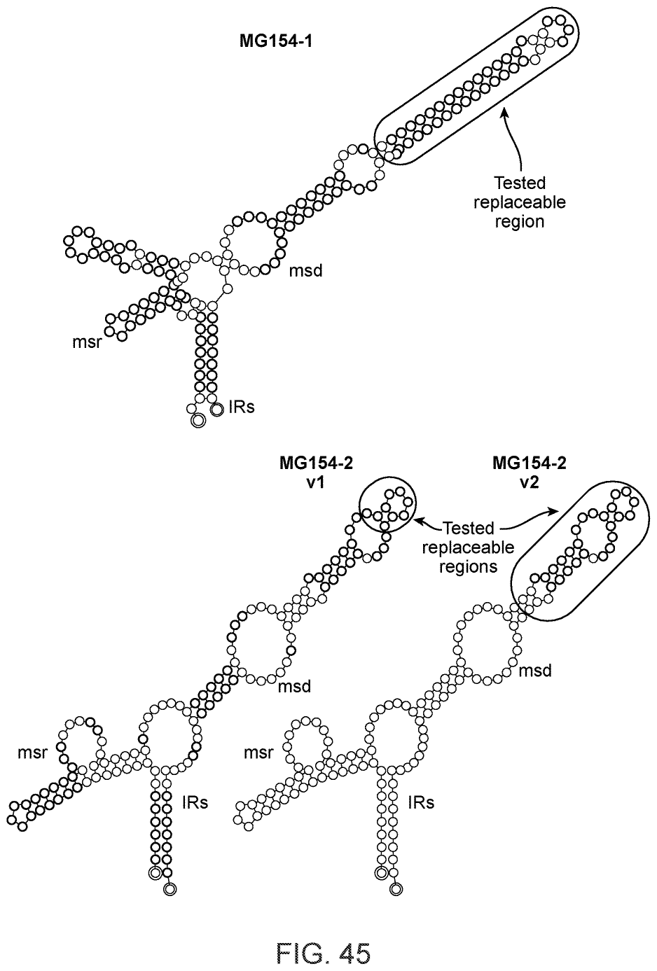

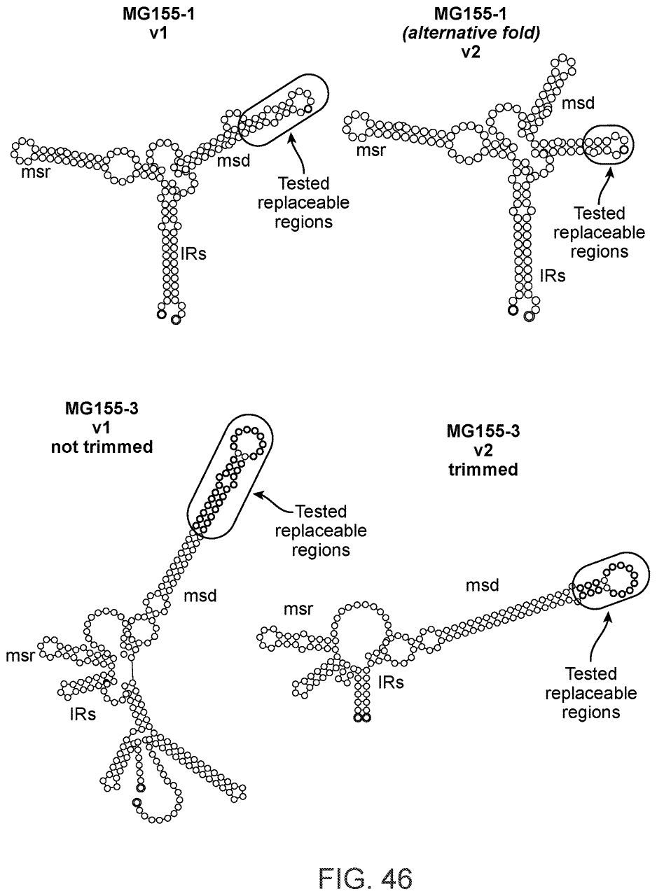

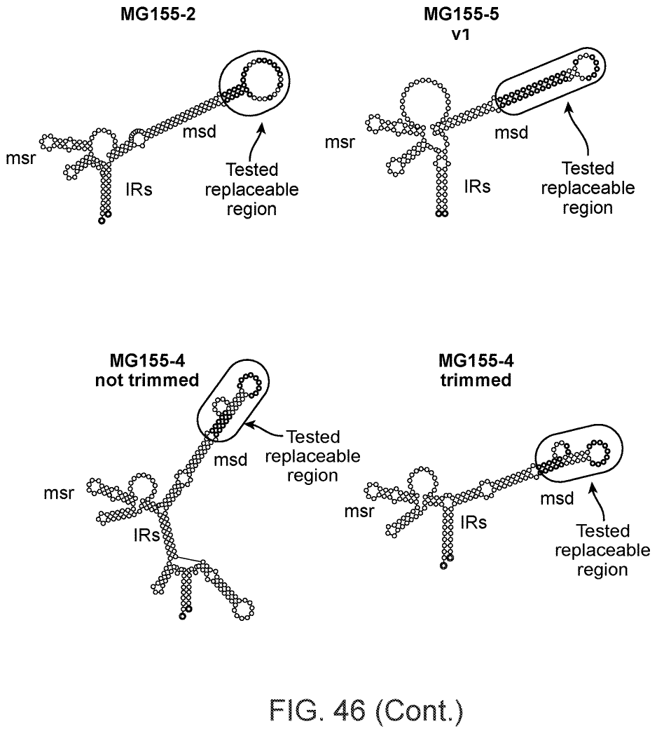

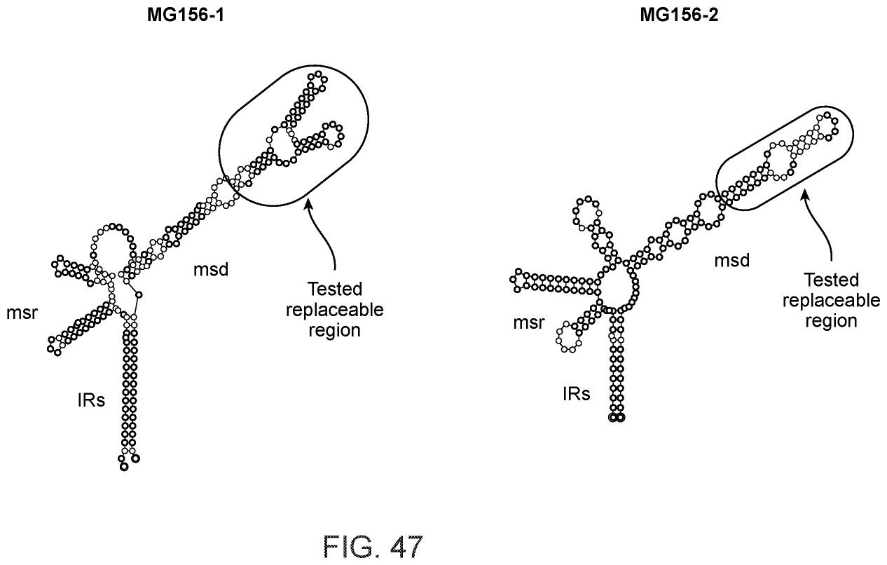

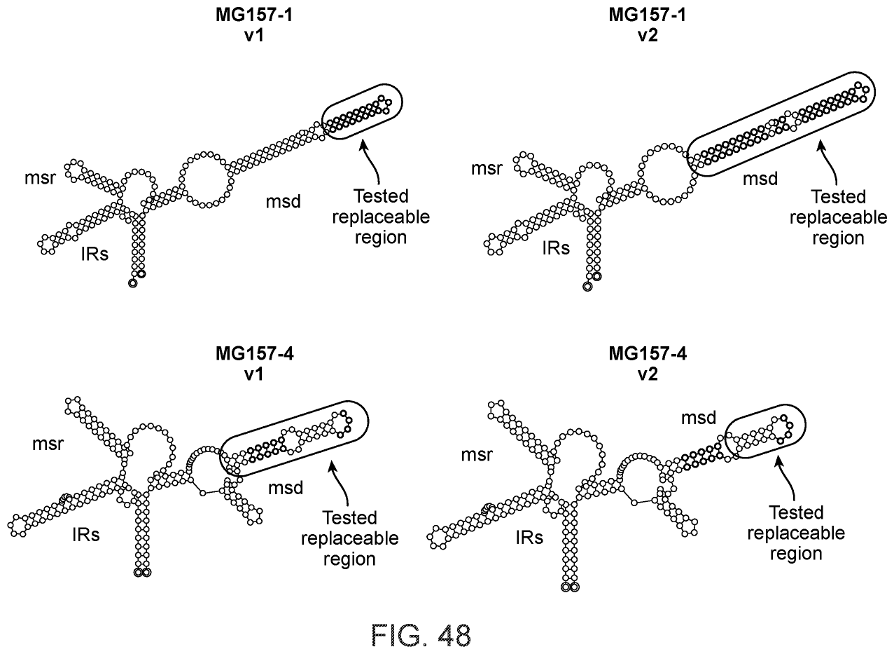

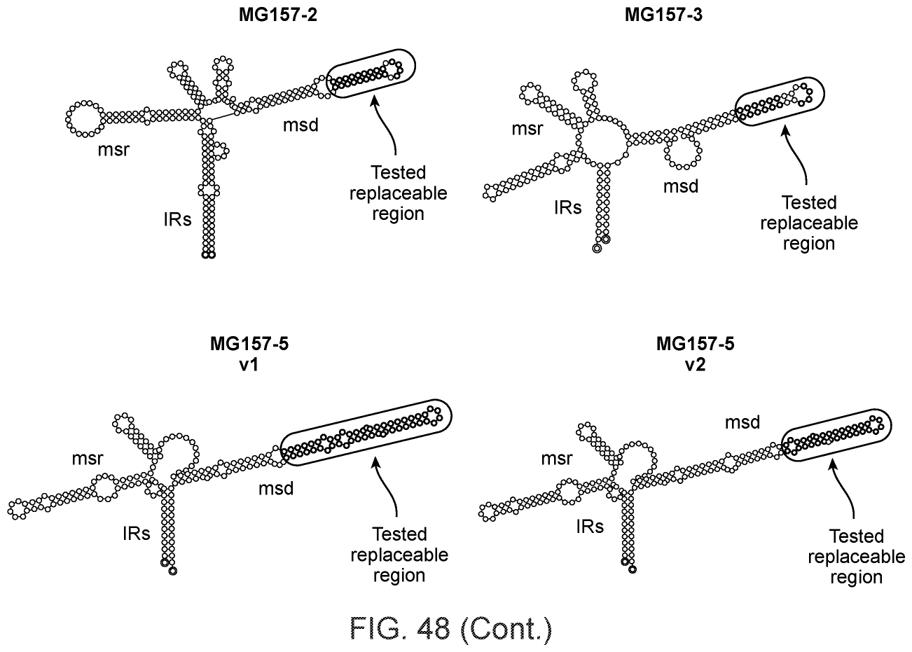

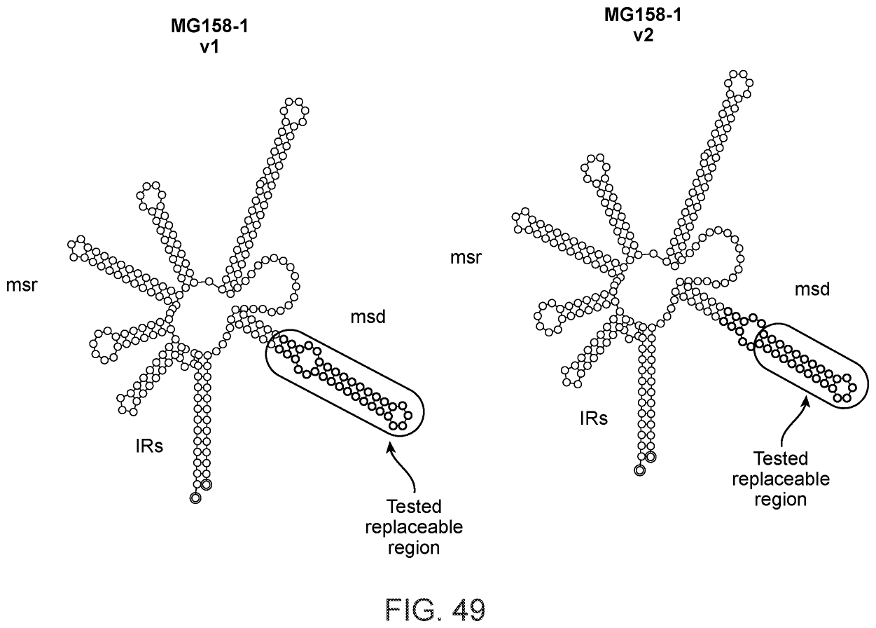

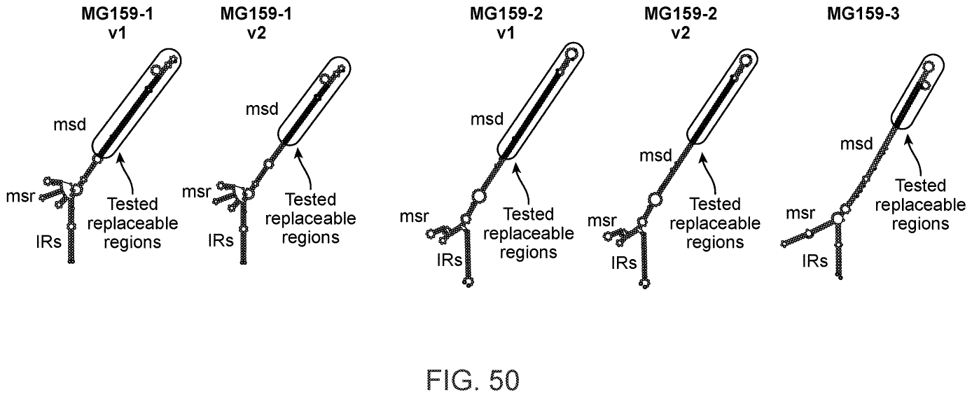

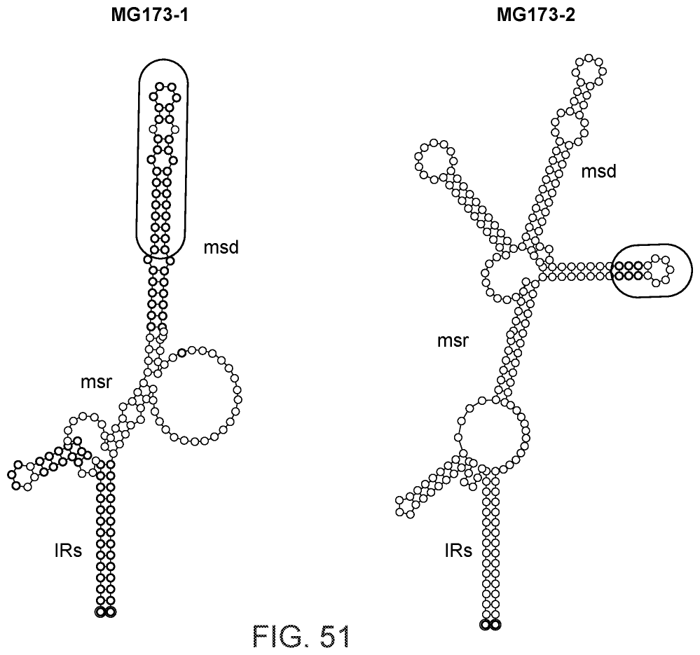

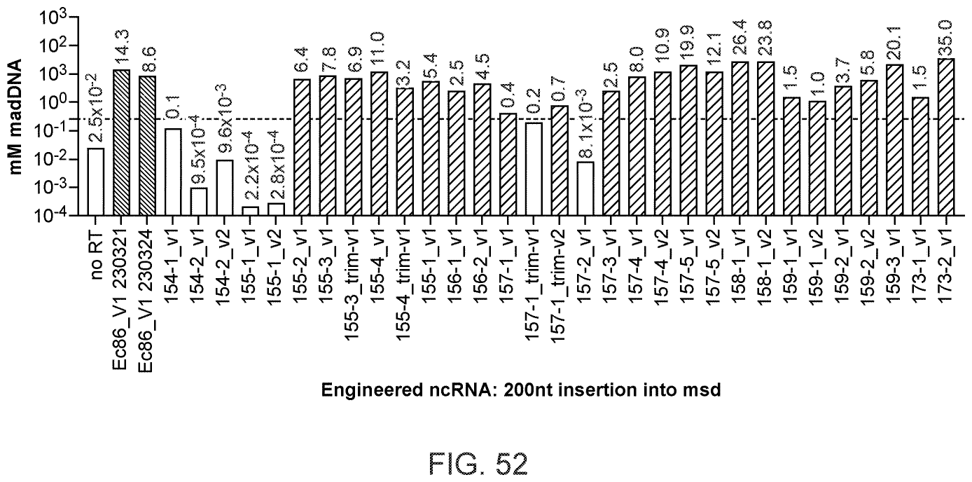

Based on the predicted secondary structure of the ncRNA, the msd stem loop was identified as the first 3′ hairpin adjacent to the inverted repeat. One or two versions of replaceable regions of the msd were identified and a ˜200nt sequence encoding a partial kanamycin gene was inserted (FIGS. 44-51; SEQ ID NOs: 1362-1393). For the cases indicated, both trimmed and untrimmed versions of the ncRNA were also designed and tested (FIG. 46). To evaluate if the retron is tolerant to insertions within the msd stem loop, the corresponding retron RT was co-expressed with the engineered ncRNA in a cell-free expression system supplemented with dNTPs, followed by heat denaturation and RNase A treatment as described above. The resulting msdDNA was then diluted prior to qPCR to ensure concentrations were within the linear range of detection. qPCR was performed using primers that amplify the partial kanamycin sequence. The amount of msdDNA was quantified by extrapolating values from a standard curve generated with a DNA template of known concentrations. Retron RTs were considered active if msdDNA production was greater than 10-fold above the no RT background control. Based on these results, the following retron systems are tolerant to insertion of the msd (FIG. 52): MG155-2, MG155-3, MG155-4, MG155-5, MG156-1, MG156-2, MG157-1, MG157-3, MG157-4, MG157-5, MG158-1, MG159-1, MG159-2, MG159-3, MG173-1, and MG173-2.

### Example 18. RTs for Short Corrections, Small Insertions, and Deletions

### Reverse Transcriptase Candidates Untethered or Tethered to MG71-2(H883A) Nickase

RT candidates (SEQ ID NOs: 1234, 1249-1250, and 1304) in the tethered system were cloned into a plasmid containing the nickase MG71-2(H883A) (SEQ ID NO: 1309) to generate a RT-nickase fusion (RT either on the C- or N-termini for MG71-2 (H883A)). The CMV promoter drove the expression of the fusion protein, which contained a 33 amino acid linker (SEQ ID NO: 103) between the nickase and the RT candidate. The fusion protein was then transfected into HEK293 T cells with liposomes. In the untethered system, RTs were cloned into a plasmid with a CMV promoter driving expression of RT. Another plasmid containing a nickase MG71-2(H883A) driven by a EF1α promoter and the RT containing plasmid were co-transfected using liposomes. Chemically synthesized pegRNAs (SEQ ID NOs: 1310-1315) containing the desired edit in the RT template were transfected using liposomes targeting AAVS1. All components (plasmid and pegRNAs) were reverse transfected into 50,000 HEK293T cells in a 24 well plate. 72 hour post transfection, cells were lysed. Primers containing barcodes for next generation sequencing (NGS) (SEQ ID NOs: 1342-1343) were used to amplify a ˜250 bp target (SEQ ID NO: 1344) with PCR. Samples were purified and sequenced. Sequencing data was processed to determine the percentage of reads with desired change.

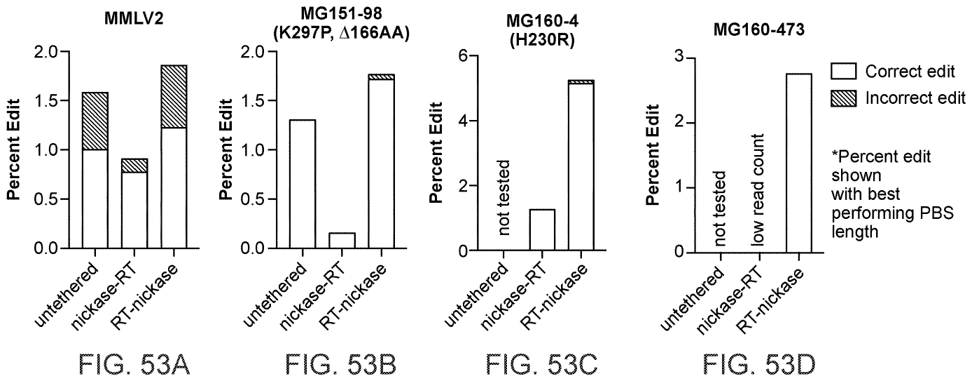

Engineered MG151-98 (K297P, Δ166AA) (SEQ ID NO: 1304) and MMLV2 (SEQ ID NO: 1249) were tested either untethered or tethered to MG71-2(H883A) (RT on C-term of MG71-2(H883A) (nickase-RT) or N-term of MG71-2(H883A) (RT-nickase)) (FIGS. 53A-53B). MG160-4 (H230R) (SEQ ID NO: 1234) and MG160-473 (SEQ ID NO: 1250) were tested tethered to MG71-2(H883A) (RT on C-term of MG71-2(H883A) (nickase-RT) or N-term of MG71-2(H883A) (RT-nickase)) (FIGS. 53C-53D). RTs were challenged to incorporate a 5 nucleotide change on the AAVS1 target (SEQ ID NO: 1344). The RTs were transfected alongside pegRNAs with PBS lengths varying (SEQ ID NOs: 1310-1315), and the data shown in FIG. 53 represents the highest levels of editing for each RT at each RT nickase configuration. MG71-2(H883A) untethered with MMLV2 and MMLV2 tethered to the N-terminus of MG71-2(H883A) showed the highest levels of editing compared to MMLV2 tethered to the C-terminus of MG71-2(H883A) (FIG. 53A). Similar results were shown for MG151-98 (K297P, Δ166AA) (SEQ ID NO: 1304) (FIG. 53B). It has previously been shown that candidates from the MG160 family show little to no activity in an untethered system. Engineered MG160-4(H230R) (SEQ ID NO: 1234) and MG160-473 (SEQ ID NO: 1250) were tested tethered to either the N-terminus or C-terminus of MG71-2(H883A). MG160-4(H230R) tethered to the N-terminus of MG71-2(H883A) gave substantially higher levels of editing than when tethered to the C-terminus of MG71-2(H883A) (FIG. 53C). MG160-473 also showed the highest levels of editing when tethered to the N-terminus of MG71-2(H883A) (FIG. 53D). Data shown in FIG. 53 represents the “correct edit” indicating intended correction with no errors found in the NGS amplicon. The “incorrect edit” refers to the intended edit being incorporated but includes errors within the NGS amplicon, either originating in misincorporation by the RT and/or scaffold incorporation of the pegRNA. The data shows that MG71-2(H883A) has a strong preference for RTs on the N-terminus. Further, it has been demonstrated that MG RTs outperform literature controls in terms of efficiency and accuracy.

### Example 19. RTs for Short Corrections, Small Insertions and Deletions

Testing Reverse Transcriptase Candidates Untethered to spCas9(H840A) Nickase

RT candidates (SEQ ID NOs: 1394-1402) in the untethered system were cloned into a plasmid with a CMV promoter driving expression of RT. Another plasmid containing a nickase spCas9(H840A) (SEQ ID NO: 1247) driven by an EF1α promoter and the RT containing plasmid were cotransfected by lipofection. Chemically synthesized pegRNAs (SEQ ID NOS: 76-83) containing the desired edit in the RT template targeting VEGFA (SEQ ID NO: 102) were transfected by high efficiency lipofection. All components (plasmid and pegRNAs) were reverse transfected into 50,000 HEK293T cells in a 24 well plate. 72 hour post transfection, cells were lysed in 100 μL of a DNA extraction solution. Primers containing barcodes for next generation sequencing (NGS) (SEQ ID NOs: 100-101) were used to amplify a ˜250 bp target (SEQ ID NO: 102) with a high fidelity polymerase and reaction solution. PCR clean-up was then performed and samples were sent for NGS sequencing. FASTQ files were then processed to determine the percentage of reads with the desired change.

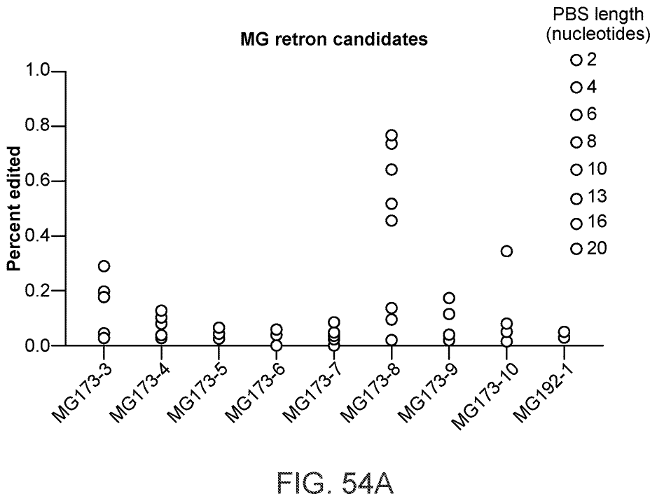

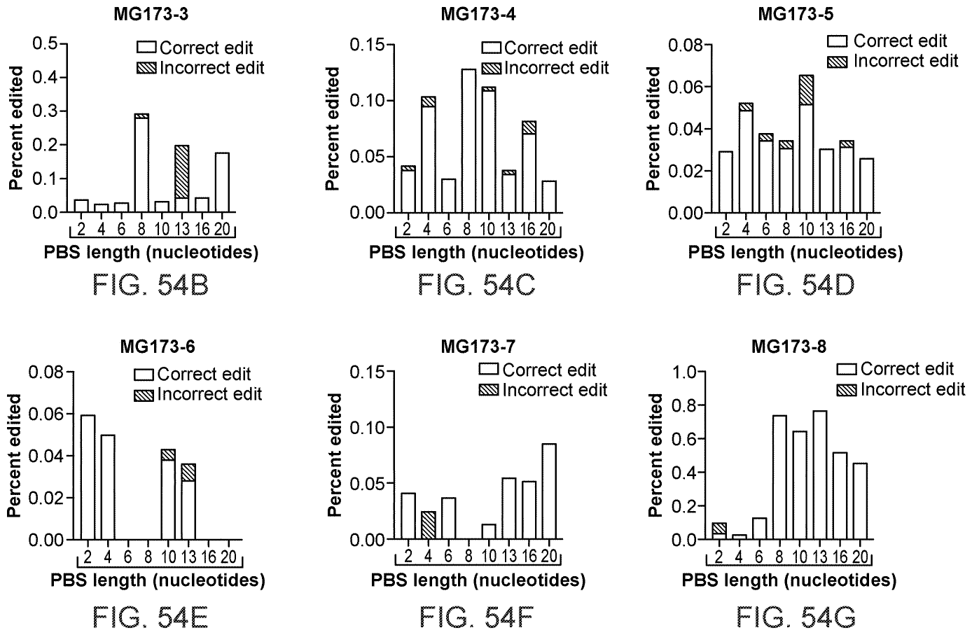

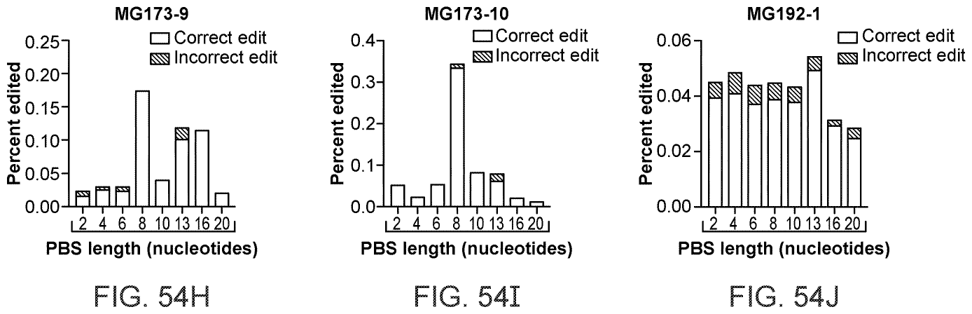

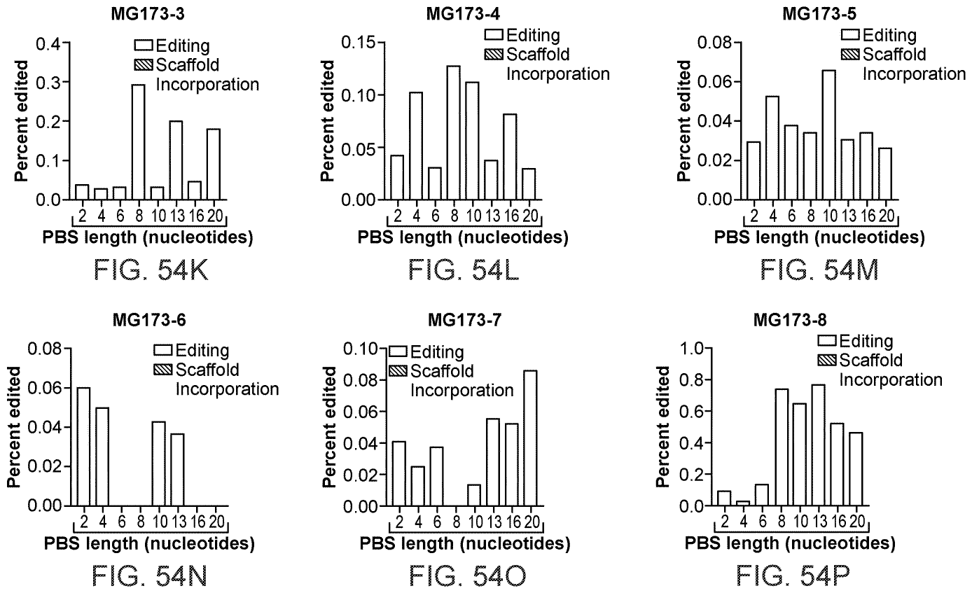

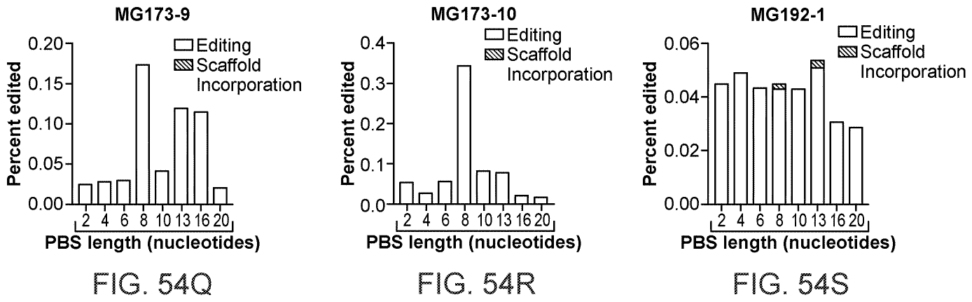

Eight candidates from the MG173 family (SEQ ID NOs: 1394-1401) and one candidate from the MG192 family (SEQ ID NO: 1402) were tested for G-to-T transversion on the VEGFA target (SEQ ID NO: 102) using pegRNAs with PBS lengths varying from 2 to 20 nucleotides and untethered spCas9(H840A) (SEQ ID NO: 1247) (FIG. 54). Reverse transcriptase candidates having editing levels above background (>0.1%) included MG173-3 (SEQ ID NO: 1394), MG173-8 (SEQ ID NO: 1399), MG173-9 (SEQ ID NO: 1400), and MG173-10 (SEQ ID NO: 1401), while the other retron candidates (SEQ ID NOs: 1395-1398 and 1402) were not active for G-to-T transversion (FIG. 54A). Percent editing was then broken down further to determine “correct edit”, “incorrect edit”, “editing”, and “scaffold incorporation” (FIGS. 54B-54S). “Correct edit” represents the intended edit with no mistakes in the NGS amplicon, while “incorrect edit” refers to the intended edit being incorporated and includes errors within the NGS amplicon, either originating in misincorporation by the RT and/or scaffold incorporation of the pegRNA. “Editing” refers to the intended edit with errors in the NGS amplicon (excluding pegRNA scaffold incorporation) and “scaffold incorporation” indicates the intended edit and scaffold incorporation of the pegRNA. MG173-8 (SEQ ID NO: 1399) showed the highest levels of editing compared to the other retron candidates (FIGS. 54A, 54G, and 54P) with the highest level of percent editing between PBS 8 through 13 nucleotides (SEQ ID NOS: 79-81).

Testing Reverse Transcriptase Candidates Tethered to spCas9(H840A) Nickase

RT candidates (SEQ ID NOs: 1403-1424) in the tethered system were cloned into a plasmid containing the nickase spCas9(H840A) (SEQ ID NO: 1247) to generate an RT-nickase fusion. The CMV promoter drove the expression of the fusion protein, which contained a 33 amino acid linker (SEQ ID NO: 103) between the nickase and the RT candidate. Transfection of these constructs, along with chemically synthesized pegRNAs, followed the transfection protocol and NGS sample preparation and data analysis mentioned above.

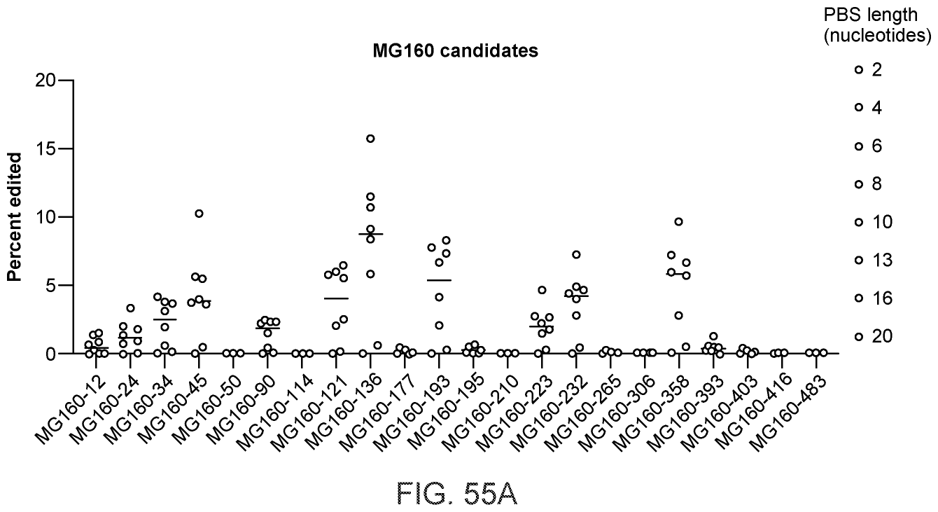

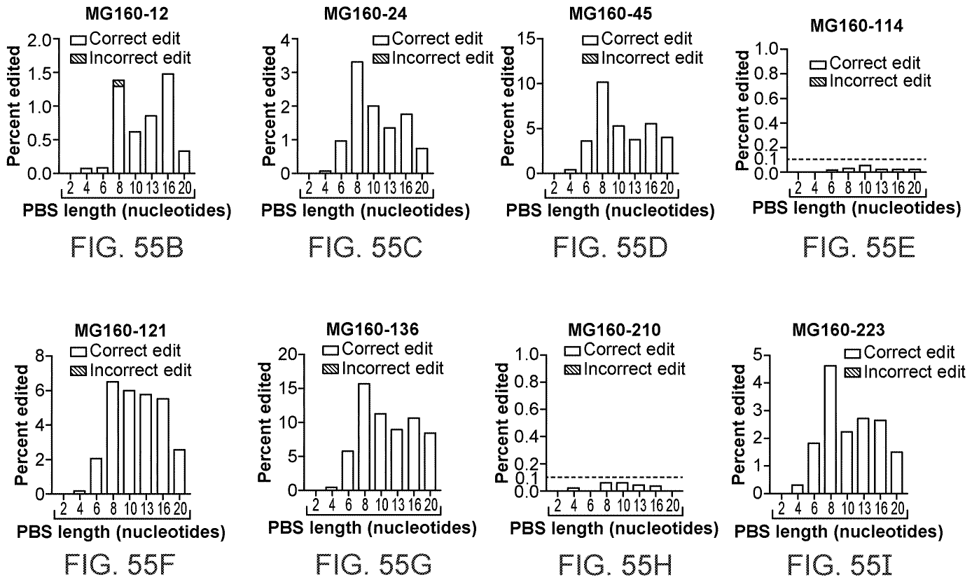

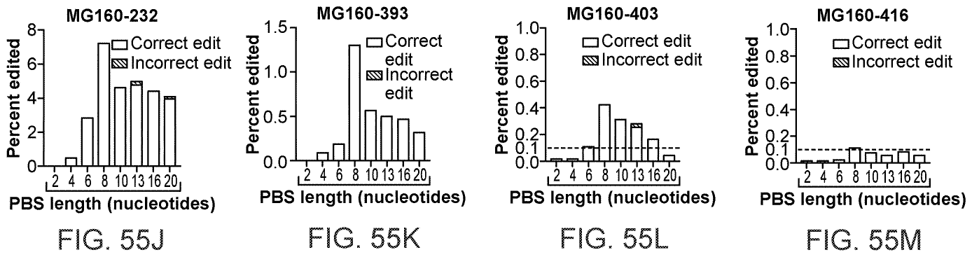

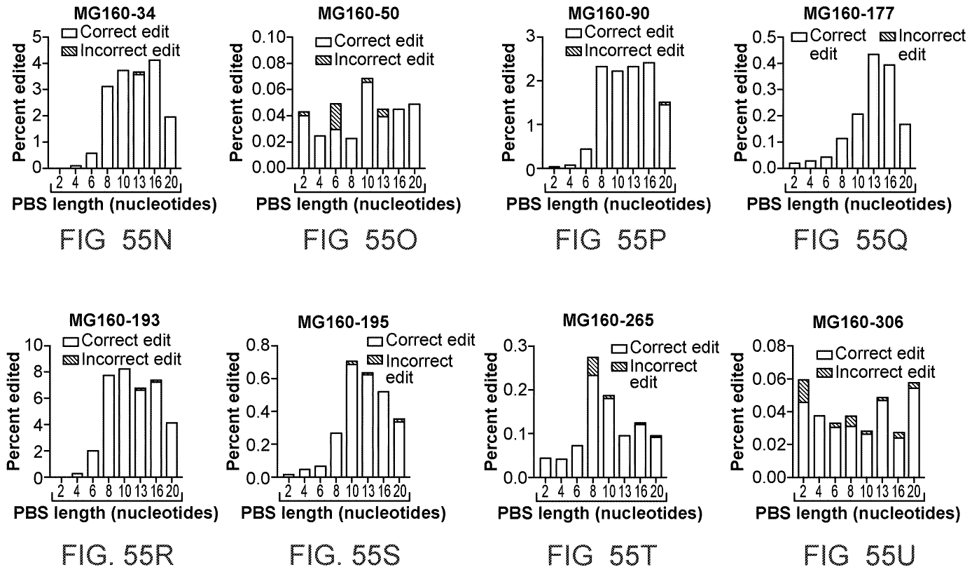

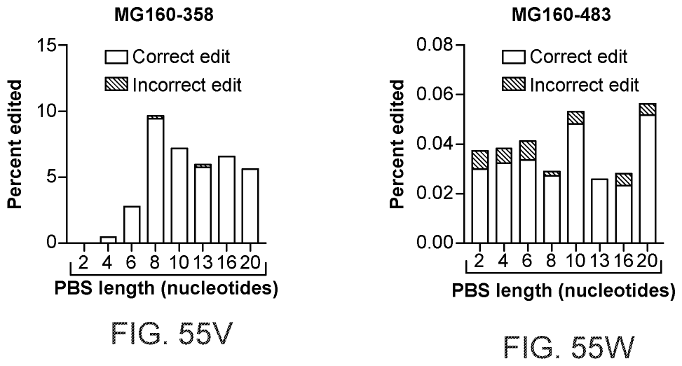

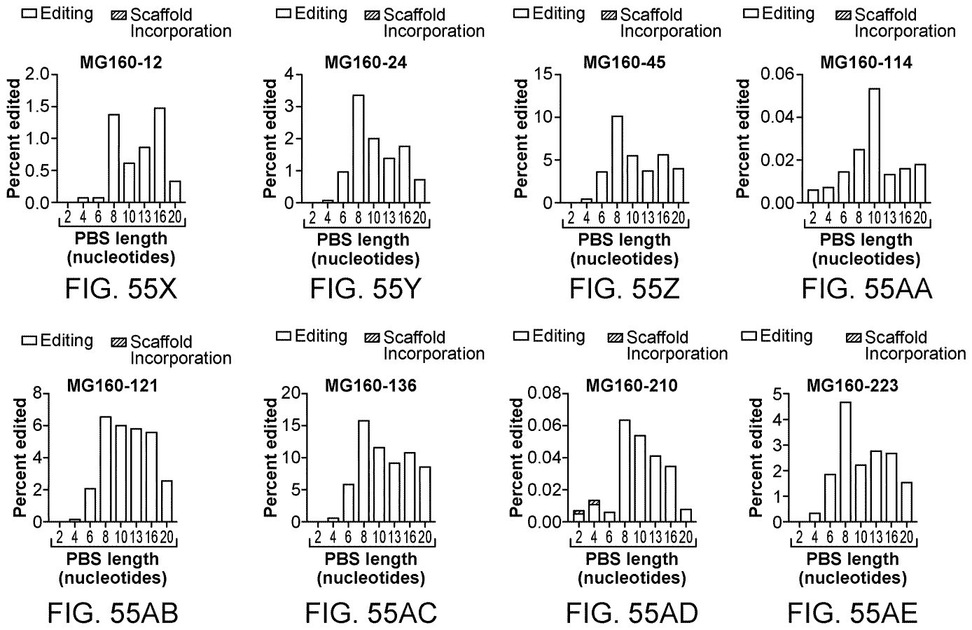

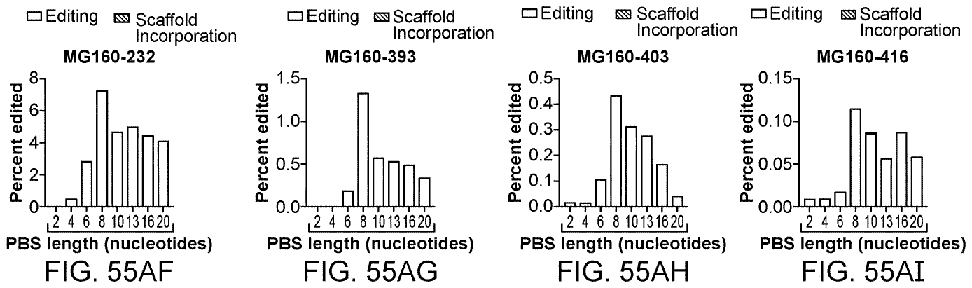

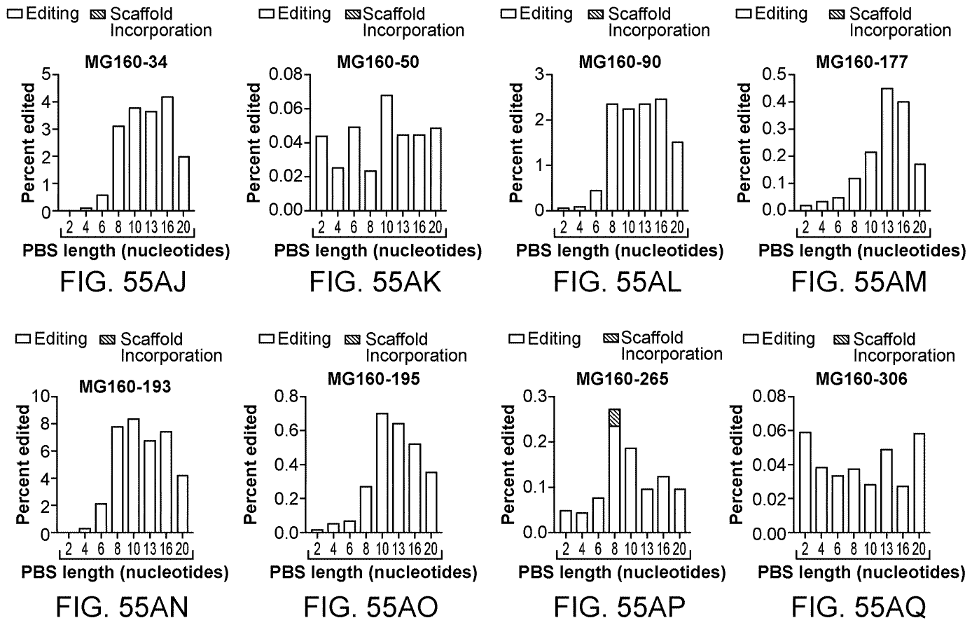

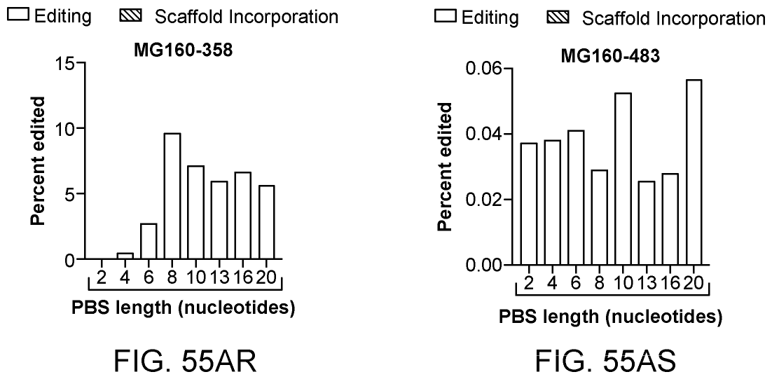

Twenty-two MG160 candidates (SEQ ID NOs: 1403-1424) were tested tethered to spCas9(H840A) (SEQ ID NO: 1247) for G-to-T transversion on the VEGFA target (SEQ ID NO: 102) across eight different pegRNAs with varying PBS lengths (SEQ ID NOS: 76-83) (FIG. 55A). Candidates that did not show activity above background under the tested conditions (>0.1%) were MG160-50 (SEQ ID NO: 1409) (FIGS. 550 and 55AK), MG160-114 (SEQ ID NO: 1404) (FIGS. 55E and 55AA), MG160-210 (SEQ ID NO: 1412) (FIGS. 55H and 55AD), MG160-306 (SEQ ID NO: 1418) (FIGS. 55U and 55AQ), MG160-416 (SEQ ID NO: 1422) (FIGS. 55M and 55AI), and MG160-483 (SEQ ID NO: 1424) (FIGS. 55W and 55AS). MG160 candidates with high levels of activity for G-to-T transversion include MG160-45 (SEQ ID NO: 1423) (FIGS. 55D and 55Z), MG160-121 (SEQ ID NO: 1405) (FIGS. 55F and 55AB), MG160-136 (SEQ ID NO: 1407) (FIGS. 55G and 55AC), MG160-193 (SEQ ID NO: 1410) (FIGS. 55R and 55AN), MG160-232 (SEQ TD NO: 1407) (FIGS. 55J and 55AF), and MG160-358(SEQ ID NO: 1419) (FIGS. 55V and 55AR). MG160-136 (SEQ ID NO: 1407) reached editing levels above 5% for PBS lengths of 6-20 nucleotides (SEQ ID NOS: 78-83), with the highest level of editing at PBS 8 (SEQ ID NO: 79) reaching ˜15% editing for G-to-T transversion (FIGS. 55G and 55AC). Percent editing was then broken down further to determine “correct edit”, “incorrect edit”, “editing”, and “scaffold incorporation” (terms described in detail above) (FIGS. 55B-55AS).

### Example 20. Short Corrections, Small Insertions and Deletions with Engineered RTs

Testing Engineered Reverse Transcriptase Candidates Untethered or Tethered to spCas9(H840A) Nickase

Selected RT candidates were subjected to rational engineering to improve editing efficiencies. Various point mutations were tested individually as well as combined to determine which engineered candidates could improve editing activity. The selected RT candidates and engineered mutants (MG151-98 (SEQ ID Nos: 1300 and 1302-1304), MG151-123 (SEQ ID NOs: 715, and 1426-1431), MG151-126 (SEQ ID NOs: 718, and 1433-1438), MG153-18 (SEQ ID Nos: 55 and 1439-1441), and MG153-20 (SEQ ID Nos: 57 and 1442-1444)) were tested untethered to spCas9(H840A) (SEQ ID NO: 1247), while MG160-473 (SEQ ID NO: 1250) and mutants (SEQ ID Nos: 1445-1446) were tested tethered to spCas9(H840A) (SEQ ID NO: 1247). Using chemically synthesized pegRNAs with varying PBS lengths and RTT (SEQ ID NOS: 78-81, 86-90, and 94-98), engineered reverse transcriptases were challenged to versatile edits (transversion, insertion, and deletion) on the VEGFA target (SEQ ID NO: 102). Engineered reverse transcriptases were tested either untethered or tethered to spCas9(H840A) (SEQ ID NO: 1247) using the same transfection protocol and NGS preparation and data analysis described in Example 19.

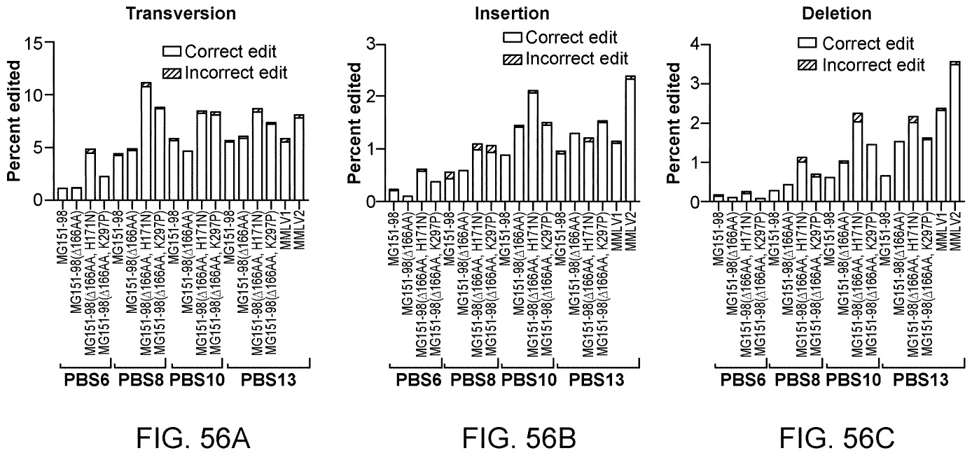

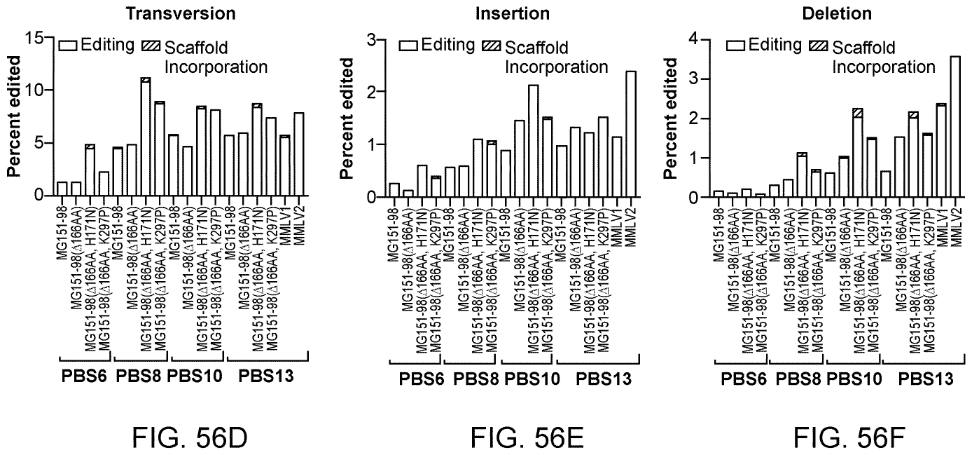

MG151-98 wild type (SEQ ID NO: 1300) and engineered mutants MG151-98 (Δ166AA) (SEQ ID NO: 1302), MG151-98 (H171N, Δ166AA) (SEQ ID NO: 1303), and MG151-98 (K297P, Δ166AA) (SEQ ID NO: 1304) were tested untethered with spCas9(H840A) (SEQ ID NO: 1247) for G-to-T transversion (FIGS. 56A and 56D), 24 nucleotide insertion (FIGS. 56B and 56E), and 15 nucleotide deletion (FIGS. 56C and 56F) on the VEGFA target (SEQ ID NO: 102) using pegRNAs with PBS lengths of 6, 8, 10, and 13 nucleotides (SEQ ID NOS: 78-81, 86-90, and 94-98). Trimming 166 amino acids from the C-terminus of MG151-98 (MG151-98 (Δ166AA) (SEQ ID NO: 1302)) resulted in no significance difference in editing levels compared to wild type (SEQ ID NO: 1300) across three different type of edits (FIG. 56). Further, single point mutants H171N and K297P combined with 166AA trimmed off the C-terminus of the reverse transcriptase (SEQ ID Nos: 1303-1304) enhanced editing compared to wild type MG151-98 (SEQ ID NO: 1300) and brought editing levels above MMLV1 (SEQ ID NO: 1248) and comparable to MMLV2 (SEQ ID NO: 1249) for some types of edits (FIG. 56). Percent editing was broken down further to determine “correct edit”, “incorrect edit”, “editing”, and “scaffold incorporation” (terms described in detail in Example 19). Even with engineered mutants of MG151-98 (SEQ ID Nos: 1302-1304) improving editing levels, there was no significant difference in “incorrect edit” and “scaffold incorporation” compared to wild type (FIG. 56).

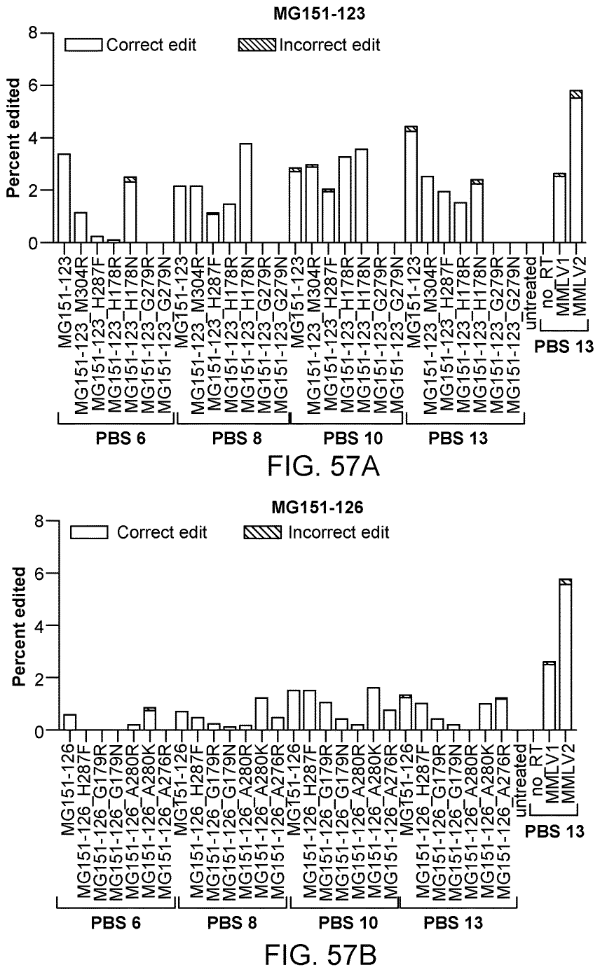

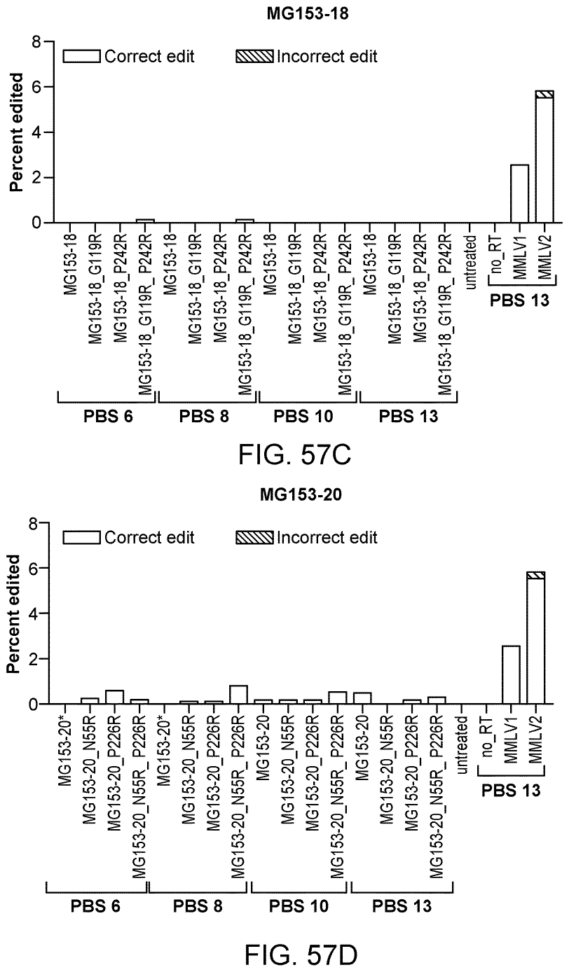

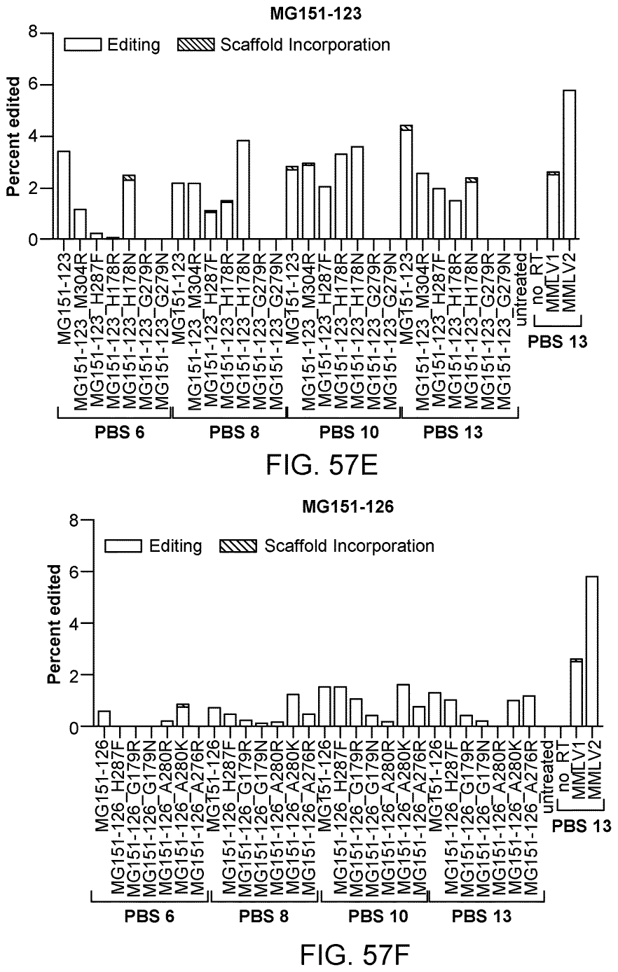

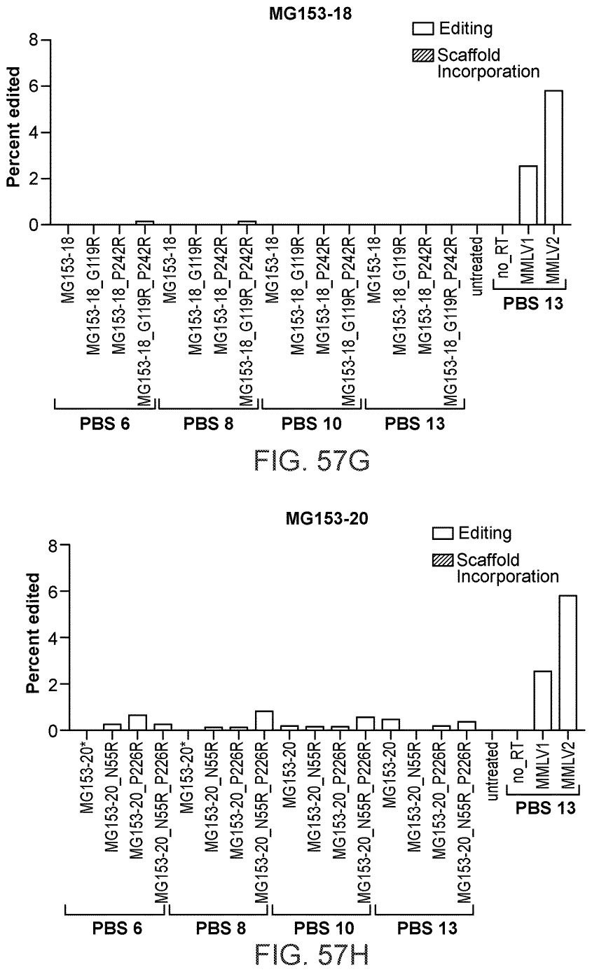

Wild type and engineered mutants of MG151-123 (SEQ ID NOs: 715, 1426-1431), MG151-126 (SEQ ID NOs: 718, and 1433-1438), MG153-18 (SEQ ID Nos: 55 and 1439-1441), and MG153-20 (SEQ ID Nos: 57 and 1442-1444) were tested for G-to-T transversion on VEGFA target (SEQ ID NO: 102) using pegRNAs with PBS lengths of 6, 8, 10, and 13 nucleotides (SEQ ID NOS: 78-81, 86-90, and 94-98) (FIG. 57). An MG151-123 mutant, MG151-123 (H178N) (SEQ ID NO: 1429), showed editing levels that improved at PBS 8 and PBS 10 compared to wild type MG151-123 (SEQ ID NO: 715) (FIGS. 57A and 57E). Other point mutations, M304R, H287F, H178R, G279R, and G279N for MG151-123 (SEQ ID Nos: 1426-1428 and 1430-1431) either significantly decreased or abolished activity for G-to-T transversion (FIGS. 57A and 57E). MG151-126 (SEQ ID NO: 718) and point mutations (SEQ ID Nos: 1433-1438) showed much lower editing levels compared to MG151-123 (SEQ ID NO:715) and were not comparable to MMLV1 (SEQ ID NO: 1248) or MMLV2 (SEQ ID NO: 1249) (FIGS. 57B and 57F). Further, for MG153-18 (SEQ ID NO: 55) and MG153-20 (SEQ ID NO: 57), single point mutations (SEQ ID Nos: 1439-1440 and 1442-1443) and double point mutations (SEQ ID Nos: 1441 and 1444) showed no editing levels above background when tested for G-to-T transversion (FIGS. 57C, 57D, 57G, and 57H).

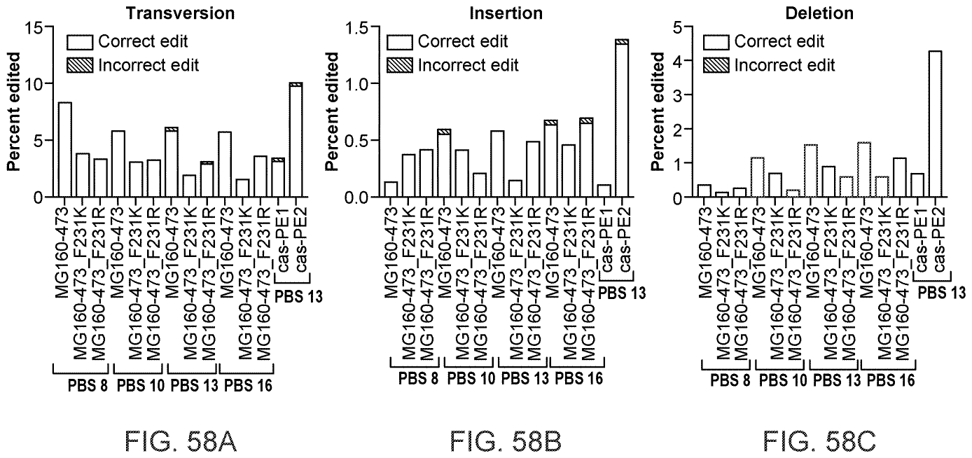

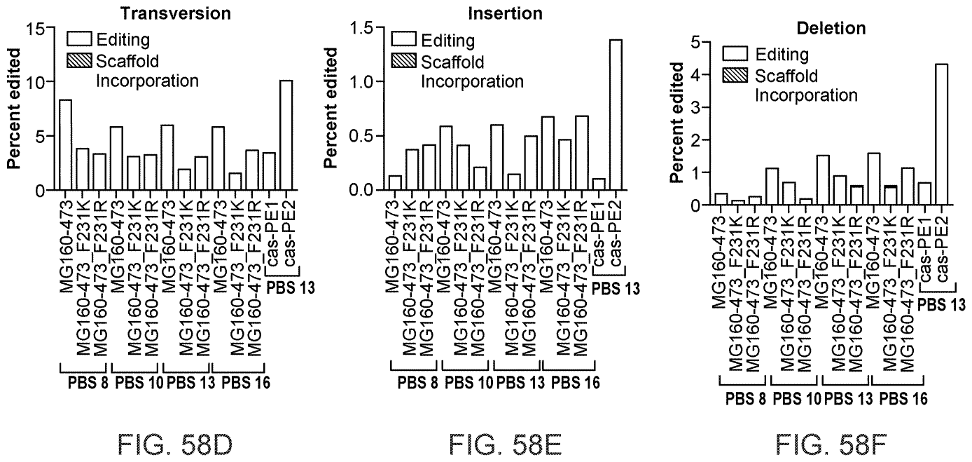

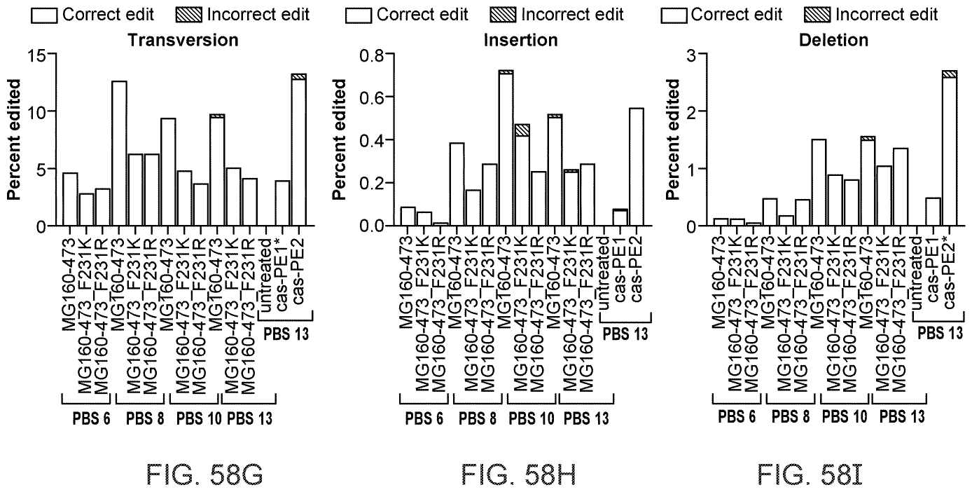

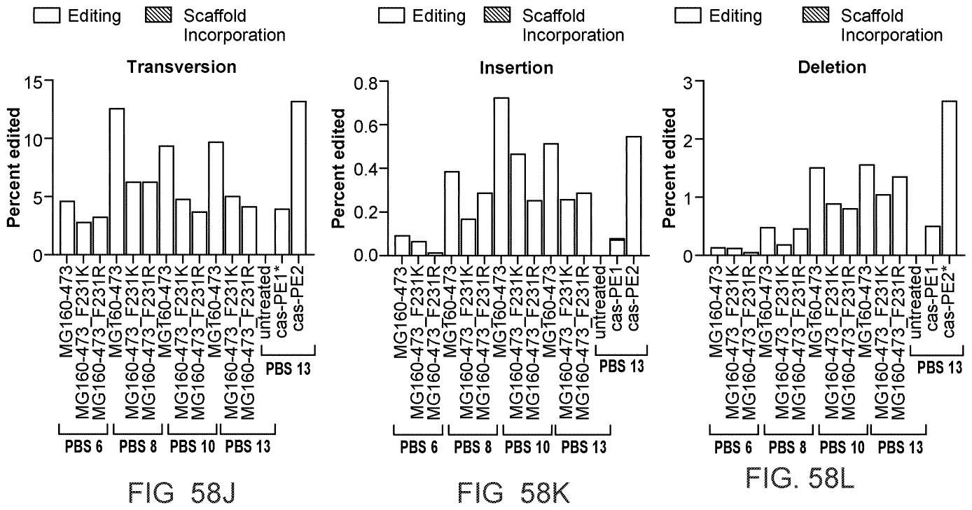

MG160-473 wild type (SEQ ID NO: 1250) and point mutants MG160-473 (F231R) (SEQ ID NO: 1445) and MG160-473 (F231K) (SEQ ID NO: 1446) were tested for G-to-T transversion (FIGS. 58A, 58D, 58G, and 58J), 24 nucleotide insertion (FIGS. 58B, 58E, 58H, and 58K), and 15 nucleotide deletion (FIGS. 58C, 58F, 58I, and 58L) on the VEGFA target (SEQ ID NO: 102) using pegRNAs with PBS lengths of 6, 8, 10, 13, and 16 nucleotides (SEQ ID NOS: 78-82, 86-90, and 94-98). Single point mutations (SEQ ID Nos: 1445-1446) did not result in improvements on editing levels compared to wild type MG160-473 (SEQ ID NO: 1250). MG160-473 (SEQ ID NO: 1250) outperformed tethered MMLV1 (SEQ ID NO: 1248) for all edits and showed comparable levels of editing for transversion compared to MMLV2 (SEQ ID NO: 1249) (FIG. 58).

### Example 21. Nickases for Mediating Short Corrections, Small Insertions and Deletions in Conjunction with Reverse Transcriptases

Installing genomic corrections, insertions, or deletions using RTs require the system to be targetable. The targetability of the system is given by the use of a Cas nickase. The Cas nickase nicks the non-target strand, creating a primer for reverse transcription. The gRNA that accompanies the Cas nickase is a modified version (pegRNA) that consists of a 3′ extension containing the RTT and the PBS. The complementarity of the PBS and the spacer can result in gRNA structure disruption, causing the pegRNA to interact with the Cas and thus inhibiting the Cas from finding the target gene. Because each Cas nuclease interacts with its own gRNA, the pegRNA design and requirements vary from system to system.

Optimizing MG71-2(H883A) Nickase with MG Reverse Transcriptases

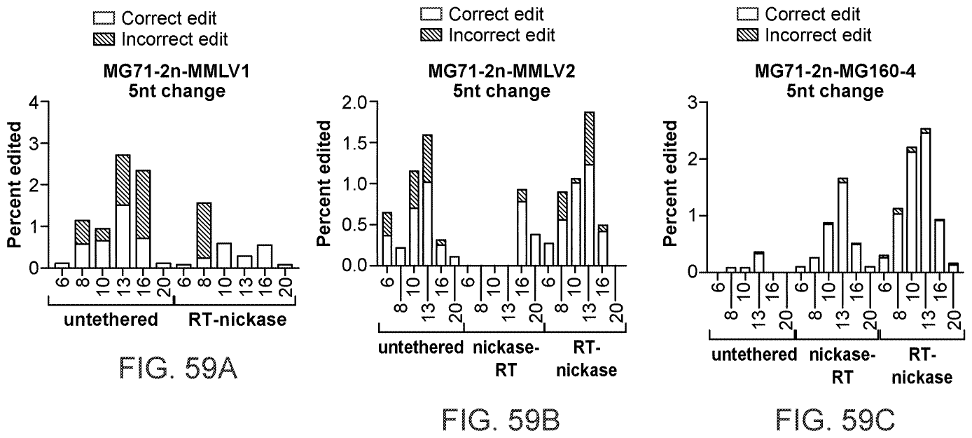

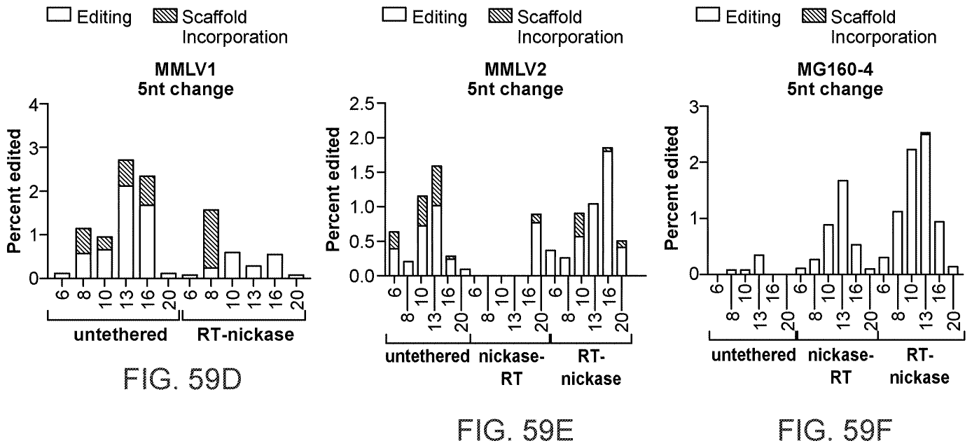

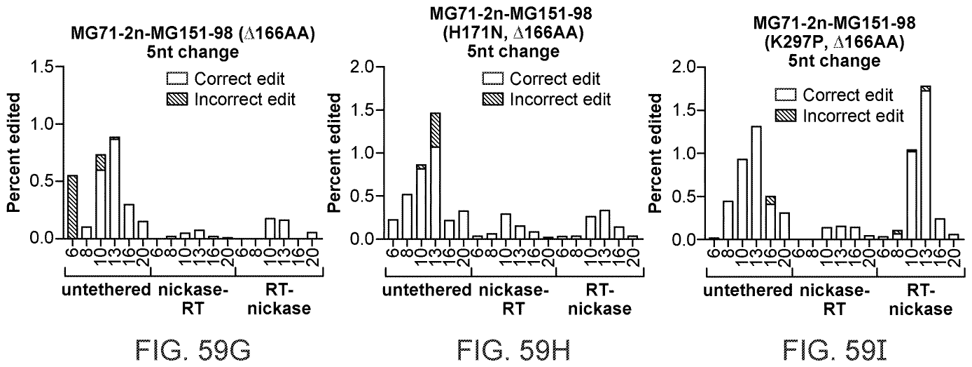

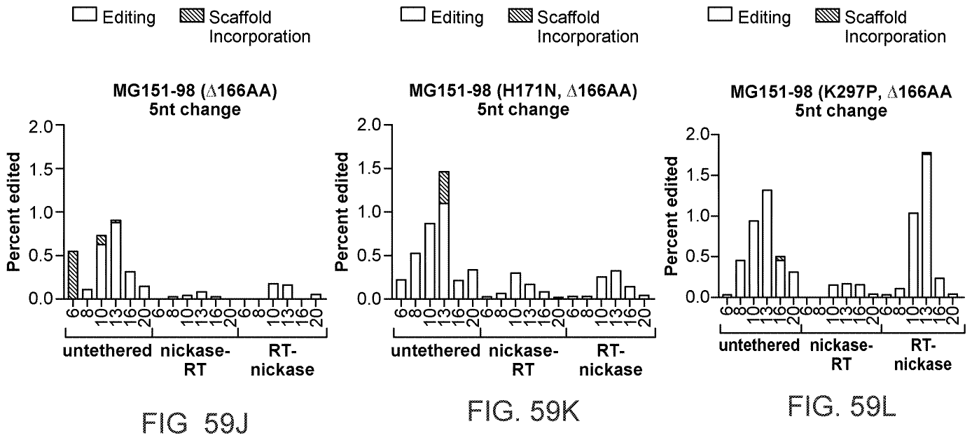

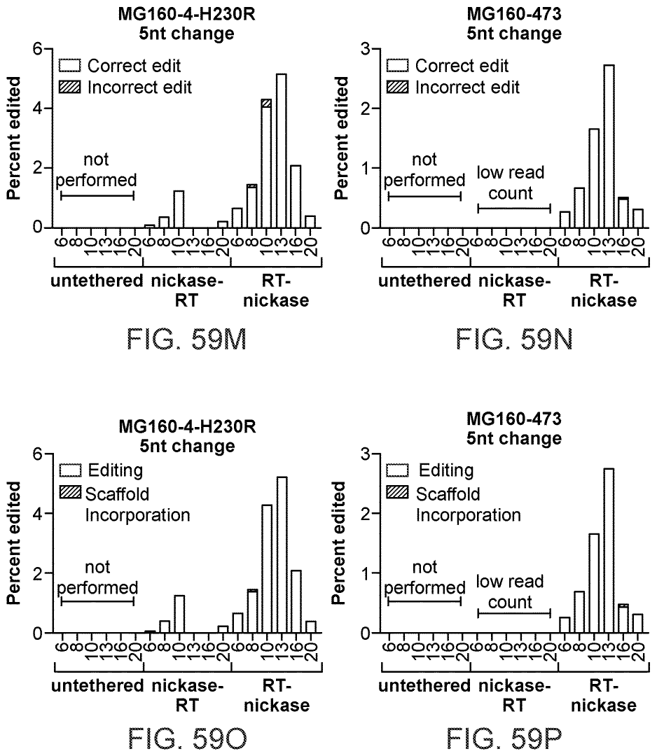

An MG71-2(H883A) nickase (MG71-2n) (SEQ ID NO: 1309) was challenged to introduce genomic corrections (a five nucleotide change, G-to-T transversion, a 24 nucleotide insertion, and a 15 nucleotide deletion) on an AAVS1 target site (SEQ ID NO: 1344) with selected MG reverse transcriptase candidates (FIGS. 59-61). Reverse transcriptases were tested either untethered with MG71-2n, tethered to the C-terminus of MG71-2n, or tethered to the N-terminus of MG71-2n with a 33 AA linker (SEQ ID NO: 103). A similar procedure to the transfection and preparation of NGS samples protocols described in Example 19 was used with the exception of different pegRNAs with PBS lengths 6 through 20 nucleotides (SEQ ID NOS: 1310-1315 and 1324-1341) and NGS primers (SEQ ID Nos: 1342-1343) to target the AAVS1 site with MG71-2n. Optimization of pegRNAs by modifying the scaffold of the pegRNA and incorporating mismatches in the PBS sequence was tested to determine if editing levels could be improved.

Selected reverse transcriptases MMLV1 (SEQ ID NO: 1248; FIGS. 59A and 59D), MMLV2 (SEQ ID NO: 1249; FIGS. 59B and 59E), MG160-4 (SEQ ID NO: 1295; FIGS. 59C and 59F), MG151-98(Δ166AA) (SEQ ID NO: 1302; FIGS. 59G and 59J), MG151-98(H178N, Δ166AA) (SEQ ID NO: 1303; FIGS. 59H and 59K), MG151-98(K297P, Δ166AA) (SEQ ID NO: 1304; FIGS. 59I and 59L), MG160-4 (H230R) (SEQ ID NO: 1234; FIGS. 59M and 59O), and MG160-473 (SEQ ID NO: 1250; FIGS. 59N and 59P) were tested either untethered or tethered to MG71-2n across six different PBS lengths (6, 8, 10, 13, 16, or 20 nucleotides) containing a reverse transcription template (RTT) encoding a five nucleotide change. In general, when using a tethered approach for MG71-2n, the reverse transcriptase on the N-terminus of MG71-2n showed higher levels of editing when compared to the reverse transcriptase on the C-terminus of MG71-2n (FIG. 59). Different reverse transcriptase candidates demonstrated preference for being tethered or untethered (FIG. 59). For example, MG160 family candidates MG160-4, MG160-4 (H230R), and MG160-473 showed much higher levels of editing when tethered compared to the untethered format (FIGS. 59C, 59F, and 59M-59P). In contrast, MG151-98 (Δ166AA) and MG151-98 (H178N, Δ166AA) showed higher levels of editing when untethered to MG71-2n (FIGS. 59G-59L), which may be due to the use of a non-optimal linker for MG151-98 (SEQ ID NO: 1300). In general, MG reverse transcriptases have fewer errors and scaffold incorporation than MMLV1 and MMLV2 when targeting this region of AAVS1 with MG71-2n.

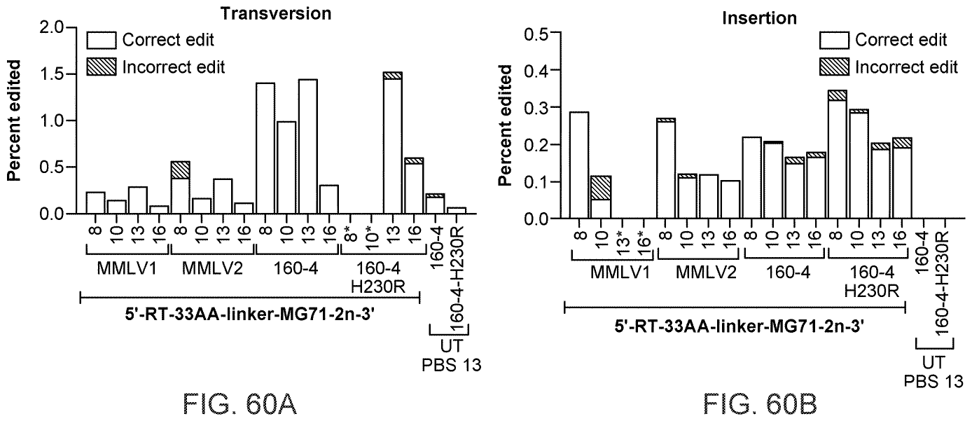

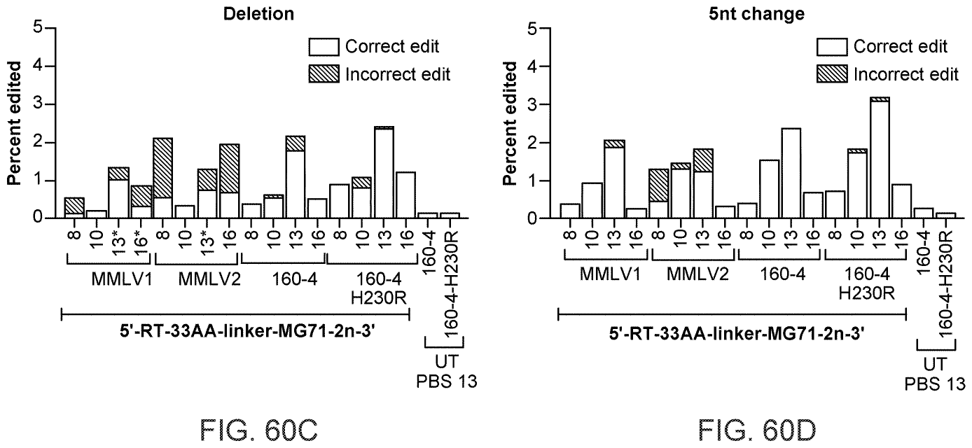

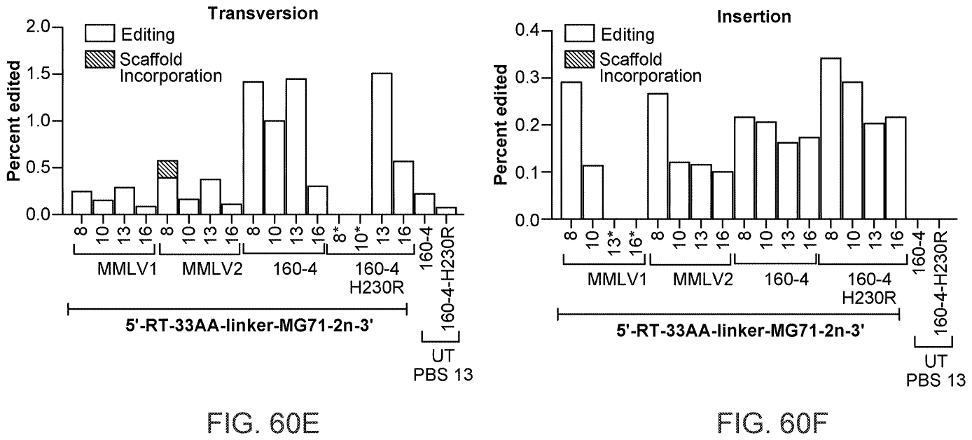

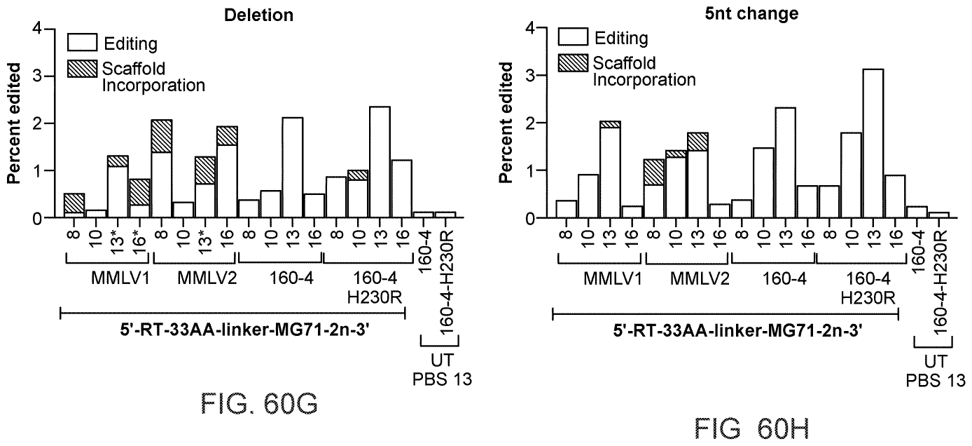

MG160-4 and MG160-4 (H230R) tethered to the N-terminus of MG71-2n was then tested to incorporate a G-to-T transversion, a 24 nucleotide insertion, a 15 nucleotide deletion, and a five nucleotide change on an AAVS1 target site using pegRNAs with PBS lengths of 8, 10, 13, and 16 nucleotides (FIGS. 60A-60H). MG160-4 (H230R) outperformed or was comparable to wild type MG160-4 depending on the intended correction. In addition, MG160-4 and MG160-4 (H230R) were comparable to or had improved editing levels compared to MMLV1 or MMLV2 tethered to the N-terminus of MG71-2n. MG160 candidates were tested untethered at only PBS 13 for all edits and in all cases, tethered MG160 candidates had higher activity when tethered than untethered. Interestingly, when performing a deletion correction, scaffold incorporation was much higher than other types of edits (FIG. 60G). However, when a reverse transcriptase was tethered to MG71-2n, scaffold incorporation seemed to decrease.

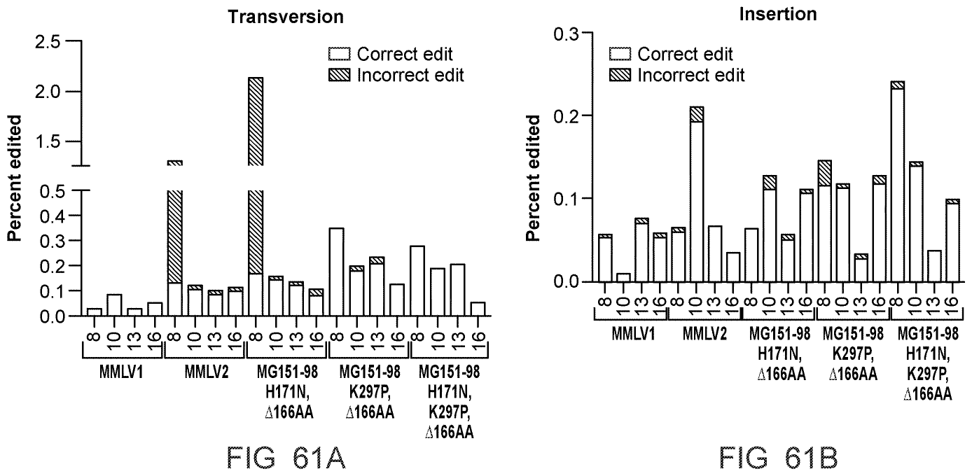

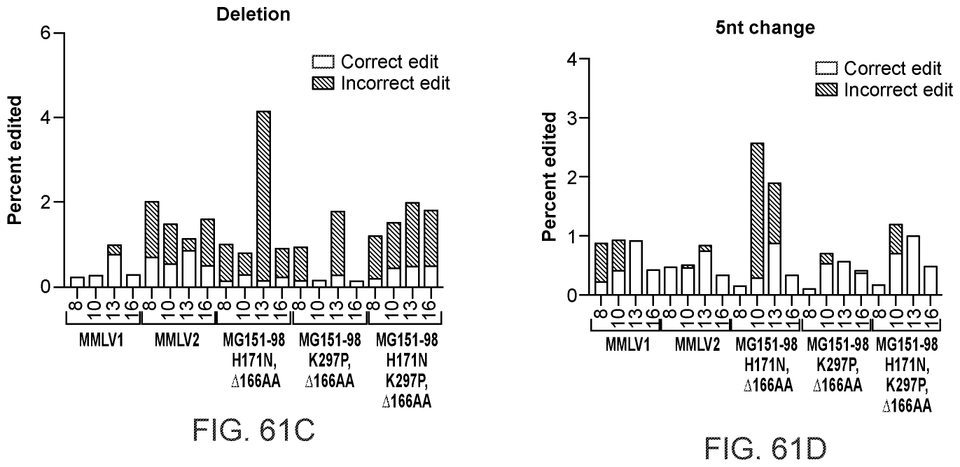

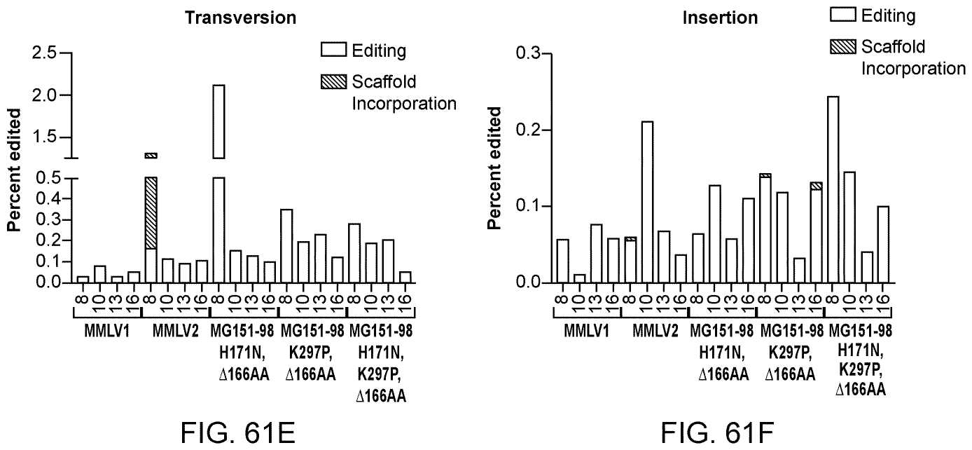

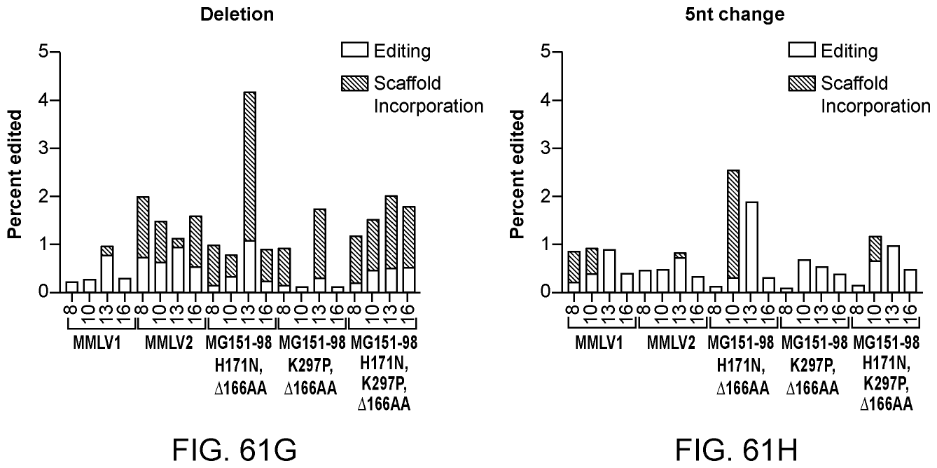

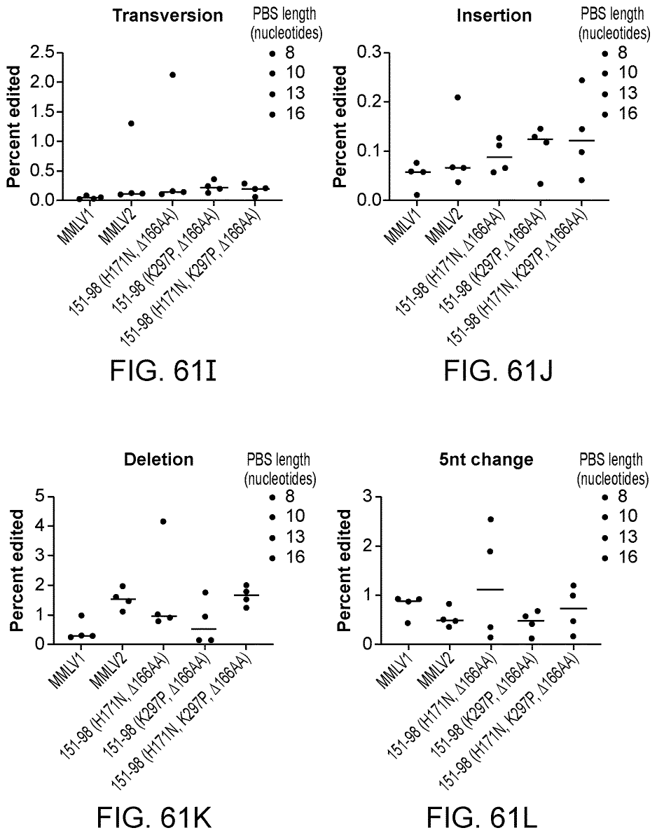

Engineered mutants of MG151-98 (H178N, Δ166AA), MG151-98 (K297P, Δ166AA), and MG151-98 (H178N, K297P, Δ166AA) (SEQ ID NO: 1447) untethered with MG71-2n showed successful editing for G-to-T transversion, a 24 nucleotide insertion, a 15 nucleotide deletion, and a five nucleotide change on the AAVS1 target site using pegRNAs with PBS lengths of 8, 10, 13, and 16 nucleotides (FIGS. 61A-61L). When compared to MMLLV1 and MMLV2 untethered to MG71-2n, the engineered MG151-98 RTs showed similar levels of editing for all corrections (FIGS. 61A-61L). When looking at the average median percent editing for each correction, single point mutant, MG151-98 (H178N, Δ166AA), MG151-98 (K297P, Δ166AA), and double mutant MG151-98 (H178N, K297P, Δ166AA) all had very similar levels of editing (FIGS. 61I-61L). A 15 nucleotide deletion continued to have the largest amount of scaffold incorporation compared to the other corrections (FIG. 61G).

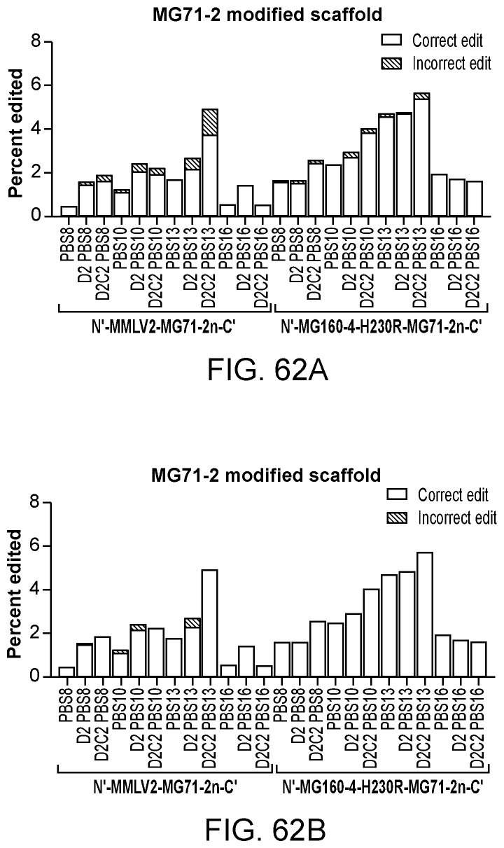

The original guide RNA for MG71-2 contains a 107 nucleotide sequence (SEQ ID NO: 1448) and a 24 nucleotide spacer. Two modified versions of the scaffold were designed: D2 (SEQ ID NO: 1449) and D2C2 (SEQ ID NO: 1450). Modified scaffold D2 removes the last hairpin in the scaffold resulting in a scaffold length of 85 nucleotides. Modified scaffold D2C2 removes the last hairpin of the original scaffold design in addition to a neighboring bulge resulting in a 79 nucleotide modified scaffold. Editing levels for a five nucleotide change were tested using constructs MMLV2 or MG160-4(H230R) tethered to the N-terminus of MG71-2n and modified pegRNAs with PBS lengths 8, 10, 13, and 16 nucleotides (SEQ ID NOs: 1451-1458) (FIG. 62). At PBS lengths 10 and 13 nucleotides, a clear improvement in increased editing levels for both tethered constructs showed higher editing levels with the smaller, modified scaffold (FIG. 62). Further, percent editing analyzed by “correct edit” and “incorrect edit” (FIG. 62A) and analyzed by “editing” and “scaffold incorporation” (FIG. 62B) showed no significant change with modified scaffold designs with respect to the original scaffold.

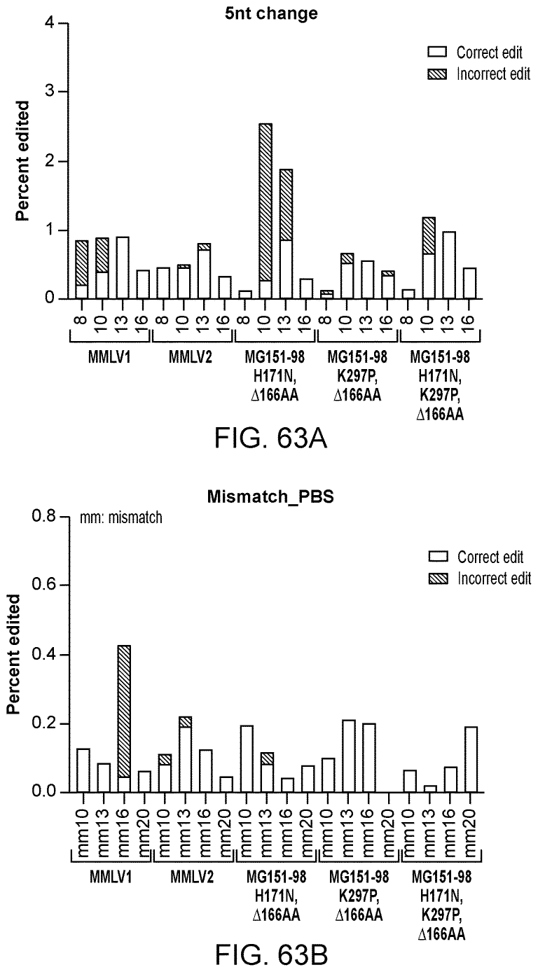

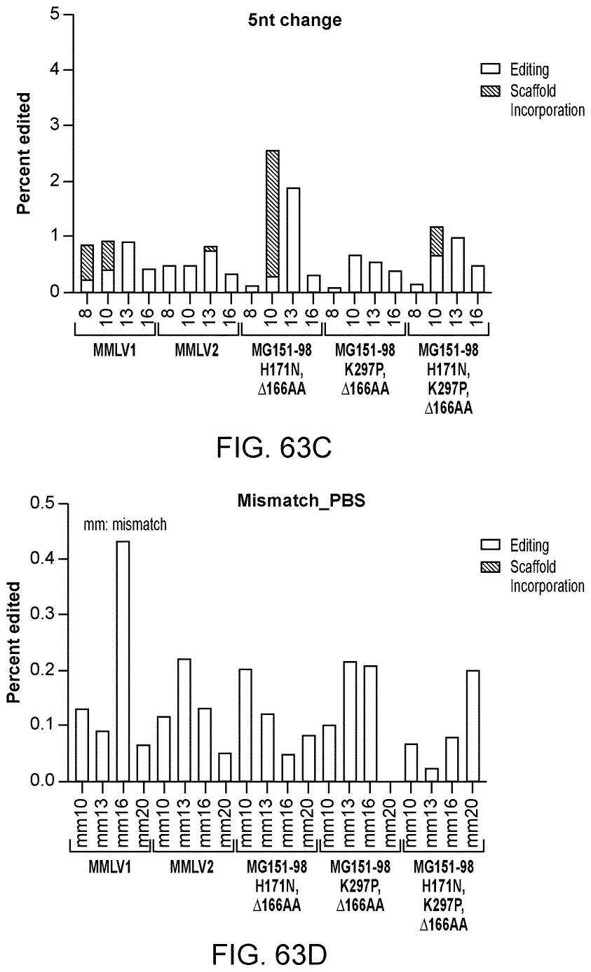

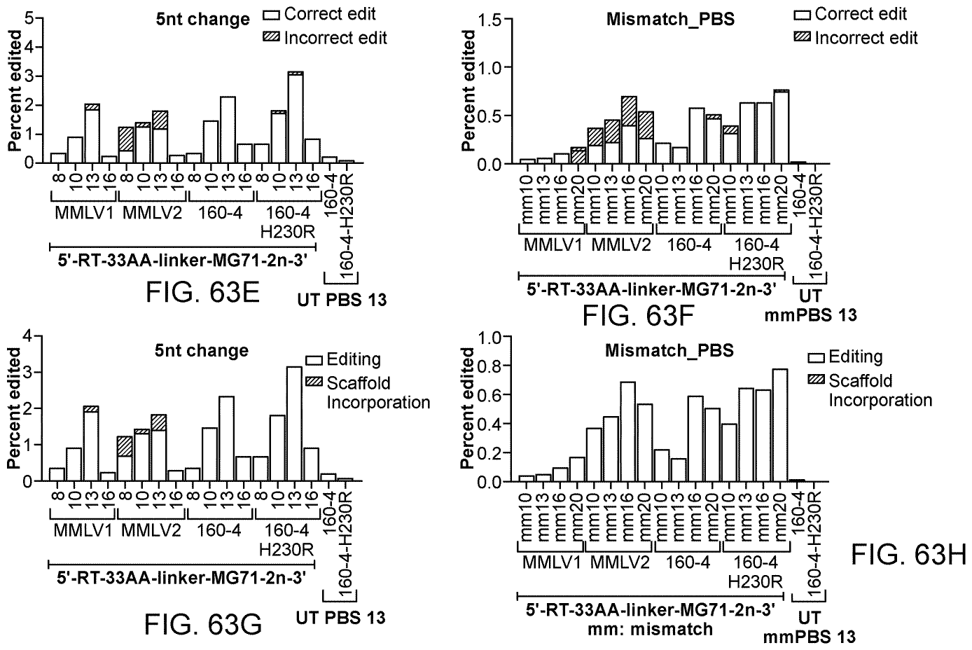

Due to high complementarity between the PBS sequence and the spacer sequence of the pegRNA, incorporation of mismatches in the PBS sequence could help facilitate higher editing levels of an intended edit. Modified mismatched pegRNAs (SEQ ID NOs: 1459-1462) for MG71-2n were designed to have eight nucleotides neighboring 3′ of the RTT having an exact match in nucleotide sequence to the target. After these eight nucleotides, mismatches were incorporated to reach the next PBS length of the pegRNA (PBS 10: 2 mismatches, PBS 13: 5 mismatches, PBS 16: 8 mismatches, and PBS 20: 12 mismatches) (SEQ ID NOs: 1459-1462). MG71-2n and untethered selected RTs (MMLV1, MMLV2, MG151-98 (H178N, Δ166AA), MG151-98 (K297P, Δ166AA), and MG151-98 (H178N, K297P, Δ166AA)) had significantly lower levels of editing when the PBS of the pegRNA contained mismatches (FIGS. 63B and 63D) compared to a PBS sequence with exact complementarity (FIGS. 63A and 63C). This was also true for selected RTs (MMLV1, MMLV2, MG160-4, and MG160-4(H230R)) tethered to the N-terminus of MG71-2n (FIGS. 63E-63H).

Optimizing MG3-6(H586A) Nickase with MG Reverse Transcriptases

To improve editing levels with selected MG RTs and MG3-6(H586A) (SEQ ID NO: 653), the scaffold sequence and the PBS sequence of the pegRNA were modified to have a varying level of GC content in stem loops of the scaffold and mismatches in the PBS sequence. A similar procedure to the above transfection and preparation of NGS samples protocols was used with the exception of different pegRNAs (SEQ ID NOs: 112-113, 116, and 1463-1474) and NGS primers (SEQ ID NOs: 698-699) to target AAVS1 sites (SEQ ID NO: 654) with MG3-6n (SEQ ID NO: 653).

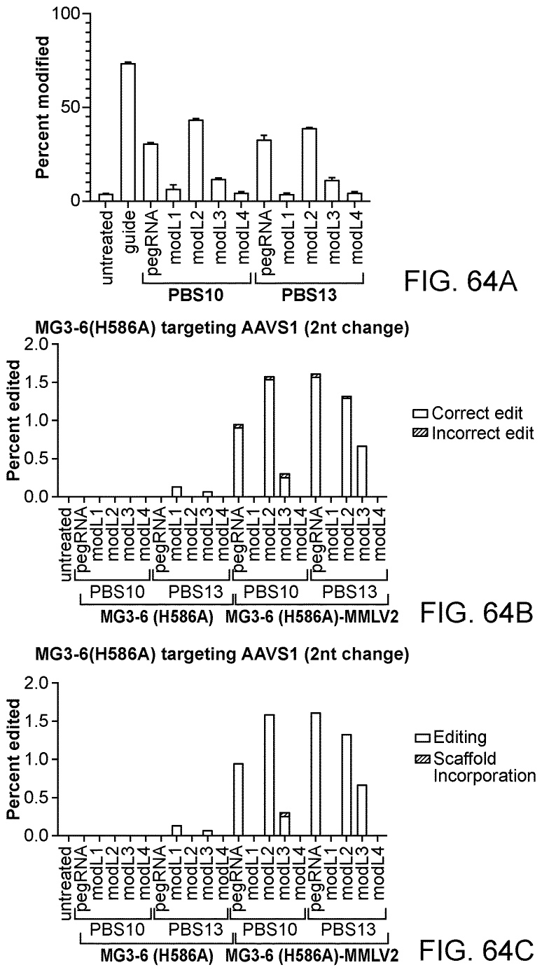

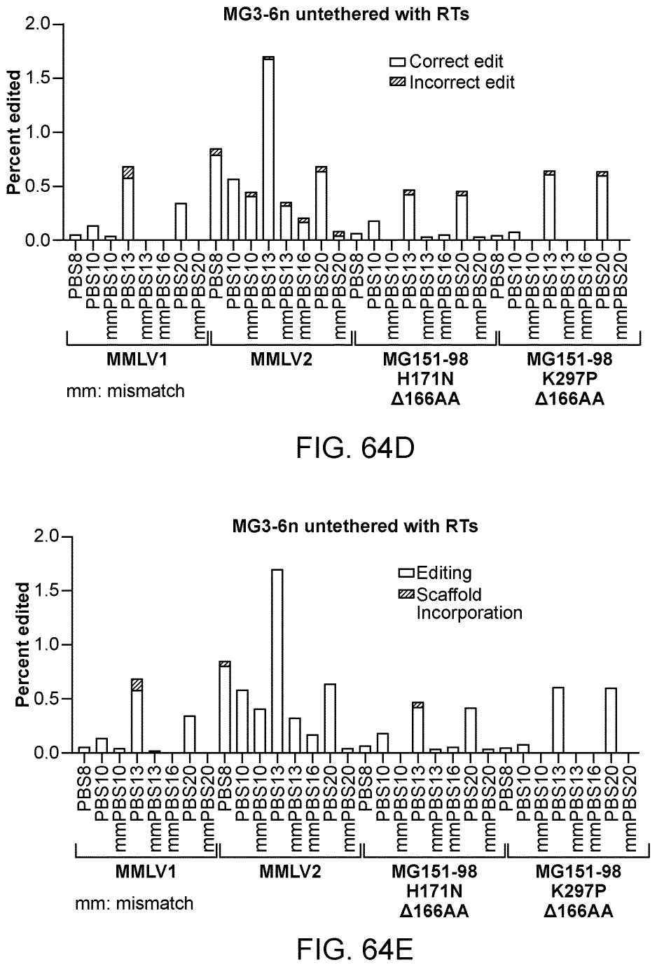

MG3-6 pegRNAs had four versions of modified scaffolds: modL1-4 (SEQ ID NOs: 1463-1470) with modL1-modL3 (SEQ ID NOs: 1463-1465 and 1467-1469) increasing G-C content on the first, second, and third hairpin, respectively, and modL4 combining modifications of all three hairpins (SEQ ID NOs: 1466 and 1470). MG3-6 wild type mRNA (SEQ ID NO: 1475) was used to determine percent modified (including SNPs and InDels) levels of target amplicon AAVS1 (SEQ ID NO: 654) in NGS amplicon. Guide RNA (SEQ ID NO: 116) reached percent modified levels of ˜75%. pegRNAs at PBS 10 (SEQ ID NO: 112) and PBS 13 (SEQ ID NO: 113) with the original MG3-6 scaffold reached about 31% and 35% modified, respectively (FIG. 64A). pegRNAs with modifications, modL1, modL3, and modL4 (SEQ ID NOs: 1463, 1465-1466, 1467, and 1469-1470) sharply dropped percent modified levels while modL2 (SEQ ID NOs: 1464 and 1468) slightly improved or remained constant with the pegRNAs containing the original scaffold design (SEQ ID NOs: 112-113) (FIG. 64A). This also translated to percent editing for a two nucleotide change in AAVS1 target (SEQ ID NO: 654) measured across PBS lengths 10 and 13 nucleotides with the original scaffold and modified scaffolds modL1-modL4 (SEQ ID NOs: 112-113 and 1463-1470) using tethered MMLV2 (SEQ ID NO: 1249) to C-terminus of MG3-6n (SEQ ID NO: 653) with percent editing levels improved for modified scaffold modL2 (SEQ ID NOs: 1464 and 1468) (FIG. 64B-64C). These results indicate the importance of the sequence of the first and third hairpin of the MG3-6 scaffold and suggests that future structural designs should avoid disrupting the first and third hairpin of the MG3-6 scaffold. The pegRNA was then modified to determine if mismatches in the PBS sequence of the pegRNA could improve editing levels. Similar to the results seen with MG71-2n (FIG. 63), MG3-6n and selected untethered RTs (MMLV1, MMLV2, MG151-98 (H178N, Δ166AA), and MG151-98 (K297P, Δ166AA)) showed a large decrease in editing levels when the pegRNA contained mismatches in the PBS sequence (SEQ ID NOs: 1471-1474) (FIGS. 64D and 64E).

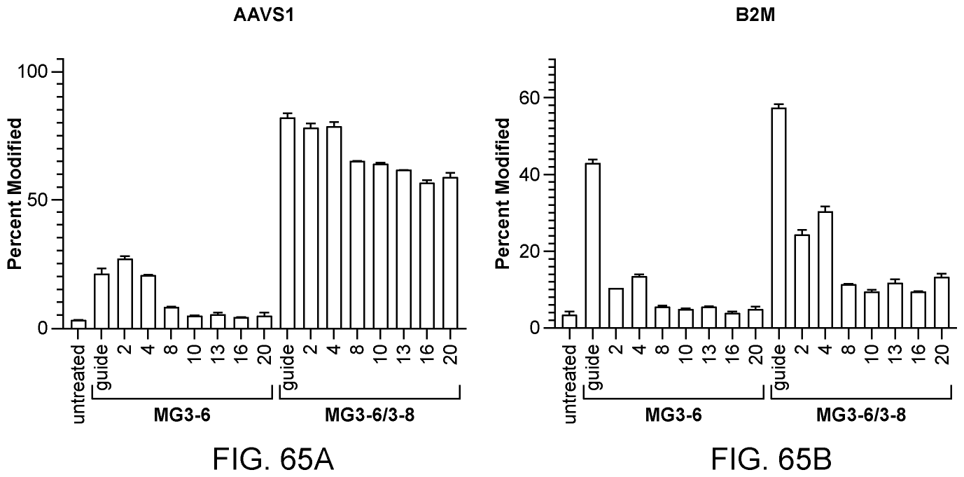

A chimera of MG3-6, MG3-6/3-8 (SEQ ID NO: 1476), was used to discover if percent modified (including SNPs and InDels) levels of target amplicon AAVS1 (SEQ ID NO: 654) (FIG. 65A) and B2M (SEQ ID NOs: 655 and 700-701) (FIG. 65B) could be improved. MG3-6 wild type (SEQ ID NO: 1475) and MG3-6/3-8 mRNA (SEQ ID NO: 1476) was used to direct InDels at target with guide RNA and pegRNA with PBS lengths 2, 4, 6, 8, 10, 13, 16, and 20 nucleotides (SEQ ID NOs: 109-124). For both AAVS1 and B2M targets (SEQ ID NOs: 654-655), MG3-6/3-8 (SEQ ID NO: 1476) shows higher levels of modifications (including InDels) on targets compared to MG3-6 (SEQ ID NO: 1475) (FIG. 65). In general, both MG3-6 and MG3-6/3-8 have decreasing InDel percentage as PBS length gets longer, however, MG3-6/3-8 has higher InDel efficiency at specific targets and recognizes target more efficiently as PBS length increases.

Discovery of MG Nickase in Conjunction with Reverse Transcriptases

MG nuclease MG14-241 (SEQ ID NO: 1477) and MG nickase MG14-241(H596A) (MG14-241n) (SEQ ID NO: 1478) were tested to determine compatibility with selected RTs for prime editing. A similar procedure to the above transfection and preparation of NGS samples protocols was used with the exception of different pegRNAs (SEQ ID NOs: 1479-1492) and NGS primers (SEQ ID NOs: 1493-1504) to target multiple AAVS1 genomic sites (SEQ ID NOs: 1505-1510) with MG14-241 (SEQ ID NOs: 1477-1478).

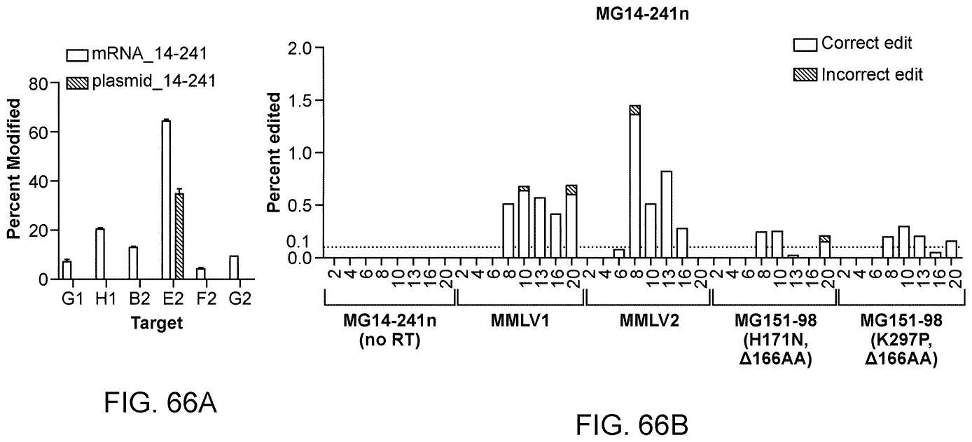

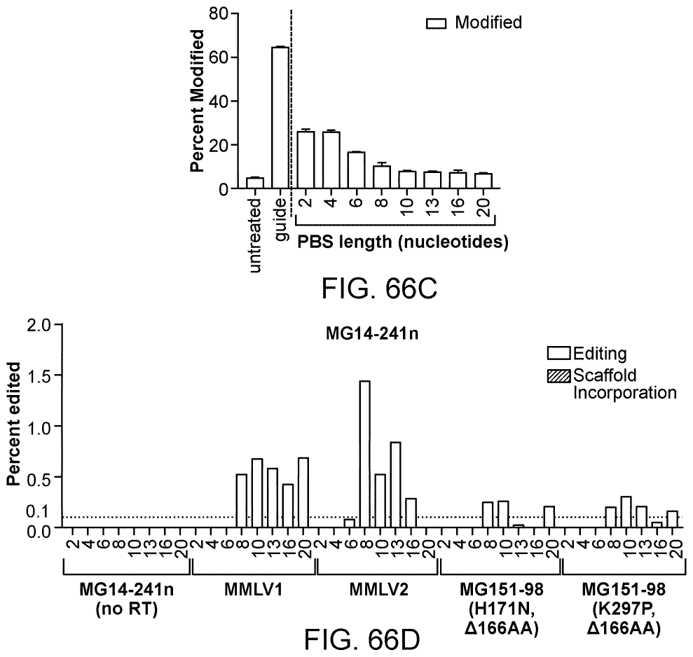

Wild type MG14-241 mRNA or plasmid (SEQ ID NO: 1477) was used to determine percent modified (including SNPs and InDels) levels of various targets (G1, H1, B2, E2, F2, and G2) (SEQ ID NOs: 1505-1510). Varying levels of InDels were seen for each target with target E2 (region of AAVS1) (SEQ ID NO: 1508) resulting in the highest levels of InDels (reaching about 60%) (FIG. 66A). mRNA of MG14-241(SEQ ID NO: 1477) was used to determine percent modified (including SNPs and InDels) levels of target amplicon E2 AAVS1 (SEQ ID NO: 1508) with guide RNA (SEQ ID NO: 1482) and pegRNAs with PBS lengths 2, 4, 6, 8, 10, 13, 16, and 20 nucleotides (SEQ ID NOs: 1485-1492) (FIG. 66B). Similarly to other MG nucleases, as PBS length increased, percent modified decreased when using MG14-241 (SEQ ID NO: 1477). MG14-241n (SEQ ID NO: 1478) with selected untethered RTs (MMLV1, MMLV2, MG151-98 (H178N, Δ166AA), and MG151-98 (K297P, Δ166AA)) was used to determine percent editing of five nucleotide change on AAVS1 target (SEQ ID NO: 1509) across all eight different PBS lengths (SEQ ID NOs: 1485-1492) (FIGS. 66C-66D). Editing levels for a 5 nucleotide change was seen for all selected RTs at a specific PBS length with untethered RTs showing the highest level of editing at PBS 8 and 10 for all selected RTs. Editing levels remained low for all selected RTs, but further optimization of MG14-241n (SEQ ID NO: 1478) and pegRNA could improve editing efficiencies at selected targets.

### Example 22. Site-Specific Integrations of Large Cargo Templates by Non-LTR Retrotransposon RTs and GII Intron RTs

Group II introns and non-LTR retrotransposases are capable of integrating large cargo into a target site via reverse transcription of an RNA template. These reverse transcriptases (RTs) integrate an RNA template via target primed reverse transcription (TPRT), a mechanism in which cDNA synthesis is primed by the free 3′ hydroxyl group at the target DNA nick. These enzymes are predicted to be active based on the presence of expected RT catalytic residues [F/Y]XDD. To evaluate the ability of these RTs to work in conjunction with a nuclease/nickase to generate programmable, site-specific integrations of cargoes of interest as opposed to their endogenous cargoes, several RT-nuclease/nickase fusion constructs were designed. Additionally, various RNA templates were also designed and tested against all RT-Cas fusion constructs to identify a combination that would successfully generate targetable integrations of large cargo.

### Large, Site-Specific Genomic Integrations Templated by RNA in Mammalian Cells

The ability of RTs to reverse transcribe and integrate cDNA from an RNA cargo into a target site specified by a nuclease/nickase was tested by expressing fusion proteins of RTs with SpCas9 WT or SpCas9 Nickase in the presence of an RNA cargo. The target site for genomic integration was specified by the addition of a sgRNA.

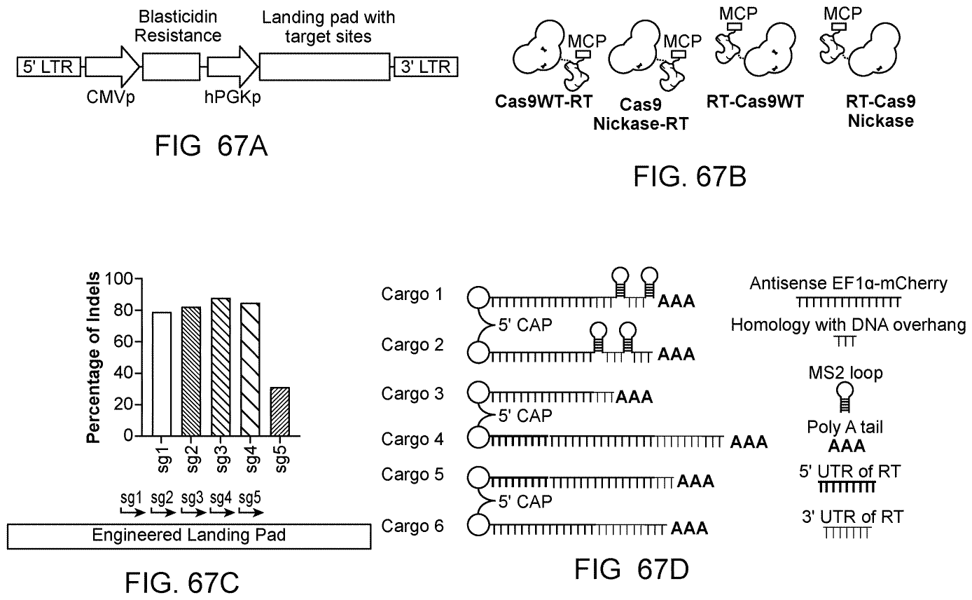

To preclude loss of integration owing to the target site being essential for the viability of the cell line, an engineered landing pad with five spacers for SpCas9 was designed (SEQ ID NO: 1511, FIG. 67A). In addition to the spacers, this landing pad also encoded a blasticidin resistance cassette. A stable cell line was generated in HEK293 cells using a lentiviral vector encoding this engineered landing pad at a low MOI and transduced cells were selected with Blasticidin (8 pg/mL) from 3 days to 10 days post-transduction. A guide screen was conducted using SpCas9 WT mRNA in the engineered cell line to determine the percentage of indels generated by guides targeting each of the five spacers. 500 ng of WT SpCas9 mRNA was transfected into 50,000 engineered cells alongside 10 pmoles of each guide (SEQ ID NOs: 696, and 1512-1516) in a 24-well plate with lipofectamine messenger MAX using the manufacturer's recommendations. Three days post transfection, cells were lysed in 100 μL DNA extraction solution and a PCR (SEQ ID NOs: 1518-1519) was set up to amplify the region flanking the spacers. The amplicon was sequenced and run through an in-house program to determine the percentage of indels generated. Guides 1 through 4 generated 79-88% indels, whereas guide 5 generated 30% indels (FIG. 67B). Owing to their high efficiency of generating indels, suggestive of high cutting ability, guides 1 through 4 were chosen for testing in the integration assay using RT-SpCas fusion proteins.

Reverse transcriptases were cloned at the N-terminus or C-terminus of SpCas WT or Cas Nickase under the CMV promoter, generating a total of 4 constructs for each RT (FIG. 67C, SEQ ID NOs: 1520-1531). A Flag-HA-SV40 NLS tag was added at the N-terminus and another SV40-NLS was added at the C-terminus of the fusion protein to ensure that it localizes to the nucleus upon expression. Additionally, an MS2 coat protein (MCP) tag was added to the RT to facilitate recognition of MS2 tagged RNA template cargoes. MCP is a protein derived from the MS2 bacteriophage that recognizes a 20 nucleotide RNA stem loop with sub-nanomolar affinity. By fusing the RTs with an MCP tag and having the MS2 loops in the RNA template, it is ensured that once the RT is translated it can find the RNA template and start synthesizing cDNA from it, using the DNA overhang generated by SpCas WT/Nickase as the primer. MG140-3 (SEQ ID NO: 163) and MG140-8 (SEQ ID NO: 168) were the non-LTR retrotransposon RTs that were tested. MG153-18 (SEQ ID NO: 463) is a GII intron RT that was tested.

Six different RNA templates were designed for testing each non-LTR retrotransposon RT for integration, (SEQ ID NOs: 1532-1540, FIG. 67D). Two of the templates contain MS2 loops for recognition by the MCP-tagged RT (cargo 1 and cargo 2), while three other template designs contain endogenous UTR elements of the RT that were tested to allow template recognition in the absence of MS2 loops (cargo 4-6). Cargo 1 has an antisense-mCherry open reading frame (ORF), driven by an EF1 alpha promoter, followed by a 10-nucleotide (nt) homology to the DNA overhang (10-nt homology) and 2 MS2 loops. Cargo 2 has an antisense-mCherry ORF, driven by an EF1 alpha promoter, followed by 2 MS2 loops and a 10-nucleotide (nt) homology to the DNA overhang (10-nt homology). Cargo 3 has antisense-mCherry ORF followed by the 10-nt homology without any MS2 loops. Cargo 4 has the antisense-mCherry ORF flanked by 5′ and 3′ UTR sequences of each non-LTR retrotransposon RT (MG140-3 and 140-8) followed by the 10-nt homology. Cargo 5 is essentially the same as cargo 4 but without the 3′ UTR. Similarly, Cargo 6 is cargo 4 without the 5′ UTR. All RNA templates were generated with a 5′ cap and a 3′ poly A tail. The DNA sequence corresponding to each template with an additional T7 promoter was PCR amplified by Flash phusion polymerase according to the manufacturer's instructions. The PCR reaction was cleaned up and 200-500 ng of cleaned PCR product was used per in vitro transcription reaction (IVT). The IVT reaction buffer contains 1×T7 buffer (40 mM Tris HCl, pH 7.5, 16.5 mM MgCl₂, 50 mM NaCl, 2.5 mM Spermidine and 1 mM DTT), 5 mM rATP, 5 mM rUTP, 5 mM rGTP, 4 mM CleanCap-AG, 0.1 unit IPPase (inorganic pyrophosphatase), 40 units RNase inhibitor and 750 units high concentration Hi-T7 RNA polymerase. The IVT reaction was incubated at 50° C. for 1 hr. This was followed by DNase I treatment with 10 units of DNaseI for 10 minutes at 37° C. The reactions were then cleaned up and the purity of RNA templates their quantities were determined.

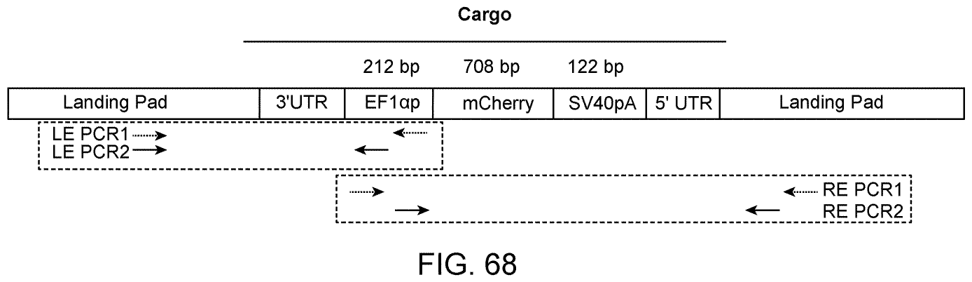

Integration assays were set up in a 6-well format with 1 million engineered cells plated per 6-well in 2 mL media. Each well was transfected with 2500 ng plasmid encoding the RT-SpCas fusion protein, 10 pmoles of chemically synthesized sgRNA, and 2400 ng of cargo pool containing 400 ng of each of 6 RNA cargoes (for non-LTR retrotransposon RTs) or 800 ng of each of 3 RNA cargoes (cargo 1, cargo 2, and cargo 3 for GII intron RTs). Lipofectamine 2000 was used to transfect the plasmid component and Lipofectamine Messenger MAX was used to transfect the RNA component according to the manufacturer's instructions. 24 hours later, cells were split into puromycin containing media (2 pg/mL) to select for cells transfected with the RT-SpCas plasmid, which contains a puromycin resistance cassette. Cells were switched to media without puromycin 3 days post-transfection and split every 2-3 days until 10 days post-transfection. Cells were collected at 4-10 days post transfection and lysed in 100 pL DNA extraction solution. Integration was detected by nested PCR (FIG. 68) at the left end junction (LE) using a forward primer on the landing pad, upstream of the target site, and two reverse primers on the EF1 alpha promoter (SEQ ID NOs: 1541-1543). Likewise, nested PCRs were performed to detect integration at the right end junction (RE) using two forward primers on the EF1 alpha promoter and two reverse primers on the landing pad downstream of the target site (SEQ ID NOs: 1544-1547). LE PCR products were run and LE and RE PCR products were sequenced by Sanger. Sequencing reads were analyzed to determine successful integration of cargo at the target site.

Integration of cargo was detected at the engineered landing pad for MG140-3, MG140-8, and MG153-18 when fused with SpCas WT. Fusion constructs with SpCas Nickases did not yield any detectable integrations. Three of four tested guides (sg1, sg3, and sg4) yielded integrations. Representative data with sg4 is shown.

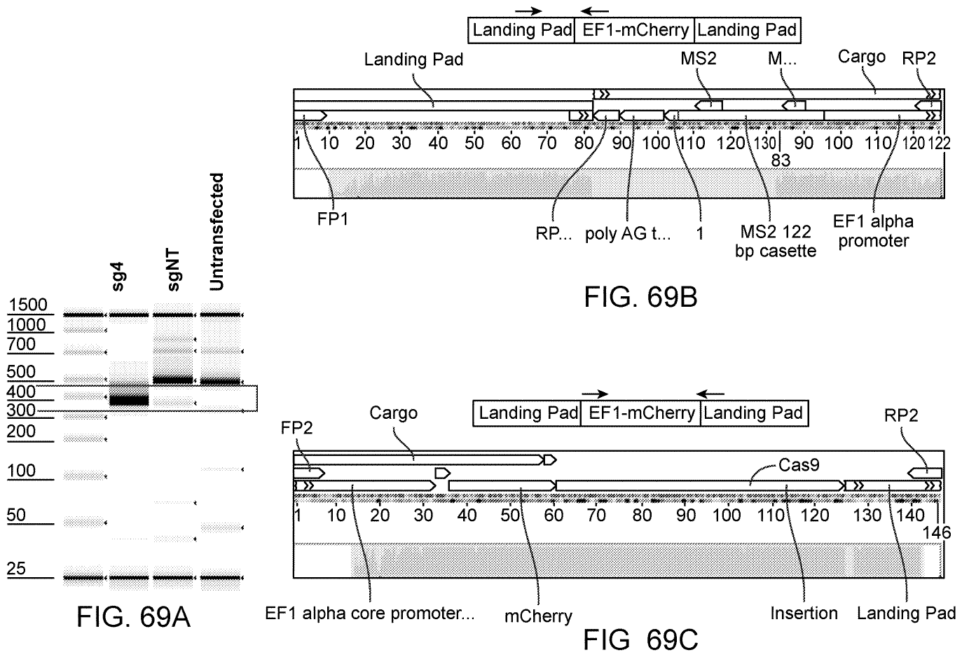

FIG. 69 shows tapestation and sanger sequencing results for the transfection of SpCas WT (N-ter) fused to MG140-3 (C-ter) with sg4 and 6 pooled RNA cargoes at 7 days post-transfection. Tapestation data for LE junction showed a band lower than −400 bp in the guided sample that was absent in the untransfected cells and non-targeting cells (FIG. 69A). This was corroborated by sequencing data that showed LE junction reads going from the landing pad to cargo 2 starting from the MS2 loop closest to the EF1 alpha promoter, with reads going into 96 bp of EF1 alpha promoter, where the reverse primer was located, for a total of 135 bp of detected integration (FIG. 69B). RE junction PCRs were designed so that the FP would be on the part of the EF1 alpha promoter detected in the LE junction and the RP would be on the landing pad. RE PCR junction reads showed 130 bp of cargo sequence going from the EF1 alpha promoter into 74 bp of mCherry sequence, with the last 8 bp being discontinuous, followed by 198 bp of SpCas sequence (FIG. 69C), suggestive of template jumping, before mapping back to the landing pad. Based on the presence of MS2 loops close to the EF1 alpha promoter at the LE junction, it was concluded that of all 6 cargoes, cargo 2 is the one that got integrated.

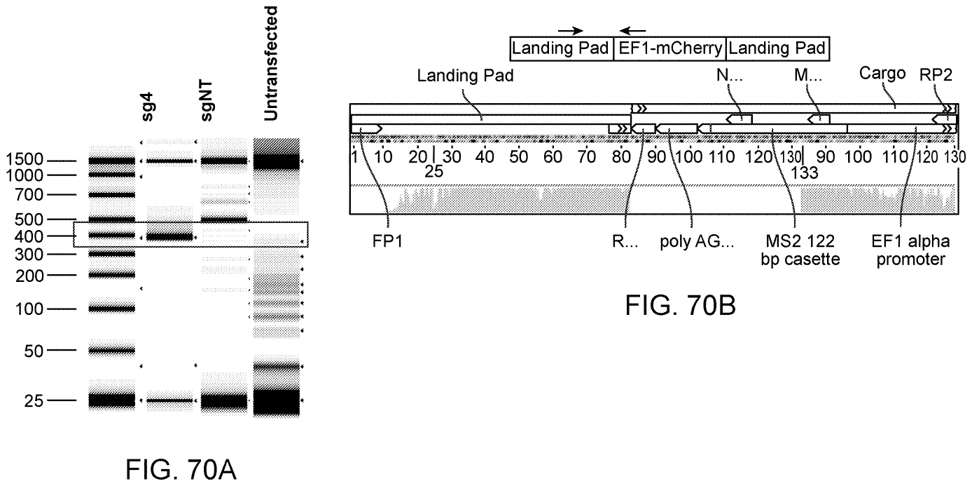

FIG. 70 shows results for the transfection of MG140-3 (N-ter) fused to SpCas WT (C-ter) with sg4 and 6 pooled RNA cargoes at 7 days post-transfection. Data for LE junction showed a band lower than −400 bp in the guided sample that was absent in the untransfected cells and non-targeting cells. This was corroborated by sequencing data that showed LE junction reads going from the landing pad to cargo 2 starting from the MS2 loop closest to the EF1 alpha promoter, with reads going into 96 bp of EF1 alpha promoter, where the reverse primer was located, for a total of 135 bp of detected integration as seen with transfection of SpCas WT (N-ter) fused to MG140-3 (C-ter) with sg4 in FIG. 69. Based on the presence of MS2 loops close to the EF1 alpha promoter at the LE junction, it was concluded that of all 6 cargoes, cargo 2 is the one that got integrated.

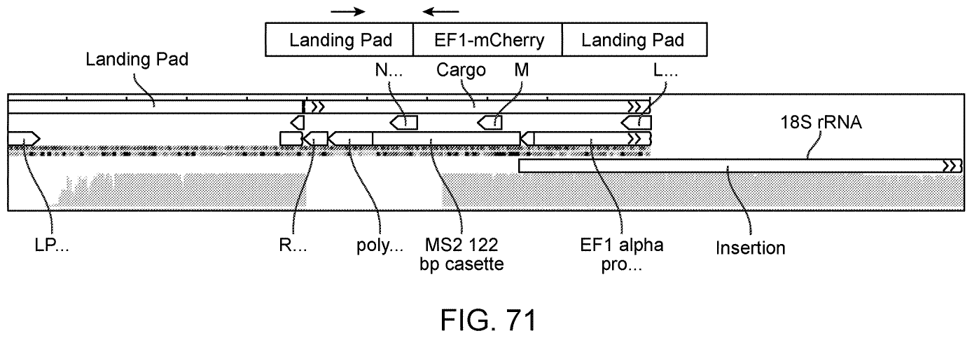

FIG. 71 shows sanger sequencing results for the transfection of SpCas WT (N-ter) fused with MG140-8 (C-ter) with sg4 and 2400 ng of Cargo 1 sequence at 4 days post-transfection. Sequencing data for LE junction shows 73 bp of cargo sequence including the MS2 loop closest to the EF1 alpha promoter and 8 nt of the 10 nt homology. Following this a 368 bp insertion mapping to 18S rRNA was detected. As in FIG. 69, the RT appears to have jumped templates, switching from the cargo template to other abundant RNAs in its vicinity such as the SpCas sequence encoded by the RT-SpCas fusion or ribosomal rRNAs that are known to be highly expressed in cells.

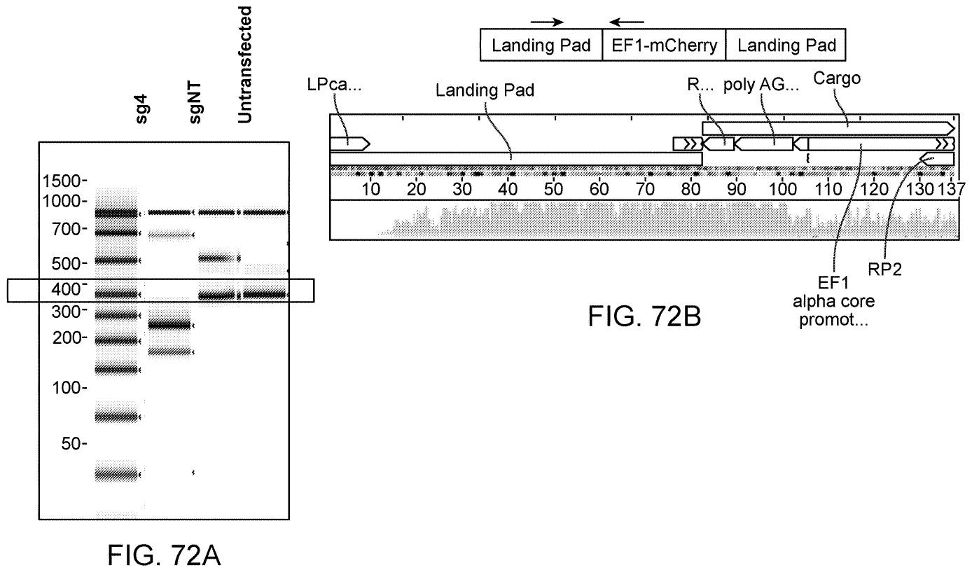

FIG. 72 shows results for the transfection of MG153-18 (N-ter) fused to SpCas WT (C-ter) with sg4 and 3 pooled RNA cargoes (cargo 1, cargo 2, and cargo 3) at 6 days post-transfection. Data for LE junction (FIG. 72A) showed a band lower than −400 bp in the guided sample that was absent in the untransfected cells and non-targeting cells. This was corroborated by sequencing data that showed LE junction reads (FIG. 72B) going from the landing pad to cargo 3 starting from the very end of the template including the RP used for in vitro transcription, the poly A tail, 10 nt-homology and 96 bp of EF1 alpha promoter sequence for a total insertion of 163 bp of cargo sequence. Based on the absence of MS2 loops at the LE junction, it was concluded that of all 3 cargoes, cargo 3 is the one that got integrated.

Altogether, these results demonstrate that MG140-3, MG140-8, and MG153-18 are capable of integrating cargo at a specified target site in conjunction with Cas nucleases.

### Example 23. Highly Processive Retron RTs on Cognate ncRNAs with 2.2 kb Cargo In Vitro

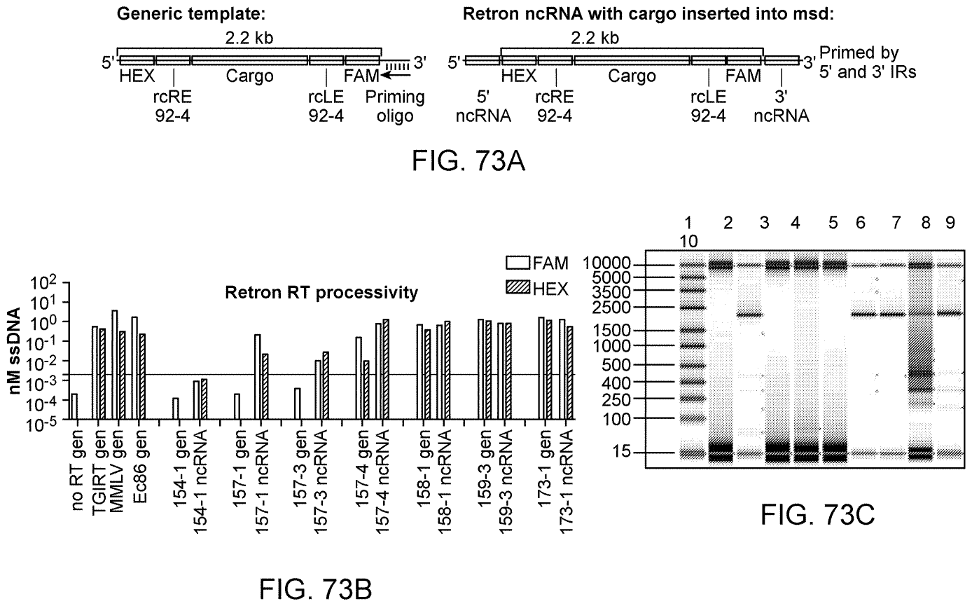

To evaluate the processivity and specificity of retron RTs on long RNA templates (2.2 kb), two substrates were designed and tested for each RT (FIG. 73A). The generic template (SEQ ID NO: 1548) was used to evaluate the extent of non-specific RT activity and was generated by annealing a ssDNA priming oligo to the 3′ end of the RNA template. For this substrate, cDNA synthesis was initiated by the free 3′ hydroxyl group of the priming oligo. The retron ncRNA was primed with the 5′ and 3′ inverted repeats (IRs) facilitated by the presence of terminal 5′ and 3′ retron ncRNA elements specific to each retron system. For this substrate (SEQ ID NOs: 1549-1555), cDNA synthesis was initiated by a 2′ hydroxyl located within the ncRNA msr. For both substrates, the 2.2 kb template consisted of a cargo sequence flanked by the reverse complement of the LE and RE recognition motifs for the ssDNA transposase MG92-4 TnpA. When the RNA sequence is reverse transcribed by the retron RT, the LE and RE motifs will be present flanking the cargo and in the correct orientation for recognition by TnpA. External to the region encoding the LE/RE TnpA motifs, the sequence contains an additional ˜100 nt buffer sequence on each end that, when converted to ssDNA, can be quantified by TaqMan qPCR. Primers and probes designed to detect the beginning 5′ end of the ssDNA (FAM) and 3′ end of the ssDNA (HEX) were used to assess how well the RT can initiate and complete synthesis of the 2.2 kb template. For each retron ncRNA, the 2.2 kb sequence was inserted into a region of the ncRNA msd determined previously to be replaceable.

Each retron RT was co-expressed with either the annealed generic template or refolded cognate ncRNA loaded with cargo in a cell-free expression system supplemented with dNTPs (NEB, 0.3 mM final). In the co-expression reaction, RNA templates were used at a final concentration of 75 nM. After incubation for 2 h at 37° C., the reaction was quenched via the addition of RNase A. Samples were then diluted prior to TaqMan qPCR to ensure ssDNA concentrations were within the linear range of detection. The amount of beginning and end of the 2.2 kb ssDNA was quantified by extrapolating values from a standard curve generated with a DNA template of known concentrations.

As expected (FIG. 73B), TGIRT (Control GII intron) is highly processive on the 2.2 kb generic template, indicated by FAM and HEX detected in near equimolar ratio. In contrast, for MMLV (Control retroviral RT), which is expected to be highly active but not processive, the FAM signal was notably higher than HEX. Interestingly, the positive control retron RT Ec86 does have appreciable non-specific activity on the generic template but is not processive. MG154-1 (SEQ ID NO: 1549) does not have appreciable non-specific activity and using its own cognate ncRNA did not dramatically improve its activity nor processivity. MG157-3 does not have detectable activity on the generic template, but is active and processive on their cognate ncRNAs (SEQ ID NO: 1550). MG157-1 similarly does not have detectable activity on the generic template, does have activity on its cognate ncRNA (SEQ ID NO: 1551), but is not processive. MG157-4 is active but not processive on the generic template but is more active and more processive on its cognate ncRNA (SEQ ID NO: 1552). MG158-1, MG159-3, and MG173-1 are active and processive on both the generic template and on their cognate ncRNAs (SEQ ID NOs: 1553-1555).

To further confirm that a full-length 2.2 kb ssDNA molecule was synthesized by the retron RT from its cognate ncRNA, co-expression reactions were diluted 1:50 and amplified with the most external TaqMan qPCR primers, specifically the forward primer for the HEX probe and the reverse primer for the FAM probe. Products were evaluated by Tapestation D5000 (Agilent). Product presence was not confirmed for MG157-3, likely due to low ssDNA quantities produced by the retron RT (FIG. 73C). However, a 2.2 kb product was confirmed for MG157-4, MG158-1, MG159-3, and MG173-1. Of note, in addition to the 2.2 kb product, other smaller products also appear to be generated by MG159-3. Altogether, these results demonstrate that MG157-4 is a highly active, processive, and specific retron RT, while MG157-3 are processive and specific retron RTs, but less active in vitro than MG157-4.

### Example 24. Highly Processive Retron RTs on Cognate ncRNAs with 2.2 kb Cargo in Mammalian Cells

The ability of retron RTs to produce cDNA in a mammalian environment was tested by expressing them in mammalian cells and detecting cDNA synthesis by qPCR. To evaluate the processivity and specificity of retron RTs on long RNA templates (2.2-4 kb), three substrates were tested for each RT. Generic 4 kb and 2 kb templates (SEQ ID NOs: 648 and 1548) were used to evaluate the extent of non-specific RT activity and were generated by annealing a ssDNA priming oligo to the 3′ end of the RNA template. The MG173-1 retron ncRNA was primed with the 5′ and 3′ inverted repeats (IRs) facilitated by the presence of terminal 5′ and 3′ retron ncRNA elements specific to MG173-1 (SEQ ID NO: 1555). For this substrate, cDNA synthesis was initiated by a 2′ hydroxyl located within the ncRNA msr.

To prepare the cDNA template, the DNA sequence corresponding to each RNA template was prepared with a T7 promoter appended to the sequence and then PCR amplified. The PCR reaction was cleaned up and 200-500 ng of cleaned PCR product was used per in vitro transcription reaction (IVT). The IVT reaction and RNA purification was performed as described above. The purity of RNA templates and their quantities were determined. Generic 4 kb and 2 kb templates were hybridized to a complementary DNA primer (SEQ ID NO: 1557) in 10 mM Tris pH 7.5, 50 mM NaCl at 95° C. for 2 min and cooled to 4° C. at the rate of 0.1° C./s. MG173-1 specific ncRNA was taken through the hybridization reaction with water in place of the complementary DNA primer.

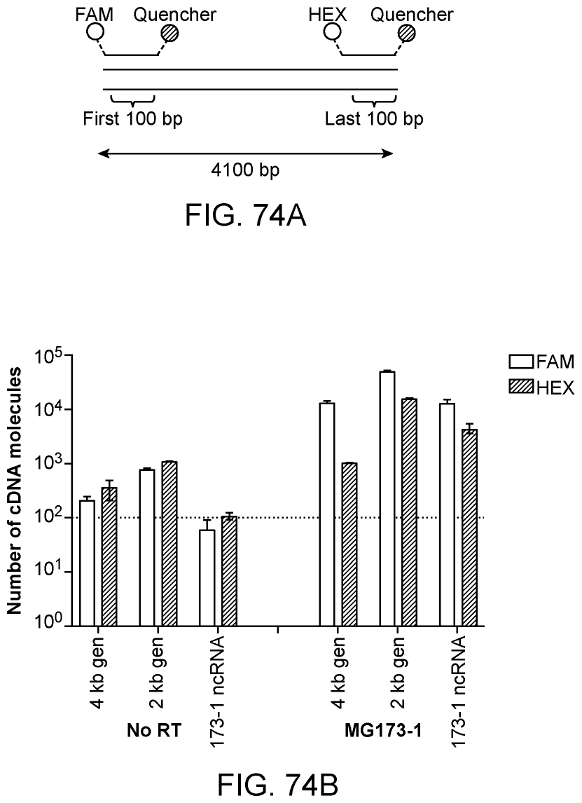

A plasmid containing MG173-1 under the CMV promoter was cloned and isolated for transfection in HEK293T cells. Plasmid transfection was performed using lipofectamine 2000 using the manufacturer's instructions. The generic RNA/DNA hybrid or mock hybridized ncRNA was transfected into HEK293T cells 6 hours after the plasmid containing the RT was transfected. 18 hours post RNA/DNA transfection, cells were lysed. 100 μL of quick extract was added per well in a 24 well plate. Primers to amplify first and last 100 bp products from the newly synthesized cDNA were designed (SEQ ID NOs: 1557-1560), along with probes (SEQ ID NOs: 1561-1562) to quantify their amplification (FIG. 74A).

Activity of MG173-1 was detected on all 3 RNA templates, as evidenced by higher FAM and HEX signals for each of the templates in presence of MG173-1 as opposed to the No RT condition (FIG. 74B). However, the FAM signal for MG173-1 on the ncRNA was about 200 times greater than its FAM signal in the No RT condition, whereas with both generic templates it is only about 64 times greater than in the No RT condition. In terms of processivity, MG173-1 is most processive on its ncRNA followed by the 2 kb generic template, and it is not processive on the 4 kb generic template. Altogether, MG173-1 is most active and processive on its cognate ncRNA as opposed to the two tested generic templates in mammalian cells. The high activity, specificity, and processivity in vitro and in mammalian cells of the retrons discovered and characterized herein demonstrate the feasibility of their use as genome editing tools.

### Example 25. TnpA Integration of ssDNA Produced by a Retron RT In Vitro

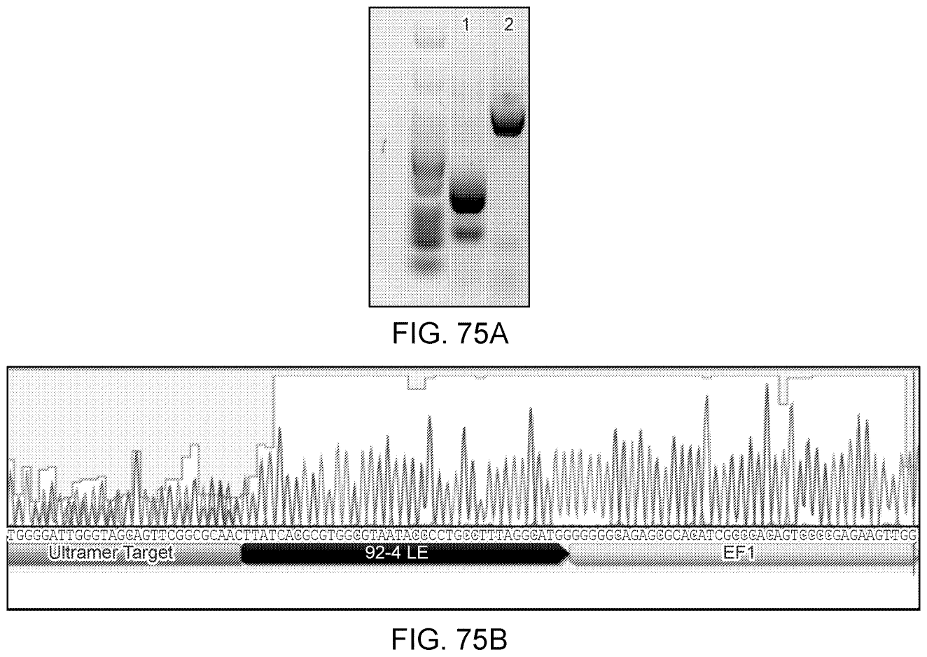

For in vitro transposition activity using a retron-produced ssDNA, TnpA candidate MG92-4 was first expressed in an in vitro transcription-translation (IVTT) kit following manufacturer's recommended conditions at 37° C. for 2 hours with a template concentration of 65.7 ng/μL. Transposition assays were set up with 1 μL of IVTT expressing MG92-4 protein, 1 μL of a retron-produced ssDNA cargo, and 50 nM of a ssDNA ultramer “target” in reaction buffer (20 mM HEPES (pH 7.5), 160 mM NaCl, 5 mM MgCl₂, 5 mM TCEP, 20 μg/mL BSA, 0.5 μg/mL of poly-dIdC, and 20% glycerol) per 10 μL reaction. The ssDNA cargo was obtained from an IVTT reaction of the retron and ncRNA that was RNAseA treated as described in Example 23. Control reactions contained a no-template control (NTC) reaction of IVTT where Tris buffer was added instead of PCR template to the IVTT. Reactions were incubated at 37° C. for 1 hour to allow transposition to occur, then the reaction was diluted 10-fold in water and transposition was detected via PCR. The LE junction was detected via a forward primer on the 5′ end of the target and reverse primer within the EF1a promoter of the retron-produced cargo. PCR products were run on an agarose gel to detect transposition (FIG. 75A), and sequenced via Sanger. Chimeric reads that contained both target and cargo sequence were analyzed to determine the junction of transposition at the known insertion motif of TnpA 92-4 (FIG. 75B). Taken together, these data indicate that single strand transposases can recognize ssDNA produced by a retron, making this process a suitable path for genome editing.

### Example 26. Identifying and Optimizing a Complete MG System (Nickase and RT) for Prime Editing on Therapeutically Relevant Targets

### Testing MG71-2 Nuclease Activity and Prime Editing on Therapeutically Relevant Targets

MG71-2 wildtype mRNA (SEQ ID NO: 1563) was transfected alongside chemically synthesized guide RNAs (SEQ ID NOs: 1564-1576) targeting therapeutically relevant sites (SEQ ID NOs: 1577-1591). 500 ng of mRNA and 120 pmoles of gRNAs were transfected into 50,000 cells. For prime editing experiments, selected RT candidates in the tethered system were cloned into a plasmid containing the nickase MG71-2(H883A)(MG71-2n) to generate an RT-nickase fusion (SEQ ID NOs: 1592-1597). The CMV promoter drove the expression of the fusion protein, which contained a 33 amino acid linker (SEQ ID NO: 103) between the nickase and the RT candidate. Plasmid was transfected. All components (plasmids and therapeutically relevant pegRNAs (SEQ ID NOs: 1598-1609)) were reverse transfected into 50,000 HEK293T cells in a 24 well plate. 72 hour post transfection, cells were lysed in 100 μL of DNA extraction solution. Primers containing barcodes for next generation sequencing (NGS) were used to amplify a ˜250 bp target for each therapeutically relevant site. PCR cleanup was then performed and samples were sequenced. The percentage of reads with desired change was determined.

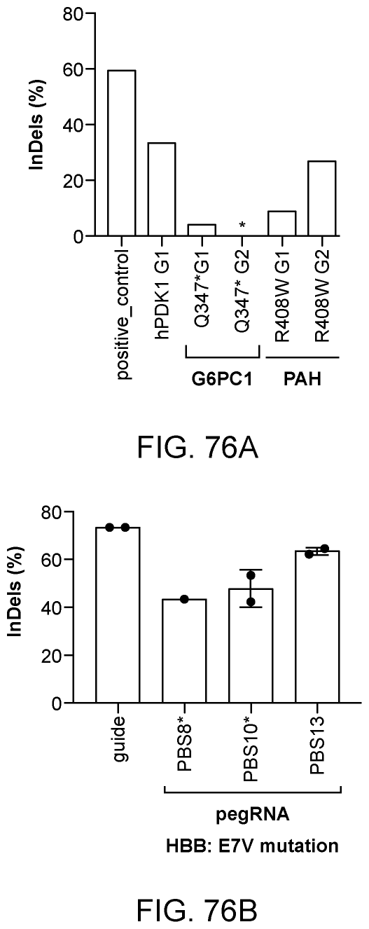

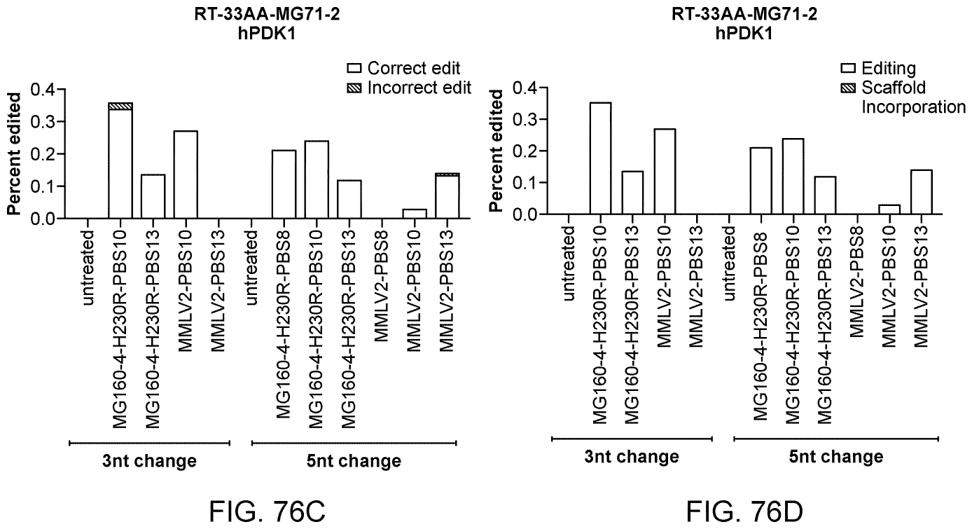

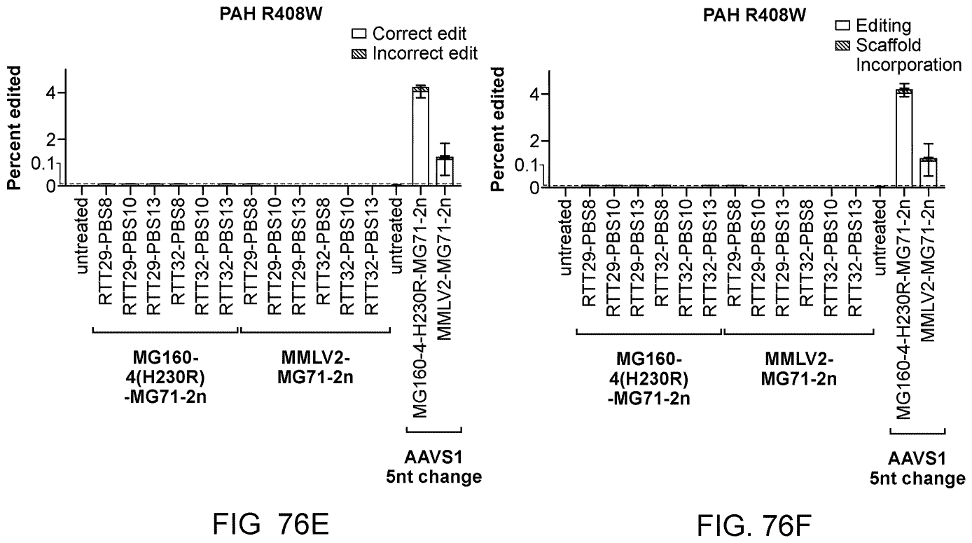

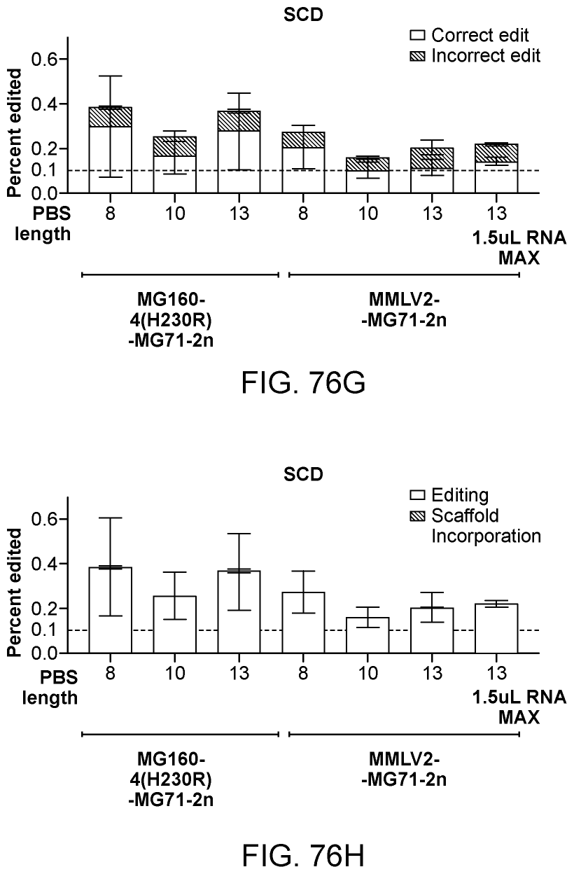

Nuclease activity of MG71-2 was tested on various guide RNAs targeting therapeutically relevant sites hPDK1, G6PC1, PAH, and HBB (FIG. 76). MG71-2 showed about 30% InDels at hPDK1 and about 25% InDels on PAH gene targeting R408W with guide 2 (FIG. 76A). Little to no nuclease activity was seen at G6PC1 targeting Q347* for two different guide RNAs. When targeting the PAH R408W therapeutically relevant site, guide 2 had about a ˜4 fold increase in InDel levels compared to guide 1. When targeting HBB E7V mutation, nuclease activity of MG71-2 had InDel levels reaching about 75% (FIG. 76B). Further, nuclease activity was tested for pegRNAs containing PBS lengths of 8, 10, and 13 nucleotides. PBS length did not have a significant decrease in InDel levels when compared to the guide RNA (FIG. 76B). When performing prime editing experiments targeting the microRNA recognition site in hPDK1, RT templates included either a 3nt or 5nt change to disrupt the micRNA recognition site. Above background levels of editing were seen for prime editing constructs MG160-4(H230R)-MG71-2n and MMLV2-MG71-2n for pegRNAs with a PBS length of 10 nt and an RTT containing a 3nt change (FIGS. 76C and 76D). Prime editing was slightly lower with pegRNAs having an RTT containing a 5 nt change. When targeting PAH R408W with MG160-4(H230R)-MG71-2n or MMLV2-MG71-2n with various pegRNAs having PBS length 8, 10, 13nt and an RTT length of 29 or 32nt, no prime editing was detected (FIGS. 76E and 76F). Moving towards targeting the HBB gene, both MG160-4(H230R)-MG71-2n and MMLV2-MG71-2n had above background levels of editing (FIGS. 76G and 76H). For all therapeutically relevant sites recognized by MG71-2, prime editing levels can be improved by optimization of pegRNAs through adjusting RTT sequence, RTT length, and PBS length, along with improving transfection efficiency and discovering compatible nicking guides.

### MG71-2n and Selected RTs for Larger Genomic Insertions

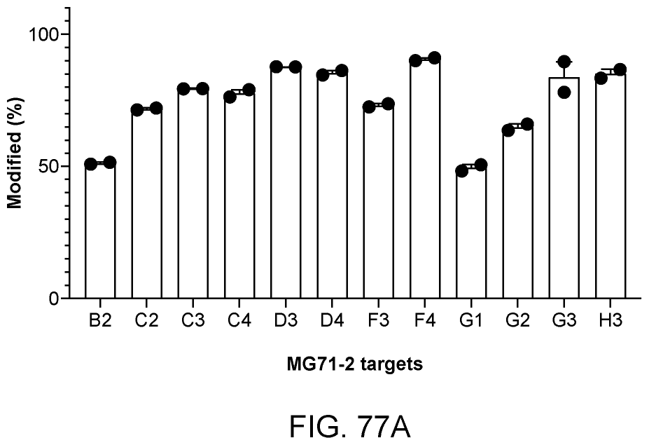

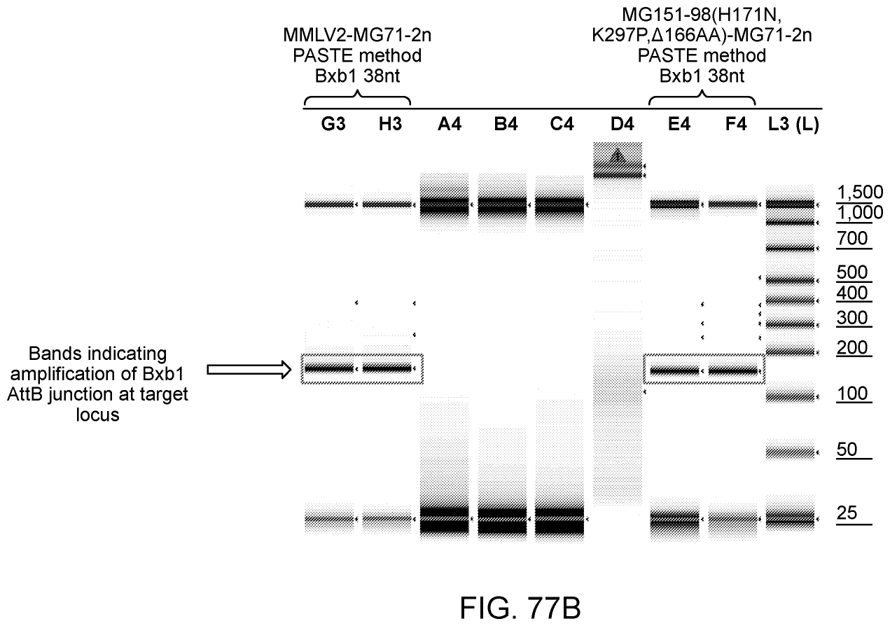

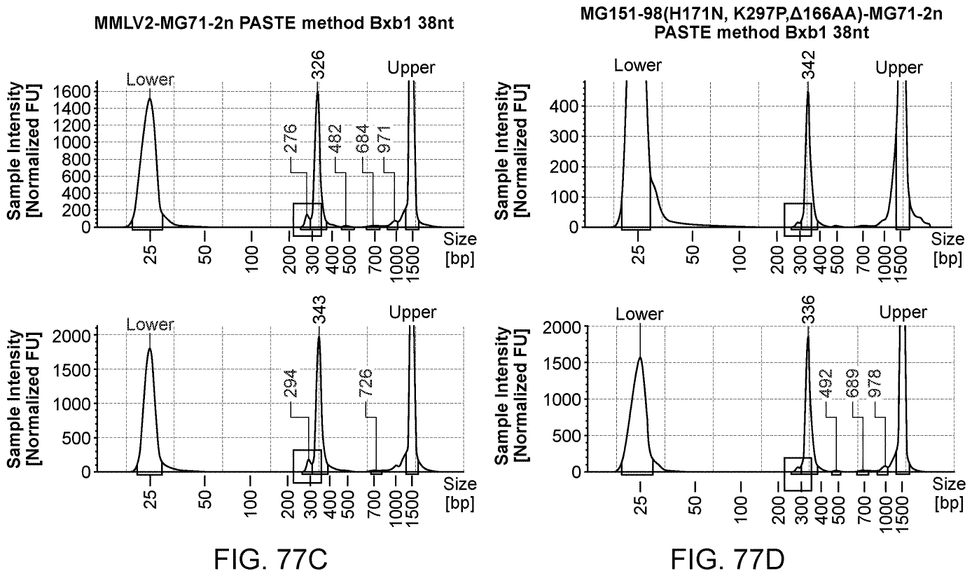

To determine insertion location of Bxb1 AttB on the AAVS1 gene, guides (SEQ ID NOs: 1610-1653) were designed to test for InDels at specific sites in the gene using wild type mRNA of MG71-2 (FIG. 77A). Two specific sites 69nt apart (guide D3 and guide D4) were used to design pegRNAs compatible for the PE2, PE3, twin-PE, and TJ-PE systems (SEQ ID NOs: 1656-1681). Correct ratios of chemically synthesized pegRNA and nicking guide RNA were transfected as described above using selected nickase-RT fusion constructs in plasmid. PCR reaction was performed with a forward primer specific to the Bxb1 38nt AttB sequence and a reverse primer downstream of the insertion site (SEQ ID NO: 1682). Amplification using these primers indicate the insertion of the AttB sequence that can be visualized on either an agarose gel electrophoresis or tape station.

MG151-98(H171N, K297P, Δ166AA)-MG71-2n (SEQ ID NOs: 1447 and 1654) was tested for the ability to incorporate a 38nt Bxb1 AttB sequence at a specific AAVS1 locus using various methods. The Bxb1 junction PCR for MG151-98(H171N, K297P, Δ166AA)-MG71-2n and MMLV2-MG71-2n was run on a tape station and showed a band indicating insertion of the Bxb1 sequence (FIG. 77B). The size difference between the two amplicons (the wild type amplicon and the Bxb1 incorporated amplicon) was analyzed on a tape station to show the relative abundance of the two amplicons (FIGS. 77C and 77D).

### Optimization of MG71-2n Prime Editing Systems Through Inlaid and Linker Designs

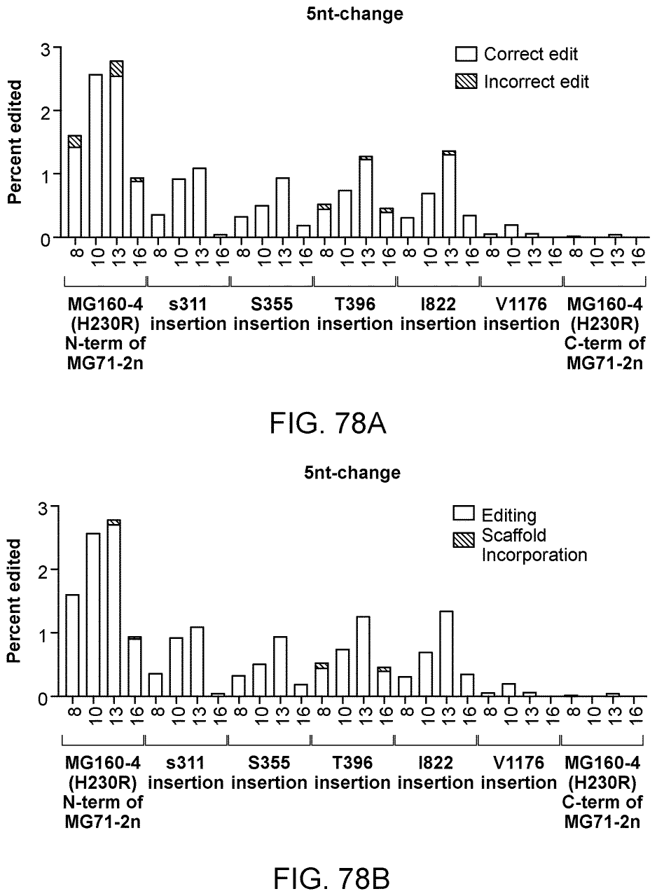

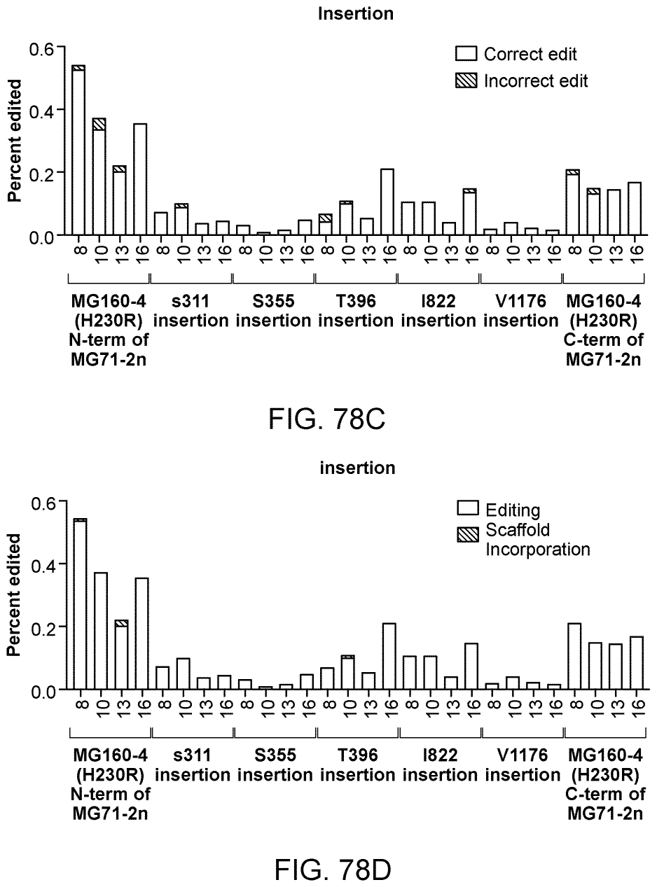

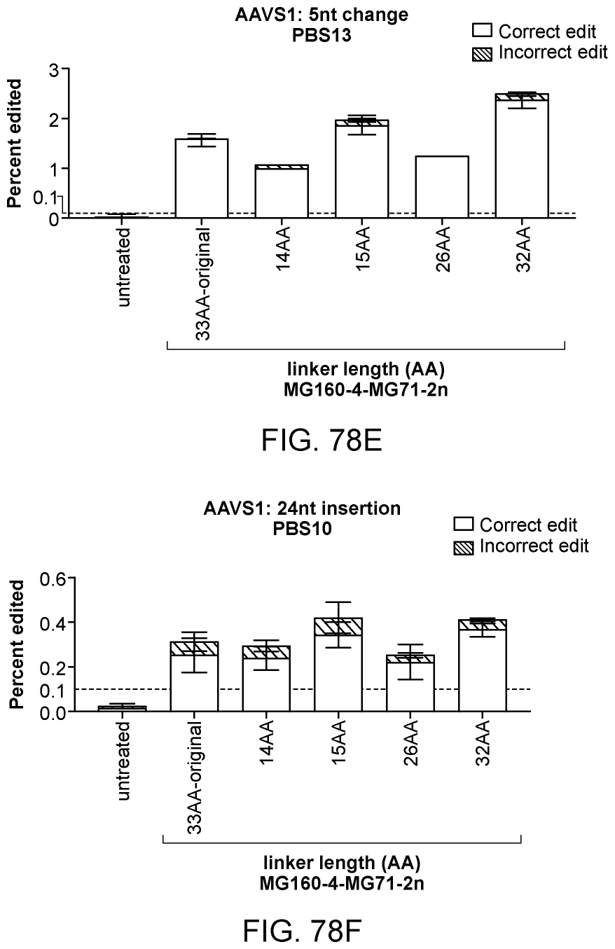

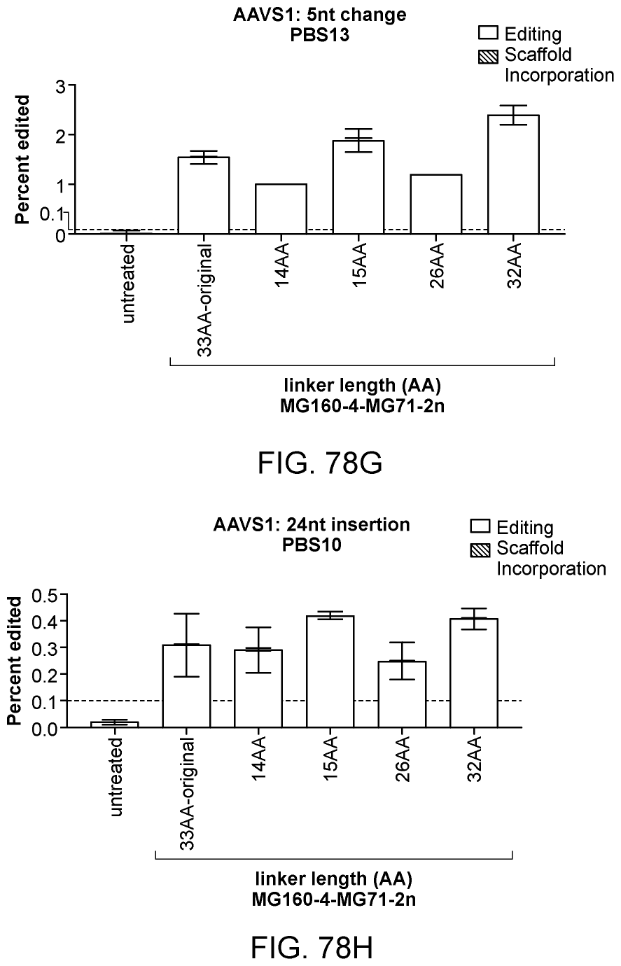

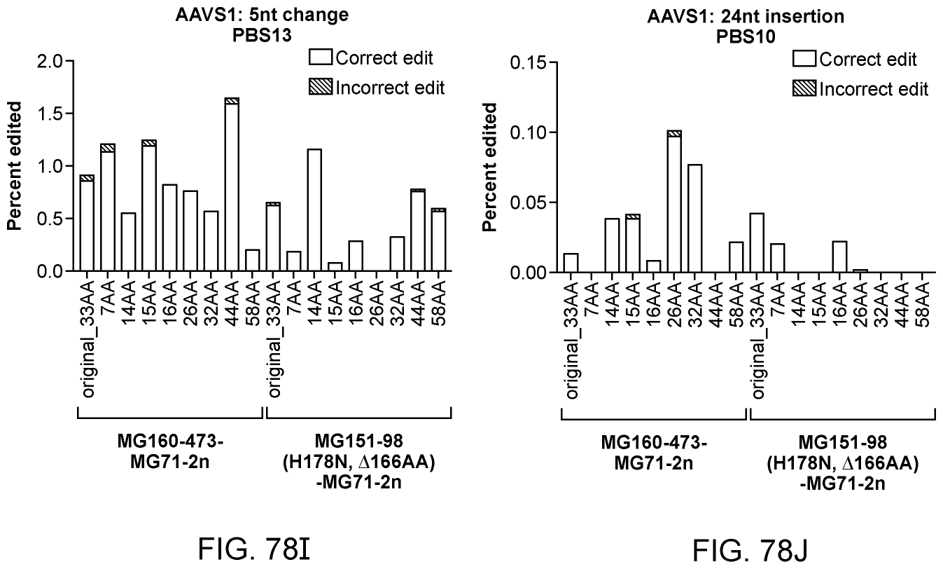

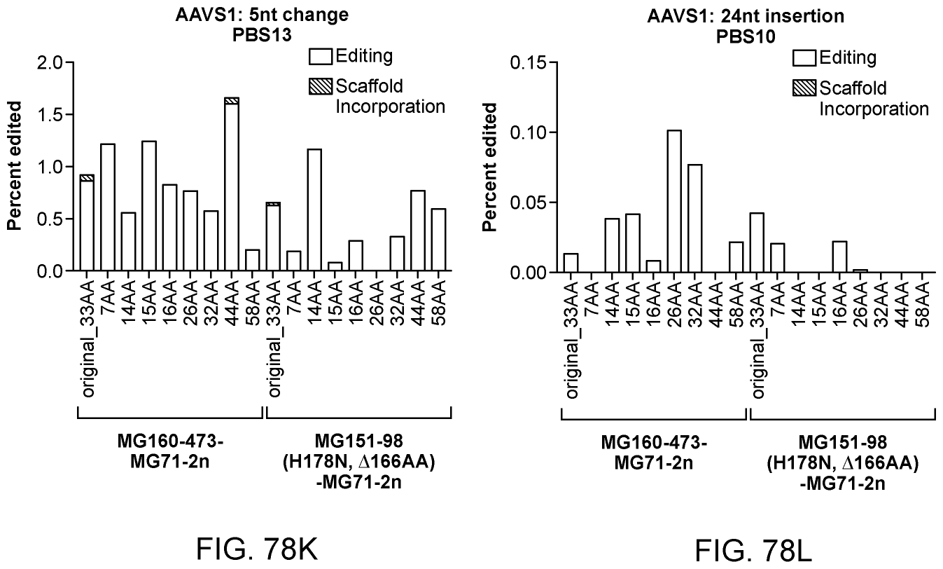

Optimization of prime editing systems with MG71-2n and selected RT, MG160-4(H230R), were rationally designed to generate five different inlaid constructs (SEQ ID NOs: 1691-1695). MG160-4(H230R) was inserted at position S311, S355, T396, 1822, and V1176 in MG71-2n. MG160-4(H230R) had a 5′ and 3′ 33 amino acid linker at the point of insertion. The inlaid fusion constructs coding region were cloned into an expression vector driven by the CMV promoter. Tethered constructs with MG160-4(H230R) on the N and C terminus of MG71-2n were also tested alongside the inlaid constructs (SEQ ID NO: 1696). Tethered constructs of MG160-4 wildtype (SEQ ID NOs: 1697-1698) on the N-term of MG71-2n were tested with the 33AA linker along with a 14AA, 15AA, 26AA, and 32AA linker (SEQ ID NOs: 1699-1702). MG160-473 and MG151-98(H171N, Δ166AA) were tested across linker lengths ranging from 7AA to 58AA (SEQ ID NOs: 1703-1720). Systems were transfected as described above with chemically synthesized pegRNAs encoding the intended edit. Inlaid designs of MG160-4(H230R) with MG71-2n were tested for prime editing of a 5nt change and a 24nt insertion with PBS lengths of 8, 10, 13, and 16nt targeting the AAVS1 locus. Out of the five inlaid designs, V1176 resulted in the poorest activity for both a 5nt change and 24nt insertion. For the other four inlaid sites, S311, S355, T396, and 1822, similar editing levels amongst the constructs was seen and the highest level of editing was reached with pegRNA having a PBS length of 13nt for a 5nt change (FIGS. 78A-78D). However, even with some activity seen for four out of the five inlaid constructs, the highest levels of editing was achieved with MG160-4(H230R) on the N-terminus of MG71-2n for both a 5nt change and a 24nt insertion. Having MG160-4(H230R) on the C-terminus of MG71-2n resulted in little to no detectable editing. These results are potentially due to the structural limitations of MG71-2n and the protein-protein interactions between MG71-2n and MG160-4(H230R). With clear preference for having the RT on the N-terminus of MG71-2n, selected RTs (MG160-4, MG160-473, and MG151-98(H171N, Δ166AA)) were tested with the original 33AA linker along with varying linker lengths and amino acid composition (FIGS. 78E-78L). Five linker lengths (14AA, 15AA, 26AA, 32AA, and the original 33AA) were tested with MG160-4 tethered to the N-terminus of MG71-2n and challenged to a 5nt change and 24nt insertion on the AAVS1 target. The 15AA, 32AA, and 33AA linker performed similarly when correcting a 5nt change with potentially the highest level of editing seen for the 32AA linker (FIGS. 78E and 78G). When these tethered constructs were incorporating a 24nt insertion, linker length seemed to have less of an effect on the editing levels (FIGS. 78F and 78H). Editing levels for a 5nt change and 24nt insertion were much lower for RTs MG160-473 and MG151-98(H171N, Δ166AA) compared to MG160-4 (FIGS. 78I-78L). When targeting a 5nt change, a pegRNA of PBS 13nt was used and depending on the RT, linker lengths resulted in different levels of prime editing. For MG160-473, the highest levels of editing for a 5nt change were seen with a 44AA linker and with MG151-98(H171N, Δ166AA) the highest levels of editing seen for a 5nt change was with a 14AA linker.

### Testing MG3-6-3-8 Nuclease Activity and MG3-6-3-8n and MG3-6n Prime Editing on Therapeutically Relevant Targets

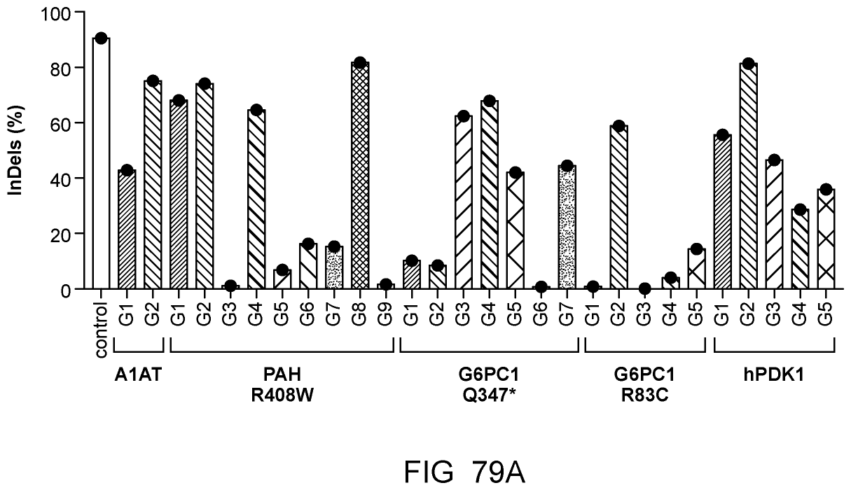

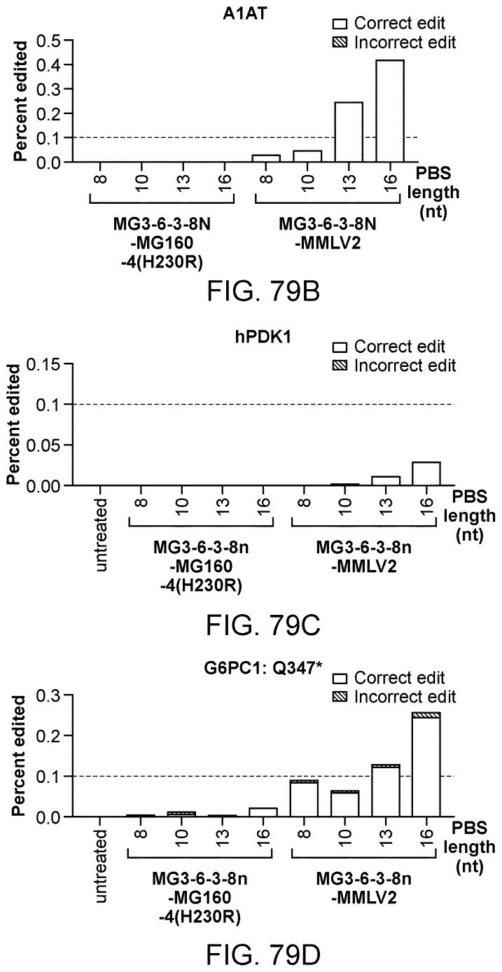

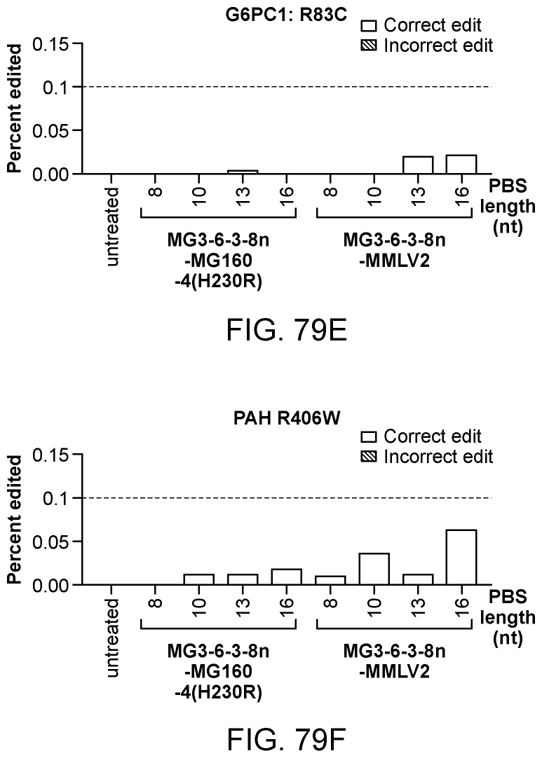

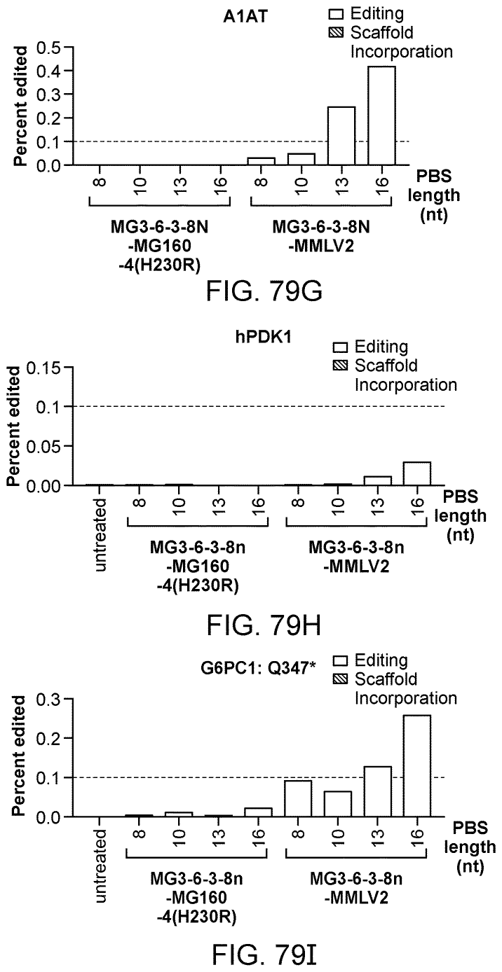

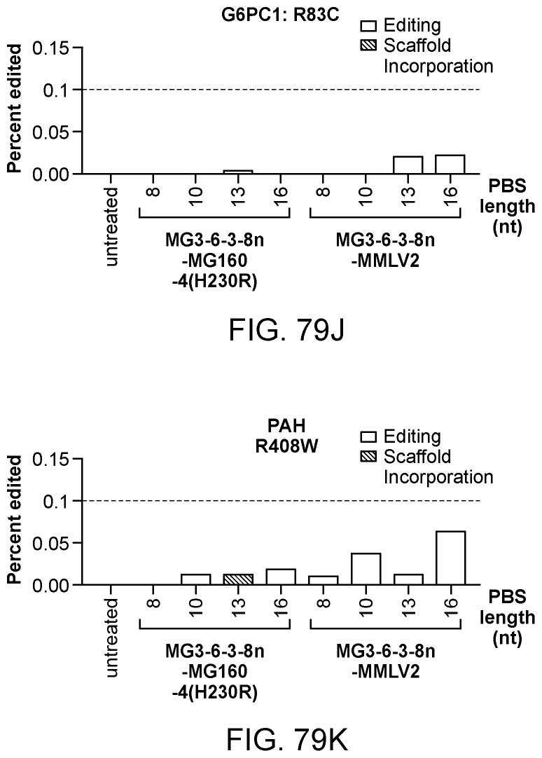

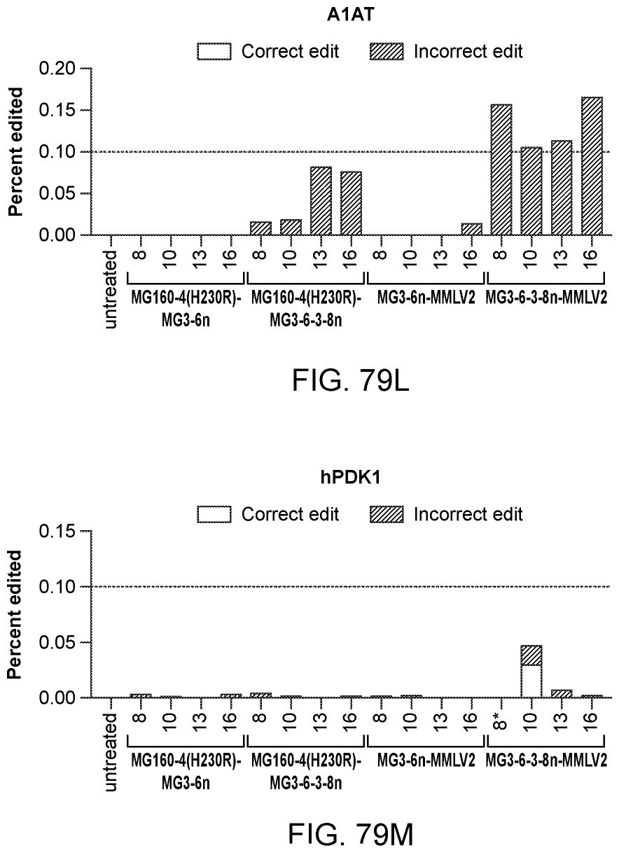

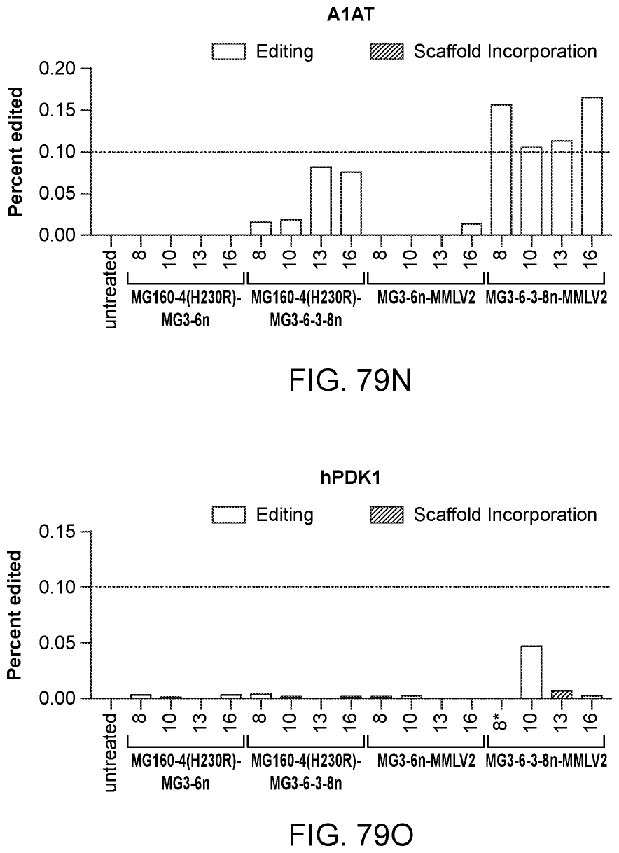

MG3-6-3-8 wildtype mRNA (SEQ ID NO: 1476) was transfected alongside chemically synthesized guide RNAs targeting therapeutically relevant sites (SEQ ID NOs: 1722-1752). RNA was transfected as described above. For prime editing experiments, selected RT candidates in the tethered system were cloned into a plasmid containing the nickase MG3-6-3-8(H586A)(MG3-6-3-8n) (SEQ ID NOs: 1753-1754) or MG3-6(H586A)(MG3-6n) (SEQ ID NOs: 653, and 1776-1778) to generate an RT-nickase fusion. The CMV promoter drove the expression of the fusion protein, which contained a 33 amino acid linker between the nickase and the RT candidate. pegRNAs (SEQ ID NOs: 1755-1774) along with nickase-RT constructs were transfected and samples were analyzed as described above. MG3-6-3-8 targeted five different therapeutically relevant sites with each therapeutically relevant site having various guide RNAs (gRNA) to determine which gRNA resulted in the highest levels of InDels at the target site (FIG. 79A). The guide resulting in the highest levels of InDels is shown in dark gray (A1AT: guide 2 (G2), PAH R408W guide 8 (G8), G6PC1 Q347* guide 4 (G4), G6PC1 R83C guide 2 (G2), and hPDK1 guide 2 (G2)); this spacer sequence was used for designing pegRNAs (FIG. 79A). Prime editing experiments were performed on each therapeutic target using MG3-6-3-8n constructs with RT (MG160-4(H230R) or MMLV2) tethered to the C-terminus of the nickase (FIGS. 79B-79K). Low to no detectable editing was seen for all targets using MG3-6-3-8n-MG160-4(H230R) (FIGS. 79B-79K). With construct MG3-6-3-8n-MMLV2, no detectable editing was seen for targets PAH R408W (FIGS. 79F and 79K), G6PC1: R83C (FIGS. 79E and 79J) and hPDK1 (FIGS. 79C and 79H). With MG3-6-3-8n-MMLV2, editing levels were slightly above background at target A1AT (FIGS. 79B and 79G), and G6PC1: Q347* (FIGS. 79D and 79I). Since editing levels were below background for MG160-4(H230R) tethered to the C-terminus of MG3-6-3-8n, constructs of MG160-4(H230R) tethered to the N-terminus of MG3-6-3-8n (MG160-4(H230R)-MG3-6-3-8n) or MG3-6n (MG3-6n-MG160-4(H230R) were generated and compared to MG3-6n-MMLV2 and MG3-6-3-8n-MMLV2 (MMLV2 on the C-terminus of MG3-6n or MG3-6-3-8n) targeting A1AT and hPDK1 (FIGS. 79L-79O).

### Optimization of MG3-6n Prime Editing Systems Through Linker and Inlaid Designs

Tethered constructs of MG160-4 wildtype on the N-term of MG3-6n were tested with the 33AA linker along with a 32AA, 44AA, and 58AA linker (SEQ ID NOs: 1780-1783). Optimization of prime editing systems with MG3-6n and selected RT, MG160-4(H230R), was rationally designed to generate five different inlaid constructs. MG160-4(H230R) was inserted at position K115, V208, K368, D550, and L881 in MG3-6n (SEQ ID NOs: 1790-1795). MG160-4(H230R) had a 5′ and 3′ 33 amino acid linker at the point of insertion. The inlaid fusion constructs coding region were cloned into an expression vector driven by the CMV promoter. Tethered constructs with MG160-4(H230R) on the N and C terminus of MG3-6n were also tested alongside the inlaid constructs. A stable cell line, in HEK293 cells, was generated using a lentiviral vector encoding hygromycin (hygro) and blue fluorescent protein (BFP) with a linker in between hygromycin and BFP containing two stop codons (SEQ ID NOs: 1784-1789). Stable cell line (Hygro-STOP-BFP) was generated at a low MOI and transduced cells were selected with hygromycin (120 pg/mL) from 7 days to 10 days post-transduction. Systems were transfected as described above with chemically synthesized pegRNAs encoding the intended edit fixing the two stop codons in the linker between hygromycin and BFP.

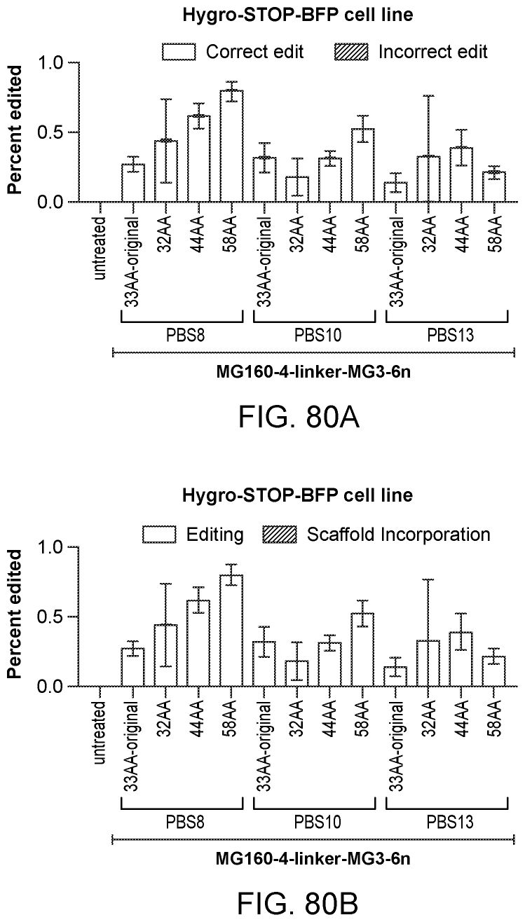

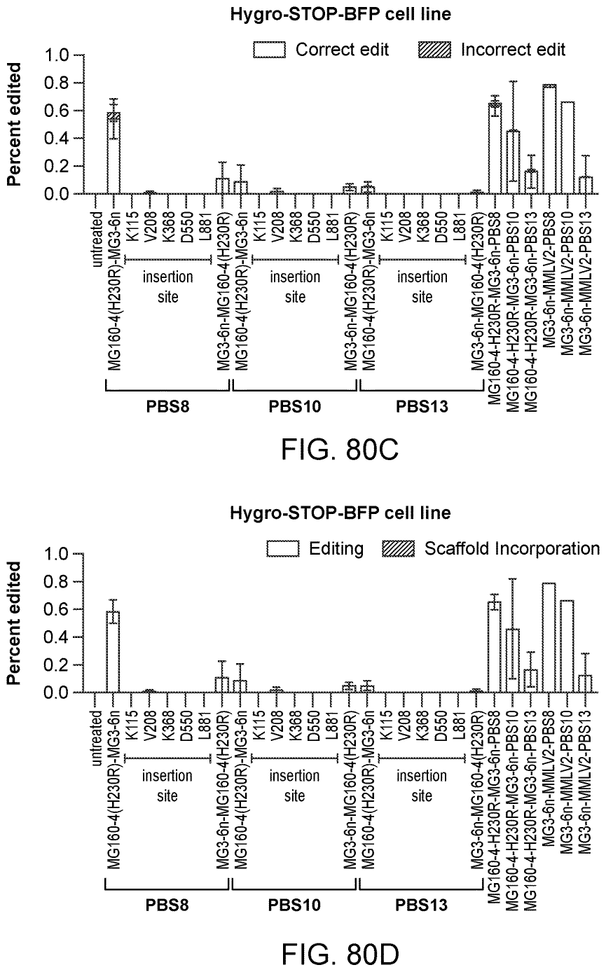

Wild type MG160-4 tethered to the N-terminus of MG3-6n with four different linker compositions targeted an engineered site using pegRNAs with PBS lengths of 8, 10, and 13 nucleotides and an RTT encoding the correction of two stop codons (FIGS. 80A and 80B). At PBS 8 nt, a clear trend showed that as linker length increased, prime editing levels improved (FIGS. 80A and 80B). However, at PBS length of 10 and 13 nt, editing levels did not have a clear trend between linkers. When testing the inlaid designs of MG3-6n and MG160-4(H230R), editing levels dropped to below background when targeting the engineered cell line (FIGS. 80C and 80D). The best fusion construct was MG160-4(H230R) on the N-terminus of MG3-6n and showed the highest level of editing with pegRNA PBS 8nt giving approximately 0.6% editing.

### Example 27. Short Corrections, Small Insertions and Deletions with Natural and Engineered RTs

### Testing Natural Reverse Transcriptase Candidates Tethered to MG71-2(H883A) Nickase

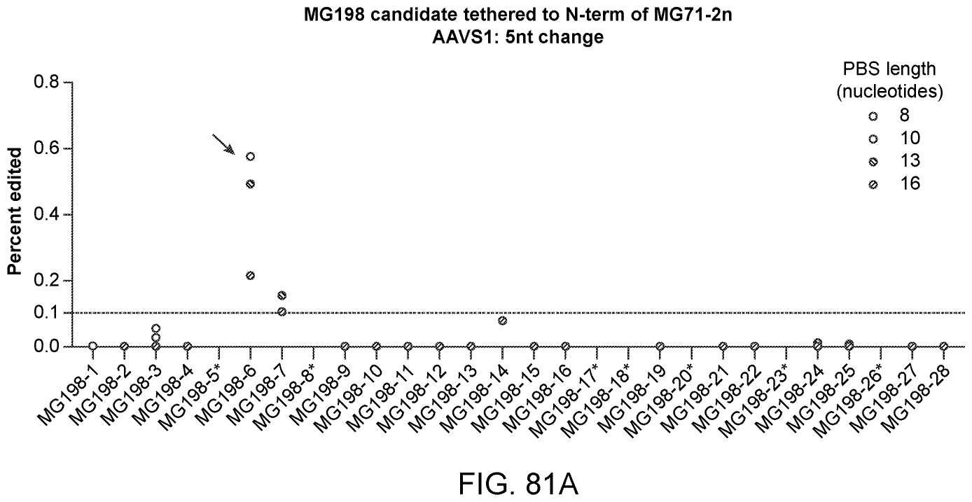

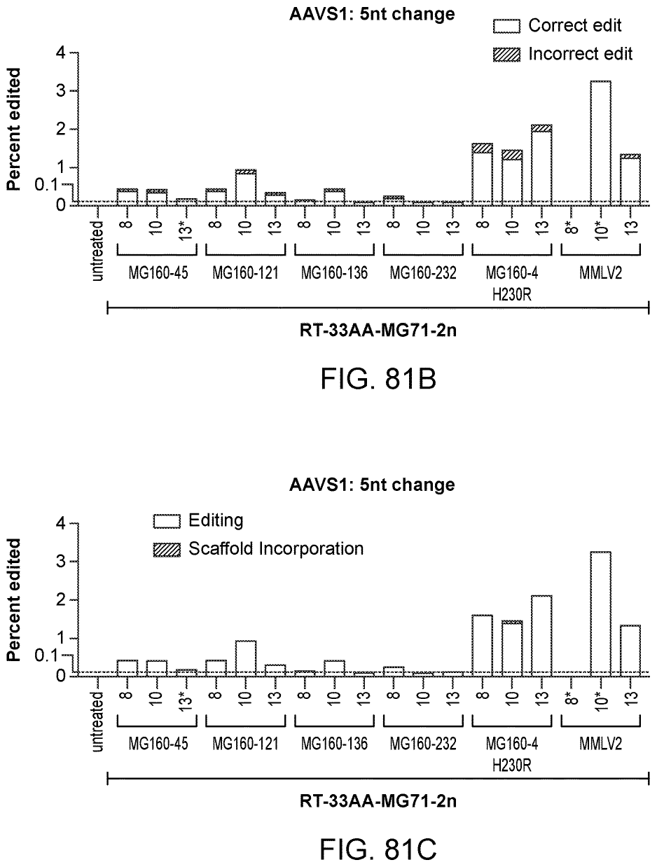

Reverse transcriptase candidates from the MG198 family (SEQ ID NOs: 1796-1823) and MG160 family (SEQ ID NOs:1405, 1407, 1414, and 1423) were tethered to the N-terminus of MG71-2n and challenged to a five nucleotide change on an AAVS1 target site (FIG. 81). A similar procedure to the above transfection and preparation of NGS samples was used.

Twenty eight candidates from the MG198 family were tested tethered to the N-terminus of MG71-2n. These tethered systems were challenged to a 5nt change and tested across PBS lengths of 8, 10, 13, and 16 nucleotides (FIG. 81A). MG198 candidates MG198-6 had editing levels above background with the best condition reaching approximately 0.6% editing at PBS 8nt. Slightly above background editing was also seen for MG198-7 with the highest level reaching almost 0.15%. All remaining MG198 candidates had no detectable editing for a 5nt change on AAVS1 target.

Four MG160 candidates, MG160-45, MG160-121, MG160-136, and MG160-232, were tested tethered to the N-terminus of MG71-2n and challenged to a five nucleotide change on AAVS1 target (FIG. 81B). Editing levels above background were seen for three out of the four candidates (MG160-232 showed no activity), with MG160-121-MG71-2n showing the highest levels of editing at PBS 10 nt reaching almost 1% editing. These candidates were directly compared to MG160-4(H230R)-MG71-2n and MMLV2-MG71-2n; selected MG160 candidates showed significantly less activity than MG160-4(H230R)-MG71-2n and MMLV2-MG71-2n.

### Testing Engineered Reverse Transcriptase Candidates Tethered to MG71-2(H883A) Nickase

Ancestral candidates were designed using selected MG160 candidates from the MG160 family. Thirteen MG160 ASRs (SEQ ID NOs: 1828-1846) were tethered to MG71-2n and tested for a 5nt change on the AAVS1 target. Selected MG160 ASRs were then tested for transversion, insertion, and deletion (peg RNA sequences SEQ ID NOs: 1848-1855) on the AAVS1 target using the same transfection protocol and NGS preparation and data analysis described above.

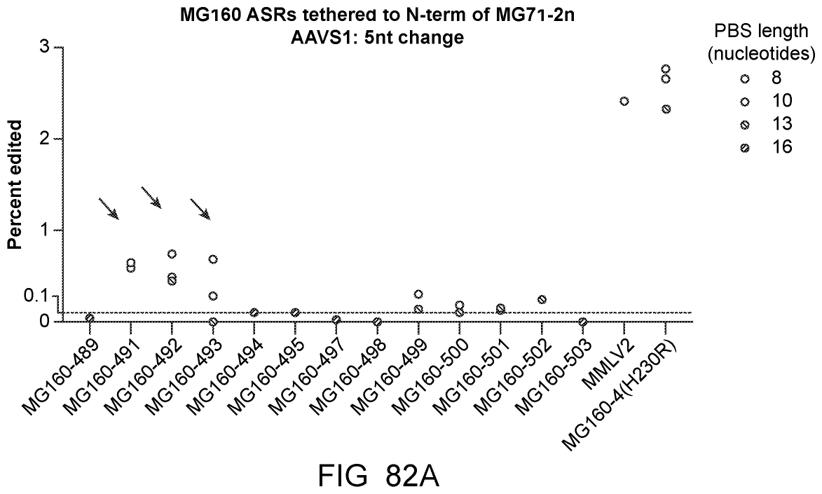

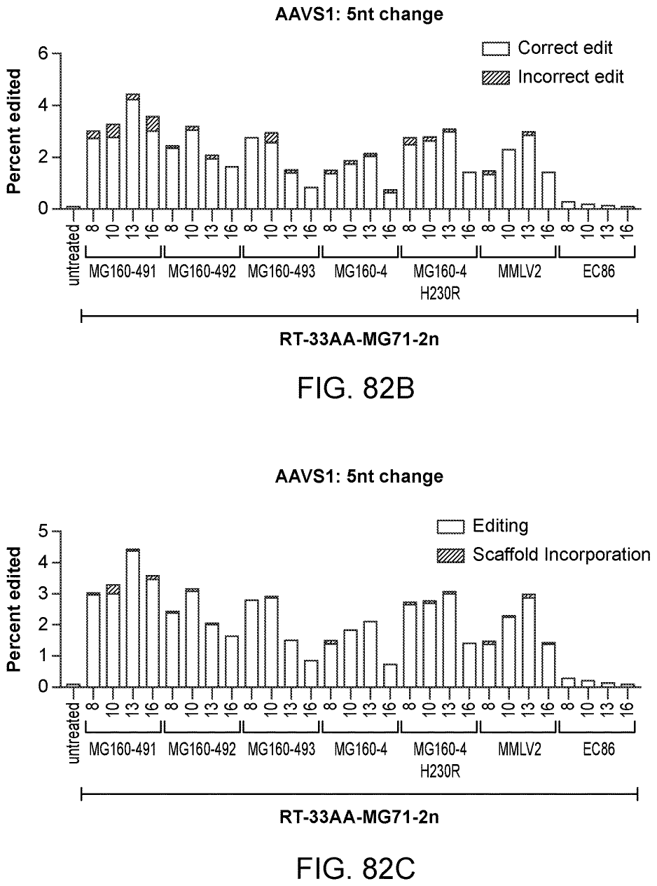

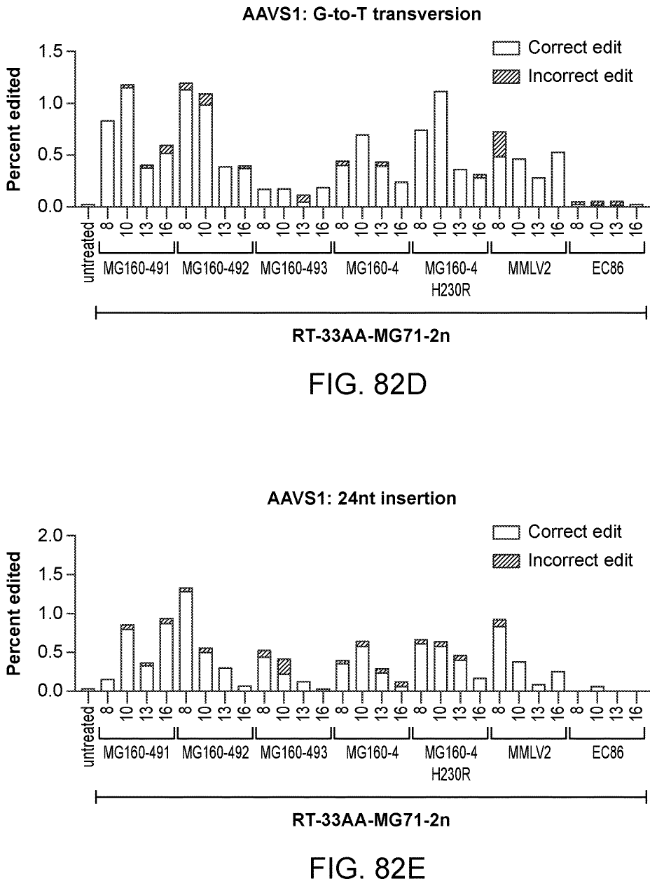

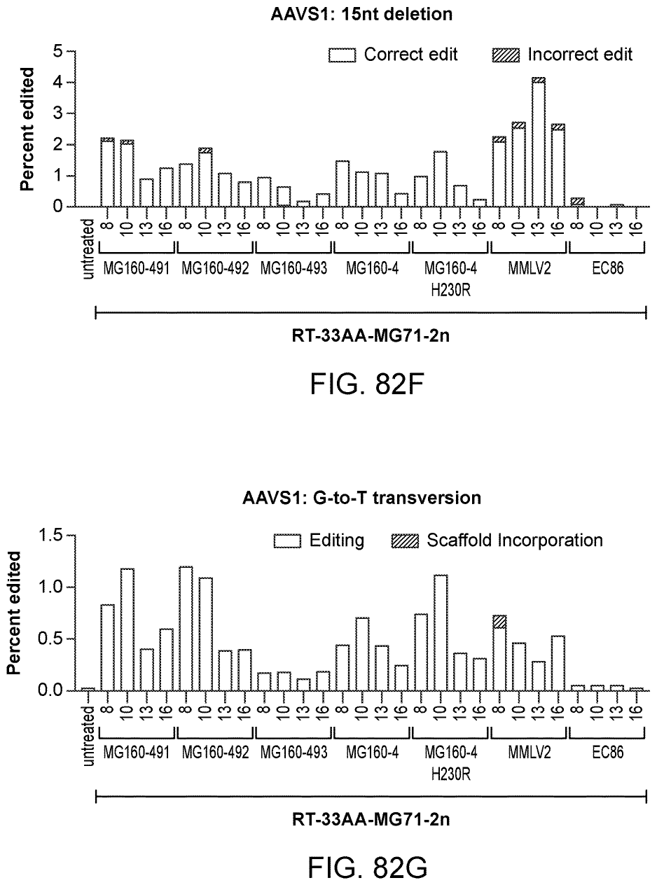

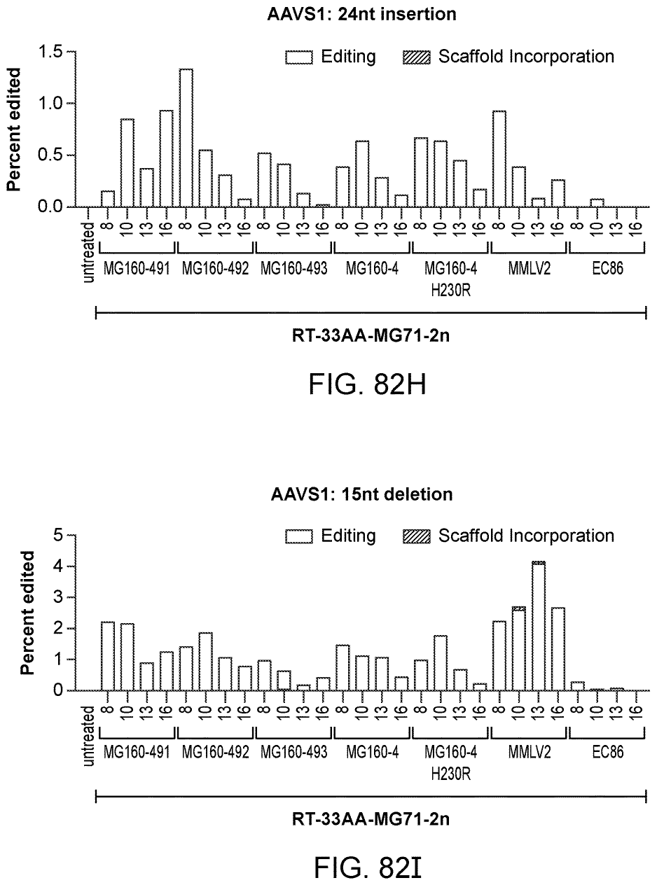

Out of the thirteen MG160 ASR candidates tested, four of the candidates (MG160-499, MG160-500, MG160-501, and MG160-502) were slightly active and three ASR candidates (MG160-491, MG160-492, and MG160-493) showed editing levels about 0.5% for a 5nt change on AAVS1 (FIG. 82A). MG160-491, MG160-492, and MG160-493 tethered to MG71-2n were then tested again for prime editing of a 5nt change on AAVS1 using pegRNAs with PBS 8, 10, 13, and 16nt (FIGS. 82B and 82C). These MG160 candidates were directly compared to MG160-4 wildtype, MG160-4(H230R), MMLV2, and EC86 (SEQ ID NO: 1847) all tethered to the N-terminus of MG71-2n. Wildtype MG160 ASRs were comparable to MG160-4(H230R) and MMLV2. In general, the highest levels of editing for a 5nt change were seen using a pegRNA of PBS 10nt or PBS 13nt and a drop off of editing was seen with a pegRNA of PBS 16nt (FIGS. 82B and 82C). This trend also holds true for MG160-4(H230R) and MMLV2. MG160-491, MG160-492, and MG160-493 were then challenged to perform a G-to-T transversion, a 24nt insertion, and a 15nt deletion (FIGS. 82D-82I). For a G-to-T transversion, MG160-491 and MG160-492 showed editing levels reaching about 1% editing, whereas MG160-493 did not reach more than 0.5% editing (FIGS. 82D and 82G). Editing levels for transversion were also comparable to MG160-4(H230R) and outperformed MMLV2 (FIGS. 82D and 82G). For a 24nt insertion, MG160-492 showed the highest levels of editing compared to all other candidates tested giving slightly over 1% editing with a pegRNA at PBS 8nt. Further, MG160-491 and MG160-492 showed comparable levels of editing as MG160-4(H230R) for a 15nt deletion reaching approximately 2% editing (FIGS. 82F and 82I). These MG160 ASR candidates did not perform better than MMLV2 for deletion but did show editing levels comparable to MMLV2 with transversion and insertion.

### Example 28. Short Corrections with the Addition of Nicking Guides to Improve Editing Efficiencies

Addition of guide targeting the opposite strand (also referred to as a nicking guide) has been employed in the PE3 prime editing system to improve the editing efficiency of pegRNAs (Anzalone et al. 2019). To test if this is an effective strategy, chemically-synthesized guides targeting the 125nt regions upstream and downstream of an AAVS1 site were designed (SEQ ID NOs: 1863-1910). These guides were evaluated across two different edits (a 5nt change (SEQ ID NO: 1685) and a single nucleotide G to T conversion (SEQ ID NOs: 1848-1851), two immortalized human cell lines (K562 and HEK293T) and three prime editing designs (MG160-4 H230R-MG71-2n, MMLV2-MG71-2n or MG151-98-DM-SL1-MG71-2n (SEQ ID NOs: 1592-1593 and 1654). Unless indicated otherwise, 5×10⁴ cells were nucleofected with IVT mRNA and either 150 pmol pegRNA alone (no nicking guide) or a combination of 150 pmol pegRNA and 50 pmol nicking guide. Cells were nucleofected using cell-type specific programs and recovered for three days at 37° C. gDNA was extracted, target regions were amplified, processed for NGS, and prime editing was analyzed.

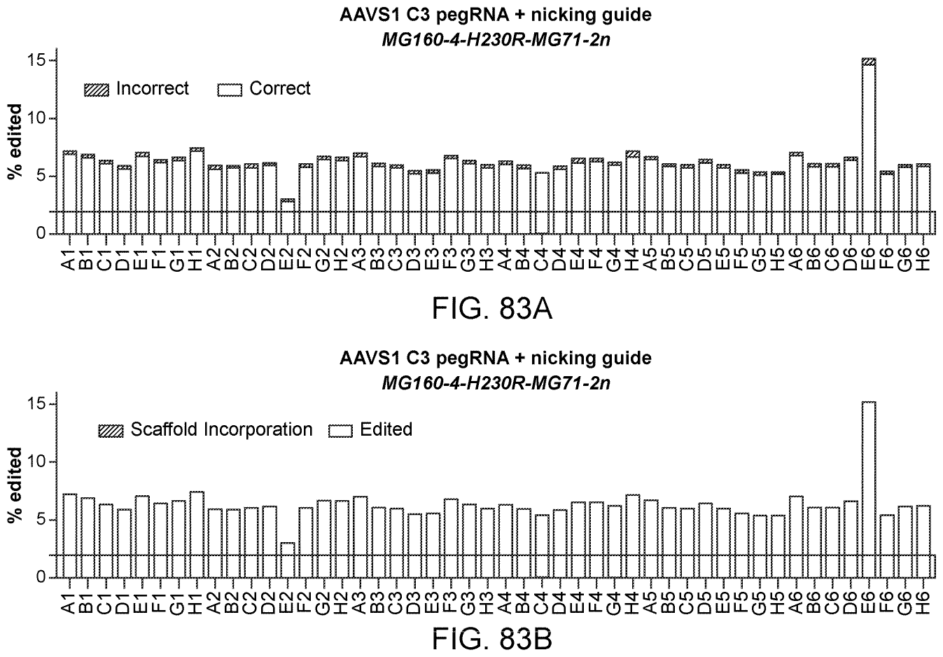

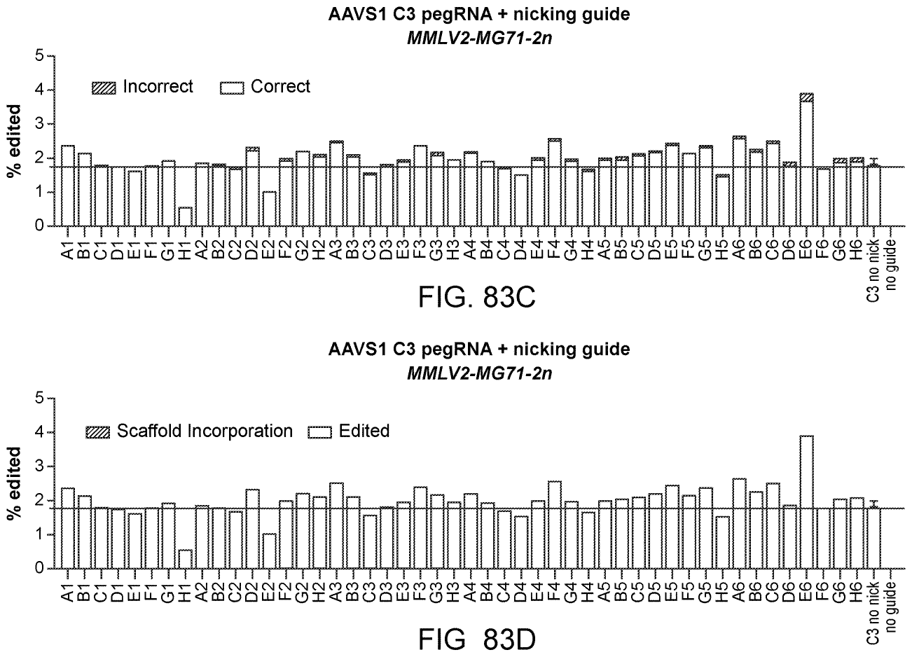

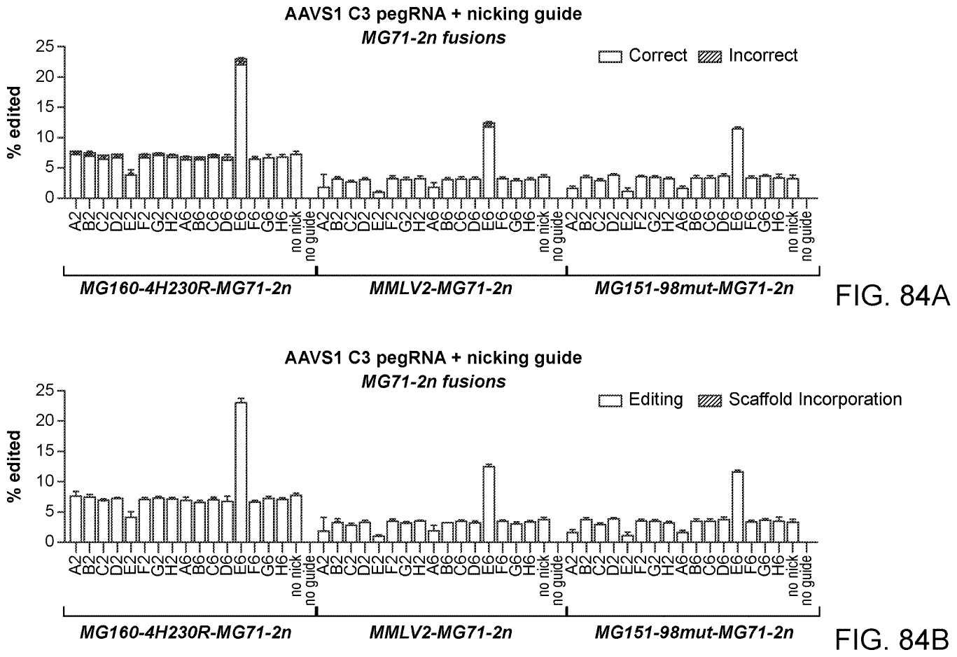

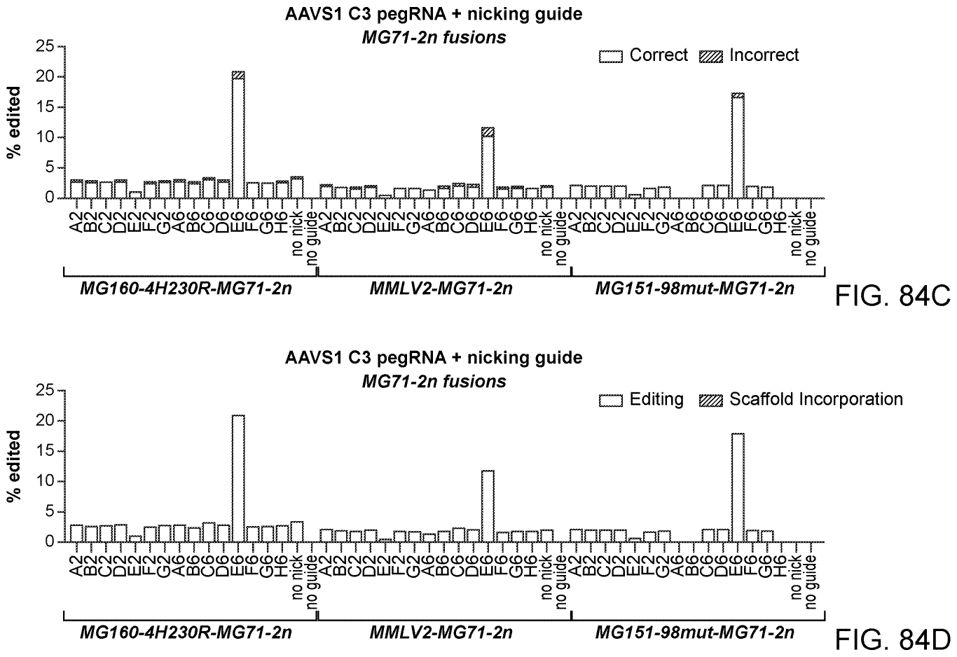

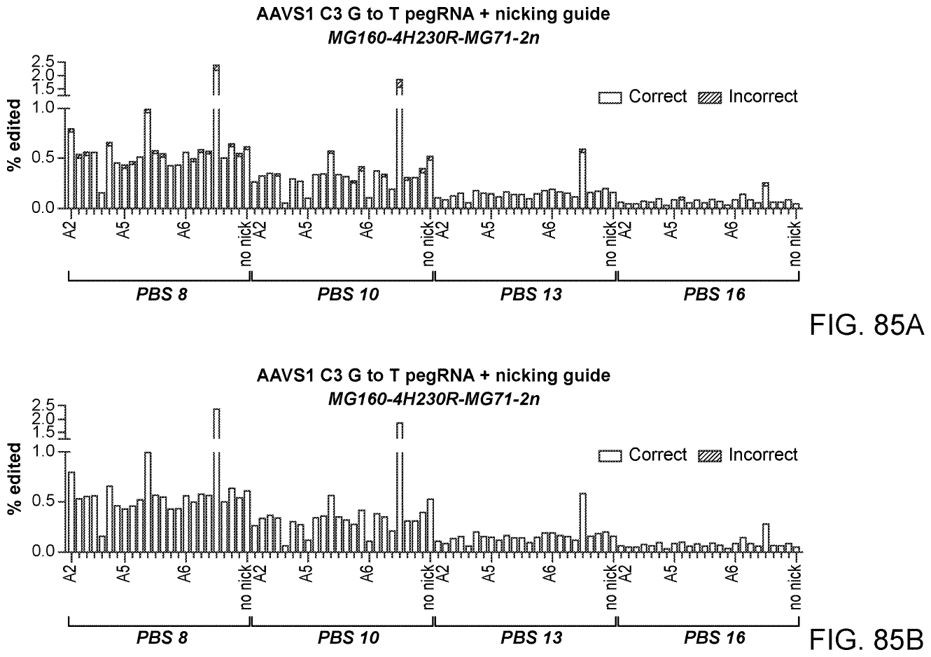

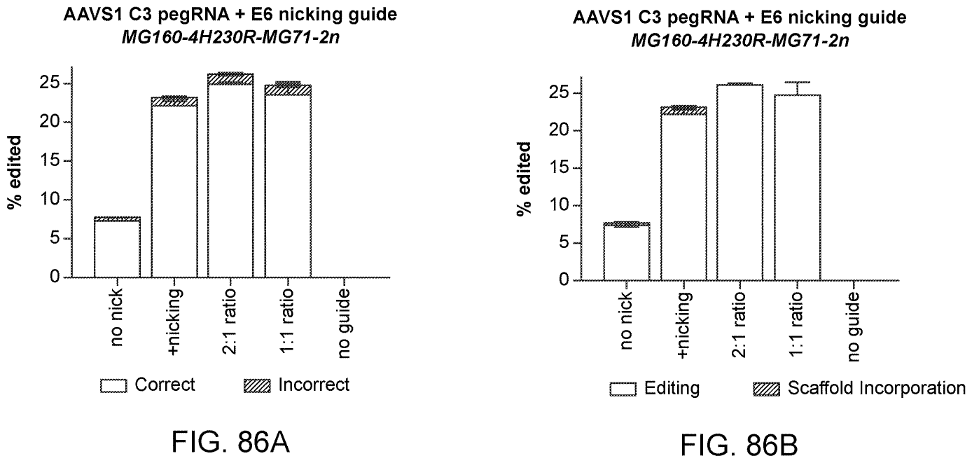

The effect of 48 nicking guides (SEQ ID NOs: 1863-1910) on prime editing efficiency with AAVS1 C3 5nt pegRNA (SEQ ID NO: 1685) in K562 cells are shown in FIG. 83. Of the 48 guides tested, guide E6 showed the highest consistent increase in editing efficiency with both MG160-4 H230R-MG71-2n (SEQ ID NO: 1592) and MMLV2-MG71-2n (SEQ ID NO: 1593). A subset of these nicking guides (SEQ ID NOs: 1871-1877 and 1903-1910) were validated with MG160-4 H230R-MG71-2n, MMLV2-MG71-2n, and MG151-98-DM-SL1-MG71-2n (SEQ ID NO: 1654). FIGS. 84A and 84B showed a similar pattern as what was observed in FIG. 83. To validate that this effect is not cell type specific, a subset of nicking guides (SEQ ID NOs: 1871-1877 and 1903-1910) were tested in HEK293T cells. FIGS. 84C and 84D shows that again guide E6 showed the highest improvement in prime editing efficiency across all constructs tested. To validate that nicking guides can be employed across multiple edits, a subset of the nicking guides (SEQ ID NOs: 1871-1877 and 1895-1910) were tested with a pegRNA encoding a G to T single nucleotide change with PBS lengths 8, 10, 13, and 16 (SEQ ID NOs: 1848-1851) FIG. 85. Guide E6 had the highest impact on editing activity. Lastly, to maximize editing activity, different ratios of pegRNA to nicking guide were tested with the best AAVS1 C3 5nt correction pegRNA and the E6 nicking guide FIG. 86. A 2:1 ratio of pegRNA:nicking guide had a marginal increase in editing. Altogether, these experiments demonstrate the addition of nicking guides as a viable strategy to increase prime editing efficiency up to 6-fold.

### REFERENCES

Anzalone A V, Gao X D, Podracky C J, Nelson A T, Koblan L W, Raguram A, Levy J M, Mercer J A M, Liu D R. Programmable deletion, replacement, integration and inversion of large DNA sequences with twin prime editing. Nat Biotechnol. 2022, 40(5):731-740. doi: 10.1038/s41587-021-01133-w. Epub 2021 Dec. 9. PMID: 34887556; PMCID: PMC9117393.
Clement K, Rees H, Canver M C, Gehrke J M, Farouni R, Hsu J Y, Cole M A, Liu D R, Joung J K, Bauer D E, Pinello L. CRISPResso2 provides accurate and rapid genome editing sequence analysis. Nat Biotechnol. 2019, 37(3):224-226. doi: 10.1038/s41587-019-0032-3. PMID: 30809026; PMCID: PMC6533916.
González-Delgado A, Mestre M R, Martinez-Abarca F, Toro N. Prokaryotic reverse transcriptases: from retroelements to specialized defense systems. FEMS Microbiol Rev. 2021, 45(6):fuab025. doi: 10.1093/femsre/fuab025. PMID: 33983378; PMCID: PMC8632793.
He S, Corneloup A, Guynet C, Lavatine L, Caumont-Sarcos A, Siguier P, Marty B, Dyda F, Chandler M, Ton Hoang B. The IS200/IS605 Family and “Peel and Paste” Single-strand Transposition Mechanism. Microbiol Spectr. 2015 August; 3(4). Doi: 10.1128/microbiolspec.MDNA3-0039-2014. PMID: 26350330.
Shimamoto, T., Hsu, M. Y., Inouye, S. and Inouye, M. Reverse transcriptases from bacterial retrons require specific secondary structures at the 5′-end of the template for the cdna priming reaction. J Biol. Chem. 1993, 268, 2684-2692.
Zhao B, Chen S-A A, Lee J, Fraser H B (2022) Bacterial retrons enable precise gene editing in human cells. CRISPR Journal 2022, 5(1), DOI: 10.1089/crispr.2021.0065
Wang, Y., Guan, Z., Wang, C. et al. Cryo-EM structures of Escherichia coli Ec86 retron complexes reveal architecture and defence mechanism. Nat Microbiol 7, 1480-1489 (2022). https://doi.org/10.1038/s41564-022-01197-7.
Kong X, Wang Z, Zhang R, Wang X, Zhou Y, Shi L, Yang H. Precise genome editing without exogenous donor DNA via retron editing system in human cells. Protein Cell. 2021 November; 12(11):899-902. doi: 10.1007/s13238-021-00862-7. Epub 2021 Aug. 17. PMID: 34403072; PMCID: PMC8563936.
Yarnall M T N, Ioannidi E I, Schmitt-Ulms C, Krajeski R N, Lim J, Villiger L, Zhou W, Jiang K, Garushyants S K, Roberts N, Zhang L, Vakulskas C A, Walker J A 2nd, Kadina A P, Zepeda A E, Holden K, Ma H, Xie J, Gao G, Foquet L, Bial G, Donnelly S K, Miyata Y, Radiloff D R, Henderson J M, Ujita A, Abudayyeh O O, Gootenberg J S. Drag-and-drop genome insertion of large sequences without double-strand DNA cleavage using CRISPR-directed integrases. Nat. Biotechnol. 2023 April; 41(4):500-512. doi: 10.1038/s41587-022-01527-4. Epub 2022 Nov. 24. PMID: 36424489; PMCID: PMC10257351.
Zheng C, Liu B, Dong X, Gaston N, Sontheimer E J, Xue W. Template-jumping prime editing enables large insertion and exon rewriting in vivo. Nat. Commun. 2023 Jun. 8; 14(1):3369. doi: 10.1038/s41467-023-39137-6. PMID: 37291100; PMCID: PMC10250319.
Anzalone, A. V., Randolph, P. B., Davis, J. R. et al. Search-and-replace genome editing without double-strand breaks or donor DNA. Nature 2019 576:149-157. https://doi.org/10.1038/s41586-019-1711-4

### EQUIVALENTS

The disclosure may be embodied in other specific forms without departing from the spirit or essential characteristics thereof. The foregoing embodiments are therefore to be considered in all respects illustrative rather than limiting on the disclosure described herein. Scope of the disclosure is thus indicated by the appended claims rather than by the foregoing description, and all changes that come within the meaning and range of equivalency of the claims are intended to be embraced therein.

1. A fusion protein comprising a nickase linked to a reverse transcriptase using a linker, wherein the reverse transcriptase comprises at least about 80% sequence identity to any one of SEQ ID NOs: 161-629, 767-1220, 1959-2522, and 2582-2585.

2. A fusion protein comprising a nuclease linked to a reverse transcriptase using a linker, wherein the reverse transcriptase comprises at least about 80% sequence identity to any one of SEQ ID NOs: 161-629, 767-1220, 1959-2522, and 2582-2585.

3. A fusion protein comprising a catalytically dead nuclease linked to a reverse transcriptase using a linker, wherein the reverse transcriptase comprises at least about 80% sequence identity to any one of SEQ ID NOs: 161-629, 767-1220, 1959-2522, and 2582-2585.

4. A gene editing system, comprising:
a) a nickase;
b) a guide nucleic acid configured to form a complex with the nickase and to hybridize to a target nucleic acid sequence; and
c) a reverse transcriptase having at least about 80% sequence identity to any one of SEQ ID NOs: 161-629, 767-1220, 1959-2522, and 2582-2585 and configured to form a complex with the nickase.

5. The gene editing system of claim 4, wherein the nickase is a modified endonuclease.

6. The gene editing system of claim 5, wherein the modified endonuclease is a Type II CRISPR endonuclease.

7. The gene editing system of claim 5, wherein the modified endonuclease is a Type V CRISPR endonuclease.

8. The gene editing system of any one of claims 6-7, wherein the Type II CRISPR endonuclease or the Type V CRISPR endonuclease has nickase activity.

9. The gene editing system of claim 5, wherein the modified endonuclease is selected from the group consisting of: spCas9 (H840A), spCas9 (D10A), nMG3-6 (D13A), nMG3-6 (H586A), nMG3-6 (N609A), Cas12a, and MG29-1.

10. The gene editing system of claim 5, wherein the modified endonuclease comprises at least about 80% sequence identity to any one of SEQ ID NOs: 152-154.

11. The gene editing system of any one of claims 4-10, wherein the nickase and the reverse transcriptase are fused.

12. The gene editing system of any one of claims 4-10, wherein the nickase and the reverse transcriptase are linked by a linker.

13. The gene editing system of claim 12, wherein the linker comprises at least 10, 20, or 30 amino acids.

14. The gene editing system of claim 12, wherein the linker comprises about 30-35 amino acids.

15. The gene editing system of claim 12, wherein the linker comprises about 30 amino acids.

16. The gene editing system of claim 12, wherein the linker comprises at least 80% sequence identity to SEQ ID NO: 103.

17. The gene editing system of claim 12, wherein the linker comprises at least 80% sequence identity to any one of SEQ ID NOs: 155-160.

18. The gene editing system of any one of claims 4-10, wherein the nickase and the reverse transcriptase are not linked.

19. The gene editing system of any one of claims 4-18, wherein the guide nucleic acid comprises a spacer sequence and a crRNA.

20. The gene editing system of any one of claims 4-19, wherein the guide nucleic acid further comprises a reverse transcriptase template (RTT).

21. The gene editing system of claim 20, wherein a base in the RTT comprises a bulky modification selected from the group of complex sugars, or complex amino groups, and/or other modifications compatible with RNA.

22. The gene editing system of any one of claims 4-21, wherein the guide nucleic acid further comprises a primer binding site.

23. The gene editing system of claim 22, wherein the primer binding site is on a 3′ end of the guide nucleic acid.

24. The gene editing system of any one of claims 22-23, wherein the primer binding site comprises at least 2, 4, 6, 8, 10, 13, 16, 20, 24, 28, 32, 36, 40, 45, 50, 55, 60, or 65 nucleotides.

25. The gene editing system of any one of claims 4-24, wherein the nuclease is non-covalently linked to the guide nucleic acid.

26. The gene editing system of any one of claims 4-24, wherein the nuclease is covalently linked to the guide nucleic acid.

27. The gene editing system of any one of claims 4-24, wherein the nuclease is fused to the guide nucleic acid.

28. The gene editing system of any one of claims 4-24, further comprising a transposase, integrase, or homing endonuclease.

29. The gene editing system of any one of claims 4-28, further comprising a retrotransposon.

30. The gene editing system of any one of claims 4-29, wherein the reverse transcriptase comprises a processivity of at least about 2-fold more than Moloney Murine Leukemia Virus (MMLV) reverse transcriptase.

31. The gene editing system of any one of claims 4-29, wherein the reverse transcriptase comprises a processivity of at least about 2-fold less than Moloney Murine Leukemia Virus (MMLV) reverse transcriptase.

32. The gene editing system of any one of claims 4-31, wherein the reverse transcriptase comprises an error rate of less than about 2.5%, 2.0%, 1.5%, 1%, 0.5%, 0.25%, 0.10%, or 0.05%.

33. The gene editing system of any one of claims 4-32, wherein the reverse transcriptase comprises an error rate of less than about 2.5%, 2.0%, 1.5%, 1%, 0.5%, 0.25%, 0.10%, or 0.05% as compared to Moloney Murine Leukemia Virus (MMLV) reverse transcriptase.

34. A gene editing system, comprising:
a) a nuclease;
b) a guide nucleic acid configured to form a complex with the nuclease and to hybridize to a target nucleic acid sequence; and
c) a reverse transcriptase having at least about 80% sequence identity to any one of SEQ ID NOs: 161-629, 767-1220, 1959-2522, and 2582-2585 and configured to form a complex with the nuclease.

35. The gene editing system of claim 34, wherein the nuclease is a double strand nuclease.

36. The gene editing system of any one of claims 34-35, wherein the nuclease is a Type II CRISPR endonuclease.

37. The gene editing system of claim 36, wherein the CRISPR endonuclease is Cas9.

38. The gene editing system of claim 37, wherein the Cas9 is catalytically dead Cas9 (dCas9).

39. The gene editing system of any one of claims 34-38, wherein the nuclease and the reverse transcriptase are fused.

40. The gene editing system of any one of claims 34-38, wherein the nuclease and the reverse transcriptase are linked by a linker.

41. The gene editing system of claim 40, wherein the linker comprises at least 10, 20, or 30 amino acids.

42. The gene editing system of claim 40, wherein the linker comprises about 30-35 amino acids.

43. The gene editing system of claim 40, wherein the linker comprises about 30 amino acids.

44. The gene editing system of claim 40, wherein the linker comprises at least 80% sequence identity to SEQ ID NO: 103.

45. The gene editing system of claim 40, wherein the linker comprises at least 80% sequence identity to any one of SEQ ID NOs: 155-160.

46. The gene editing system of any one of claims 34-38, wherein the nuclease and the reverse transcriptase are not linked.

47. The gene editing system of any one of claims 34-46, wherein the guide nucleic acid further comprises a primer binding site.

48. The gene editing system of claim 47, wherein the primer binding site is on a 3′ end of the guide nucleic acid.

49. The gene editing system of any one of claims 47-48, wherein the primer binding site comprises at least 2, 4, 6, 8, 10, 13, 16, 20, 24, 28, 32, 36, 40, 45, 50, 55, 60, or 65 nucleotides.

50. The gene editing system of any one of claims 34-49, wherein the nuclease is non-covalently linked to the guide nucleic acid.

51. The gene editing system of any one of claims 34-49, wherein the nuclease is covalently linked to the guide nucleic acid.

52. The gene editing system of any one of claims 34-49, wherein the nuclease is fused to the guide nucleic acid.

53. The gene editing system of any one of claims 34-52, further comprising a transposase, integrase, or homing endonuclease.

54. The gene editing system of any one of claims 34-53, further comprising a retrotransposon.

55. The gene editing system of any one of claims 34-54, wherein the reverse transcriptase comprises a processivity of at least about 2-fold more than Moloney Murine Leukemia Virus (MMLV) reverse transcriptase.

56. The gene editing system of any one of claims 34-54, wherein the reverse transcriptase comprises a processivity of at least about 2-fold less than Moloney Murine Leukemia Virus (MMLV) reverse transcriptase.

57. The gene editing system of any one of claims 34-56, wherein the reverse transcriptase comprises an error rate of less than about 2.5%, 2.0%, 1.5%, 1%, 0.5%, 0.25%, 0.10%, or 0.05%.

58. The gene editing system of any one of claims 34-56, wherein the reverse transcriptase comprises an error rate of less than about 2.5%, 2.0%, 1.5%, 1%, 0.5%, 0.25%, 0.10%, or 0.05% as compared to Moloney Murine Leukemia Virus (MMLV) reverse transcriptase.

59. A gene editing system, comprising:
a) a nickase;
b) a guide nucleic acid configured to form a complex with the nickase and to hybridize to a target nucleic acid sequence; and
c) a reverse transcriptase configured to form a complex with the nickase, the reverse transcriptase having a X₁X₂DD motif, wherein X₁ is F or Y, and wherein when X₁ is Y, X₂ is A, R, N, D, C, E, Q, G, H, I, L, K, M, F, P, S, T, V, W, or Y.

60. The gene editing system of claim 59, wherein the X₂ is A or I.

61. The gene editing system of claim 59, wherein the X₁X₂DD motif is YADD (SEQ ID NO: 2572) or YIDD (SEQ ID NO: 2573).

62. The gene editing system of claim 59, wherein the X₁X₂DD motif is FADD (SEQ ID NO: 2574), FVDD (SEQ ID NO: 2575), FIDD (SEQ ID NO: 2576), or FLDD (SEQ ID NO: 2577).

63. The gene editing system of any one of claims 59-62, wherein the reverse transcriptase has at least about 80% sequence identity to any one of SEQ ID NOs: 161-629, 767-1220, 1959-2522, and 2582-2585.

64. A gene editing system, comprising:
a) a nuclease;
b) a guide nucleic acid configured to form a complex with the nuclease and to hybridize to a target nucleic acid sequence; and
c) a reverse transcriptase configured to form a complex with the nuclease, the reverse transcriptase having a X₁X₂DD motif, wherein X₁ is F or Y, and wherein when X₁ is Y, X₂ is A, R, N, D, C, E, Q, G, H, I, L, K, M, F, P, S, T, V, W, or Y.

65. The gene editing system of claim 64, wherein the X₂ is A or I.

66. The gene editing system of claim 64, wherein the X₁X₂DD motif is YADD (SEQ ID NO: 2572) or YIDD (SEQ ID NO: 2573).

67. The gene editing system of claim 64, wherein the X₁X₂DD motif is FADD (SEQ ID NO: 2574), FVDD (SEQ ID NO: 2575), FIDD (SEQ ID NO: 2576), or FLDD (SEQ ID NO: 2577).

68. The gene editing system of any one of claims 64-67, wherein the reverse transcriptase has at least about 80% sequence identity to any one of SEQ ID NOs: 161-629, 767-1220, 1959-2522, and 2582-2585.

69. An isolated reverse transcriptase having at least about 80% sequence identity to any one of SEQ ID NOs: 161-629, 767-1220, 1959-2522, and 2582-2585.

70. A nucleic acid encoding for the fusion protein of any one of claims 1-3 or the gene editing system of any one of claims 4-68.

71. The nucleic acid of claim 70, wherein the nucleic acid is a DNA or an RNA.

72. The nucleic acid of claim 71, wherein the RNA is an mRNA.

73. A vector comprising the nucleic acid of any one of claims 70-72.

74. An adeno-associated virus or a lipid nanoparticle comprising the nucleic acid of any one of claims 70-72 or the vector of claim 73.

75. A cell comprising the nucleic acid of any one of claims 70-72 or the vector of claim 73.

76. The cell of claim 75, wherein the cell is a human cell.

77. The cell of claim 75, wherein the cell is a eukaryotic cell.

78. The cell of claim 75, wherein the cell is a mammalian cell.

79. The cell of claim 75, wherein the cell is an immortalized cell.

80. The cell of claim 75, wherein the cell is an insect cell.

81. The cell of claim 75, wherein the cell is a yeast cell.

82. The cell of claim 75, wherein the cell is a plant cell.

83. The cell of claim 75, wherein the cell is a fungal cell.

84. The cell of claim 75, wherein the cell is a prokaryotic cell.

85. The cell of claim 75, wherein the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, primary cell, or a derivative thereof.

86. The cell of claim 75, wherein the cell is an engineered cell.

87. The cell of claim 75, wherein the cell is a stable cell.

88. A method for modifying a double- and/or single-stranded nucleic acid, comprising contacting a cell using the fusion protein of any one of claims 1-3 or the gene editing system of any one of claims 4-68.

89. A method for modifying a double- and/or single-stranded nucleic acid, comprising:
a) providing a cell with a guide nucleic acid to bind to a target strand of the nucleic acid;
b) providing the cell with a nuclease or nickase to cleave the nucleic acid at a location of binding of the guide nucleic acid;
c) providing the cell with a reverse transcriptase to synthesize a modification in the target strand of the nucleic acid at a location of cleavage by the nickase and/or nuclease.

90. The method of claim 89, wherein the reverse transcriptase has at least about 80% sequence identity to any one of SEQ ID NOs: 161-629, 767-1220, 1959-2522, and 2582-2585.

91. The method of claim 89, wherein the modification is an insertion, deletion, or mutation.

92. The method of claim 89, further comprising providing an RNA or DNA template.

93. The method of claim 89, wherein the nucleic acid is a genome or a vector.

94. The method of claim 89, further comprising providing the cell with a transposase, integrase, or homing endonuclease.

95. The method of claim 89, further comprising providing the cell with a retrotransposon.
